(19) United States
(12) Patent Application Publication
(10) Pub. No.: US 2014/0121123 A1
(43) Pub. Date: May 1, 2014

(54) METHODS FOR DIVERSIFYING ANTIBODIES, ANTIBODIES DERIVED THEREFROM AND USES THEREOF

(71) Applicants: Kevin Caili Wang, Palo Alto, CA (US); Yan Li, San Jose, CA (US); Mark Hsieh, San Mateo, CA (US); Xinwei Wang, Germantown, MD (US); Xinwei Wang, Germantown, MD (US); Feng Dong, Lansdale, PA (US); Peizhi (Peter) Luo, Lansdale, PA (US); Pingyu Zhong, Blue Bell, PA (US)

(72) Inventors: Kevin Caili Wang, Palo Alto, CA (US); Yan Li, San Jose, CA (US); Mark Hsieh, San Mateo, CA (US); Xinwei Wang, Germantown, MD (US); Xinwei Wang, Germantown, MD (US); Feng Dong, Lansdale, PA (US); Peizhi (Peter) Luo, Lansdale, PA (US); Pingyu Zhong, Blue Bell, PA (US)

(21) Appl. No.: 13/882,285

(22) PCT Filed: Oct. 24, 2011
(86) PCT No.: PCT/US11/57426

§ 371 Date: Oct. 15, 2013

Related U.S. Application Data
(60) Provisional application No. 61/408,393, filed on Oct. 29, 2010.

(51) Int. Cl. C12N 15/10

(52) U.S. Cl. CPC C12N 15/1055
USPC 506/9; 435/252.33; 435/320.1; 506/18; 506/23; 530/387.1; 536/23.53

(57) ABSTRACT

The invention provides methods of introducing diversity into antibody molecules comprising introducing or substituting at least one amino acid sequence in the CDR of the target antibody together with at least one amino acid in the FW region spanning the 3 amino acid adjoining the CRD on each side. The resulting diverse antibodies with variant CDRs and FW region sequences comprising diverse amino acid sequences are also described. These polypeptides regions, herein referred to as 3+CDR3+, that form the gist of the invention contribution described herein provide a flexible and simple source of sequence diversity that can be used as a source for expressing and identifying diverse antibodies or antigen binding polypeptides. Libraries comprising a plurality of these polypeptides are also provided. In addition, methods of and compositions for generating and using these polypeptides and libraries are provided. A method of producing diverse antibodies comprising amino acid substitutions in one of the CDR region and the FW region comprising 3 contiguous FW sequence amino acids adjacent to each CDR on either side is described herein. The substitutions are relative to database or germline sequences. Not all substitution are preserved in conservative regions.

The present application claims the benefit of U.S. provisional patent application No. 61/408,393, filed Oct. 29, 2010, which is herein incorporated by reference in its entirety.

### FIELD OF THE INVENTION

The invention generally relates to methods of increasing the overall diversity of a target antibody including libraries of diversified antibodies or antibody variable domains. The libraries include a plurality of different antibody variable domains generated by creating diversity in a heavy or light chain variable framework domain—the so called junctional region optionally with a mutation in one or more complementary determining regions (CDRs). The junctional region essentially comprises the three amino acids resident in the framework sequence juxtaposed to each CDR on either side. In particular, diversity in the junctional region together with the CDR region is designed to maximize antigen binding while minimizing the structural perturbations of the antibody variable domain. The invention also relates to fusion polypeptides of one or more antibody variable domain and a heterologous protein such as a coat protein of a virus. The invention also relates to replicable expression vectors which include a gene encoding the fusion polypeptide, host cells containing the expression vectors, a virus which displays the fusion polypeptide on the surface of the virus, libraries of the virus displaying a plurality of different fusion polypeptides on the surface of the virus and methods of using those compositions. The methods and compositions of the invention are useful for identifying novel antibodies and antibody variable domains that can be used therapeutically or as reagents.

### BACKGROUND OF THE INVENTION

Maintaining an arsenal against the unexpected requires diversity. The humoral immune response recognizes novel molecular surfaces by exposure to a vast repertoire of potential binding partners. When confronted with a novel antigen, the chance that any given antibody in the pool will bind is low. As such, it is primarily the diversity of the antibody repertoire that determines whether a specific complementary paratope will be recovered. Vertebrate organisms appear to be capable of synthesizing thousands of different antibody sequences, each presumably encoded by a different antibody gene. Vertebrates have achieved antibody diversity by several strategies. One is the maintenance of a large repertoire of germline V (variable) genes. The others are somatic processes that shuffle germline V genes and possibly introduce denovo sequence diversity. Gene conversion has also been suggested as a process in the creation and maintenance of antibody diversity. “Conversion and Antibody diversity—Annu. Rev. Biochem./989. 58:509-3/.

The vertebrate immune system can evolve antibodies capable of recognizing essentially any macromolecule with high affinity and specificity. Analyses of natural antibody sequences together with structural studies have been instrumental in revealing how antibodies work (Chothia et al., 1992, J. Mol. Biol., 227: 799-817; Kabat, 1982, Pharmacological Rev., 34: 23-38; Kabat, 1987, Sequences of Proteins of Immunological Interest (National Institutes of Health, Bethesda, Md.)). These studies have revealed that antigen recognition is primarily mediated by complementarity determining regions (CDRs) that are located at one end of the antibody variable domain and are connected by a .beta.-sheet framework (Wu & Kabat, 1970, J. Exp. Med., 132: 211-250; Kabat & Wu, 1971, Annals New York Acad. Sci., 190: 382-393). The sequence diversity of natural antibodies shows that the CDRs are hypervariable in comparison with the framework, and it is the CDR sequences that determine the antigen specificity of a particular antibody (Jones et al., 1986, Nature, 321: 522-5; Amit et al., 1986, Science, 233: 747-53). These studies have also revealed that the natural sequence diversity at most CDR positions is not completely random, as biases for particular amino acids occur in both a site-specific manner and in terms of overall CDR composition (Davies & Cohen, 1996, Proc. Natl. Acad. Sci. USA, 93: 7-12; Kabat et al., 1977, J. Biol. Chem., 252: 6609-16; Zemlin et al., 2003, J. Mol. Biol., 334: 733-49; Mian et al., 1991, J. Mol. Biol., 217: 133-51; Padlan, 1994, Mol. Immunol., 31: 169-217).

Studying the incredible capability of the mammalian adaptive immune system to generate and manipulate billions of different binding specificities (antibodies and T cell receptors) has been a fulcrum of modern immunology. The diversity of antibody variable domains is generated by several distinct genetic mechanisms (Zemlin et al., 2003; Bassing et al., 2002, Cell, 109: S45-55; Tonegawa, 1983, Nature, 302: 575-81; Padlan, 1997, Mol. Immunol., 34: 765-70; Wilson, 2000, J. Exp. Med., 191: 1881-94). Diversity is encoded in the collection of V genes in the germline and further generated through recombination of V, D and J segments in B cells. Recombination generates variability in amino acid content in all CDRs and variation in the length of the loop portion of CDR3. Finally, somatic hypermutation during the secondary immune response generates diversity throughout variable domains. When antibodies are generated by the immune system, sequence diversity of the variable domains is initially created by combinatorial shuffling of germline-encoded gene segments. After exposure to an antigen, lymphocytes with antigen recognition are selected and further somatic mutations in the V segments are generated, providing the basis for clonal selection for mutations that enhance the affinity of the antibody-antigen interactions. Antibody paratopes, the agents of humoral molecular recognition, mediate specific binding through a protein-antigen interface that varies dramatically between molecules. Antibody paratopes are found at the hypervariable region of a light and heavy chain heterodimer. The antigen-binding site comprises six complementarity determining regions (CDR); the hypervariable regions of the variable light chain (VL) and variable heavy chain (VH) each contribute three CDRs to the complete binding pocket (Kortt et al., 2001). Each chain contributes 3 loops to a spatial cluster of complementarity determining regions (CDRs). CDRs 1 and 2 are encoded in germline V-segment loci: 51 V_{H} and 70 V_{κ/λ} loci, each with unique amino acid encodings, exist in a typical human haplotype. Diversity in each chain is determined by combinatorial V_{H}-(D_{H})-J_{H} (for the heavy) or V_{κ/λ}-J_{κ/λ} (for the light) rearrangements, P and N-addition, junctional flexibility, and somatic hypermutation of variable domain nucleotides, with a concentration on CDR encoding regions. This series of events leads to the generation of the initial, naïve immune repertoire. The naïve repertoire is modified during the ‘educational stage’ of development of the adaptive immune system, and following antigenic stimulation by somatic mutations, receptor editing and further gene rearrangements result in mutated antibody genes that comprises an immune repertoire. The combinatorial association of such stochastically generated light and heavy chains has the potential to generate many orders of magnitude more diversity than can be uniquely displayed on the 10” B-cells in a single individual's lymphocyte population. With each antibody variable fragment (Fv) encoded by at least 650 base pairs, the presented repertoire is potentially 4 orders of magnitude larger than the entire human diploid genome (6.4×10⁹ bp). While these genetic mechanisms allow sufficient diversity to generate a biologically competent immune repertoire, they also likely constrain natural diversity as compared to the level of diversity created through a purely random process.

Antibodies (eg, IgG, which is the most commonly used immunoglobulin form) are unique proteins with dual functionality. All naturally occurring antibodies are multivalent, with IgG having two binding ‘arms.’ Antigen-binding specificity is encoded by three complementarity-determining regions (CDRs), while the Fc-region is responsible for binding to serum proteins (eg, complement) or cells. An antibody itself usually is not responsible for killing target cells, but instead marks the cells that other components or effector cells of the body's immune system should attack, or it can initiate signaling mechanisms in the targeted cell that leads to the cell's self-destruction. The former two attack mechanisms are referred to as antibody-dependent complement-mediated cytotoxicity (CMC) and antibody-dependent cellular cytotoxicity (ADCC). ADCC involves the recognition of the antibody by immune cells that engage the antibody-marked cells and either through their direct action, or through the recruitment of other cell types, lead to the tagged-cell's death. CMC is a process where a cascade of different complement proteins become activated, usually when several IgGs are in close proximity to each other, either with one direct outcome being cell lysis, or one indirect outcome being attracting other immune cells to this location for effector cell function. Antibodies, when bound to key substances found on the cell surface, also can induce cells to undergo programmed cell death, or apoptosis.

The ability of mAbs to bind with both high affinity and high specificity to molecular targets that are prevalent in a diseased condition, but not in normal cells, has been the foundation for choosing mAb therapeutics when conventional small molecule approaches have failed. The development of antibody therapeutics is a dynamic discipline that only seems to gain momentum as the technology matures. Indeed, the achievements of the past decade in antibody engineering rejuvenated the entire sector of drug development of biological compounds (Reichert et al., 2005). Two decades after the generation of the first mouse monoclonal antibodies (mAbs) by Köhler and Milstein, the first therapeutic antibody for a cancer indication was approved by the FDA for marketing in 1997. Technology improvements since then have provided several approaches to manufacturing human and humanized antibodies with high affinity for biologically relevant targets. Recombinant antibody fragments include the monovalent antibody fragments Fab, scFv, disulfide-stabilized Fv, single-domain VH, VhH, domain antibody and the multimeric formats, such as minibodies, bis-scFv, diabodies, triabodies and tetrabodies. Most of these formats have been used in antibody libraries. Minimal binding proteins, such as Fab, scFv, and single variable domains are the preferred targeting elements for some investigational drugs, with the scFv being by far the most common format used in recombinant antibody libraries, with Fab in second place. However, antibodies are large macromolecules that pose numerous challenges. While further improvements in discovery technologies, such as phage display, ribosome display, and transgenic animals continue to advance our capacity to rapidly screen and refine optimal binding molecules, antibody engineers have been focusing more of their efforts on improving protein production and stability, as well as engineering improved biological properties in the effector domains of monoclonal antibodies. Another challenge for mAb therapeutics that has not been realized is attempts at producing more diverse novel and known antibodies. Most strategies for anti-cancer therapies employ full length mAbs whose function supersedes target binding. Known mechanisms of action of marketed mAbs and other well characterized antibodies include: (1) antagonist action via blocking a cell receptor interaction with its ligand, (2) Fc domain based recruitment of immune cells to the targeted tissue and subsequent antibody-dependent cellular cytotoxicity (ADCC) or complement-dependent cytotoxicity (CDC), (3) receptor-mediated apoptosis, (4) receptor down regulation, and (5) delivery of radioisotopes or chemotherapeutic drugs in the context of mAb bioconjugates.

While traditional hybridoma technology, which relies on immunization of animals, is still the dominant source of antibodies, powerful combinatorial technologies have allowed for the development of in vitro immune repertoires and selection methodologies that can be used to derive antibodies without the need for direct immunization of a living host. These technologies are an attractive and expanding alternative to hybridoma technology, particularly for drug discovery and development, because they allow for the production of human proteins that are likely to show low immunogenicity. For the generation of diversity, antibody library technology have been designed to mimic the natural process and, if possible, surpass it by introducing sequence diversity that is alien to natural antibodies. Indeed, the discovery of antibodies for therapeutic, diagnostic, analytical or affinity chromatograph purposes and the process of generating antibodies with exquisitely tailored characteristics relies increasingly on the use of large combinatorial libraries encoding repertoires of antibody variants. Three distinct types of antibody libraries have been generated: (i) libraries that sample the antibody diversity available in immunized animals, (ii) libraries that mimic the diversity of the antibodies in non-immunized individuals and (iii) synthetic libraries. In the latter, control over both library design and selection conditions enables unprecedented precision in antibody engineering. Synthetic libraries have been used to gain insights into the mechanisms of antibody structure and function, to tackle particularly difficult therapeutic challenges and to expand the utility of antibodies to novel areas of research. As well, synthetic antibody libraries, in which the antigen-binding sites are entirely man-made, have come of age and now rival or even exceed the potential of natural immune repertoires. Using the latter approach, antibodies that recognize and bind to a target with the requisite affinity and specificity are isolated from a library by high throughput screening techniques such as phage, bacterial, yeast or ribosome display. Indeed, the sequence of one or more antibody scaffolds is diversified so that it encodes all or a subset of the 20 natural amino acids at multiple positions known to be critical for antigen recognition. The theoretical diversity of synthetic antibody libraries in which multiple hypervariable loops are randomized far exceeds practical limitations in library construction and screening.

The methods used to generate diversity are fundamentally different for natural and synthetic antibody libraries. In the case of natural repertoires, molecular biology techniques are used to amplify and clone pools of light- and heavy-chain variable domains directly from immune tissues. A major advantage of this approach is that it only requires knowledge of the antibody gene sequences, which are now readily available for many species, and most of the displayed antibodies are likely to be correctly folded, as they have been derived from lymphocytes producing functional antibodies. However, sequence diversity is limited to the scope of the natural immune system, and also, it has been found that natural antibodies vary tremendously in terms of expression and stability. Furthermore, although natural-repertoire libraries are a rich source of specific antibodies, affinity maturation or stability improvement of selected antibodies is often difficult, because of the undefined nature of the library. The generation of library diversity is in fact a two-tier process. The first level is during library construction. The second level involves modifying already isolated antibodies, usually with the purpose of increasing their affinity, which is also known as ‘in vitro affinity maturation’. Over the years, many approaches for introduction of diversity into pre-existing antibodies have been applied and can be divided into ‘targeted’ versus ‘non-targeted’ approaches. An excellent recent review of the latest technologies for the enhancement of antibody affinity appears in Wark et al., Adv. Drug. Deliv. Rev.; 58:657-670 (2006)

Targeted approaches introduce diversity into positions that are supposed to contribute to antigen binding. Examples include: CDR walking, as described above; CDR randomization, which is an extension of the former; hotspot mutagenesis, which introduces randomness in positions that frequently undergo mutagenesis during the natural process of affinity maturation]; and codon-based mutagenesis, which targets individual CDR positions based on structural information. See, for example, Itai Benhar, Expert Opin. Biol. Ther., 7>763-779 (2007) As noted in Benhar, supra, the extent to which the original antibody is diversified is usually dictated by the display platform as well as the ambition and foresight of the investigators.

Non-targeted approaches introduce sequence diversity at random throughout the antibody genes of the antibody. As a result, these appear to be much less ‘accurate’ and the process takes very large repertoires to obtain affinity-matured antibodies. Examples of non-targeted approaches for in vitro affinity maturation include error-prone PCR mutagenesis (introducing random mutations using a low-fidelity DNA polymerase during PCR amplification), antibody chain shuffling, DNA shuffling (sexual PCR, which is a process involving digesting mutant libraries of closely related DNA sequences with deoxyribonuclease I and reassembling them into shuffled intact fragments by primer-less PCR) and mutator E. coli strains (such as MutD5-FIT or XL-1 Red, which carry mutD. In addition to affinity maturation, secondary libraries have been built from existing antibodies to improve the properties that can be summed up in ‘robustness’. These include expression level, solubility and stability.

As noted supra, a particularly promising branch of in vitro antibody research is concerned with the development of ‘synthetic’ antibody libraries with antigen-binding sites constructed entirely from man-made diversity. Even within the in vitro antibody field, this represents a highly specialized area of research, as most libraries are derived by transferring natural antibody genes to in vitro display formats. This can be readily accomplished by using PCR to amplify functional antibody genes from immune tissues, and thus, library construction from natural repertoires is relatively facile, as it requires only knowledge of the genetic sequences encoding antibody diversity. In contrast, synthetic repertoires are not derived from natural antibody genes, but rather are built from scratch by introducing diversity with precisely designed synthetic DNA. The design of synthetic repertoires requires detailed structural and functional knowledge of the antibody molecule, and consequently the development of effective libraries of this type may prove to be considerably more difficult than the development of libraries from natural diversity. In spite of these shortcomings, research in the synthetic antibody field has been spurred by the numerous advantages that come with a high-precision approach. Over the last few years, these efforts have been rewarded with technical advances that have allowed the development of highly functional synthetic antibody libraries that rival or even exceed the recognition potential of natural immune systems. Effective in vitro libraries have been constructed using either the entire antigen-binding fragment (Fab), the single-chain variable fragment (scFv) consisting of the light- and heavy-chain variable domains (VL and VH), or the VH domain alone, which represents the smallest domain capable of mediating antigen recognition.

All the selection platforms have three features in common: phenotype (displayed antibody with antigen binding capability) and genotype are linked and co-selected; a selective pressure is applied to enrich antibodies having the desired properties from the entire population; and they need amplification of the enriched population following selection. The three major types of selection platforms for antibody libraries are phage display, cell display and ribosome display. Antibody arrays are starting to appear as appealing tools for high-throughput screening of antibody libraries for functional genomics. All in vitro display technologies depend on the establishment of a linkage between the displayed protein (phenotype) and the encoding DNA (genotype). Pools containing billions of unique antibodies can be subjected to selective pressure for antigen recognition, selected clones can be amplified and the displayed protein sequences can be decoded by sequencing of the linked DNA. Phage display libraries have been used to generate human antibodies from immunized and non-immunized humans, germ line sequences, or naive B cell Ig repertories (Barbas & Burton, Trends Biotech (1996), 14:230; Griffiths et al., EMBO J. (1994), 13:3245; Vaughan et al., Nat. Biotech. (1996), 14:309; Winter EP 0368 684 131).

Phage display libraries allow an antibody repertoire to be queried with a candidate antigen directly, without the need to proceed through in vivo immunization. A number of strategies for introducing repertoire diversity during library construction have been proposed Phage display is a powerful technique that has been utilized to identify novel antigen binding antibody variable domains. The ability to identify and isolate high affinity antibodies from a phage display library is important in isolating novel human antibodies for therapeutic use. Isolation of high affinity antibodies from a library is dependent on the size of the library, the efficiency of production in cells, and the diversity of the library. See, for e.g., Knappik et al., J. Mol. Biol. (1999), 296:57. The size of the library is decreased by inefficiency of production due to improper folding of the antibody or antigen binding protein and the presence of stop codons. Expression in bacterial cells can be inhibited if the antibody or antigen binding domain is not properly folded. In some cases, expression can be improved by mutating residues in turns at the surface of the variable/constant interface, or at selected CDR residues. (Deng et al., J. Biol. Chem. (1994), 269; 9533, Ulrich et al., PNAS (1995), 92:11907-11911; Forsberg et al., J. Biol. Chem. (1997), 272:12430). The sequence of the framework region is important in providing for proper folding when antibody phage libraries are produced in bacterial cells. However, not all strategies provide a library of diverse but structurally stable antibody variable domains.

Over the years, several alternative display platforms have been developed, and these include systems for display on yeast or bacterial cells, on retroviruses, or through linkage to mRNA or DNA. Naive, or nonimmune, antigen binding libraries have been generated using a variety of lymphoidal tissues. Some of these libraries are commercially available, such as those developed by Cambridge Antibody Technology and Morphosys (Vaughan et al., Nature Biotech 14:309 (1996); Knappik et al., J. Mol. Biol. 296:57 (1999)). However, many of these libraries have limited diversity.

As noted, supra, the ability to isolate binding specificities against a broad variety of antigenic determinants represents one of the most important pre-requisites for a library for most practical applications. Other desirable features include the ability to rapidly affinity mature antibody clones, if required for special applications, as well as the performance of individual antibody clones (expression yields, stability, solubility, oligomeric format, etc.). The size of antibody libraries (i.e., the number of antibody clones) is an important factor contributing to the successful isolation of good quality binders against a variety of different antigens. Furthermore, library design greatly contributes to the performance of the antibody selection process. For example, in phage display there is a limit to the number of phage that can be used in practical selection experiments, and the strategy chosen for library design and construction directly influences the percentage of functional clones in the library and the fraction of antibodies that are displayed on the phage as fusion to the minor coat protein pill. Modular libraries, e.g. synthetic libraries based on conserved antibody scaffolds, provide yet another alternative display mechanism and may facilitate the isolation of high affinity antibodies. However, the fraction of a library that is actually displayed as correctly folded antibodies is more difficult to assess and depends critically on the design principles employed for library construction.

A number of possible combinatorial antibody libraries can be considered, for example those derived from antibody genes amplified from peripheral blood lymphocytes with suitable oligonucleotide primers. By contrast, synthetic antibody libraries are constructed in the laboratory using carefully designed antibody gene segments. Combinatorial mutagenesis of judiciously chosen amino acid positions can be used to construct such synthetic libraries. The advantages and disadvantages of synthetic libraries are essentially opposite those of natural-repertoire libraries. With synthetic libraries, highly stable and well-expressed frameworks can be used to fortify the overall stability of library members, and design features can be incorporated to allow facile affinity maturation. For example, synthetic antibody libraries are particularly useful for practical pharmaceutical applications. First, the antibody genes in these libraries have never undergone negative selection in vivo against antigens present in an organism. Secondly, the design of the library allows a careful control of the genetic structural elements, which can be introduced in the library. Third, library construction can use certain antibody genes that confer certain beneficial properties such as excellent stability, expression, low immunogenicity, tolerance to amino acids substitution, and performance in selections. Last but not least, certain antibody genes are characterized by the fact that the corresponding protein is capable of binding to protein A, thus facilitating the purification of corresponding antibody fragments by affinity chromatography. The primary disadvantage of synthetic libraries resides in the fact that the introduction of synthetic diversity can lead to protein misfolding and aggregation, if inappropriate chemical diversity or structural sites are used in the library design. Synthetic antibody libraries are created by introducing degenerate, synthetic DNA into the regions encoding the complementarity-determining regions (CDRs) of defined variable-domain frameworks. The design of an antibody library begins with the generation of diversity, where a choice is made about the source of antibody-coding genes. The antibody genes, with or without added non-natural diversity, are than assembled into an intact expression format, most frequently a scFv or Fab. In the next step, the recombinant repertoire is mounted on a display platform for panning, such as phage or ribosome display, or a platform for screening, such as bacterial and yeast display. By applying a selective pressure, usually for binding affinity and specificity, individual antibodies are isolated from such primary libraries. Indeed, by varying the selection platform, different antibodies are isolated from identical gene repertoires. These are usually cloned into a more appropriate expression system in a format suitable for the downstream application. Antibody library technology is also used to reintroduce sequence diversity into existing antibodies that may be themselves library-derived or come from other sources, such as hybridomas, with the intent of optimizing them. The selection strategies are similar to those applied with primary libraries, but with secondary libraries it is more frequent to see selective pressure applied for robustness (stability, solubility, thermo tolerance, etc.). Antibody libraries have provided for a large part of the existing pipeline of potential antibody-based therapeutics, as well as diagnostics, and will no doubt continue to make a large contribution to antibody discovery and development in the future. All of these are physical selection methods and require significant quantities of the selector to perform selection and screening. In addition, systems have also been developed for in vivo selections inside bacteria or yeast. Each of these systems has advantages and disadvantages that make them more or less suitable for particular applications, and the relative merits of the different technologies have been described elsewhere.

Several groups have constructed synthetic antibody libraries that are capable of generating high-affinity antibodies against a wide variety of antigens. First, the diversities of the newer libraries greatly exceed those of earlier examples, and it has been shown that naive repertoires that contain greater than a billion unique members are sufficient, and likely necessary, for generating antibodies with affinities in the nanomolar range. A second improvement has been the expansion of synthetic diversity beyond CDR-H3 (the major binding determinant in most antibodies) to additional CDR loops in both the heavy and light chains. Additional improvements have also been made by using advanced mutagenesis methods that allow for the introduction of highly precise, tailored diversity designed to mimic the types of amino acids that are abundant in natural antigen-binding sites. Indeed, some researchers have attempted to obtain the best of both worlds by constructing semi-synthetic libraries that combine synthetic diversity in some CDR loops with natural diversity in others. A better understanding of the contributions of the framework regions to antigen recognition has also been useful, but this knowledge has led to different schools of thought on how best to optimize frameworks.

Some groups have chosen to reduce the frameworks to a limited number or even a single, highly stable VH/VL pair, believing that this is likely to lead to stable and well-behaved antibodies. The single framework option also has the advantage of simplicity for library design, construction and analysis. In contrast, other researchers hold the opinion that increased framework diversity provides a greater diversity of antibody function, and thus libraries containing multiple combinations of VH and VL frameworks have also been constructed. At present, it is not clear which of these strategies for framework and CDR design is preferable.

Generating a diverse library of antibodies or antigen binding proteins is also important to isolation of high affinity antibodies. Libraries with diversification in limited CDRs have been generated using a variety of approaches. See, e.g., Tomlinson, Nature Biotech. (2000), 18:989-994. CDR3 regions are of interest in part because they often are found to participate in antigen binding. CDR3 regions on the heavy chain vary greatly in size, sequence and structural conformation, Others have also generated diversity by randomizing CDR regions of the variable heavy and light chains using all 20 amino acids at each position. It was thought that using all 20 amino acids would result in a large diversity of sequences of variant antibodies and increase the chance of identifying novel antibodies. (Barbas, PNAS 91:3809 (1994); Yelton, D E, J. Immunology, 155:1994 (1995); Jackson, J. R., J. Immunology, 154:3310 (1995) and Hawkins, R E, J. Mol. Biology, 226:889 (1992)).

Synthetic antibody libraries have also been used to explore the principles governing antigen recognition. Natural CDR sequences are highly diverse, but they are biased in favor of tyrosine and serine. According to Sidhu et al., their results showed that tyrosine was particularly well-suited to mediate productive interactions with antigen, especially when combined with small serine residues which likely have an auxiliary role by providing space and flexibility. The binary binding sites according to the authors represented a minimal benchmark into which additional diversity could be introduced to study antigen recognition in detail. Sidhu et al., Nature Chemical Biology, Vol. 2, December 2006, pp. 682-688.

There have also been attempts to create diversity by restricting the group of amino acid substitutions in some CDRs to reflect the amino acid distribution in naturally occurring antibodies. See, Garrard & Henner, Gene (1993), 128:103; Knappik et al., J. Mol. Biol. (1999), 296:57. However, these attempts have had varying success and have not been applied in a systematic and quantitative manner. Creating diversity in the CDR regions while minimizing the number of amino acid changes has been a challenge. Furthermore, in some instances, once a first library has been generated according to one set of criteria, it may be desirable to further enhance the diversity of the first library. However, this requires that the first library has sufficient diversity and yet remain sufficiently small in size such that further diversity can be introduced without substantially exceeding practical limitations such as yield, etc. There have also been attempts to create diversity by restricting the group of amino acid substitutions in some CDRs to reflect the amino acid distribution in naturally occurring antibodies. See, Garrard & Denner, Gene (1993), 128:103; Knappik et al., J. Mol. Biol. (1999), 296:57. However, these attempts have had varying success and have not been applied in a systematic and quantitative manner. Creating diversity in the CDR regions while minimizing the number of amino acid changes has been a challenge. As well, some groups have reported theoretical and experimental analyses of the minimum number of amino acid repertoire that is needed for generating proteins. However, these analyses have generally been limited in scope and nature, and substantial skepticism and questions remain regarding the feasibility of generating polypeptides having complex functions using a restricted set of amino acid types. See, e.g., Riddle et al., Nat. Struct. Biol. (1997), 4(10):805-809; Shang et al., Proc. Natl. Acad. Sci. USA (1994), 91:8373-8377; Heinz et al., Proc. Natl. Acad. Sci. USA (1992), 89:3751-3755; Regan & Degrado, Science (1988), 241:976-978; Kamteker et al., Science (1993), 262:1680-1685; Wang & Wang, Nat. Struct. Biol. (1999), 6(11):1033-1038; Xiong et al., Proc. Natl. Acad. Sci. USA (1995), 92:6349-6353; Heinz et al., Proc, Natl. Acad. Sci. USA (1992), 89:3751-3755; Cannata et al., Bioinformatics (2002), 18(8):1102-1108; Davidson et al., Nat. Struct. Biol. (1995), 2(10):856-863; Murphy et al., Prot. Eng. (2000), 13(3):149-152; Brown & Sauer, Proc. Natl. Acad. Sci. USA (1999), 96:1983-1988; Akanuma et al., Proc. Natl. Acad. Sci. (2002), 99(21):13549-13553; Chan, Nat. Struct. Biol. (1999), 6(11):994-996.

Library approaches have also utilized as a tool for antibody humanization. Basically, antibody humanization often requires the replacement of key residues in the framework regions with corresponding residues from the parent non-human antibody. These changes are in addition to grafting the antigen-binding loops. Assessment of which framework changes are beneficial to antigen binding usually requires the analysis of many different antibody mutants.

The goal of antibody humanization is to engineer a monoclonal antibody (MAb) raised in a nonhuman species into one that is less immunogenic when administered to humans. The development of this technology drastically transformed the stagnant state of antibody therapeutics in the 1980s, when the major obstacle was the human anti-murine antibody (HAMA) response. The HAMA response occurred in up to 50% of patients upon the administration of murine hybridoma-derived antibodies and severely compromised the safety, efficacy, and biological half-life of these reagents. In addition, murine antibody constant regions are inefficient in directing suitable human immune effector functions for therapeutic effects. Efforts to produce human antibodies by hybridoma technology and Epstein-Barr virus (EBV)-mediated B-lymphocyte transformation have met with limited success but their widespread application is hampered by the lack of robust human hybridoma fusion partners and the instability of EBV-transformed clones, respectively. Consequently, humanization technology was well-placed to exploit the plethora of murine MAbs against a variety of disease targets and turn them into effective clinical reagents.

Two methods were developed in the mid-1980s as attempts to reduce the immunogenicity of murine antibodies: chimerization (yielding Chimeric antibodies) and humanization by CDR grafting In mouse/human chimeric antibodies, the immunogenic murine constant domains (66% of total sequence) are replaced by the human counterpart to reduce the likelihood of eliciting the HAMA response. At the same time, intact murine variable domains are preserved to maintain the intrinsic antigen-binding affinity. Effector functions of chimeric antibodies may also be programmed by choosing constant domains of different human immunoglobulin isotypes.

An alternative approach designated “complementarity-determining region (CDR) grafting technology” pioneered by Dr. Greg Winter and colleagues at the Medical Research Council, Cambridge proposed transferring the antigen-binding specificity of a murine antibody to a human antibody by replacing the CDR loops and selected variable-region framework residues (FR). Since greater than 90% of the original murine residues are replaced by residues of human origin, one hopes that the immunogenicity is greatly reduced. However, because the affinity for an antigen is controlled by both the CDR sequences and their conformations, changes in the framework might that might affect antigen affinity wee also proposed. Since, the reshaped, humanized antibody only retained the essential binding elements from the murine antibody (5-10% of total sequence) it was predicted to be minimally immunogenic. Indeed, the in vivo tolerance and efficacy of many humanized antibodies have been shown to be more favorable than murine antibodies. Depending on the original antibody, the mouse and human FR sequences can differ at 5-50 positions per chain. Many of these changes are at exposed sites well removed from the CDRs and thus would not affect CDR conformation. Differences that occur at FR sites contacting one or more residues of the CDR do, however, have the potential to alter antigen affinity. The importance of these residues was postulated by Chothia and Lesk. In general, if the murine and human sequences differ at these important positions, the human residue in the framework should be replaced by the original murine residue to maintain the original murine CDR conformation. The meaning of the term reshaping has grown from simple CDR grafting to encompas the grafting of both CDRs and CDR contact regions One approach to minimize this problem is to select as a scaffold a human framework that is as homologous as possible to the original murine framework, especially at the CDR-contact regions. These frameworks can be selected from known structures, known VH/VL pairs, single mature sequences, germline sequences or even synthetically constructed subgroup consensus sequences. Typically, up to five further changes in the framework region are required for activity to be restored to within threefold that of the original antibody. The greater the similarity between the two framework sequences, the less likely it is that a key framework residue will need to be changed. Further changes sometimes involve the elimination of residues that rarely occur in human antibodies. The rationale for this approach is to eliminate potential immunogenicity, but recent results demonstrate that the presence of such rare residues can also have effects on the stability, and possibly the amyloidgenicity, of the variable domains.

The key to a successful CDR grafting experiment lies in the preservation of the murine CDR conformations in the reshaped antibody for antigen binding. This can benefit from a detailed analysis of the structure and sequence of the murine Fv. The antibody Fv region comprises variable domains from the light chain (VL) and heavy chain (VH) and confers antibodies with antigen-binding specificity and affinity. The variable domains adopt the immunoglobulin fold in which two antiparallel beta-sheet framework scaffolds support three hypervariable CDRs. Structural variation between antibodies is dependent on the length, sequence, conformation, and relative disposition of the CDRs, and the pairing of CDRs from repertoires of VL and VH sequences. These parameters create the unique topography of each combining site and form the basis for specific antigen recognition. Although antigen binding is predominantly regulated by CDR residues, framework residues may also contribute, either through direct antigen contact (22) or indirectly through packing with CDR residues. The implication for CDR grafting is that it may be necessary to revert one or more human framework residues to the murine equivalents in the reshaped antibody (so-called framework back-mutations; see FIG. 1B) to restore the native biomolecular environment for antigen binding (13). Benny K. C. Lo; Methods in Molecular Biology, Vol. 248: Antibody Engineering: Methods and Protocols Edited by: B. K. C. Lo© Humana Press Inc., Totowa, N.J.

The news media is littered with recent setbacks relative to therapeutic antibodies for use in humans, prompting greater attention to the identification and selection of therapeutic antibodies going forward. A phase I clinical trial in the UK of the CD28 targeted mAb, TGN1412, led to disaster as six healthy volunteers were admitted to intensive care (Sheridan, 2006a C. Sheridan, TeGenero fiasco prompts regulatory rethink, Nat. Biotechnol. 24 (2006), pp. 475-476. Although immune cells and their modulators are very appealing targets for mAbs (Korman et al., 2006), there are inevitable perils associated with intervention of the immune signaling network. It remains to be seen whether a CD28 targeted drug could ultimately prove to be useful in immunosuppressed individuals, which is the patient population initially proposed to benefit from this therapeutic. In a separate case, Tysabri is a mAb targeting the α4-subunit of α4β1 and α4β7 integrins, thereby inhibiting β4 mediated adhesion to receptors on endothelial cells (Bartt, 2006). Tysabri first entered the market for therapy of relapsing forms of MS in 2004. However, when three patients developed progressive multifocal leukoencephalopathy (PML), the drug was withdrawn. After subsequent clinical trials and further FDA review, Tysabri returned to the market in 2006 under a special restricted distribution program (Sheridan, 2006a and Sheridan, 2006b). In this context, PML is also one risk factor that is recorded on the label for the blockbuster anti-CD20 mAb drug,

Thus, there remains a need to improve methods of generating diverse antibodies including antibody libraries that comprise functional polypeptides having a sufficient degree of sequence diversity, yet are sufficiently amenable for further manipulations directed at further diversification etc. The invention described herein meets this need and provides other benefits.

In fact, contray to the teachings of the art, supra, the invention provides a flexible and simple source of sequence diversity that can be used as a source for expressing and identifying diverse antibodies or antigen binding polypeptides. The method proposes diversifying antibody sequences by specifically introducing amino acid substitutions, additions, deletions or mutation in one of three framework amino acid sequences adjacent to a CDR sequence and/or optionally introducing amino acid substitutions, deletions, additions or mutations in specific amino acid residues contained within a CDR region.

### SUMMARY OF THE INVENTION

The present invention provides methods of systematically and efficiently generating polypeptides (e.g. variant antibody) comprising diversified FR and/or CDRs. Unlike conventional methods that propose that adequate diversity of target binders can be generated only if a particular CDR(s), or all CDRs should be diversified, and unlike conventional notions that adequate diversity is dependent upon the broadest range of amino acid substitutions (generally by substitution using all or most of the 20 amino acids) and unlike conventional notions that prohibit diversifying framework residues closest to the CDR as a means of introducing diversity in an antibody, the invention provides methods capable of generating high quality target binders e.g., antibodies that are not necessarily dependent upon diversifying a particular CDR(s) or a particular number of CDRs of a reference polypeptide or source antibody.

Indeed, the invention is based, at least in part, on the surprising and unexpected findings that highly diverse libraries of high quality antibodies can be generated inter alia by limiting the diversity at amino acid positions limited to three amino acids in the framework region adjoining or adjacent to a CDR sequence on at least one of the heavy and/or light chain together with or optionally with a variation in at least one CDR region on another one of the heavy or light chain. Preferably, the diversity comprises diversifying at least one of three framework residues in at least one of the two regions that envelope/surround each CDR. Methods of the invention are convenient, based on objective and systematic criteria, and rapid. The methods and polypeptides of the invention are useful in the isolation of high affinity binding molecules to target antigens and to provide for well folded antibody variable domains that can readily be adapted to large scale production. The restricted sequence diversity, and thus generally smaller size of the populations (e.g., libraries) of antibody polypeptides generated by methods of the invention allows for further diversification of these populations, where necessary or desired. This is an advantage generally not provided by conventional methods. Candidate binder polypeptides generated by the invention possess high-quality target binding characteristics and have structural characteristics that provide for high yield of production in cell culture. The invention provides methods for generating these binder polypeptides, methods for using these polypeptides, and compositions comprising the same.

One aspect of the invention involves generating a plurality of antibody variable domains that have at least a portion of a framework region that is diversified wherein this region is limited to 3 continuous/contiguous amino acids adjoining each CDR on each side. For clarity, if assuming arguendo, the target antibody comprises CDR1 on the light chain (CDRL1) as being defined by a sequence of amino acids starting at position 8 and ending at position 14, then the methods of the invention propose in certain embodiments, increasing diversity by introducing an amino acid substitution, addition, deletion or mutation either inside the CDR defined by positions 8-14 or in addition to the CDR, substituting, adding, deleting or mutating an amino acid in least one of the framework residue defined by the three amino acids (junctional region) in the framework sequence defined by amino acids at positions 5-7 (3+) and/or 15-17 (3+). In some embodiments, the framework region residues are those residues that are solvent accessible and may form part of an antigen binding domain or loop. For clarity, the junctional region comprises each of the 3 contiguous amino acid s adjoining each CDR on one or both sides. Said another way, the CDR according to the invention thus encompasses and includes the 3 contiguous amino acids on either side of each CDR-junctional region.

In naturally occurring antibody variable domains, the CDRs participate in antigen binding and vary between antibodies. The framework regions form β sheet structures that help to form the antigen binding pocket and typically have less diversity. An antibody antigen-binding site composed of a VH domain and a VL domain is typically formed by six loops of polypeptide: three from the VL and three from the VH. Analysis of antibodies of known atomic structure has elucidated relationships between the sequence and three-dimensional structure of antibody combining sites. These relationships imply that, except for the third region (loop) in VH domains, binding site loops have one of a small number of main-chain conformations: canonical structures. The canonical structure formed in a particular loop has been shown to be determined by its size and the presence of certain residues at key sites in both the loop and in framework regions.

A broad aspect of the invention provides for variant antibodies and antigen variable domains and compositions thereof prepared according to the methods of the invention, polynucleotides encoding the polypeptides and antigen variable domains prepared according to the methods of the invention, replicable expression vectors containing the polynucleotides, and host cells containing the vectors. In an embodiment, a plurality of the vectors encodes a library of a plurality of polypeptides or antigen binding domains of the invention. In a further embodiment, a virus displays on its surface the plurality of polypeptides or antigen binding domains encoded by the vectors. The methods of the invention are useful for identifying novel polypeptides, antibody, antibody fragments, and antibody variable domains that may be used therapeutically or as reagents.

The invention is based, at least in part, on the surprising and unexpected finding that highly diverse libraries of high quality comprising functional polypeptides capable of binding target antigens can be generated by diversifying a minimal number of amino acid positions with a highly restricted number of amino acid residues. Methods of the invention are rapid, convenient and flexible, based on using restricted codon sets that encode a low number of amino acids.

In one aspect, the invention provides a variant antibody or fragment thereof comprising at least one variably mutated domain.

In another aspect of the invention, a method is provided for selecting antibody against an antigen in vitro. The method comprises: contacting a library of antibodies with an antigen that is a fusion protein between a peptide fragment of a target membrane protein and a heterologous chaperon protein that stabilizes the peptide fragment; and selecting the antibodies that bind to the antigen.

By variably mutated it is meant that different antibody molecules in the library have different mutations at those residues. A residue position in the JR, VH or VL domain is variably mutated if different residues are present at that position when the library is considered as a whole. For example there may be at least 2, at least 4, at least 10, at least 15 or at least 20 different residues at a variably mutated position in the library. The different residues at variably mutated positions may comprise non-germline residues, or in some cases may consist only of non-germline residues. Variability may be achieved by random or targeted mutation of the residues. Techniques such as site directed mutagenesis and error prone PCR may be used to produce variable mutation. Oligonucleotide primers may be used in which the codons corresponding to variably mutated residues are designed with the first two bases in each triplet being each selected from A, T, C and G, and the third base of each triplet selected from T and G only. Stop codons TAA and TGA are therefore not encoded.

One aspect of the invention is a library of antibody molecules, wherein each antibody molecule comprises a variant VH domain consisting of a set of VH complementarity determining regions CDR1, CDR2 and CDR3, and at least one framework region, wherein the VII domain amino acid sequence corresponds to a human germline antibody heavy chain sequence in which a residue in VH CDR1, CDR2 and/or CDR3 is variably mutated from germline in addition to a variable mutation in at least one junctional region. In certain embodiments, the JR is left untouched. In another embodiments, the CDR is left untouched and only the JR is diversified—variably mutated.

The invention also provides a library of antibody molecules wherein each antibody molecule comprises a VH domain, and optionally a VL domain forming a VH-VL domain pair, where sequence diversity in the VH complementarity determining regions is restricted to the JR region adjoining each CDR and optionally diversifying at least one CDR and sequence diversity in the VL complementarity determining regions is restricted to one of CDR1, CDr2 or CDR3. Other residues in the VH and VL domain CDRs may be germline residues and/or may be the same in all or most (e.g. at least 90%) of the antibody molecules in the library. VH and VL domain framework regions may be human germline framework regions.

In some embodiments, the amino acid residues in the JR can be varied randomly. In some embodiments, these amino acid positions can be substituted randomly using a codon set that encode all amino acids. In other embodiments, the variant amino acids can be encoded by a nonrandom codon set.

The antibodies of the invention may comprise an antibody variable domain, an antibody or antibody fragment. The antibody fragment may be a Fab, F(ab′).sub.2, scFv, or Fv.

The invention also provides polynucleotides encoding the polypeptides and antigen binding domains of the invention, replicable expression vectors containing the polynucleotides, and host cells containing the vectors. In an embodiment, a plurality of the vectors encodes a library of a plurality of polypeptides or antigen binding domains of the invention. In a further embodiment, a virus displays on its surface the plurality of polypeptides or antigen binding domains encoded by the vectors.

Another aspect of the invention includes methods of generating a plurality of polypeptides that have variant framework residues (junctional region residues). A method comprises generating a plurality of antibody variable domains, each antibody variable domain comprising a variant JR, said method comprising: a) replacing an amino acid in at least one amino acid position at the N terminus of the JR with from 1-8 different amino acids; b) replacing an amino acid in at least one amino acid position at the C terminus of the JR with from 1-12, or from 1-8 amino acids and optionally c) replacing an amino acid in at least one amino acid position within a CDR region.

In some embodiments, the method may optionally include steps for generating polypeptides with variant CDR1, CDR2 or CDR3. The method includes identifying at least one CDR amino acid position that form(s) a loop of an antigen binding pocket; and generating a population of antibody polypeptides with a variant CDR1, CDR2 and/or CDR3 region by replacing the amino acid at said position with about Ito 8, preferably 1-6 of the most commonly occurring amino acids at that position in a randomly generated population and/or replacing the amino acid position with any of the naturally occurring amino acids or with a set of amino acids encoded by a nonrandom codon set to generate a population of polypeptides with different amino acid sequences in one of CDR1, CDR2 or CDr3.

In one aspect, the invention provides fusion polypeptides comprising diversified CDR(s) and/or JR's and a heterologous polypeptide sequence (in certain embodiments, that of at least a portion of a viral polypeptide), as single polypeptides and as a member of a plurality of unique individual polypeptides that are candidate binders to targets of interest. Compositions (such as libraries) comprising such polypeptides find use in a variety of applications, for example, as pools of candidate immunoglobulin polypeptides (for example, antibodies and antibody fragments) that bind to targets of interest. Such antibodies may also be generated using non-immunoglobulin scaffolds (for example, proteins, such as human growth hormone, etc.).

In one aspect, the antibodies comprise at least one variant CDR in its variable domains optionally with a variant JR, wherein the variant CDR comprises at least one variant amino acid in a highly diverse amino acid position, wherein at least 70% of the amino acids are target amino acids for that position in known antibody variable domains. The antibody may have a heavy chain variable domain which comprises at least 1, 2 or 3 variant CDRs selected from the group consisting of CDR H1, H2 and H3 in addition to a variant JR. The antibody may also have a light chain variable domain which comprises at least one JR optionally with 1, 2 or 3 variant CDRs selected from the group consisting of CDR L1 L2 and L3.

Also contemplated are variants of the diversified antibodies with improved binding affinities to its natural binding partner of human or non-human species, or both. Various forms of the antibody and variants thereof are contemplated herein. For example, the antibody mutant may be a full length antibody (e.g. having a human immunoglobulin constant region) or an antibody fragment (e.g. a Fab or F(ab′).sub.2). Furthermore, the antibody mutant may be labeled with a detectable label, immobilized on a solid phase and/or conjugated with a heterologous compound (such as a cytotoxic agent).

In one aspect, the invention provides a method of generating an antibody polypeptide comprising at least one variant JR and at least one, two, three, four, five or all variant CDRs selected from the group consisting of H1, H2, H3, L1, L2 and L3, wherein said antibody is capable of binding a target antigen of interest, said method comprising identifying at least one (or any number up to all) solvent accessible and highly diverse amino acid position in a reference CDR corresponding to the variant CDR; and (ii) varying the amino acid at the solvent accessible and high diverse position by generating variant copies of the CDR using a restricted codon set. A similar method applied to a variant JR.

In another example, the invention provides a method comprising: constructing a library of phage or phagemid particles displaying a plurality of polypeptides of the invention; contacting the library of particles with a target antigen under conditions suitable for binding of the particles to the target antigen; and separating the particles that bind from those that do not bind to the target antigen.

Designing diversity in CDRs may involve designing diversity in the length and/or in sequence of the CDR. For example, CDRH3 may be diversified in length to be, e.g., 7 to 21 amino acids in length, and/or in its sequence, for example by varying highly diverse and/or solvent accessible positions with amino acids encoded by a restricted codon set. In some embodiments, a portion of CDRH3 has a length ranging from 5 to 21, 7 to 20, 9 to 15, or 11 to 13 amino acids, and has a variant amino acid at one or more positions encoded by a restricted codon set that encodes a limited number of amino acids such as codon sets encoding no more than 19, 15, 10, 8, 6, 4 or 2 amino acids.

In some embodiments, polypeptides of the invention can be in a variety of forms as long as the target binding function of the polypeptides is retained. In some embodiments, a polypeptide of the invention is a fusion polypeptide (i.e. a fusion of two or more sequences from heterologous polypeptides). Polypeptides with diversified CDRs according to the invention can be prepared as fusion polypeptides to at least a portion of a viral coat protein, for example, for use in phage display. Viral coat proteins that can be used for display of the polypeptides of the invention comprise protein p III, major coat protein pVIII, Soc (T4 phage), Hoc (T4 phage), gpD (lambda phage), pVI, or variants or fragments thereof. In some embodiments, the fusion polypeptide is fused to at least a portion of a viral coat protein, such as a viral coat protein selected from the group consisting of pill, pvIII, Soc, Hoc, gpD, pVI, and variants or fragments thereof.

In some embodiments, in which the polypeptide with diversified CDRs is one or more antibody variable domains, the antibody variable domains can be displayed on the surface of the virus in a variety of formats including ScFv, Fab, SeFv.sub.2, F(ab′).sub.2 and F(ab).sub.2.

For display of the polypeptides in bivalent manner, the fusion protein in certain embodiments includes a dimerization domain. The dimerization domain can comprise a dimerization sequence and/or a sequence comprising one or more cysteine residues. The dimerization domain can be linked, directly or indirectly, to the C-terminal end of a heavy chain variable or constant domain (e.g., CH1). The structure of the dimerization domain can be varied depending on whether the antibody variable domain is produced as a fusion protein component with the viral coat protein component (e.g., without an amber stop codon after dimerization domain) or whether the antibody variable domain is produced predominantly without the viral coat protein component (e.g., with an amber stop codon after dimerization domain). When the antibody variable domain is produced predominantly as a fusion protein with the viral coat protein component, one or more disulfide bonds and/or a single dimerization sequence provides for bivalent display. For antibody variable domains predominantly produced without being fused to a viral coat protein component (e.g. with an amber stop codon), the dimerization domain can comprise both a cysteine residue and a dimerization sequence.

In addition, optionally, a fusion polypeptide can comprise a tag that may be useful in purification, detection and/or screening such as FLAG, poly-his, gD tag, c-myc, fluorescence protein or B-galactosidase. In one embodiment, a fusion polypeptide comprises a light chain variable or constant domain fused to a polypeptide tag.

In another aspect of the invention, a polypeptide such as an antibody variable domain is obtained from a single source or template molecule. The source or template molecule can be selected or designed for characteristics such as good yield and stability when produced in prokaryotic or eukaryotic cell culture, and/or to accommodate CDR regions of varying lengths. The sequence of the template molecule can be altered to improve folding and/or display of the variable domain when presented as a fusion protein with a phage coat protein component. For example, a source antibody may comprise the amino acid sequence of the variable domains of humanized antibody PCSK9. For example, in an antibody variable domain of a heavy or light chain, framework region residues can be modified or altered from the source or template molecule to improve folding, yield, display or affinity of the antibody variable domain. In some embodiments, framework residues may be selected to be modified from the source or template molecule when the amino acid in the framework position of the source molecule is different from the amino acid or amino acids commonly found at that position in naturally occurring antibodies or in a subgroup consensus sequence. The amino acids at those positions can be changed to the amino acids most commonly found in the naturally occurring antibodies or in a subgroup consensus sequence at that position.

The methods of the invention provide populations of variant antibodies (for example, libraries of antibody variable domains with one or more diversified junctional regions in addition to or optionally with at least one variant CDR region. The variant junctional region may comprise the region at the N terminal of the CDR region and/or C terminal of the CDR region or a combination of both. These libraries are sorted (selected) and/or screened to identify high affinity binders to a target antigen. In one aspect, antibodies from the library are selected for binding to target antigens, and for affinity. The variant antibodies selected using one or more of these selection strategies, may then be screened for affinity and/or for specificity (binding only to target antigen and not to non-target antigens). Methods of the invention are capable of generating a large variety of antibody sequences comprising a diverse set of CDR and/or JR sequences. In an embodiment, a one or more libraries are formed using the methods of the invention as described herein. The libraries are screened for binding to a target antigen, e.g. human Her3, PCSK9 etc.

Immunoglobulin heavy chain variable domains randomized to provide diversity are provided.

In another embodiment, a method of generating a composition comprising a plurality of polypeptides is provided, comprising: (a) generating a plurality of polypeptides comprising: an amino acid substitution in a framework sequence optionally with at least one or more additional substitutions in s CDR region on one of a heavy or light chain.

In one aspect, the plurality of polypeptides are encoded by a plurality of polynucleotides.

In a broad aspect, a method of the invention comprises generating a plurality of variant antibodies with one or more diversified JR and/or CDR regions, sorting the plurality of variant antibodies for binders to a target antigen by contacting the plurality of variant antibodies with a target antigen under conditions suitable for binding; separating the binders to the target antigen from those that do not bind; isolating the binders; and identifying the high affinity binders (or any binders having a desired binding affinity). The affinity of the binders that bind to the target antigen can be determined using a variety of techniques known in the art, for example, competition ELISA such as described herein. Optionally, the polypeptides can be fused to a polypeptide tag, such as gD, poly his or FLAG, which can be used to sort binders in combination with sorting for the target antigen.

Another general embodiment provides a method of isolating or selecting for an antibody variable domain that binds to a target antigen from a library of antibody variable domains, said method comprising: a) contacting a population comprising a plurality of variant antibodies of the invention with an immobilized target antigen under conditions suitable for binding to isolate target antigen antibody binders; b) separating the binders from nonbinders, and eluting the binders from the target antigen; c) optionally, repeating steps a-b at least once (in some embodiments, at least twice).

In some embodiments, a method may further comprise: d) incubating the polypeptide binders with a concentration of labeled target antigen in the range of 0.1 nM to 1000 nM under conditions suitable for binding to form a mixture; e) contacting the mixture with an immobilized agent that binds to the label on the target antigen; f) eluting the binders from the labeled target antigen; g) optionally, repeating steps d) to f) at least once (in some embodiments, at least twice), using a successively lower concentration of labeled target antigen each time. Optionally, the method may comprise adding an excess of unlabelled target antigen to the mixture and incubating for a period of time sufficient to elute low affinity binders from the labeled target antigen.

In one embodiment, a method of generating one or more of variant antibodies comprising at least one framework sequence which differs from a wild type sequence and at least one, two, three, four, five or all variant CDRs selected from the group consisting of H1, H2, H3, L1, L2 and L3, wherein said antibody is capable of binding a target antigen of interest is provided, comprising: (a) constructing an expression vector comprising a polynucleotide sequence which encodes a light chain variable domain, a heavy chain variable domain, or both of a source antibody comprising at least one, two, three, four, five or all CDRs of the source antibody selected from the group consisting of CDRL1, CDRL2, CDRL3, CDRH1, CDRH2, and CDRH3 and at least one framework sequence; and (b) mutating at least one, two three, four, five or all CDRs of the source antibody and said one framework sequence to generate one or more of the above-described hypervariable regions.

The invention also includes a method of screening a population comprising a plurality of polypeptides of the invention, said method generally comprising: a) incubating a first sample of the population of variant antibodies with a target antigen under conditions suitable for binding of the variant antibodies to the target antigen; b) subjecting a second sample of the population of variant antibodies to a similar incubation but in the absence of the target antigen; (c) contacting each of the first and second sample with immobilized target antigen under conditions suitable for binding of the variant antibodies to the immobilized target antigen; d) detecting amount of variant antibodies bound to immobilized target antigen for each sample; e) determining affinity of a particular variant antibody for the target antigen by calculating the ratio of the amount of the particular variant antibody that is bound in the first sample over the amount of the particular variant antibody that is bound in the second sample.

The libraries generated as described herein may also be screened for binding to a specific target and for lack of binding to nontarget antigens. In one aspect, the invention provides a method of screening for a variant antibody, such as an antibody variable domain of the invention, that binds to a specific target antigen from a library of antibody variable domains, said method comprising: a) generating a population comprising a plurality of polypeptides of the invention; b) contacting the population of variant antibodies with a target antigen under conditions suitable for binding; c) separating a binder polypeptide in the library from nonbinder polypeptides; d) identifying a target antigen-specific binder by determining whether the binder binds to a non-target antigen; and e) isolating a target antigen-specific binder polypeptide. In some embodiments, step (e) comprises eluting the binder polypeptide from the target antigen, and amplifying a replicable expression vector encoding said binder polypeptide. In some embodiments, one or more of the libraries, clones or variant antibodies are screened against a panel of antigens including the target antigen. In some embodiments, those clones or variant antibodies that specifically bind to the target antigen and do not substantially crossreact with any of the other antigen on the panel are selected. The panel of antigens can include at least three and up to 100 different antigens. In some cases, the panel of antigens includes 3 to 100, 3 to 50, 3 to 25, or 3 to 10 different antigens.

Combinations of any of the sorting/selection methods described above may be combined with the screening methods. For example, in one embodiment, polypeptide binders (e.g. variant antibodies) are first selected for binding to an immobilized target antigen. Polypeptide binders that bind to the immobilized target antigen can then be screened for binding to the target antigen and for lack of binding to nontarget antigens. Polypeptide binders that bind specifically to the target antigen can be amplified as necessary. These polypeptide binders can be selected for higher affinity by contact with a concentration of a labeled target antigen to form a complex, wherein the concentration range of labeled target antigen is from about 0.1 nM to about 1000 nM, and the complexes are isolated by contact with an agent that binds to the label on the target antigen. A polypeptide binder can then be eluted from the labeled target antigen and optionally, the rounds of selection are repeated, and each time a lower concentration of labeled target antigen is used. The binder polypeptides that can be isolated using this selection method can then be screened for high affinity using conventional methods known in the art. Populations of variant antibodies of the invention used in methods of the invention can be provided in any form suitable for the selection/screening steps. For example, the variant antibodies can be in free soluble form, attached to a matrix, or present at the surface of a viral particle such as phage or phagemid particle. In some embodiments of methods of the invention, the plurality of variant antibodies are encoded by a plurality of replicable vectors provided in the form of a library. In selection/screening methods described herein, vectors encoding a binder polypeptide may be further amplified to provide sufficient quantities of the variant antibodies for use in repetitions of the selection/screening steps (which, as indicated above, are optional in methods of the invention).

In one embodiment, the invention provides a method of selecting for a polypeptide that binds to a target antigen comprising: a) generating a composition comprising a plurality of polypeptides of the invention as described herein; b) selecting a polypeptide binder that binds to a target antigen from the composition; c) isolating the polypeptide binder from the nonbinders; d) identifying binders of the desired affinity from the isolated polypeptide binders.

In another embodiment, the invention provides a method of selecting for an antigen binding variable domain that binds to a target antigen from a library of antibody variable domains comprising: a) contacting the library of antibody variable domains of the invention (as described herein) with a target antigen; b) separating binders from nonbinders, and eluting the binders from the target antigen and incubating the binders in a solution with decreasing amounts of the target antigen in a concentration from about 0.1 nM to 1000 nM; c) selecting the binders that can bind to the lowest concentration of the target antigen and that have an affinity of about 0.1 nM to 200 nM.

In one embodiment, the invention provides a method of selecting for a polypeptide that binds to a target antigen from a library of polypeptides comprising: a) isolating polypeptide binders to a target antigen by contacting a library comprising a plurality of polypeptides of the invention (as described herein) with an immobilized target antigen under conditions suitable for binding; b) separating the polypeptide binders in the library from nonbinders and eluting the binders from the target antigen to obtain a subpopulation enriched for the binders; and c) optionally, repeating steps a-b at least once (in some embodiments at least twice), each repetition using the subpopulation of binders obtained from the previous round of selection.

In some embodiments, methods of the invention further comprise the steps of d) incubating the subpopulation of polypeptide binders with a concentration of labeled target antigen in the range of 0.1 nM to 1000 nM under conditions suitable for binding to form a mixture; e) contacting the mixture with an immobilized agent that binds to the label on the target antigen; f) detecting the polypeptide binders bound to labeled target antigens and eluting the polypeptide binders from the labeled target antigen; g) optionally, repeating steps d) to f) at least once (in some embodiments, at least twice), each repetition using the subpopulation of binders obtained from the previous round of selection and using a lower concentration of labeled target antigen than the previous round.

In some embodiments, these methods further comprise adding an excess of unlabelled target antigen to the mixture and incubating for a period of time sufficient to elute low affinity binders from the labeled target antigen. In one embodiment, a method of selecting for a polypeptide that binds to a target antigen is provided, comprising: (a) generating a composition with a plurality of one or more of the above-described polypeptides; (b) selecting one or more polypeptides from the composition that binds to a target antigen; (c) isolating the one or more polypeptides that bind to the target antigen from polypeptides that do not bind to the target antigen; and (d) identifying the one or more polypeptides that bind to the target antigen that have a desired affinity for the target antigen.

In one embodiment, a method of selecting for an antigen binding variable domain that binds to a target antigen from a library of antibody variable domains is provided, comprising: (a) contacting one or more of the herein described antibody libraries with a target antigen; (b) separating one or more antibodies that specifically bind to the target antigen from antibodies that do not specifically bind to the target antigen, recovering the one or more antibodies that specifically bind to the target antigen, and incubating the one or more antibodies that specifically bind to the target antigen in a series of solutions comprising decreasing amounts of the target antigen in a concentration from about 0.1 nM to about 1000 nM; and (c) selecting the one or more antibodies that specifically bind to the target antigen and that can bind to the lowest concentration of the target antigen or that have an affinity of about 0.1 nM to about 200 nM. In one aspect, the target antigen is as described herein. In one aspect, the concentration of the target antigen is about 100 to about 250 nM. In one aspect, the concentration of target antigen is about 25 to about 100 nM. In some embodiments, one or more of the libraries, clones or antibodies are screened against a panel of antigens including the target antigen. In some embodiments, those clones or antibodies that specifically bind to the target antigen and do not substantially crossreact with any of the other antigen on the panel are selected. The panel of antigens can include at least three and up to 100 different antigens. In some cases, the panel of antigens includes 3 to 100, 3 to 50, 3 to 25, or 3 to 10 different antigens.

In one embodiment, a method of selecting for a variant antibody that binds to a target antigen from a library of antibodies is provided, comprising: (a) isolating one or more antibodies that specifically bind to the target antigen by contacting a library comprising a plurality of any of the above-described antibodies with an immobilized target antigen under conditions suitable for binding; (b) separating the one or more antibodies that specifically bind to the target antigen from antibodies that do not specifically bind to the target antigen, and recovering the one or more antibodies that specifically bind to the target antigen to obtain a subpopulation enriched for the one or more antibodies that specifically bind to the target antigen; and (c) optionally, repeating steps (a)-(b) at least twice, each repetition using the subpopulation enriched for the one or more antibodies that specifically bind to the target antigen obtained from the previous round of selection. In one aspect, the method further comprises: (d) incubating the subpopulation with a concentration of labeled target antigen in the range of about 0.1 nM to about 1000 nM to form a mixture, under conditions suitable for binding; (e) contacting the mixture with an immobilized agent that binds to the label on the target antigen; (f) detecting the one or more antibodies that specifically bind to the labeled target antigen, and recovering the one or more antibodies that specifically bind to the labeled target antigen from the labeled target antigen; and (g) optionally, repeating steps (d) to (0 at least twice, each repetition using the subpopulation enriched for the one or more antibodies that specifically bind to the labeled target antigen obtained from the previous round of selection, and using a lower concentration of labeled target antigen than the previous round of selection.

In one aspect, the method further comprises adding an excess of unlabeled target antigen to the mixture and incubating the mixture for a period of time sufficient to recover one or more antibodies that specifically bind to the target antigen with low affinity. In some embodiments, in any of the methods described herein, one or more of the libraries, clones or antibodies are screened against a panel of antigens including the target antigen. In some embodiments, those clones or antibodies that specifically bind to the target antigen and do not substantially crossreact with any of the other antigen on the panel are selected. The panel of antigens can include at least three and up to 100 different antigens.

In one embodiment, a method of isolating one or more antibodies that specifically bind to a target antigen with high affinity is provided, comprising: (a) contacting a library comprising a plurality of any of a variant antibodies with a target antigen at a concentration of at least about 0.1 nM to about 1000 nM to isolate one or more antibodies that specifically bind to the target antigen; (b) recovering the one or more antibodies that specifically bind to the target antigen from the target antigen to obtain a subpopulation enriched for the one or more antibodies that specifically bind to the target antigen; and (c) optionally repeating steps (a) and (b) at least twice, each repetition using the subpopulation obtained from the previous round of selection and using a decreased concentration of target antigen from that used in the previous round to isolate one or more antibodies that bind specifically to the target antigen at the lowest concentration of target antigen.

In one embodiment, a method of screening a library comprising a plurality of any of the herein described antibodies is provided, comprising: (a) incubating a first sample of the library with a target antigen under conditions suitable for binding of the antibodies to the target antigen; (b) incubating a second sample of the library in the absence of a target antigen; (c) contacting each of the first sample and the second sample with immobilized target antigen under conditions suitable for binding of the antibodies to the immobilized target antigen; (d) detecting the antibodies bound to immobilized target antigen for each sample; and (e) determining the affinity of the antibodies for the target antigen by calculating the ratio of the amounts of bound antibodies from the first sample over the amount of bound antibodies from the second sample.

In some embodiments, a polypeptide of the invention comprises a light chain and a heavy chain antibody variable domain, wherein the light chain variable domain comprises at least 1 JR and may further comprise at least 1, 2 or 3 variant CDRs selected from the group consisting of CDR L1, L2 and L3, and the heavy chain variable domain comprises at least 1, 2 or 3 variant CDRs selected from the group consisting of CDR H1, H2 and H3.

In some embodiments, a polypeptide of the invention further comprises a light chain constant domain fused to a light chain variable domain, which in some embodiments comprises at least one, two or three variant CDRs. In some embodiments of antibodies of the invention, the antibody comprises a heavy chain constant domain fused to a heavy chain variable domain, which in some embodiments comprises at least one, two or three variant CDRs.

As described herein, a variant CDR refers to a CDR with a sequence variance as compared to the corresponding CDR of a single reference polypeptide/source antibody. Accordingly, the CDRs of a single antibody of the invention can in certain embodiments correspond to the set of CDRs of a single reference antibody or source antibody. Antibodies of the invention may comprise any one or combinations of variant CDRs. For example, a variant antibody of the invention may comprise a variant CDRH1 and variant CDRH2 and/or a variant JR. An antibody of the invention may comprise a variant CDRH1, variant CDRH2 and a variant CDRH3. In another example, a polypeptide of the invention may comprise a variant JR, a variant CDRH1, variant CDRH2, variant CDRH3 and variant CDRL3. In another example, a variant antibody of the invention comprises a variant JR, a variant CDRL1, variant CDRL2 and variant CDRL3. Any polypeptide of the invention may further comprise a variant CDRL3. Any antibody of the invention may further comprise a variant CDRH3 and/or a variant JR.

In one aspect, the invention provides a polynucleotide encoding a variant antibody of the invention as described herein. In another aspect, the invention provides a vector comprising a sequence encoding a variant antibody of the invention. The vector can be, for example, a replicable expression vector (for example, the replicable expression vector can be M13, fl, fd, Pf3 phage or a derivative thereof, or a lambdoid phage, such as lambda, 21, phi80, phi81, 82, 424, 434, etc., or a derivative thereof). The vector can comprise a promoter region linked to the sequence encoding a polypeptide of the invention. The promoter can be any suitable for expression of the variant antibody, for example, the lac Z promoter system, the alkaline phosphatase pho A promoter (Ap), the bacteriophage I.sub.PL promoter (a temperature sensitive promoter), the tac promoter, the tryptophan promoter, and the bacteriophage T7 promoter. Thus, the invention also provides a vector comprising a promoter selected from the group consisting of the foregoing promoter systems.

Polypeptides or variant antibodies of the invention can be displayed in any suitable form in accordance with the need and desire of the practitioner. For example, an antibody of the invention can be displayed on a viral surface, for example, a phage or phagemid viral particle. Accordingly, the invention provides viral particles comprising a variant antibody of the invention and/or polynucleotide encoding a variant antibody of the invention.

In one aspect, the invention provides a population comprising a plurality of a variant antibodies or polynucleotides of the invention, wherein each type of variant antibody or polynucleotide is a variant antibody or polynucleotide of the invention as described herein.

In some embodiments, variant antibodies and/or polynucleotides are provided as a library, for example, a library comprising a plurality of at least about 1.times.10.sup.4, 1.times.10.sup.5, 1.times.10.sup.6, 1.times.10.sup.7, 1.times.10.sup.8 distinct variant antibody and/or polynucleotide sequences of the invention. A library of the invention may comprise viruses or viral particles displaying any number of distinct variant antibody (sequences), for example, at least about 1.times.10.sup.4, 1.times.10.sup.5, 1.times.10.sup.6, 1.times.10.sup.7, 1.times.10.sup.8 distinct variant antibodies.

In another aspect, the invention provides host cells comprising a polynucleotide or vector comprising a sequence encoding a variant antibody of the invention.

Diagnostic and therapeutic uses for the variant antibodies of the invention are also contemplated. In one diagnostic application, the invention provides a method for determining the presence of a protein of interest comprising exposing a sample suspected of containing the protein to a variant antibody of the invention and determining binding of the variant antibody to the sample. For this use, the invention provides a kit comprising the variant antibodies and instructions for using the variant antibodies to detect the protein.

The invention further provides: isolated nucleic acid encoding the binder polypeptide; a vector comprising the nucleic acid, optionally, operably linked to control sequences recognized by a host cell transformed with the vector; a host cell transformed with the vector; a process for producing the binder polypeptide comprising culturing this host cell so that the nucleic acid is expressed and, optionally, recovering the binder polypeptide from the host cell culture (e.g. from the host cell culture medium).

The invention also provides a composition comprising a variant antibody of the invention and a carrier (e.g., a pharmaceutically acceptable carrier) or diluent. This composition for therapeutic use is sterile and may be lyophilized. Also contemplated is the use of a variant antibody of this invention in the manufacture of a medicament for treating an indication described herein. The composition can further comprise a second therapeutic agent such as a chemotherapeutic agent, a cytotoxic agent or an anti-angiogenic agent.

The invention further provides a method for treating a mammal, comprising administering an effective amount of a variant antibody of the invention to the mammal, The mammal to be treated in the method may be a nonhuman mammal, e.g. a primate suitable for gathering preclinical data or a rodent (e.g., mouse or rat or rabbit). The nonhuman mammal may be healthy (e.g. in toxicology studies) or may be suffering from a disorder to be treated with the binder polypeptide of interest.

In one embodiment, the mammal is suffering from or is at risk of developing abnormal angiogenesis (e.g., pathological angiogenesis). In one embodiment, the disorder is a cancer. The amount of the variant antibody of the invention that is administered will be a therapeutically effective amount to treat the disorder. In dose escalation studies, a variety of doses of the binder polypeptide may be administered to the mammal. In another embodiment, a therapeutically effective amount of the variant antibody is administered to a human patient to treat a disorder in that patient. In one embodiment, the variant antibody according to the invention are Fab or scFv antibodies.

For the sake of clarity, in the description herein, unless specifically or contextually indicated otherwise, all amino acid numberings are according to Kabat et al. (see further elaboration in “Definitions” below).

### BRIEF DESCRIPTION OF THE DRAWINGS

FIG. 1 shows the consistency of FR backbones among several hundred known crystal structures, and the diversity of structural folds for various CDR lengths.

FIG. 2 shows PDL1 library framework sequences of VH, VK, and Vlambda.

FIG. 3 shows the VH-CDR length distribution and CDR loop structure clusters.

FIG. 4 shows the PDL1 library design process.

FIG. 5 shows the amino acid sequences of VH-CDR1 libraries of PDL1 library.

FIG. 6 shows the amino acid sequences of VH-CDR2 libraries of PDL1 library.

FIGS. 7A, 7B, and 7C show the amino acid sequences of VH-CDR3 libraries of PDL1 library.

FIGS. 8A and 8B show the amino acid sequences of VK-CDR1 libraries of PDL1 library.

FIGS. 9A and 9B show the amino acid sequences of VK-CDR3 libraries of PDL1 library.

FIGS. 10A and 10B show the amino acid sequences of Vλ-CDR3 libraries of PDL1 library.

FIG. 11 shows examples to reduce the possibility of cyteines from 54% (before split) to 0% (post split), as well as the size of libraries.

FIG. 12 illustrates the adapter-directed phage display system.

FIG. 13 shows the vector map of helper phage GMCT.

FIG. 14 shows the vector map of pABMX492 for Fab display.

FIG. 15 shows the display vectors for PDL1 library with K and lambda light chains.

FIG. 16 illustrates the PDL1 library construction process. PDL1 library is a fully synthetic human antibody library. Construction of this synthetic library consist of the following four major consecutive steps: 1) syntheses of library degenerated oligos; 2) assembly of building blocks and chain libraries from the oligos; 3) ORF filtering of building blocks to remove open-reading frame shifts; 4) Fab library construction by cloning light chain and heavy chain into Fab display vectors.

FIG. 17 illustrates the oligo quality assessments. FIG. 17A shows the purity analysis of library oligos. FIG. 17B shows the library oligo fidelity.

FIGS. 18A and 18B show examples of assembly of library building blocks.

FIG. 19 shows the vector map of pMAS3 vector.

FIG. 20 shows the ELISA data that illustrates the binding of PDL1 Fabs to human PCSK9.

FIG. 21A shows the sequences of VH from a panel of anti-human PCSK9 antibodies isolated from PDL1 library panning.

FIG. 21B shows the sequences of VK from a panel of anti-human PCSK9 antibodies isolated from PDL1 library panning.

FIG. 22 shows the ELISA data that illustrated the binding of PDL1-Fabs to mouse PCSK9.

FIGS. 23A and 23B show the sequences of VH from a panel of anti-mouse PCSK9 antibodies isolated from PDL1 library panning.

FIGS. 23C and 23D show the sequences of VK from a panel of anti-mouse PCSK9 antibodies isolated from PDL1 library panning.

FIG. 24 shows the ELISA data that illustrated the binding of PDL1-Fabs to human Her3 protein.

FIGS. 25A and 25B show the sequences of VH from a panel of anti-human Her3 antibodies isolated from PDL1 library panning.

FIGS. 25C and 25D show the sequences of VK from a panel of anti-human Her3 antibodies isolated from PDL1 library panning.

FIG. 26 shows the ELISA data that illustrated the binding of PDL1-Fabs to mouse IL13R-Fc protein.

FIGS. 27A and 27B show the sequences of VH from a panel of anti-mouse IL13R-Fc antibodies isolated from PDL1 library panning.

FIGS. 27C and 27D show the sequences of VK from a panel of anti-mouse IL13R-Fc antibodies isolated from PDL1 library panning.

FIG. 28 shows the ELISA data that illustrated the binding of PDL1-Fabs to mouse EPHA2 protein.

FIG. 29A shows the sequences of VH from a panel of anti-mouse EPHA2 antibodies isolated from PDL1 library panning.

FIG. 29B shows the sequences of VK from a panel of anti-mouse EPHA2 antibodies isolated from PDL1 library panning.

FIG. 30 shows the ELISA data that illustrated the binding of PDL1-Fabs to HSV viral protein gE protein.

FIG. 31 shows the sequences of VH and Vk from a panel of anti-HSV viral protein gE antibodies isolated from PDL1 library panning.

FIG. 32 shows the ELISA data that illustrated the binding of PDL1-Fabs to TrkA protein.

FIG. 33 shows the sequences of VH, Vk, and Vλ from a panel of anti-TrkA antibodies isolated from PDL1 library panning.

FIG. 34 illustrates the impact of PDL1 Fabs on PCSK9-LDL receptor interaction. This Biacore-based assay shows that binding of AX1, AX9, and AX114 to PCSK9 inhibits the interaction of PCSK9-LDLR and PCSK9-EGF_AB domain. EGF_AB domain in LDLR involves the interaction with PCSK9.

FIG. 35 shows the distribution of Fab affinities against human PCSK9.

FIG. 36 illustrates a library designed for AX114 antibody engineering. MABL83 library is a light chain library with amino acid diversity in VK-CDR1, CDR2 and CDR3 regions. The amino acid substitutions at each position are provided.

FIG. 37 illustrates the library designed for AX114 antibody engineering. MABL85 library is a heavy chain library with amino acid diversity in VH-CDR1, CDR2 and CDR3 regions. The amino acid substitutions at each position are provided.

FIG. 38 illustrates the library designed for AX114 antibody engineering. MABL87 library is a heavy chain library with amino acid diversity in CDR3 regions. The amino acid substitutions at each position are provided.

FIG. 39 illustrates the library designed for AX114 antibody engineering. MABL88 library is a heavy chain and light library with amino acid diversity in VH-CDR1, 2 and Vk-CDR1, 2, 3 regions. The amino acid substitutions at each position are provided.

FIG. 40 illustrates the library designed for AX114 antibody engineering. MABL89 library is a heavy chain library with amino acid diversity in VH-CDR1, 2 regions. The amino acid substitutions at each position are provided.

FIG. 41 illustrates the library designed for AX114 antibody engineering. MABL90 library is a light chain library with amino acid diversity in Vk-CDR1, 2 regions. The amino acid substitutions at each position are provided.

FIG. 42 illustrates the library designed for AX114 antibody engineering. MABL92 library is a heavy and light chain library with amino acid diversity in VH-CDR3 and Vk-CDR3 regions. The amino acid substitutions at each position are provided.

FIG. 43 illustrates the allowed amino acid substitutions in VK-CDR1, 2, 3 regions from 134 AX114 variants, which are isolated from 10 optimization libraries listed in Table 19. For each variable position, the allowed amino acids are provided.

FIG. 44 illustrates the allowed amino acid substitutions in VH-CDR1, 2, 3 regions from 134 AX114 variants, which are isolated from 10 optimization libraries listed in Table 19. For each variable position, the allowed amino acids are provided.

FIG. 45 illustrates the process of affinity measurement for yeast hits in E. coli system.

### DETAILED DESCRIPTION OF THE INVENTION

The invention provides novel, unconventional, greatly simplified and flexible methods for diversifying CDR sequences (including antibody variable domain sequences), and libraries comprising a multiplicity, generally a great multiplicity of diversified CDRs (including antibody variable domain sequences). Such libraries provide combinatorial libraries useful for, for example, selecting and/or screening for synthetic antibody clones with desirable activities such as binding affinities and avidities. These libraries are useful for identifying immunoglobulin polypeptide sequences that are capable of interacting with any of a wide variety of target antigens. For example, libraries comprising diversified immunoglobulin polypeptides of the invention expressed as phage displays are particularly useful for, and provide a high throughput, efficient and automatable systems of, selecting and/or screening for antigen binding molecules of interest. The methods of the invention are designed to provide high affinity binders to target antigens with minimal changes to a source or template molecule and provide for good production yields when the antibody or antigens binding fragments are produced in cell culture.

Methods and compositions of the invention provide numerous additional advantages. For example, relatively simple variant CDR sequences can be generated, using codon sets encoding a restricted number of amino acids (as opposed to the conventional approach of using codon sets encoding the maximal number of amino acids), while retaining sufficient diversity of unique target binding sequences. The simplified nature (and generally relatively smaller size) of sequence populations generated according to the invention permits further diversification once a population, or sub-population thereof, has been identified to possess the desired characteristics.

The simplified nature of sequences of target antigen binders obtained by methods of the invention leaves significantly greater room for individualized further sequence modifications to achieve the desired results. For example, such sequence modifications are routinely performed in affinity maturation, humanization, etc. By basing diversification on restricted codon sets that encode only a limited number of amino acids, it would be possible to target different epitopes using different restricted codon sets, thus providing the practitioner greater control of the diversification approach as compared with randomization based on a maximal number of amino acids. An added advantage of using restricted codon sets is that undesirable amino acids can be eliminated from the process, for example, methionine or stop codons, thus improving the overall quality and productivity of a library. Furthermore, in some instances, it may be desirable to limit the conformational diversity of potential binders. Methods and compositions of the invention provide the flexibility for achieving this objective. For example, the presence of certain amino acids, such as tyrosine, in a sequence results in fewer rotational conformations.

### DEFINITIONS

Amino acids are represented herein as either a single letter code or as the three letter code or both.

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “antibody” is used in the broadest sense and specifically covers single monoclonal antibodies (including agonist and antagonist antibodies), antibody compositions with polyepitopic specificity, affinity matured antibodies, humanized antibodies, chimeric antibodies, as well as antigen binding fragments (e.g., Fab, F(ab′).sub.2, scFv and Fv), so long as they exhibit the desired biological activity. In one embodiment, the term “antibody” also includes human, chimeric and humanized antibodies.

A “variable region” has the structure of an antibody variable region from a heavy or light chain. Antibody heavy and light chain variable regions contain three complementarity determining regions (“CDRs”) interspaced onto a framework (“FW”). The CDRs are primarily responsible for recognizing a particular epitope. It is well known that epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The hypervariable regions are generally referred to as complementarity determining regions (“CDR”) and are interposed between more conserved flanking regions referred to as framework regions (“FW”). There are four (4) FW regions and three (3) CDRs that are arranged from the NH2 terminus to the COOH terminus as follows: FW1, CDR1, FW2, CDR2, FW3, CDR3, FW4. Amino acids associated with framework regions and CDRs can be numbered and aligned by approaches described by Kabat et al., Sequences of Proteins of Immunological Interest, U.S. Department of Health and Human Services, 1991; C. Chothia and A. M. Lesk, Canonical structures for the hypervariable regions of immunoglobulins, Journal of Molecular Biology 196(4):901 (1987); or B. Al-Lazikani, A. M. Lesk and C. Chothia, Standard conformations for the canonical structures of immunoglobulins <http://dx.doi.org/10.1006/jmbi.1997.1354>, Journal of Molecular Biology 273(4): 27, 1997. For example, the framework regions and CDRs can be identified from consideration of both the Kabat and Chothia definitions.

CDRs are primarily responsible for binding to a particular epitope. Within a particular CDR, there are a few specificity determining residues (SDRs) which are of greater importance for binding to an epitope (see Kashmiri et al., Methods 36:25-34, 2005; Presta, Advanced Drug Delivery Reviews 58:640-656, 2006). SDRs can be identified, for example, through the help of target protein-antibody three-dimensional structures and mutational analysis of antibody combining sites. (Kashmiri et al., 2005, supra.) Thus, the PD-1 binding proteins of the present invention do not always require both a variable heavy chain and light chain domain to render PD-1 specificity but may only need a single CDR loop or a fragment of a functional antibody (see, e.g., Xu and Davis, 2000, Immunity 13:37-45 and Levi et al., 1993, Proc. Natl. Acad. Sci. USA 90:4374-78 (for CDR3 specificity); Williams et al., 1989, Proc. Natl. Acad. Sci. USA 86:5537-41 (CDR2 specificity); and, Welling et al., 1991, 3. Chromatography 548:235-42 (10 amino acid miniantibody).

For the purposes of this application, the term “CDR” is expanded to includes not only the standard definition of a CDR but also includes the three contiguous amino acid residues from the Fr region that flank each CDR. Thus CDR as defined in the application can be defined schematically as +3CDR+3 wherein each +3 refers to the 3FR amino acids flanking each CDR on each side. Alternatively, a CDR may include one or both JR's appended on each end—the N and/or C-terminal end of the CDR. Thus, in some embodiments, the heavy chain comprises 3 CDRs, each CDR comprising a JR_{N} and a JR_{C}. The same holds true for a light chain. It is understood that when introducing a mutation via a addition, substitution, addition, mutation etc into one or more JR's, one need not necessarily also mutate the other JR or the CDR shared by the respective JR's. In other words, if a framework residue in a JR was diversified via an amino acid substitution into the N-terminal JR relative to the CDRH1, the methods of the invention do not propose necessarily diversifying either CDRH1 and/or JF at the C terminal of CDRH1. Thus a JR_{N} of CDHR1 may be diversified and combined with a CDR diversification of say CDRH2 etc in which the JR's remain untouched.

In general, the term “Complementarity Determining Regions (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (113) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

“Junctional region” “variable framework region” “JR” “VFR” as used herein refers to the contiguous set of three framework residues adjoining a CDR region on at least one side (N or C terminal end of the CDR) or preferably on each side each of a CDR region. More, it refers to the region spanning 3 contiguous framework residues in contact with the CDR on the C- or N-terminal end of the CDR. The three-dimensional structure may be analyzed for solvent accessible amino acid positions as such positions are likely to form a loop and/or provide antigen contact in an antibody variable domain. Some of the solvent accessible positions can tolerate amino acid sequence diversity and others (eg structural positions) will be less diversified. The three dimensional structure of the antibody variable domain may be derived from a crystal structure or protein modeling. In some embodiments, the VFR comprises, consist essentially of, or consists of amino acid positions corresponding to amino acid positions 21-23 and/or 35-37 relative to CDRL1 (24-34) of the light chain variable domain, the positions defined according to Kabat et al., 1991. Likewise, CDRL2 comprises 50-56 and thus the VFR comprises 47-49 and/or 57-59 (L2); CDR L3 comprises positions 89-97 (L3) in the light chain variable and thus the corresponding VFR for L3 includes residues at positions 86-88 and/or 98-100. The same rules are applicable to the CDR's on the heavy chain−([3+-28-30] 31-35 (H1)), 3+50-65 3+(H2) and 3+95-102 3+(H3).

“Antibody fragments” comprise only a portion of an intact antibody, generally including an antigen binding site of the intact antibody and thus retaining the ability to bind antigen. Non-limiting examples of antibody fragments encompassed by the present definition include: (1) the Fab fragment, having VL, CL, VH and CH1 domains having one interchain disulfide bond between the heavy and light chain; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment which consists of a VH domain; (vii) hingeless antibodies including at least VL, VH, CL, CH1 domains and lacking hinge region; (viii) F(ab′).sub.2 fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) single arm antigen binding molecules comprising a light chain, a heavy chain and a N-terminally truncated heavy chain constant region sufficient to form a Fc region capable of increasing the half life of the single arm antigen binding domain; (xii) “linear antibodies” comprising a pair of tandem Fd segments (VH-CH1-VH-CH1) which, together with complementary light chain polypeptides, form a pair of antigen binding regions.

“Framework regions” (hereinafter “FR”) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 3549 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues can be adjusted accordingly. For example, when CDRH1 includes amino acids H26-H35, the heavy chain FR1 residues are at positions 1-25 and the FR2 residues are at positions 36-49

As used herein, “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein. A codon set typically is represented by 3 capital letters in italics, e.g. NNK, NNS, XYZ, DVK and the like. Synthesis of oligonucleotides with selected nucleotide “degeneracy” at certain positions is well known in that art, for example the TRIM approach (Knappek et al.; J. Mol. Biol. (1999), 296:57-86); Garrard & Henner, Gene (1993), 128:103). Such sets of oligonucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can, but does not necessarily, include restriction enzyme sites useful for, for example, cloning purposes.

The term “restricted codon set”, and variations thereof, as used herein refers to a codon set that encodes a much more limited number of amino acids than the codon sets typically utilized in art methods of generating sequence diversity. In one aspect of the invention, restricted codon sets used for sequence diversification encode from 2 to 10, from 2 to 8, from 2 to 6, from 2 to 4, or only 2 amino acids. In some embodiments, a restricted codon set used for sequence diversification encodes at least 2 but 10 or fewer, 8 or fewer, 6 or fewer, 4 or fewer amino acids. Determination of suitable restricted codons, and the identification of specific amino acids encoded by a particular restricted codon, is well known and would be evident to one skilled in the art, Determination of suitable amino acid sets to be used for diversification of a CDR sequence can be empirical and/or guided by criteria known in the art (e.g., inclusion of a combination of hydrophobic and hydrophilic amino acid types, etc.)

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V.sub.H-V.sub.L dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

The “Fab” fragment contains a variable and constant domain of the light chain and a variable domain and the first constant domain (CH1) of the heavy chain. F(ab′).sub.2 antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V.sub.H and V.sub.L domains of antibody, wherein these domains are present in a single polypeptide chain. Generally the Fv polypeptide further comprises a polypeptide linker between the V.sub.H and V.sub.L domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V.sub.H) connected to a light chain variable domain (V.sub.L) in the same polypeptide chain (V.sub.H and V.sub.L). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V.sub.H-C.sub.H1-V.sub.H-C.sub.H1) which, together with complementary light chain polypeptides, form a pair of antigen binding regions. Linear antibodies can be bispecific or monospecific.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The term “monoclonal antibody” as used herein refers to an antibody obtained from a population of substantially homogeneous antibodies, i.e., the individual antibodies comprising the population are essentially identical except for variants that may arise during production of the antibody. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The term “monobody” as used herein, refers to an antigen binding molecule with a heavy chain variable domain and no light chain variable domain. A monobody can bind to an antigen in the absence of light chains and typically has three CDR regions designated CDRH1, CDRH2 and CDRH3. A heavy chain IgG monobody has two heavy chain antigen binding molecules connected by a disulfide bond. The heavy chain variable domain comprises one or more CDR regions, preferably a CDRH3 region. A “VhH” or “VHH” refers to a variable domain of a heavy chain antibody such as a monobody.

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (K.sub.d) value of no more than about 1.times.10.sup.-9 M, for example no more than about 1.times.10.sup.-8 M and as a further example no more than about 1.times.10.sup.-9 M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, for example at least 80%, for example at least 85%, for example at least 90%, and for example at least 95%. Identity or similarity with respect to this sequence is defined herein as the percentage of amino acid residues in the candidate sequence that are identical (i.e. same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity. Biologically active fragments, deletional, insertional, or substitutional variants of the any one of more of the variant antibodies described herein or produced by the methods of the invention are also included.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody as determined by the Lowry method, e.g., to more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “antagonist” is used in the broadest sense, and includes any molecule that partially or fully blocks, inhibits, or neutralizes one or more biological activities of target molecules described herein (e.g. murine Her3, PCSK9 etc.) in vitro, in situ, or in vivo. For instance, the antagonist may function to partially or fully block, inhibit or neutralize one or more biological activities of a ligand of target molecule, in vitro, in situ, or in vivo as a result of its direct binding to the target molecule. The antagonist may also function indirectly to partially or fully block, inhibit or neutralize one or more biological activities of target molecule, in vitro, in situ, or in vivo as a result of, e.g., blocking or inhibiting another effector molecule. The antagonist molecule may comprise a “dual” antagonist activity wherein the molecule is capable of partially or fully blocking, inhibiting or neutralizing a biological activity of target molecule.

The term “agonist” is used in the broadest sense, and includes any molecule that partially or fully enhances, stimulates or activates one or more biological activities of target molecule described herein (e.g. Her3, PCSK9 etc), in vitro, in situ, or in vivo. An agonist may function in a direct or indirect manner. For instance, the agonist may function to partially or fully enhance, stimulate or activate one or more biological activities of the target molecule, in vitro, in situ, or in vivo as a result of its direct binding to the target molecule, which causes receptor activation or signal transduction. The agonist may also function indirectly to partially or fully enhance, stimulate or activate one or more biological activities of the target molecule, in vitro, in situ, or in vivo as a result of, e.g., stimulating another effector molecule which then causes target molecule activation or signal transduction. It is contemplated that an agonist may act as an enhancer molecule which functions indirectly to enhance or increase target molecule activation or activity.

“Cell”, “cell line”, and “cell culture” are used interchangeably herein and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Heterologous DNA” is any DNA that is introduced into a host cell. The DNA may be derived from a variety of sources including genomic DNA, cDNA, synthetic DNA and fusions or combinations of these. The DNA may include DNA from the same cell or cell type as the host or recipient cell or DNA from a different cell type, for example, from a mammal or plant. The DNA may, optionally, include marker or selection genes, for example, antibiotic resistance genes, temperature resistance genes, etc.

As used herein, “library” refers to a plurality of antibody, antibody fragment sequences, or antibody variable domains (for example, polypeptides of the invention), or the nucleic acids that encode these sequences, the sequences being different in the combination of variant amino acids that are introduced into these sequences according to the methods of the invention.

“Oligonucleotides” are short-length, single- or double-stranded polydeoxynucleotide that are prepared by known methods such as chemical synthesis (e.g. phosphotriester, phosphite, or phosphoramidite chemistry, using solid-phase techniques such as described in EP 266,032 published 4 May 1988, or via deoxynucleoside H-phosphonate intermediates as described by Froeshler et al., Nucl. Acids, Res., 14:5399-5407 (1986)). Further methods include the polymerase chain reaction defined below and other autoprimer methods and oligonucleotide syntheses on solid supports. All of these methods are described in Engels et al., Agnew. Chem. Int. Ed. Engl., 28:716-734 (1989). These methods are used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation.

DNA is “purified” when the DNA is separated from non-nucleic acid impurities. The impurities may be polar, non-polar, ionic, etc.

A “source antibody”, as used herein, refers to an antibody or antigen binding polypeptide whose antigen binding determinant sequence serves as the template sequence upon which diversification according to the criteria described herein is performed. An antigen binding determinant sequence generally includes an antibody variable region, preferably at least one CDR, and preferably including at least one framework regions. A source antibody variable domain can include an antibody, antibody variable domain, antigen binding fragment or polypeptide thereof, a monobody, VHH, a monobody or antibody variable domain obtained from a nave or synthetic library, naturally occurring antibody or monobody, synthetic antibody or monobody, recombinant antibody or monobody, humanized antibody or monobody, germline derived antibody or monobody, chimeric antibody or monobody, and affinity matured antibody or monobody.

As used herein, “solvent accessible position” refers to a position of an amino acid residue in the variable region of a heavy and/or light chain of a source antibody or antigen binding polypeptide that is determined, based on structure, ensemble of structures and/or modeled structure of the antibody or antigen binding polypeptide, as potentially available for solvent access and/or contact with a molecule, such as an antibody-specific antigen. These positions are typically found in the CDRs, but can also be found in FR and on the exterior of the protein. The solvent accessible positions of an antibody or antigen binding polypeptide, as defined herein, can be determined using any of a number of algorithms known in the art. Preferably, solvent accessible positions are determined using coordinates from a 3-dimensional model of an antibody or antigen binding polypeptide, preferably using a computer program such as the InsightII program (Accelrys, San Diego, Calif.). Solvent accessible positions can also be determined using algorithms known in the art (e.g., Lee and Richards, J. Mol. Biol. 55, 379 (1971) and Connolly, J. Appl. Cryst. 16, 548 (1983)). Determination of solvent accessible positions can be performed using software suitable for protein modeling and 3-dimensional structural information obtained from an antibody. Software that can be utilized for these purposes includes SYBYL Biopolymer Module software (Tripos Associates). Generally and preferably, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers.” Comput. Chem. 18(4): 377-386; and (1995). “Variations of Surface Areas and Volumes in Distinct Molecular Surfaces of Biomolecules.” J. Mol. Model. 1: 46-53.)

The phrase “structural amino acid position” as used herein refers to an amino acid of a polypeptide that contributes to the stability of the structure of the polypeptide such that the polypeptide retains at least one biological function such as specifically binding to a molecule such as an antigen and/or binds to a target molecule that binds to folded polypeptide and does not bind to unfolded polypeptide such as Protein A. Structural amino acid positions are identified as amino acid positions less tolerant to amino acid substitutions without affecting the structural stability of the polypeptide. Amino acid positions less tolerant to amino acid substitutions can be identified using a method such as alanine scanning mutagenesis or shotgun scanning as described in WO 01/44463 and analyzing the effect of loss of the wild type amino acid on structural stability. If a wild type amino acid is replaced with a scanning amino acid in a position, and the resulting variant exhibits poor binding to a target molecule that binds to folded polypeptide, then that position is important to maintaining the structure of the polypeptide. A structural amino acid position is a position in which, preferably, the ratio of polypeptides with wild type amino acid at a position to a variant substituted with a scanning amino acid at that position is at least about 3 to 1, about 5 to 1, about 5 to 1, about 10 to 1 or greater. In a further embodiment, structural amino acid positions are positions that have a weighted hydrophobicity value of greater than −0.5 as determined using the method of Kyte and Doolittle (cited supra) when the population is randomized in the VFR or CDRs. For example, in one embodiment the boundaries of CDR1 in the heavy chain are selected at amino acids 24 and 34 as these positions show a strong preference for hydrophobes.

The term “stability” as used herein refers to the ability of a molecule to maintain a folded state under physiological conditions such that it retains at least one of its normal functional activities, for example, binding to an antigen or to a molecule like Protein A. The stability of the molecule can be determined using standard methods. For example, the stability of a molecule can be determined by measuring the thermal melt (“TM”) temperature. The TM is the temperature in degrees Celsius at which ½ of the molecules become unfolded. Typically, the higher the TM, the more stable the molecule.

The phrase “randomly generated population” as used herein refers to a population of polypeptides wherein one or more amino acid positions in a domain has a variant amino acid encoded by a random codon set which allows for substitution of all 20 naturally occurring amino acids at that position. For example, in one embodiment, a randomly generated population of polypeptides having randomized VFR or portions thereof include a variant amino acid at each position in VFR that is encoded by a random codon set.

A “functional epitope” according to this invention refers to amino acid residues of an antigen that contribute energetically to the binding of an antibody. Mutation of any one of the energetically contributing residues of the antigen (for example, mutation of wild-type VEGF by alanine or homolog mutation) will disrupt the binding of the antibody such that the relative affinity ratio (IC₅₀mutant VEGFIC50wild-type VEGF) of the antibody will be greater than 5 (see Example 2). In a preferred embodiment, the relative affinity ratio is determined by a solution binding phage displaying ELISA. Briefly, 96-well Maxisorp immunoplates (NUNC) are coated overnight at 4.degree. C. with an Fab form of the antibody to be tested at a concentration of 2 μg/ml in PBS, and blocked with PBS, 0.5% BSA, and 0.05% Tween20 (PBT) for 211 at room temperature. Serial dilutions of phage displaying hVEGF alanine point mutants (residues 8-109 form) or wild type hVEGF (8-109) in PBT are first incubated on the Fab-coated plates for 15 min at room temperature, and the plates are washed with PBS, 0.05% Tween20 (PBST). The bound phage is detected with an anti-M13 monoclonal antibody horseradish peroxidase (Amersham Pharmacia) conjugate diluted 1:5000 in PBT, developed with 3,3′, 5,5′-tetramethylbenzidine (TMB, Kirkegaard & Perry Labs, Gaithersburg, M D) substrate for approximately 5 min, quenched with 1.0 M H.sub.3PO.sub.4, and read spectrophotometrically at 450 nm. The ratio of IC50 values (IC50,ala/IC50,wt) represents the fold of reduction in binding affinity (the relative binding affinity).

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell. The coat protein may be the major coat protein or may be a minor coat protein. A “major” coat protein is generally a coat protein which is present in the viral coat at least about 5, at least about 7, at least about 10 copies of the protein or more. A major coat protein may be present in tens, hundreds or even thousands of copies per virion. An example of a major coat protein is the p8 protein of filamentous phage.

The “detection limit” for a chemical entity in a particular assay is the minimum concentration of that entity which can be detected above the background level for that assay. For example, in the phage ELISA, the “detection limit” for a particular phage displaying a particular antigen binding fragment is the phage concentration at which the particular phage produces an ELISA signal above that produced by a control phage not displaying the antigen binding fragment.

As used herein “Vh3” refers to a subgroup of antibody variable domains. The sequences of known antibody variable domains have been analyzed for sequence identity and divided into groups. Antibody heavy chain variable domains in subgroup III are known to have a Protein A binding site.

A “plurality” or “population” of a substance, such as a polypeptide or polynucleotide of the invention, as used herein, generally refers to a collection of two or more types or kinds of the substance. There are two or more types or kinds of a substance if two or more of the substances differ from each other with respect to a particular characteristic, such as the variant amino acid found at a particular amino acid position. For example, there is a plurality or population of polypeptides of the invention if there are two or more polypeptides of the invention that are substantially the same, preferably identical, in sequence except for the sequence of a variant VFR or except for the variant amino acid at a particular solvent accessible amino acid position. In another example, there is a plurality or population of polynucleotides of the invention if there are two or more polynucleotides of the invention that are substantially the same, preferably identical, in sequence except for the sequence that encodes a variant VFR or except for the sequence that encodes a variant amino acid for a particular solvent accessible acid position or structural amino acid position.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. In certain embodiments, the two portions of the polypeptide are obtained from heterologous or different polypeptides.

As used herein, “highly diverse position” refers to a position of an amino acid located in the variable regions of the light and heavy chains that have a number of different amino acids represented at the position when the amino acid sequences of known and/or naturally occurring antibodies or antigen binding fragments are compared. The highly diverse positions are typically in the CDR regions. In one aspect, the ability to determine highly diverse positions in known and/or naturally occurring antibodies is facilitated by the data provided by Kabat, Sequences of Proteins of Immunological Interest (National Institutes of Health, Bethesda, Md., 1987 and 1991). An internet-based database located at http/www.bioinforg.uk.abs.structures.html provides an extensive collection and alignment of light (http/www.bioinf org.uk.abs.lc.align and heavy chain (http/www.bioinf.org.uk.abs.hc.align sequences and facilitates determination of highly diverse positions in these sequences. According to the invention, an amino acid position is highly diverse if it has from about 2 to about 11, from about 4 to about 9, and/or from about 5 to about 7 different possible amino acid residue variations at that position. In some embodiments, an amino acid position is highly diverse if it has at least about 2, at least about 4, at least about 6, and/or at least about 8 different possible amino acid residue variations at that position.

As used herein, “library” refers to a plurality of antibody or antibody fragment sequences (for example, polypeptides of the invention), or the nucleic acids that encode these sequences, the sequences being different in the combination of variant amino acids that are introduced into these sequences according to the methods of the invention.

A “mutation” is a deletion, insertion, or substitution of a nucleotide(s) relative to a reference nucleotide sequence, such as a wild type sequence.

As used herein, “natural” or “naturally occurring” antibodies, refers to antibodies identified from a nonsynthetic source, for example, from a differentiated antigen-specific B cell obtained ex vivo, or its corresponding hybridoma cell line, or from antibodies obtained from the serum of an animal. These antibodies can include antibodies generated in any type of immune response, either natural or otherwise induced. Natural antibodies include the amino acid sequences, and the nucleotide sequences that constitute or encode these antibodies, for example, as identified in the Kabat database. As used herein, natural antibodies are different than “synthetic antibodies”, synthetic antibodies referring to antibody sequences that have been changed from a source or template sequence, for example, by the replacement, deletion, or addition, of an amino acid, or more than one amino acid, at a certain position with a different amino acid, the different amino acid providing an antibody sequence different from the source antibody sequence.

“Phage display” is a technique by which variant polypeptides are displayed as fusion proteins to at least a portion of coat protein on the surface of phage, e.g., filamentous phage, particles. A utility of phage display lies in the fact that large libraries of randomized protein variants can be rapidly and efficiently sorted for those sequences that bind to a target antigen with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods. A companion to Methods in Enzymology, 3:205-0216 (1991).

A “phagemid” is a plasmid vector having a bacterial origin of replication, e.g., Co1E1, and a copy of an intergenic region of a bacteriophage. The phagemid may be used on any known bacteriophage, including filamentous bacteriophage and lambdoid bacteriophage.

The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle.

The term “phage vector” means a double stranded replicative form of a bacteriophage containing a heterologous gene and capable of replication. The phage vector has a phage origin of replication allowing phage replication and phage particle formation. In certain embodiments, the phage is a filamentous bacteriophage, such as an M13, fl, fd, Pf3 phage or a derivative thereof, or a lambdoid phage, such as lambda, 21, phi80, phi81, 82, 424, 434, etc., or a derivative thereof.

A “transcription regulatory element” will contain one or more of the following components: an enhancer element, a promoter, an operator sequence, a repressor gene, and a transcription termination sequence. These components are well known in the art. U.S. Pat. No. 5,667,780.

A “transformant” is a cell which has taken up and maintained DNA as evidenced by the expression of a phenotype associated with the DNA (e.g., antibiotic resistance conferred by a protein encoded by the DNA).

“Transformation” means a process whereby a cell takes up DNA and becomes a “transformant”. The DNA uptake may be permanent or transient.

An “affinity matured” antibody is one with one or more alterations in one or more CDRs thereof which result in an improvement in the affinity of the antibody for antigen, compared to a parent antibody which does not possess those alteration(s). In certain embodiments, affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

The “Kd” or “Kd value” is the dissociation constant for the interaction of one molecule with another. In one embodiment, the Kd value is measured by a radiolabeled protein binding assay (RIA). In one embodiment, an RIA for a her3 or PCSK9 or Trk can be performed with the Fab version of an anti-her3 or PCSK9 antibody or any other antibody and a her3 or PCSk9 molecule respectively as described by the following assay that measures solution binding affinity of Fabs for her3 or PCSk9 by equilibrating a Fab with a minimal concentration of (.sup.125I)-labeled Her3 or PCSk9 in the presence of a titration series of unlabeled her3 or PCSK9 molecule respectively, then capturing bound Her3 or PCSK9 molecule respectively with an anti-Fab antibody-coated plate (Chen, et al., (1999) J. Mol. Biol. 293:865-881). To establish conditions for the assay, microtiter plates (Dynex) are coated overnight with 5 .mu·g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23.degree. C.). In a non-absorbent plate (Nunc #269620), 100 pM or 26 pM [.sup.125I] her3 or PCSK9 are mixed with serial dilutions of a Fab of interest, e.g., Fab-12 (Presta et al., (1997) Cancer Res. 57:4593-4599), The Fab of interest is then incubated overnight; however, the incubation may continue for 65 hours to insure that equilibrium is reached. Thereafter, the mixtures are transferred to the capture plate for incubation at room temperature for one hour. The solution is then removed and the plate washed eight times with 0.1% Tween-20 in PBS. When the plates had dried, 150 .mu·l/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays.

According to another embodiment the Kd or Kd value can be measured by using surface plasmon resonance assays using a BIAcore™-2000 or a BIAcore™-3000 instrument (BIAcore, Inc., Piscataway, N.J.). In certain embodiments, the Kd value of anti-her3 or PCSk9 molecule antibodies for her3 or PCSK9 molecule respectively is determined using BIAcore™ analysis according to the following protocol. Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Human or murine her3 or PCSK9 molecule is diluted with 10 mM sodium acetate, pH 4.8, to 5 .mu·g/ml (.about.0.2 .mu·M) before injection at a flow rate of 5 .mu·l/minute to achieve approximately 10 response units (RU) of coupled protein. Following the injection of human her3 or PCSK9, 1M ethanolamine is injected to block unreacted groups. For kinetics measurements, two-fold serial dilutions of Fab (0.78 nM to 500 nM) are injected in PBS with 0.05% Tween 20 (PBST) at 25.degree. C. at a flow rate of approximately 25 .mu·l/min. Association rates (k.sub.on) and dissociation rates (k.sub.off) are calculated using a simple one-to-one Langmuir binding model (BIAcore Evaluation Software version 3.2) by simultaneously fitting the association and dissociation sensorgram. The equilibrium dissociation constant (Kd) was calculated as the ratio k.sub.off/k.sub.on. See, e.g., Chen, Y., et al., (1999) J. Mol. Biol. 293:865-881.

A “disorder” is any condition that would benefit from treatment with a substance/molecule or method of the invention. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic related disorders.

The term abnormal angiogenesis occurs when new blood vessels either grow excessively, insufficiently or inappropriately (e.g., the location, timing or onset of the angiogenesis being undesired from a medical standpoint) in a diseased state or such that it causes a diseased state. Excessive, inappropriate or uncontrolled angiogenesis occurs when there is new blood vessel growth that contributes to the worsening of the diseased state or causes a diseased state, such as in cancer, especially vascularized solid tumors and metastatic tumors (including colon, lung cancer (especially small-cell lung cancer), or prostate cancer), diseases caused by ocular neovascularisation, especially diabetic blindness, retinopathies, primarily diabetic retinopathy or age-induced macular degeneration and rubeosis; psoriasis, psoriatic arthritis, haemangioblastoma such as haemangioma; inflammatory renal diseases, such as glomerulonephritis, especially mesangioproliferative glomerulonephritis, haemolytic uremic syndrome, diabetic nephropathy or hypertensive neplirosclerosis; various imflammatory diseases, such as arthritis, especially rheumatoid arthritis, inflammatory bowel disease, psorsasis, sarcoidosis, arterial arteriosclerosis and diseases occurring after transplants, endometriosis or chronic asthma and more than 70 other conditions. The new blood vessels can feed the diseased tissues, destroy normal tissues, and in the case of cancer, the new vessels can allow tumor cells to escape into the circulation and lodge in other organs (tumor metastases). Insufficient angiogenesis occurs when there is inadequate blood vessels growth that contributes to the worsening of a diseased state, e.g., in diseases such as coronary artery disease, stroke, and delayed wound healing. Further, ulcers, strokes, and heart attacks can result from the absence of angiogenesis that normally required for natural healing. The present invention contemplates treating those patients that are at risk of developing the above-mentioned illnesses.

Other patients that are candidates for receiving the antibodies or polypeptides of this invention have, or are at risk for developing, abnormal proliferation of fibrovascular tissue, acne rosacea, acquired immune deficiency syndrome, artery occlusion, atopic keratitis, bacterial ulcers, Bechets disease, blood bone tumors, carotid obstructive disease, choroidal neovascularization, chronic inflammation, chronic retinal detachment, chronic uveitis, chronic vitritis, contact lens overwear, corneal graft rejection, corneal neovascularization, corneal graft neovascularization, Crohn's disease, Bales disease, epidemic keratoconjunctivitis, fungal ulcers, Herpes simplex infections, Herpes zoster infections, hyperviscosity syndromes, Kaposi's sarcoma, leukemia, lipid degeneration, Lyme's disease, marginal keratolysis, Mooren ulcer, Mycobacteria infections other than leprosy, myopia, ocular neovascular disease, optic pits, Osler-Weber syndrome (Osler-Weber-Rendu, osteoarthritis, Pagets disease, pars planitis, pemphigoid, phylectenulosis, polyarteritis, post-laser complications, protozoan infections, pseudoxanthoma elasticum, pterygium keratitis sicca, radial keratotomy, retinal neovascularization, retinopathy of prematurity, retrolental fibroplasias, sarcoid, scleritis, sickle cell anemia, Sogrens syndrome, solid tumors, Stargarts disease, Steven's Johnson disease, superior limbic keratitis, syphilis, systemic lupus, Terrien's marginal degeneration, toxoplasmosis, trauma, tumors of Ewing sarcoma, tumors of neuroblastoma, tumors of osteosarcoma, tumors of retinoblastoma, tumors of rhabdomyosarcoma, ulcerative colitis, vein occlusion, Vitamin A deficiency and Wegeners sarcoidosis, undesired angiogenesis associated with diabetes, parasitic diseases, abnormal wound healing, hypertrophy following surgery, injury or trauma, inhibition of hair growth, inhibition of ovulation and corpus luteum formation, inhibition of implantation and inhibition of embryo development in the uterus.

Anti-angiogenesis therapies are useful in the general treatment of graft rejection, lung inflammation, nephrotic syndrome, preeclampsia, pericardial effusion, such as that associated with pericarditis, and pleural effusion, diseases and disorders characterized by undesirable vascular permeability, e.g., edema associated with brain tumors, ascites associated with malignancies, Meigs' syndrome, lung inflammation, nephrotic syndrome, pericardial effusion, pleural effusion, permeability associated with cardiovascular diseases such as the condition following myocardial infarctions and strokes and the like.

Other angiogenesis-dependent diseases according to this invention include angiofibroma (abnormal blood of vessels which are prone to bleeding), neovascular glaucoma (growth of blood vessels in the eye), arteriovenous malformations (abnormal communication between arteries and veins), nonunion fractures (fractures that will not heal), atherosclerotic plaques (hardening of the arteries), pyogenic granuloma (common skin lesion composed of blood vessels), scleroderma (a form of connective tissue disease), hemangioma (tumor composed of blood vessels), trachoma (leading cause of blindness in the third world), hemophilic joints, vascular adhesions and hypertrophic scars (abnormal scar formation).

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

The term “immune related disease” means a disease in which a component of the immune system of a mammal causes, mediates or otherwise contributes to morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or I cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimotols thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barc syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohns disease), gluten-sensitive enteropathy, and Whipples disease, autoimmune or immune-mediated skin diseases including bullous skin diseases, erythema multiform and contact dermatitis, psoriasis, allergic diseases such as asthma, allergic rhinitis, atopic dermatitis, food hypersensitivity and urticaria, and immunologic diseases of the lung such as eosinophilic pneumonias.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

“Mammal” for purposes of treatment refers to any animal classified as a mammal, including humans, domestic and farm animals, nonhuman primates, and zoo, sports, or pet animals, such as dogs, horses, cats, cows, etc.

The term “anti-neoplastic composition” refers to a composition useful in treating cancer comprising at least one active therapeutic agent, e.g., “anti-cancer agent.” Examples of therapeutic agents (anti-cancer agents) include, but are not limited to, e.g., chemotherapeutic agents, growth inhibitory agents, cytotoxic agents, agents used in radiation therapy, anti-angiogenesis agents, apoptotic agents, anti-tubulin agents, and other-agents to treat cancer such as, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the targets molecule and other bioactive and organic chemical agents, etc. Combinations thereof are also included in the invention.

The term “epitope tagged” when used herein refers to an antibody mutant fused to an “epitope tag”. The epitope tag polypeptide has enough residues to provide an epitope against which an antibody thereagainst can be made, yet is short enough such that it does not interfere with activity of the antibody mutant. The epitope tag preferably also is fairly unique so that the antibody thereagainst does not substantially cross-react with other epitopes. Suitable tag polypeptides generally have at least 6 amino acid residues and usually between about 8-50 amino acid residues (in certain embodiments between about 9-30 residues). Examples include, but are not limited to, the flu HA tag polypeptide and its antibody 12CA5 (Field et al. Mol. Cell. Biol. 8:2159-2165 (1988)); the c-myc tag and the 8F9, 3C7, 6E10, G4, B7 and 9E10 antibodies thereagainst (Evan et al., Mol. Cell. Biol. 5(12):3610-3616 (1985)); and the Herpes Simplex virus glycoprotein D (gD) tag and its antibody (Paborsky et al., Protein Engineering 3(6):547-553 (1990)). In certain embodiments, the epitope tag is a “salvage receptor binding epitope”.

The term “cytotoxic agent” as used herein refers to a substance that inhibits or prevents the function of cells and/or causes destruction of cells. The term is intended to include radioactive isotopes (e.g., At.sup.211, I.sup.131, I.sup.125, Y.sup.90, Re.sup.186, Re.sup.188, Sm.sup.153, Bi.sup.212, P.sup.32 and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin I and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma II and calicheamicin omegaI1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN®(doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL®paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “variant” or “mutant” of a starting or reference polypeptide (e.g., a source antibody or its variable domain(s)/CDR(s)), such as a fusion protein (polypeptide) or a heterologous polypeptide (heterologous to a phage), is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a restricted codon set that encodes a sequence with a variant amino acid (with respect to the amino acid found at the corresponding position in a source antibody/antigen binding fragment) would be a variant polypeptide with respect to a source antibody and/or antigen binding fragment and/or CDR/VFR. Thus, a variant CDR refers to a CDR comprising a variant sequence with respect to a starting or reference polypeptide sequence (such as that of a source antibody and/or antigen binding fragment and/or CDR). Likewise, a variant “Junctional region” mutant comprises a variant sequence with respect to a starting or reference polypeptide sequence (such as that of a source antibody and/or antigen binding fragment and/or Framework sequence). A variant amino acid, in this context, refers to an amino acid different from the amino acid at the corresponding position in a starting or reference polypeptide sequence (such as that of a source antibody and/or antigen binding fragment and/or CDR). Any combination of deletion, insertion, and substitution may be made to arrive at the final variant or mutant construct, provided that the final construct possesses the desired functional characteristics. In some of the examples described herein, binder sequences contain point mutations such as deletions or additions. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

In one aspect, the restricted repertoire of amino acids intended to occupy one or more of the highly diverse positions in one of more of the VFR's or CDRs of a target antibody, e.g., PCSK9 or her3 are determined (based on the desire of the practitioner, which can be based on any of a number of criteria, including specific amino acids desired for particular positions, specific amino acid(s) desired to be absent from a particular position, size of library desired, characteristic of antigen binders sought, etc.).

Heavy chain CDR3s (CDRH3s) in known antibodies have diverse sequences, structural conformations, and lengths. CDRH3s are often found in the middle of the antigen binding pocket and often participate in antigen contact. The design of CDRH3 may be developed separately from that of the other CDRs because it can be difficult to predict the structural conformation of CDRH3 and the amino acid diversity in this region is especially diverse in known antibodies. In accordance with the present invention, CDRH3 is designed to generate diversity at specific positions within CDRH3, for e.g., positions X1, X2, X3 (for e.g., according to Kabat numbering). In some embodiments, diversity is also generated by varying CDRH3 length using restricted codon sets. Length diversity can be of any range determined empirically to be suitable for generating a population of polypeptides containing substantial proportions of antigen binding proteins. For example, polypeptides comprising variant CDRH3 can be generated having the sequence (X1)_{.n-A-M}, wherein X1 is an amino acid encoded by a restricted codon set, and n is of various lengths, for example, n=3-20, 5-20, 7-20, 5-18 or 7-18. Other examples of possible n values are 5, 6, 7, 8, 9, 10, 11, 12, 13, 14, 15, 16, 17, 18, 19 and 20. Illustrative embodiments of oligonucleotides are detailed in the experimental section below. The same applied to the design of the VFR or junctional regions.

The inventors contemplate that the sequence diversity of libraries created by introduction of variant amino acids in a particular Junctional Region in addition to or alternative to a particular CDR, for e.g., CDRH1, 2 or 3, can be increased by combining the variant CDR or JR with other CDRs comprising variations in other regions of the antibody, specifically in other CDRs of either the light or heavy chain variable sequences or other junctional regions. It is contemplated that the nucleic acid sequences that encode members of this set can be further diversified by introduction of other variant amino acids in the CDRs of either the light or heavy chain sequences, via codon sets or junctional regions. Thus, for example, in one embodiment, CDRH3 sequences or the attendant JR in contact with the CDRH3 from fusion polypeptides that bind a target antigen can be combined with diversified CDRL3, CDRH1, or CDRH2 sequences, or any combination of diversified CDRs or variant JR's/VFR's.

It should be noted that contrary to conventional wisdom, in certain embodiments, framework residues in a junctional region (VFR/JR) may be varied relative to the sequence of a source antibody or antigen binding fragment, for example, to reflect a consensus sequence or to improve stability or display. Heavy chain framework residues may be changed to reflect framework consensus sequence.

Typically, a codon set is represented by three capital letters eg. KMT, TMT and the like. TUB Codes

G Guanine

A Adenine

T Thymine

C Cytosine

R (A or G)

Y (C or T)

M (A or C)

K (G or T)

S(C or G)

W (A or T)

H (A or C or T)

B (C or G or T)

V (A or C or O)

D (A or G or T)

N (A or C or G or T)

For example, in the codon set TMT, T is the nucleotide thymine; and M can be A or C. This codon set can present multiple codons and can encode only a limited number of amino acids, namely tyrosine and serine.

In one aspect, the invention provides vector constructs for generating fusion polypeptides that bind with significant affinity to potential ligands. These constructs may further comprise a dimerizable domain that when present in a fusion polypeptide provides for increased tendency for heavy chains to dimerize to form dimers of Fab or Fab′ antibody fragments/portions. These dimerization domains may include, eg. a heavy chain hinge sequence that may be present in the fusion polypeptide. Dimerization domains in fusion phage polypeptides bring two sets of fusion polypeptides (LC/HC-phage protein/fragment (such as pIII)) together, thus allowing formation of suitable linkages (such as interheavy chain disulfide bridges) between the two sets of fusion polypeptide. Vector constructs containing such dimerization domains can be used to achieve divalent display of antibody variable domains, for example the diversified fusion proteins described herein, on phage. Preferably, the intrinsic affinity of each monomeric antibody fragment (fusion polypeptide) is not significantly altered by fusion to the dimerization domain. Preferably, dimerization results in divalent phage display which provides increased avidity of phage binding, with significant decrease in off-rate, which can be determined by methods known in the art and as described herein. Dimerization domain-containing vectors of the invention may or may not also include an amber stop codon after the dimerization domain.

A “wild type” or “reference” sequence or the sequence of a “wild type” or “reference” protein/polypeptide, such as a coat protein, or a CDR or variable domain of a source antibody, maybe the reference sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

A “plurality” of a substance, such as a polypeptide or polynucleotide of the invention, as used herein, generally refers to a collection of two or more types or kinds of the substance. There are two or more types or kinds of a substance if two or more of the substances differ from each other with respect to a particular characteristic, such as the variant amino acid found at a particular amino acid position. For example, there is a plurality of polypeptides of the invention if there are two or more polypeptides of the invention that are substantially the same, or are identical in sequence except for the sequence of a variant CDR or except for the variant amino acid at a particular solvent accessible and highly diverse amino acid position. In another example, there is a plurality of polynucleotides of the invention if there are two or more polynucleotides of the invention that are substantially the same or identical in sequence except for the sequence that encodes a variant CDR and/or Junctional region.

The invention provides methods for generating and isolating novel target antigen binding polypeptides, such as antibodies or antigen binding fragments, that can have a high affinity for a selected antigen. A plurality of different binder polypeptides are prepared by mutating (diversifying) one or more selected amino acid positions in a source antibody light chain variable domain and/or heavy chain variable domain with restricted codon sets to generate a library of binder polypeptides with variant amino acids in at least one CDR sequence, wherein the number of types of variant amino acids is kept to a minimum (i.e., 19 or fewer, 15 or fewer, 10 or fewer, 8 or fewer, 6 or fewer, 4 or fewer, or only 2, but generally at least 2). The amino acid positions include those that are solvent accessible, for example as determined by analyzing the structure of a source antibody, and/or that are highly diverse among known and/or natural occurring immunoglobulin polypeptides. A further advantage afforded by the limited nature of diversification of the invention is that additional amino acid positions other than those that are highly diverse and/or solvent accessible can also be diversified in accordance with the need or desire of the practitioner; examples of these embodiments are described herein.

The diversity of the library of the polypeptides comprising variant CDRs and/or junctional regions may be designed using codon sets that encode only a limited number of amino acids, such that a minimum but sufficient amount of sequence diversity is introduced into a CDR or Junctional region. The number of positions mutated in the CDR or Junctional region is minimized and the variant amino acids at each position are designed to include a limited number of amino acids, independent of the amino acids that deemed to be commonly occurring at that position in known and/or naturally occurring CDRs or framework sequences. In certain embodiments, a single antibody, including at least one CDR and one Framework sequence, is used as the source antibody. It is surprising that a library of antibody variable domains having diversity in sequences and size can be generated using a single source antibody as a template and targeting diversity to particular positions using an unconventionally limited number of amino acid substitutions.

### MODES OF THE INVENTION

A diverse library of antibody variable domains particular variant CDRs and/or variant JR's is useful to identify novel antigen binding molecules having high affinity. Generating a library with antibody variable domains with a high level of diversity and that are structurally stable allows for the isolation of high affinity binders and for antibody variable domains that can more readily be produced in cell culture on a large scale. The present invention is based on the showing that regions of an antibody variable domain particularly a JR region with or without the attendant CDR with which it is in contact with can be varied, preferably utilizing one of a targeted or random approach.

In one aspect of the invention, high quality libraries of antibody variable domains are generated. The libraries have restricted diversity of different sequences of JR and optionally CDR sequences, for example, diversity of the antibody variable domains. The libraries include high affinity binding antibody variable domains for one or more antigens, including, for example, human PCSK9NCBI Reference Sequence: NP_{—}777596.2), murine PCSK9 (NCBI Reference Sequence: NP_{—}705793.1), mouse IL-13 (R) (GenBank: AAB50695.1); Her3 (NCBI Reference Sequence: NP_{—}001973.2); mouse EphA2 (NCBI Reference Sequence: NP_{—}034269.2), Human herpesvirus 1 glycoprotein E (g) (NCBI Reference Sequence: NP_{—}044538.1); TrkA (GenBank: BAA34355.1). In certain aspects, the diversity in the library is designed by selecting highly diverse amino acid positions within a target JR and optionally a target CDR in a single source antibody and mutating those positions in at least one JR, and optionally at least one CDR using restricted codon sets. The restricted codon set can in certain embodiments encode fewer than 19, 15, 10, 8, 6, or 4 amino acids, or encodes only 2 amino acids.

While the source antibodies disclosed herein include any one or more of the above referenced antibodies, the methods for diversification can be applied to other source antibodies whose sequence is known. A source antibody can be a naturally occurring antibody, synthetic antibody, recombinant antibody, humanized antibody, germ line derived antibody, chimeric antibody, affinity matured antibody, or antigen binding fragment thereof. The antibodies can be obtained from a variety of mammalian species including humans, mice and rats.

In some embodiments, a source antibody is an antibody that is obtained after one or more initial affinity screening rounds, but prior to an affinity maturation step(s). A source antibody may be selected or modified to provide for high yield and stability when produced in cell culture.

1. Generating Diversity in Junctional Region (JR's/VFRs)

High quality polypeptide libraries of antibody variable domains may be generated by diversifying a heavy chain variable framework residues corresponding to a junctional region as defined herein (JR_{N} or JR_{C}), and optionally one or more CDRs, of a source antibody or antibody fragment. Thus, each CDR includes or is connected to at least one JR, preferably two JR's, each JR comprising no more than 3 contiguous amino acids. The polypeptide libraries comprise a plurality of variant polypeptides comprising at least one CDR flanked by at least one of a JR_{N} and/or a JR_{C}, preferably each of a JR_{N} and a JR_{C}. Preferably, the JR and/or CDR is designed to provide for amino acid sequence diversity at certain positions while minimizing structural perturbations.

The diversity of the library or population of the antibody variable domains is designed to maximize diversity while minimizing structural perturbations of the antibody variable domain to provide for increased ability to isolate high affinity antibodies. The number of positions mutated in the antibody variable domain, e.g., JR_{N} and/or JR_{C} and/or CDR is minimized or specifically targeted. In some embodiments, structural amino acid positions may be identified and diversity may be minimized or maximized at those positions to ensure a well-folded polypeptide. Preferably, a single antibody or antigen binding polypeptide including at least one CDR and/or FW with at least one JR, is used as the source polypeptide.

The source polypeptide may be any antibody, antibody fragment, or antibody variable domain whether naturally occurring or synthetic. A polypeptide or source antibody variable domain can include an antibody, antibody variable domain, antigen binding fragment or polypeptide thereof, a monobody, VHH, a monobody or antibody variable domain obtained from a naive or synthetic library, naturally occurring antibody or monobody, synthetic antibody or monobody, recombinant antibody or monobody, humanized antibody or monobody, germline derived antibody or monobody, chimeric antibody or monabody, and affinity matured antibody or monabody.

Source antibody variable domains include but are not limited to antibody variable domains previously used to generate phage display libraries, such as VHH-RIG, VHH-VLK, VHH-LLR, and VHH-RLV (Bond et al., 2003, J. MA Biol., 332:643-655), and humanized antibodies or antibody fragments of known antibodies, eg. Her3, PCSK9 or any other polypeptide antigens disclosed herein. The sequences of many source antibody domains are know to those of skill in the art. In an embodiment, the library is generated using the heavy chain variable domain (VHH) of a monobody. The small size and simplicity make monobodies attractive scaffolds for peptidomimetic and small molecule design, as reagents for high throughput protein analysis, or as potential therapeutic agents. The diversified VHH domains are useful, inter alia, in the design of enzyme inhibitors, novel antigen binding molecules, modular binding units in bispecific or intracellular antibodies, as binding reagents in protein arrays, and as scaffolds for presenting constrained peptide libraries.

One criterion for generating diversity in the polypeptide library is selecting amino acid positions that may form an antigen binding pocket or groove in a single source antibody variable domain. One way of determining whether the amino acid position is part of an antigen binding site is to examine the three dimensional structure of the antibody variable domain, for example, for solvent accessible positions. If such information is available, amino acid positions that are in proximity to the antigen can also be determined.

Another criterion for selecting positions to be mutated are those positions which show variability in amino acid sequence when the sequences of known and/or natural antibodies are compared. A highly diverse position refers to a position of an amino acid located in the variable regions of the light or heavy chains that have a number of different amino acids represented at the position when the amino acid sequences of known and/or natural antibodies/antigen binding fragments are compared. The highly diverse positions are either in the JR and/or in the CDR regions. The positions of CDRH3 are all considered highly diverse.

JR and CDR diversity need not be limited at structural amino acid positions. A structural amino acid position refers to an amino acid position in a JR or CDR of a polypeptide that contributes to the stability of the structure of the polypeptide such that the polypeptide retains at least one biological function such as specifically binding to a molecule such as an antigen, or preferably, specifically binds to a target molecule that binds to folded polypeptide and does not bind to unfolded polypeptide such as Protein A. Structural amino acid positions of a JR of CDR are identified as amino acid positions less tolerant to amino acid substitutions without affecting the structural stability of the polypeptide.

In some embodiments, a plurality of antibody variable domains is generated, wherein each antibody variable domain comprises a variant JR region alone or in addition to a variant CDR. As noted supra, JR comprises at least 1 amino acid or up to 3 amino acids located at the N- or C-terminal of each CDR. The number of substituted amino acids may be less than all 20 amino acids, preferably 1 to 6 different amino acids. In some embodiments, the amino acids substituted in a CDR loop may not include cysteine.

In one aspect of the invention, the highly diverse residues in at least one, two, three, four, five or all of CDRs selected from the group consisting of CDRL1, CDRL2, CDRL3, CDRH1, CDRH2, CDRH3, and mixtures thereof are mutated (i.e., randomized using restricted codon sets as described herein). In one aspect of the invention, the highly diverse framework residues in at least one, two, three, four, five or all of JR's and mixtures thereof are mutated (i.e., randomized or targeted). For example, a population of polypeptides may be generated by diversifying at least one framework residue in a JR—either at the C or N-terminal of a CDR and/or highly diverse residue in CDRL3 and CDRH3. Accordingly, the invention provides for a large number of novel antibody sequences formed by replacing at least one framework residue in at least one or more JR's and highly diverse position of at least one or more CDRs of the source antibody variable domain with variant amino acids encoded their respective codons.

As discussed above, the variant amino acids are encoded by restricted codon sets. A codon set is a set of different nucleotide triplet sequences which can be used to form a set of oligonucleotides used to encode the desired group of amino acids. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

Typically, when the variant JR is inserted into a source or wild type framework region, the variant VFR replaces all or a part of the source or wild type framework region which it replaces. The location of insertion of the JR can be determined by comparing the location of JR's in naturally occurring antibody variable domains.

It is contemplated that the sequence diversity of libraries created by introduction of variant amino acids in a particular CDR, for e.g., CDRH3, can be increased by combining the variant CDR with other CDRs comprising variations in other regions of the antibody, specifically in other CDRs of either the light or heavy chain variable sequences or a combination with a particular variant JR. It is contemplated that the nucleic acid sequences that encode members of this set can be further diversified by introduction of other variant amino acids in the CDRs of either the light or heavy chain sequences, via codon sets or in the JR's. Thus, for example, in an exemplary embodiment, CDRH3 sequences from fusion polypeptides that bind a target antigen can be combined with diversified CDRL3, CDRH1, or CDRH2 sequences or variant JR from either chain, or any combination of diversified CDRs or JR's.

In another embodiment, JR diversity is generated using the degenerate codon sets. However, there can be individual preferences for one codon set or the other, depending on the various factors known in the art, such as efficiency of coupling in oligonucleotide synthesis chemistry.

In some embodiments, the practitioner of methods of the invention may wish to modify the amount/proportions of individual nucleotides (G, A, T, C) for a codon set.

Oligonucleotide or primer sets can be synthesized using standard methods. A set of oligonucleotides can be synthesized, for example, by solid phase synthesis, containing sequences that represent all possible combinations of nucleotide triplets provided by the restricted codon set and that will encode the desired restricted group of amino acids. Synthesis of oligonucleotides with selected nucleotide “degeneracy” at certain positions is well known in that art. Such sets of oligonucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a CDR (for e.g., as contained within a variable domain) nucleic acid template and also can include restriction enzyme sites for cloning purposes.

In one method, nucleic acid sequences encoding variant amino acids can be created by oligonucleotide-mediated mutagenesis of a nucleic acid sequence encoding a source or template polypeptide such as the antibody variable domain of 4D5. This technique is well known in the art as described by Zoller et al. Nucleic Acids Res. 10:6487-6504 (1987). Briefly, nucleic acid sequences encoding variant amino acids are created by hybridizing an oligonucleotide set encoding the desired restricted codon sets to a DNA template, where the template is the single-stranded form of the plasmid containing a variable region nucleic acid template sequence. After hybridization, DNA polymerase is used to synthesize an entire second complementary strand of the template that will thus incorporate the oligonucleotide primer, and will contain the restricted codon sets as provided by the oligonucleotide set. Nucleic acids encoding other source or template molecules are known or can be readily determined.

Generally, oligonucleotides of at least 25 nucleotides in length are used. An optimal oligonucleotide will have at least 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that described by Crea et al., Proc. Natl. Acad. Sei. USA, 75:5765 (1978).

The DNA template is generated by those vectors that are either derived from bacteriophage M13 vectors (the commercially available M13 mp18 and M13 mp19 vectors are suitable), or those vectors that contain a single-stranded phage origin of replication as described by Viera et al., Meth. Enzymol., 153:3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with a 32-Phosphate to identify the bacterial colonies that contain the mutated DNA.

The method described immediately above may be modified such that a homoduplex molecule is created wherein both strands of the plasmid contain the mutation(s).

The modifications are as follows: The single stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously the sequence of the oligonucleotide set is of sufficient length to hybridize to the template nucleic acid and may also, but does not necessarily, contain restriction sites. The DNA template can be generated by those vectors that are either derived from bacteriophage M13 vectors or vectors that contain a single-stranded phage origin of replication as described by Viera et al. ((1987) Meth. Enzymol., 153:3). Thus, the DNA that is to be mutated must be inserted into one of these vectors in order to generate single-stranded template. Production of the single-stranded template is described in sections 4.21-4.41 of Sambrook et al., supra.

According to another method, a library can be generated by providing upstream and downstream oligonucleotide sets, each set having a plurality of oligonucleotides with different sequences, the different sequences established by the codon sets provided within the sequence of the oligonucleotides. The upstream and downstream oligonucleotide sets, along with a variable domain template nucleic acid sequence, can be used in a polymerase chain reaction to generate a “library” of PCR products. The PCR products can be referred to as “nucleic acid cassettes”, as they can be fused with other related or unrelated nucleic acid sequences, for example, viral coat protein components and dimerization domains, using established molecular biology techniques.

The sequence of the PCR primers includes one or more of the designed codon sets for the JR's and highly diverse positions in a CDR region. As described above, a codon set is a set of different nucleotide triplet sequences used to encode desired variant amino acids.

Oligonucleotide sets can be used in a polymerase chain reaction using a variable region nucleic acid template sequence as the template to create nucleic acid cassettes. The variable region nucleic acid template sequence can be any portion of the light or heavy immunoglobulin chains containing the target nucleic acid sequences (ie., nucleic acid sequences encoding amino acids targeted for substitution). The variable region nucleic acid template sequence is a portion of a double stranded DNA molecule having a first nucleic acid strand and complementary second nucleic acid strand. The variable region nucleic acid template sequence contains at least a portion of a variable domain e.g., JR regions and has at least one CDR. In some cases, the variable region nucleic acid template sequence contains more than one JR and CDR. An upstream portion and a downstream portion of the variable region nucleic acid template sequence can be targeted for hybridization with members of an upstream oligonucleotide set and a downstream oligonucleotide set.

A first oligonucleotide of the upstream primer set can hybridize to the first nucleic acid strand and a second oligonucleotide of the downstream primer set can hybridize to the second nucleic acid strand. The oligonucleotide primers can include one or more codon sets and be designed to hybridize to a portion of the variable region nucleic acid template sequence. Use of these oligonucleotides can introduce two or more codon sets into the PCR product (ie., the nucleic acid cassette) following PCR. The oligonucleotide primer that hybridizes to regions of the nucleic acid sequence encoding the antibody variable domain includes portions that encode CDR residues that are targeted for amino acid substitution.

The upstream and downstream oligonucleotide sets can also be synthesized to include restriction sites within the oligonucleotide sequence. These restriction sites can facilitate the insertion of the nucleic acid cassettes [ie., PCR reaction products] into an expression vector having additional antibody sequences. Preferably, the restriction sites are designed to facilitate the cloning of the nucleic acid cassettes without introducing extraneous nucleic acid sequences or removing original CDR or framework nucleic acid sequences.

Nucleic acid cassettes can be cloned into any suitable vector for expression of a portion or the entire light or heavy chain sequence containing the targeted amino acid substitutions generated. According to methods detailed in the invention, the nucleic acid cassette is cloned into a vector allowing production of a portion or the entire light or heavy chain sequence fused to all or a portion of a viral coat protein (ie., creating a fusion protein) and displayed on the surface of a particle or cell. While several types of vectors are available and may be used to practice this invention, phagemid vectors are the preferred vectors for use herein, as they may be constructed with relative ease, and can be readily amplified. Phagemid vectors generally contain a variety of components including promoters, signal sequences, phenotypic selection genes, origin of replication sites, and other necessary components as are known to those of ordinary skill in the art.

In another embodiment, wherein a particular variant amino acid combination is to be expressed, the nucleic acid cassette contains a sequence that is able to encode all or a portion of the heavy or light chain variable domain, and is able to encode the variant amino acid combinations. For production of antibodies containing these variant amino acids or combinations of variant amino acids, as in a library, the nucleic acid cassettes can be inserted into an expression vector containing additional antibody sequence, for example all or portions of the variable or constant domains of the light and heavy chain variable regions. These additional antibody sequences can also be fused to other nucleic acid sequences, such as sequences which encode viral coat protein components and therefore allow production of a fusion protein.

Once the libraries with diversified JR regions are prepared they can be selected and/or screened for binding to one or more target antigens. In addition, the libraries may be selected for improved binding affinity to particular target antigen. The target antigens may be any type of antigenic molecule but preferably are a therapeutic target molecule.

The libraries of the invention may be generated by mutating the amino acids that comprise at least one JR, and optionally one or more CDRs. Thus, a library of antibody variable domains can be generated, for example, having mutations in the at least one or more JR's on one of the heavy or light chains, and optionally, CDRH1, CDRH2, and/or CDRH3. Another library can be generated having mutations in CDRL1, CDRL2 and CDRL3 in addition to or alternative to a mutation in an attendant JR. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

Another aspect of the invention includes compositions of the polypeptides, fusion proteins or libraries of the invention. Compositions comprise a polypeptide, a fusion protein, or a population of polypeptides or fusion proteins in combination with a physiologically acceptable carrier.

### 2. Variant VFRs

As discussed above, randomized or targeted JR or CDR variants can generate polypeptide libraries that bind to a variety of target molecules, including antigens. These variant CDRs or JR's can be incorporated into other antibody molecules or used to form a single chain mini-antibody with an antigen binding domain comprising a heavy chain variable domain but lacking a light chain.

Polypeptides comprising a variant JR having such a structure include VHH, antibody or monobody variable domain obtained from a naive or synthetic library, naturally occurring antibody or monobody, recombinant antibody or monobody, humanized antibody or monobody, germline derived antibody or monobody, chimeric antibody or monobody, and affinity matured antibody or monobody.

A number of different combinations of amino acid positions structural and/or nonstructural amino acid positions can be designed in a JR template. In general, the amino acids to the left of the central portion of contiguous amino acids (CDR) are referred to as the N terminal amino acids, and the amino acids to the right of the contiguous sequence (CDR) are referred to as C terminal amino acids. The amino acid positions at any one of the three positions within the JR can be any of the 20 naturally occurring amino acids, preferably L-amino acids. In some embodiments, the selected amino acids can be encoded by a nonrandom codon set that encodes six or less amino acids. The nonrandom codon set preferably encodes amino acids found or commonly occurring at those positions in randomly generated and/or naturally occurring antibodies.

### 3. Diversity in CDR Regions

The diversity of the library or population of the antibody variable domains is designed to maximize diversity while minimizing structural perturbations of the antibody variable domain to provide for increased ability to isolate high affinity antibodies. The number of positions mutated in the antibody variable domain is minimized or specifically targeted. In some embodiments, structural amino acid positions are identified and diversity is minimized at those positions to ensure a well-folded polypeptide. The positions mutated or changed include positions in FR and/or one or more of the CDR regions and combinations thereof.

The source polypeptide may be any antibody, antibody fragment, or antibody variable domain whether naturally occurring or synthetic. A polypeptide or source antibody variable domain can include an antibody, antibody variable domain, antigen binding fragment or polypeptide thereof, a monobody, VHH, a monobody or antibody variable domain obtained from a nave or synthetic library, naturally occurring antibody or monobody, synthetic antibody or monobody, recombinant antibody or monobody, humanized antibody or monobody, germline derived antibody or monobody, chimeric antibody or monobody, and affinity matured antibody or monobody. Representative sources opf polypepetides include her3, PCSK9, IL-13 (R), EphA2, antigen E or TrkA. Of course, any other polypeptide may be used as the source.

Source antibody variable domains include but are not limited to antibody variable domains previously used to generate phage display libraries, such as VHH-RIG, VHH-VLK, VHH-LLR, and VHH-RLV (Bond et al., 2003, 3. Mol. Biol., 332:643-655), and humanized antibodies or antibody fragments, such as mAbs 4D5, 2C4, and A.sub.4.6.1. In an embodiment, the library is generated using the heavy chain variable domain (VHH) of a monobody. The small size and simplicity make monobodies attractive scaffolds for peptidomimetic and small molecule design, as reagents for high throughput protein analysis, or as potential therapeutic agents. The diversified VHH domains are useful, inter alia, in the design of enzyme inhibitors, novel antigen binding molecules, modular binding units in bispecific or intracellular antibodies, as binding reagents in protein arrays, and as scaffolds for presenting constrained peptide libraries.

One criterion for generating diversity in the polypeptide library is selecting amino acid positions that form an antigen binding pocket or groove in a single source antibody variable domain whether or not the residues actually contact the antigen. In some embodiments, the amino acids position may form all or part of a loop. One way of determining whether the amino acid position is part of a loop in an antigen binding site is to examine the three dimensional structure of the antibody variable domain, for example, for solvent accessible residues. If available, amino acids positions in proximity to antigen can also be selected. Three dimensional structure information of antibody variable domains are available for many antibodies or can be prepared using available molecular modeling programs. Solvent accessible amino acid positions can be found in FR and CDRs, and typically form loops on the exterior of the protein. Preferably, solvent accessible positions are determined using coordinates from a 3-dimensional model of an antibody, using a computer program such as the InsightI1 program (Accelrys, San Diego, Calif.). Solvent accessible positions can also be determined using algorithms known in the art (e.g., Lee and Richards, J. Mol. Biol. 55, 379 (1971) and Connolly, J, Appl. Cryst. 16, 548 (1983)). Determination of solvent accessible positions can be performed using software suitable for protein modeling and 3-dimensional structural information obtained from an antibody. Software that can be utilized for these purposes includes SYBYL Biopolymer Module software (Tripos Associates). Generally and preferably, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers”, Comput. Chem. 18(4): 377-386; and “Variations of Surface Areas and Volumes in Distinct Molecular Surfaces of Biomolecules.” J. Mol. Model. (1995), 1: 46-53). The location of amino acid positions involved in forming antigen binding pockets may vary in different antibody variable domains, but typically involve at least one or a portion of a CDR and/or a portion of the FR (JR) region.

Amino acid positions less tolerant to amino acid substitutions can be identified using a method such as alanine scanning mutagenesis or shotgun scanning as described in WO 01/44463 and analyzing the effect of loss of the wild type amino acid on structural stability at positions in the CDR. An amino acid position is important to maintaining the structure of the polypeptide if a wild type amino acid is replaced with a scanning amino acid in an amino acid position in a CDR and the resulting variant exhibits poor binding to a target molecule that binds to folded polypeptide. A structural amino acid position is, preferably, a position in which the ratio of sequences with the wild type amino acid at a position to sequences with the scanning amino acid at that position is at least about 3 to 1.5 to 1.8 to 1, or about 10 to 1 or greater.

Initially, a CDRH1 region can include amino acid positions as defined by Chothia including amino acid positions 26 to 32. Additional amino acid positions can also be randomized on either side of the amino acid positions in CDRH1 as defined by Chothia, typically 1 to 3 amino acids at the N and/or C terminal end. The N terminal flanking region, central portion, and C-terminal flanking region is determined by selecting the length of CDRH1, randomizing each position and identifying the structural amino acid positions at the N and C-terminal ends of the CDR to set the boundaries of the CDR. The length of the N and C terminal flanking sequences should be long enough to include at least one structural amino acid position in each flanking sequence. In some embodiments, the length of the N-terminal flanking region is at least about from 1 to 4 contiguous amino acids, the central portion of one or more nonstructural positions can vary about 1 to 20 contiguous amino acids, and the C-terminal portion is at least about from 1 to 6 contiguous amino acids. In some embodiments, the central portion of contiguous amino acids can comprise, consist essentially of or consist of about 9 to 17 amino acids, about 9 to about 15 amino acids, and more preferably about 9 to 12 amino acids. The structural amino acid positions are less diversified than the central portion of the CDRH1 which can be completely randomized if desired. At the structural amino acid positions, up to six, and preferably no more than six different amino acids are substituted, more preferably about 1 to 6 different amino acids, more preferably about 1 to 5 different amino acids, more preferably about 1 to 4 different amino acids, more preferably about 1-3 different amino acids, and most preferably about 1-2 different amino acids. In some embodiments, the structural amino acid position is substituted with one or more hydrophobic amino acids and is encoded by a nonrandom codon set encoding six or less amino acids.

In another embodiment, CDR diversity is generated using any one of the codon sets described herein that encode the same amino acid group. However, there can be individual preferences for one codon set or the other, depending on the various factors known in the art, such as efficiency of coupling in oligonucleotide synthesis chemistry.

In some embodiments, the practitioner of methods of the invention may wish to modify the amount/proportions of individual nucleotides (G, A, T, C) for a codon set, such as the N nucleotide in a codon set such as in NNS. This is illustratively represented as XYZ codons. This can be achieved by, for example, doping different amounts of the nucleotides within a codon set instead of using a straight, equal proportion of the nucleotides for the N in the codon set. Such modifications can be useful for various purposes depending on the circumstances and desire of the practitioner. For example, such modifications can be made to more closely reflect the amino acid bias as seen in a natural diversity profile, such as the profile of CDR. Once the libraries with diversified CDR regions are prepared they can be selected and/or screened for binding one or more target antigens. In addition, the libraries may be selected for improved binding affinity to particular target antigen. The target antigens may include any type of antigenic molecule but preferably is antibody to therapeutic target molecule. The target antigen can also include a molecule that preferentially binds to folded antibody variable domains and does not bind as well as to unfolded antibody variable domains.

Antibody variable domains with targeted diversity in one or more CDRs can be combined with targeted diversity in a JR region as well. Combination of regions may be diversified in order to provide for high affinity antigen binding molecules or to improve the affinity of a known antibody such as a humanized antibody.

### 4. Fusion Polypeptides

Fusion polypeptide constructs can be prepared for generating fusion polypeptides that bind with significant affinity to potential ligands.

In particular, fusion polypeptides comprising diversified JR and/or CDR(s) and a heterologous polypeptide sequence (preferably that of at least a portion of a viral polypeptide) are generated, individually and as a plurality of unique individual polypeptides that are candidate binders to targets of interest. Compositions (such as libraries) comprising such polypeptides find use in a variety of applications, in particular as large and diverse pools of candidate immunoglobulin polypeptides (in particular, antibodies and antibody fragments) that bind to targets of interest.

In some embodiments, a fusion protein comprises an antibody variable domain, or an antibody variable domain and a constant domain, fused to all or a portion of a viral coat protein. Examples of viral coat proteins include infectivity protein PIII, major coat protein PVIII, p3, Sac, Hoc, gpD (of bacteriophage lambda), minor bacteriophage coat protein 6 (pVI) (filamentous phage; J Immunol Methods. 1999 Dec. 10; 231(1-2):39-51), variants of the M13 bacteriophage major coat protein (P8) (Protein Sci 2000 April; 9(4):647-54). The fusion protein can be displayed on the surface of a phage and suitable phage systems include M13KO7 helper phage, M13R408, M13-VCS, and Phi X 174, pJuFo phage system (J. Virol. 2001 August; 75(15):7107-13.v), hyperphage (Nat. Biotechnol. 2001 January; 19(1):75-8). The preferred helper phage is M13KO7, and the preferred coat protein is the M13 Phage gene III coat protein.

Tags useful for detection of antigen binding can also be fused to either an antibody variable domain not fused to a viral coat protein or an antibody variable domain fused to a viral coat protein. Additional peptides that can be fused to antibody variable domains include gD tags, c-Myc epitopes, poly-histidine tags, fluorescence proteins (e.g., GFP), or .beta.-galactosidase protein which can be useful for detection or purification of the fusion protein expressed on the surface of the phage or cell.

These constructs may also comprise a dimerizable sequence that when present as a dimerization domain in a fusion polypeptide provides for increased tendency for heavy chains to dimerize to form dimers of Fab or Fab′ antibody fragments/portions. These dimerization sequences may be in addition to any heavy chain hinge sequence that may be present in the fusion polypeptide. Dimerization domains in fusion phage polypeptides bring two sets of fusion polypeptides (LC/HC-phage protein/fragment (such as pill)) together, thus allowing formation of suitable linkages (such as interheavy chain disulfide bridges) between the two sets of fusion polypeptide. Vector constructs containing such dimerization sequences can be used to achieve divalent display of antibody variable domains, for example the diversified fusion proteins described herein, on phage. Preferably, the intrinsic affinity of each monomeric antibody fragment (fusion polypeptide) is not significantly altered by fusion to the dimerization sequence. Preferably, dimerization results in divalent phage display which provides increased avidity of phage binding, with significant decrease in off-rate, which can be determined by methods known in the art and as described herein. Dimerization sequence-containing vectors of the invention may or may not also include an amber stop codon 5′ of the dimerization sequence. Dimerization sequences are known in the art, and include, for example, the GCN4 zipper sequence (GRMKQLEDKVEELLSKNYHLENEVARLKKLVGERG).

### Generation of Libraries Comprising Variant CDR-Containing Polypeptides

Libraries of variant CDR polypeptides can be generated by mutating the framework residues in a JR and/or highly diverse positions in at least one CDR of an antibody variable domain. Some or all of the CDRs can be mutated using the methods of the invention. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in at least one JR and/or highly diverse positions of CDRH1, CDRH2 and CDRH3. Another library can be generated having mutations in CDRL1, CDRL2 and CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

In one embodiment, a library is created by substitution of original amino acids with a limited set of variant amino acids in the CDRH1, CDRH2, and/or CDRH3 region of the variable region of the heavy chain sequence and/or the CDRL3 region of the variable region of the light chain sequence or in at least JR on either the heavy or light chain. According to the invention, this library can contain a plurality of antibody sequences, wherein the sequence diversity is primarily in the CDRH3 region of the heavy chain sequence.

Multiple libraries can be pooled and sorted using solid support selection and solution sorting methods as described herein. Multiple sorting strategies may be employed. For example, one variation involves sorting on target bound to a solid, followed by sorting for a tag that may be present on the fusion polypeptide (e.g. anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield. Any combination of codon sets and CDRs can be diversified according to methods of the invention.

### 6. Polynucleotides, Vectors, Host Cells, and Recombinant Methods

### Vectors

One aspect of the invention includes a replicable expression vector comprising a nucleic acid sequence encoding a gene fusion, wherein the gene fusion encodes a fusion protein comprising a CDR-containing polypeptide (such as an antibody variable domain), or an antibody variable domain and a constant domain, fused to all or a portion of a viral coat protein. Also included is a library of diverse replicable expression vectors comprising a plurality of gene fusions encoding a plurality of different fusion proteins including a plurality of the fusion polypeptides generated with diverse sequences as described above. The vectors can include a variety of components and may be constructed to allow for movement of antibody variable domain between different vectors and/or to provide for display of the fusion proteins in different formats.

Examples of vectors include phage vectors and phagemid vectors. A phage vector generally has a phage origin of replication allowing phage replication and phage particle formation. The phage is generally a filamentous bacteriophage, such as an M13, fl, fd, Pf3 phage or a derivative thereof, or a lambdoid phage, such as lambda, 21, phi80, phi81, 82, 424, 434, etc., or a derivative thereof.

Examples of viral coat proteins include infectivity protein P111 (sometimes also designated p3), major coat protein PVII1, Soc (T4), Hoc (T4), gpD (of bacteriophage lambda), minor bacteriophage coat protein 6 (pVI) (filamentous phage; Immunol Methods. 1999 Dee. 10; 231 (1-2):39-51), variants of the M13 bacteriophage major coat protein (P8) (Protein Sci 2000 April; 9(4):647-54). The fusion protein can be displayed on the surface of a phage and suitable phage systems include M13KO7 helper phage, M13R408, M13-VCS, and Phi X 174, pJuFo phage system (J. Virol. 2001 August; 75(15):7107-13.v), hyperphage (Nat. Biotechnol. 2001 January; 19(1):75-8). In certain embodiments, the helper phage is M13KO7, and the coat protein is the M13 Phage gene III coat protein. In certain embodiments, the host is E. coli, and protease deficient strains of E. coli. Vectors, such as the fth 1 vector (Nucleic Acids Res. 2001 May 15; 29(10):E50-0) can be useful for the expression of the fusion protein.

The expression vector also can have a secretory signal sequence fused to the DNA encoding a CDR-containing fusion polypeptide (e.g., each subunit of an antibody, or fragment thereof). This sequence is typically located immediately 5′ to the gene encoding the fusion protein, and will thus be transcribed at the amino terminus of the fusion protein. However, in certain cases, the signal sequence has been demonstrated to be located at positions other than 5′ to the gene encoding the protein to be secreted. This sequence targets the protein to which it is attached across the inner membrane of the bacterial cell. The DNA encoding the signal sequence may be obtained as a restriction endonuclease fragment from any gene encoding a protein that has a signal sequence. Suitable prokaryotic signal sequences may be obtained from genes encoding, for example, LamB or OmpF (Wong et al., Gene, 68:1931 (1983), MalE, PhoA and other genes. In one embodiment, a prokaryotic signal sequence for practicing this invention is the E. coli heat-stable enterotoxin II (STII) signal sequence as described by Chang et al., Gene 55:189 (1987), and/or malE.

As indicated above, a vector also typically includes a promoter to drive expression of the fusion polypeptide. Promoters most commonly used in prokaryotic vectors include the lac Z promoter system, the alkaline phosphatase pho A promoter (Ap), the bacteriophage I.sub.PL promoter (a temperature sensitive promoter), the tac promoter (a hybrid trp-lac promoter that is regulated by the lac repressor), the tryptophan promoter, and the bacteriophage T7 promoter. For general descriptions of promoters, see section 17 of Sambrook et al. supra. While these are the most commonly used promoters, other suitable promoters may be used as well.

The vector can also include other nucleic acid sequences, for example, sequences encoding gD tags, c-Myc epitopes, poly-histidine tags, fluorescence proteins (e.g., GFP), or beta-galactosidase protein which can be useful for detection or purification of the fusion protein expressed on the surface of the phage or cell. Nucleic acid sequences encoding, for example, a gD tag, also provide for positive or negative selection of cells or virus expressing the fusion protein. In some embodiments, the gD tag is fused to an antibody variable domain which is not fused to the viral coat protein component. Nucleic acid sequences encoding, for example, a polyhistidine tag, are useful for identifying fusion proteins including antibody variable domains that bind to a specific antigen using immunohistochemistry. Tags useful for detection of antigen binding can be fused to either an antibody variable domain not fused to a viral coat protein component or an antibody variable domain fused to a viral coat protein component.

Another useful component of the vectors used to practice this invention is phenotypic selection genes. Typical phenotypic selection genes are those encoding proteins that confer antibiotic resistance upon the host cell. By way of illustration, the ampicillin resistance gene (amp), and the tetracycline resistance gene (tetr) are readily employed for this purpose.

The vector can also include nucleic acid sequences containing unique restriction sites and suppressible stop codons. The unique restriction sites are useful for moving antibody variable domains between different vectors and expression systems, especially useful for production of full-length antibodies or antigen binding fragments in cell cultures. The suppressible stop codons are useful to control the level of expression of the fusion protein and to facilitate purification of soluble antibody fragments. For example, an amber stop codon can be read as Gln in a supE host to enable phage display, while in a non-supE host it is read as a stop codon to produce soluble antibody fragments without fusion to phage coat proteins. These synthetic sequences can be fused to one or more antibody variable domains in the vector.

It is sometimes beneficial to use vector systems that allow the nucleic acid encoding an antibody sequence of interest, for example a CDR having variant amino acids, to be easily removed from the vector system and placed into another vector system. For example, appropriate restriction sites can be engineered in a vector system to facilitate the removal of the nucleic acid sequence encoding an antibody or antibody variable domain having variant amino acids. The restriction sequences are usually chosen to be unique in the vectors to facilitate efficient excision and ligation into new vectors. Antibodies or antibody variable domains can then be expressed from vectors without extraneous fusion sequences, such as viral coat proteins or other sequence tags.

Between nucleic acid encoding antibody variable or constant domain (gene I) and the viral coat protein component (gene 2), DNA encoding a termination or stop codon may be inserted, such termination codons including UAG (amber), UAA (ocher) and UGA (ope1). (Microbiology, Davis et al., Harper & Row, New York, 1980, pp. 237, 245-47 and 374). The termination or stop codon expressed in a wild type host cell results in the synthesis of the gene protein product without the gene 2 protein attached. However, growth in a suppressor host cell results in the synthesis of detectable quantities of fused protein. Such suppressor host cells are well known and described, such as E. coli suppressor strain (Bullock et al., BioTechniques 5:376-379 (1987)). Any acceptable method may be used to place such a termination codon into the mRNA encoding the fusion polypeptide.

The suppressible codon may be inserted between the first gene encoding an antibody variable or constant domain, and a second gene encoding at least a portion of a phage coat protein. Alternatively, the suppressible termination codon may be inserted adjacent to the fusion site by replacing the last amino acid triplet in the antibody variable domain or the first amino acid in the phage coat protein. The suppressible termination codon may be located at or after the C-terminal end of a dimerization domain. When the plasmid containing the suppressible codon is grown in a suppressor host cell, it results in the detectable production of a fusion polypeptide containing the polypeptide and the coat protein. When the plasmid is grown in a non-suppressor host cell, the antibody variable domain is synthesized substantially without fusion to the phage coat protein due to termination at the inserted suppressible triplet UAG, UAA, or UGA. In the non-suppressor cell the antibody variable domain is synthesized and secreted from the host cell due to the absence of the fused phage coat protein which otherwise anchored it to the host membrane.

In some embodiments, the CDR or JR being diversified (randomized or targeted approach) may have a stop codon engineered in the template sequence (referred to herein as a “stop template”). This feature provides for detection and selection of successfully diversified sequences based on successful repair of the stop codon(s) in the template sequence due to incorporation of the oligonucleotide(s) comprising the sequence(s) for the variant amino acids of interest.

The light and/or heavy chain antibody variable or constant domains can also be fused to an additional peptide sequence, the additional peptide sequence providing for the interaction of one or more fusion polypeptides on the surface of the viral particle or cell. These peptide sequences are herein referred to as “dimerization domains”. Dimerization domains may comprise at least one or more of a dimerization sequence, or at least one sequence comprising a cysteine residue or both. Suitable dimerization sequences include those of proteins having amphipathic alpha helices in which hydrophobic residues are regularly spaced and allow the formation of a dimer by interaction of the hydrophobic residues of each protein; such proteins and portions of proteins include, for example, leucine zipper regions. Dimerization domains can also comprise one or more cysteine residues (e.g. as provided by inclusion of an antibody hinge sequence within the dimerization domain). The cysteine residues can provide for dimerization by formation of one or more disulfide bonds. In one embodiment, wherein a stop codon is present after the dimerization domain, the dimerization domain comprises at least one cysteine residue. In some embodiments, the dimerization domains are located between the antibody variable or constant domain and the viral coat protein component.

In some cases the vector encodes a single antibody-phage polypeptide in a single chain form containing, for example, both the heavy and light chain variable regions fused to a coat protein. In these cases the vector is considered to be “monocistronic”, expressing one transcript under the control of a certain promoter. For example, a vector may utilize a promoter (such as the alkaline phosphatase (AP) or Tac promoter) to drive expression of a monocistronic sequence encoding VL and VH domains, with a linker peptide between the VL and VH domains. This cistronic sequence may be connected at the 5′ end to a signal sequence (such as an E. coli malE or heat-stable enterotoxin II (STII) signal sequence) and at its 3′ end to all or a portion of a viral coat protein (such as the bacteriophage pIII protein). The fusion polypeptide encoded by a vector of this embodiment is referred to herein as “ScFv-pIII”. In some embodiments, a vector may further comprise a sequence encoding a dimerization domain (such as a leucine zipper) at its 3′ end, between the second variable domain sequence (e.g., VH) and the viral coat protein sequence. Fusion polypeptides comprising the dimerization domain are capable of dimerizing to form a complex of two scFv polypeptides (referred to herein as “(ScFv).sub.2-pIII)”).

In other cases, the variable regions of the heavy and light chains can be expressed as separate polypeptides, the vector thus being “bicistronic”, allowing the expression of separate transcripts. In these vectors, a suitable promoter, such as the Ptac or PhoA promoter, is used to drive expression of a bicistronic message. A first cistron encoding, for example, a light chain variable and constant domain, may be connected at the 5′ end to a signal sequence, such as E. coli malE or heat-stable enterotoxin II (STII) signal sequence, and at the 3′ end to a nucleic acid sequence encoding a tag sequence, such as gD tag. A second cistron, encoding, for example, a heavy chain variable domain and constant domain CH1, is connected at its 5′ end to a signal sequence, such as E. coli malE or heat-stable enterotoxin II (STII) signal sequence, and at the 3′ end to all or a portion of a viral coat protein.

In one embodiment of a vector which provides a bicistronic message and for display of F(ab′).sub.2-pIII, a suitable promoter, such as Ptac or PhoA (AP) promoter, drives expression of a first cistron encoding a light chain variable and constant domain operably linked at 5′ end to a signal sequence such as the E. coli malE or heat stable enterotoxin II (STII) signal sequence, and at the 3′ end to a nucleic acid sequence encoding a tag sequence such as gD tag. The second cistron encodes, for example, a heavy chain variable and constant domain operatively linked at 5′ end to a signal sequence such as E. coli malE or heat stable enterotoxin II (STII) signal sequence, and at 3′ end has a dimerization domain comprising IgG hinge sequence and a leucine zipper sequence followed by at least a portion of viral coat protein.

Introduction of Vectors into Host Cells

Vectors constructed as described in accordance with the invention are introduced into a host cell for amplification and/or expression. Vectors can be introduced into host cells using standard transformation methods including electroporation, calcium phosphate precipitation and the like. If the vector is an infectious particle such as a virus, the vector itself provides for entry into the host cell. Transfection of host cells containing a replicable expression vector which encodes the gene fusion and production of phage particles according to standard procedures provides phage particles in which the fusion protein is displayed on the surface of the phage particle.

Replicable expression vectors are introduced into host cells using a variety of methods. In one embodiment, vectors can be introduced into cells using electroporation as described in WO/00106717. Cells are grown in culture in standard culture broth, optionally for about 6-48 hours (or to OD.sub.600=0.6-0.8) at about 37.degree. C., and then the broth is centrifuged and the supernatant removed (e.g. decanted). In some embodiments, initial purification includes resuspending the cell pellet in a buffer solution (e.g. 1.0 mM HEPES pH 7.4) followed by recentrifugation and removal of supernatant. The resulting cell pellet is resuspended in dilute glycerol (e.g. 5-20% v/v) and again recentrifuged to form a cell pellet and the supernatant removed. The final cell concentration is obtained by resuspending the cell pellet in water or dilute glycerol to the desired concentration.

In certain embodiments, the recipient cell is the electroporation competent E. coli strain of the present invention, which is E. coli strain SS320 (Sidhu et al., Methods Enzymol. (2000), 328:333-363). Strain SS320 was prepared by mating MC1061 cells with XL1-BLUE cells under conditions sufficient to transfer the fertility episome (F′ plasmid) or XL1-BLUE into the MC1061 cells. Strain SS320 has been deposited with the American Type Culture Collection (ATCC), 10801 University Boulevard, Manassas, Va. USA, on Jun. 18, 1998 and assigned Deposit Accession No, 98795. Any F′ episome which enables phage replication in the strain may be used in the invention. Suitable episomes are available from strains deposited with ATCC or are commercially available (C.1236, CSH18, DHF′, JM101, JM103, JM105, JM107, JM109, JM110), KS1000, XL1-BLUE, 71-18 and others).

The use of higher DNA concentrations during electroporation (about 10.times.) increases the transformation efficiency and increases the amount of DNA transformed into the host cells. The use of high cell concentrations also increases the efficiency (about 10.times.). The larger amount of transferred DNA produces larger libraries having greater diversity and representing a greater number of unique members of a combinatorial library, Transformed cells are generally selected by growth on antibiotic containing medium.

### Selection (Sorting) and Screening for Binders to Targets of Choice

Use of phage display for identifying target antigen binders, with its various permutations and variations in methodology, are well established in the art. One approach involves constructing a family of variant replicable vectors containing a transcription regulatory element operably linked to a gene fusion encoding a fusion polypeptide, transforming suitable host cells, culturing the transformed cells to form phage particles which display the fusion polypeptide on the surface of the phage particle, followed by a process that entails selection or sorting by contacting the recombinant phage particles with a target antigen so that at least a portion of the population of particles bind to the target with the objective to increase and enrich the subsets of the particles which bind from particles relative to particles that do not bind in the process of selection. The selected pool can be amplified by infecting host cells, such as fresh XL1-Blue cells, for another round of sorting on the same target with different or same stringency. The resulting pool of variants are then screened against the target antigens to identify novel high affinity binding proteins. These novel high affinity binding proteins can be useful as therapeutic agents as antagonists or agonists, and/or as diagnostic and research reagents.

Fusion polypeptides such as antibody variable domains comprising the variant amino acids can be expressed on the surface of a phage, phagemid particle or a cell and then selected and/or screened for the ability of members of the group of fusion polypeptides to bind a target antigen which is typically an antigen of interest. The processes of selection for binders to target can also be include sorting on a generic protein having affinity for antibody variable domains such as protein L or a tag specific antibody which binds to antibody or antibody fragments displayed on phage, which can be used to enrich for library members that display correctly folded antibody fragments (fusion polypeptides).

Target proteins, such as for example her3 or PCSK9 may be isolated from natural sources or prepared by recombinant methods by procedures known in the art. Sequences of the various target polypepetidse again which variant antibodies made in accordance with the methods of the invention are known to a skilled artisan. Target antigens can include a number of molecules of therapeutic interest.

A variety of strategies of selection (sorting) for affinity can be used. One example is a solid-support method or plate sorting or immobilized target sorting. Another example is a solution-binding method.

For the solid support method, the target protein may be attached to a suitable solid or semi solid matrix. Such matrices are known in the art, such as agarose beads, acrylamide beads, glass beads, cellulose, various acrylic copolymers, hydroxyalkyl methacrylate gels, polyacrylic and polymethacrylic copolymers, nylon, neutral and ionic carriers, and the like. Attachment of the target protein to the matrix may be accomplished by methods described, e.g., in Methods in Enzymology, 44 (1976), or by other means known in the art. After attachment of the target antigen to the matrix, the immobilized target is contacted with the library expressing the fusion polypeptides under conditions suitable for binding of at least a subset of the phage particle population with the immobilized target antigen. Normally, the conditions, including pH, ionic strength, temperature and the like will mimic physiological conditions. Bound particles (“binders”) to the immobilized target are separated from those particles that do not bind to the target by washing. Wash conditions can be adjusted to result in removal of all but the high affinity binders. Binders may be dissociated from the immobilized target by a variety of methods. These methods include competitive dissociation using the wild-type ligand (e.g. excess target antigen), altering pH and/or ionic strength, and methods known in the art. Selection of binders typically involves elution from an affinity matrix with a suitable elution material such as acid like 0.1M HCl or ligand. Elution with increasing concentrations of ligand could elute displayed binding molecules of increasing affinity.

The binders can be isolated and then re-amplified in suitable host cells by infecting the cells with the viral particles that are binders (and helper phage if necessary, e.g., when the viral particle is a phagemid particle) and the host cells are cultured under conditions suitable for amplification of the particles that display the desired fusion polypeptide. The phage particles are then collected and the selection process is repeated one or more times until binders of the target antigen are enriched. Any number of rounds of selection or sorting can be utilized. One of the selection or sorting procedures can involve isolating binders that bind to a generic affinity protein such as protein L or an antibody to a polypeptide tag present in a displayed polypeptide such as antibody to the gD protein or polyhistidine tag. Counterselection may be included in one or more rounds of selection or sorting to isolate binders that also exhibit undesired binding to one or more non-target antigens.

One aspect of the invention involves selection against libraries of the invention using a novel selection method which is termed “solution-binding method”. The invention allows solution phase sorting with much improved efficiency over conventional solution sorting methods. The solution binding method may be used for finding original binders from a random library or finding improved binders from a library that was designated to improve affinity of a particular binding clone or group of clones. The method comprises contacting a plurality of polypeptides, such as those displayed on phage or phagemid particles (library), with a target antigen labeled or fused with a tag molecule. The tag could be biotin or other moieties for which specific binders are available. The stringency of the solution phase can be varied by using decreasing concentrations of labeled target antigen in the first solution binding phase. To further increase the stringency, the first solution binding phase can be followed by a second solution phase having high concentration of unlabelled target antigen after the initial binding with the labeled target in the first solution phase. Usually, 100 to 1000 fold of unlabelled target over labeled target is used in the second phase (if included). The length of time of incubation of the first solution phase can vary from a few minutes to one to two hours or longer to reach equilibrium. Using a shorter time for binding in this first phase may bias or select for binders that have fast on-rate. The length of time and temperature of incubation in second phase can be varied to increase the stringency. This provides for a selection bias for binders that have slow rate of coming off the target (off-rate). After contacting the plurality of polypeptides (displayed on the phage/phagemid particles) with a target antigen, the phage or phagemid particles that are bound to labeled targets are separated from phage that do not bind. The particle-target mixture from solution phase of binding is isolated by contacting it with the labeled target moiety and allowing for its binding to, a molecule that binds the labeled target moiety for a short period of time (e.g., 2-5 minutes). The initial concentration of the labeled target antigen can range from about 0.1 nM to about 1000 nM. The bound particles are eluted and can be propagated for next round of sorting. In certain embodiments, multiple rounds of sorting are performed using a lower concentration of labeled target antigen with each round of sorting.

For example, an initial sort or selection using about 100 to 250 nM labeled target antigen should be sufficient to capture a wide range of affinities, although this factor can be determined empirically and/or to suit the desire of the practitioner. In the second round of selection, about 25 to 100 nM of labeled target antigen may be used. In the third round of selection, about 0.1 to 25 nM of labeled target antigen may be used. For example, to improve the affinity of a 100 nM binder, it may be desirable to start with 20 nM and then progress to 5 and 1 nM labeled target, then, followed by even lower concentrations such as about 0.1 nM labeled target antigen.

The conventional solution sorting involves use of beads like streptavidin-coated beads, which is very cumbersome to use and often results in very low efficiency of phage binder recovery. The conventional solution sorting with beads takes much longer than 2-5 minutes and is less feasible to adapt to high throughput automation than the invention described above.

As described herein, combinations of solid support and solution sorting methods can be advantageously used to isolate binders having desired characteristics. After selection/sorting on target antigen for a few rounds, screening of individual clones from the selected pool generally is performed to identify specific binders with the desired properties/characteristics. In some embodiments, the process of screening is carried out by automated systems to allow for high-throughput screening of library candidates.

Two major screening methods are described below. However, other methods known in the art may also be used in the methods of the invention. The first screening method comprises a phage ELISA assay with immobilized target antigen, which provides for identification of a specific binding clone from a non-binding clone. Specificity can be determined by simultaneous assay of the clone on target coated well and BSA or other non-target protein coated wells. This assay is automatable for high throughput screening.

One embodiment provides a method of selecting for an antibody variable domain that binds to a specific target antigen from a library of antibody variable domain by generating a library of replicable expression vectors comprising a plurality of polypeptides; contacting the library with a target antigen and at least one nontarget antigen under conditions suitable for binding; separating the polypeptide binders in the library from the nonbinders; identifying the binders that bind to the target antigen and do not bind to the nontarget antigen; eluting the binders from the target antigen; and amplifying the replicable expression vectors comprising the polypeptide binder that bind to a specific antigen.

The second screening assay is an affinity screening assay that provides for screening for clones that have high affinity from clones that have low affinity in a high throughput manner. In the assay, each clone is assayed with and without first incubating with target antigen of certain concentration for a period of time (e.g., 30-60 minutes) before application to target coated wells briefly (e.g., 5-15 minutes). Then bound phage is measured by usual phage ELISA method, e.g. using anti-M13 HRP conjugates. The ratio of binding signal of the two wells, one well having been preincubated with target and the other well not preincubated with target antigen is an indication of affinity. The selection of the concentration of target for first incubation depends on the affinity range of interest. For example, if binders with affinity higher than 10 nM are desired, 100 nM of target in the first incubation is often used. Once binders are found from a particular round of sorting (selection), these clones can be screened with an affinity screening assay to identify binders with higher affinity. Combinations of any of the sorting/selection methods described above may be combined with the screening methods. For example, in one embodiment, polypeptide binders are first selected for binding to immobilized target antigen. Polypeptide binders that bind to the immobilized target antigen can then be amplified and screened for binding to the target antigen and for lack of binding to nontarget antigens. Polypeptide binders that bind specifically to the target antigen are amplified. These polypeptide binders can then selected for higher affinity by contact with a concentration of a labeled target antigen to form a complex, wherein the concentration ranges of labeled target antigen from about 0.1 nM to about 1000 nM, the complexes are isolated by contact with an agent that binds to the label on the target antigen. The polypeptide binders are then eluted from the labeled target antigen and optionally, the rounds of selection are repeated, each time a lower concentration of labeled target antigen is used. The high affinity polypeptide binders isolated using this selection method can then be screened for high affinity using a variety of methods known in the art, some of which are described herein. These methods can provide for finding clones with high affinity without having to perform long and complex competition affinity assays on a large number of clones. The intensive aspect of doing complex assays of many clones often is a significant obstacle to finding best clones from a selection. This method is especially useful in affinity improvement efforts where multiple binders with similar affinity can be recovered from the selection process. Different clones may have very different efficiency of expression/display on phage or phagemid particles. Those clones more highly expressed have better chances being recovered. That is, the selection can be biased by the display or expression level of the variants. The solution-binding sorting method of the invention can improve the selection process for finding binders with high affinity. This method is an affinity screening assay that provides a significant advantage in screening for the best binders quickly and easily.

The antibodies or antigen binding fragments can be further selected for functional activity, for example, antagonist or agonist activity. For example, anti-her3 antibodies can be selected for the ability to inhibit tyrosine phosphorylation of HER-2, dimerization with Her2, proliferation of cancer cells or to induce apoptosis of cancer cells. Assays for identifying and measuring these activities are known. See for example in WO98/17797.

After binders are identified by binding to the target antigen, and/or functional assays the nucleic acid can be extracted. Extracted DNA can then be used directly to transform E. coli host cells or alternatively, the encoding sequences can be amplified, for example using PCR with suitable primers, and sequenced by any typical sequencing method. Variable domain DNA of the binders can be restriction enzyme digested and then inserted into a vector for protein expression.

Populations comprising polypeptides having CDR(s) with restricted sequence diversity generated according to methods of the invention can be used to isolate binders against a variety of targets, including those listed in this application. These binders may comprise one or more variant CDRs comprising diverse sequences generated using restricted codons. In some embodiments, a variant CDR is CDRH3 comprising sequence diversity generated by amino acid substitution with restricted codon sets and/or amino acid insertions resulting from varying CDRH3 lengths. One or more variant CDRs may be combined. In some embodiments, only CDRH3 is diversified. In other embodiments, two or more heavy chain CDRs, including CDRH3, are variant. In other embodiments, one or more heavy chain CDRs, excluding CDRH3, are variant. In some embodiments, at least one heavy chain and at least one light chain CDR are variant. In some embodiments, at least one, two, three, four, five or all of CDRs H1, H2, H3, L1, L2 and L3 are variant.

In some cases, it can be beneficial to combine one or more diversified light chain CDRs with novel binders isolated from a population of polypeptides comprising one or more diversified heavy chain CDRs. This process may be referred to as a 2-step process. An example of a 2-step process comprises first determining binders (generally lower affinity binders) within one or more libraries generated by randomizing one or more CDRs, wherein the CDRs randomized in each library are different or, where the same CDR is randomized, it is randomized to generate different sequences. Binders from a heavy chain library can then be randomized with CDR diversity in a light chain CDRs by, for example, a mutagenesis technique such as that of Kunkel, or by cloning (cut-and-paste (e.g. by ligating different CDR sequences together)) the new light chain library into the existing heavy chain binders that has only a fixed light chain. The pool can then be further sorted against one or more targets to identify binders possessing increased affinity. For example, binders (for example, low affinity binders) obtained from sorting an H1/H2/H3 may be fused with library of an L1 L2/L3 diversity to replace its original fixed L1/L2/L3, wherein the new libraries are then further sorted against a target of interest to obtain another set of binders (for example, high affinity binders). Novel antibody sequences can be identified that display higher binding affinity to any of a variety of target antigens.

In some embodiments, libraries comprising polypeptides of the invention are subjected to a plurality of sorting rounds, wherein each sorting round comprises contacting the binders obtained from the previous round with a target antigen distinct from the target antigen(s) of the previous round(s). Preferably, but not necessarily, the target antigens are homologous in sequence, for example members of a family of related but distinct polypeptides, such as, but not limited to, cytokines (for example, alpha interferon subtypes).

### Activity Assays

The antibodies of the present invention can be characterized for their physical/chemical properties and biological functions by various assays known in the art. The purified immunoglobulins can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In certain embodiments, the immunoglobulins produced herein are analyzed for their biological activity. In some embodiments, the immunoglobulins of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays.

The antibodies or antigen binding fragments can be further selected for functional activity, for example, antagonist or agonist activity. For example, anti-HER-2 antibodies can be selected for the ability to inhibit tyrosine phosphorylation of HER-2, inhibit proliferation of cancer cells or to induce apoptosis of cancer cells. Assays for identifying and measuring these activities are described for example in WO98/17797.

In one embodiment, the present invention contemplates an altered antibody that possesses some but not all effector functions, which make it a desired candidate for many applications in which the half life of the antibody in vivo is important yet certain effector functions (such as complement and ADCC) are unnecessary or deleterious. In certain embodiments, the Pc activities of the produced immunoglobulin are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc.gamma.R binding (hence likely lacking ADCC activity), but retains FcRn binding ability. The primary cells for mediating ADCC, NK cells, express Fc.γRIII only, whereas monocytes express Fc.gamma.RI, Fc.gamma.RII and Fc.gamma.RIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. No. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). C1q binding assays may also be carried out to confirm that the antibody is unable to bind C1q and hence lacks CDC activity. To assess complement activation, a CDC assay, for example as described in Gazzano-Santoro et al., J. Immunol. Methods 202:163 (1996), may be performed. FcRn binding and in viva clearance/half life determinations can also be performed using methods known in the art.

### Antibody Variants

In one aspect, the invention provides antibody fragments comprising modifications in the interface of Fc polypeptides comprising the Fc region, wherein the modifications facilitate and/or promote heterodimerization. These modifications comprise introduction of a protuberance into a first Fc polypeptide and a cavity into a second Fc polypeptide, wherein the protuberance is positionable in the cavity so as to promote complexing of the first and second Fc polypeptides. Methods of generating antibodies with these modifications are known in the art, e.g., as described in U.S. Pat. No. 5,731,168.

In some embodiments, amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

To increase the half-life of the antibodies or polypeptide containing the amino acid sequences of this invention, one can attach a salvage receptor binding epitope to the antibody (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG.sub.1, IgG.sub.2, IgG.sub.3, or IgG.sub.4) that is responsible for increasing the in vivo serum half-life of the IgG molecule (e.g., Ghetie, V et al., (2000) Ann. Rev. Immunol. 18:739-766, Table 1). Antibodies with substitutions in an Fc region thereof and increased serum half-lives are also described in WO00/42072 (Presta, L.), WO 02/060919; Shields, R. L., et al., (2001) JBC 276(9):6591-6604; Hinton, P. R., (2004) JBC 279(8):6213-6216). In another embodiment, the serum half-life can also be increased, for example, by attaching other polypeptide sequences. For example, antibodies of this invention or other polypeptide containing the amino acid sequences of this invention can be attached to serum albumin or a portion of serum albumin that binds to the FcRn receptor or a serum albumin binding peptide so that serum albumin binds to the antibody or polypeptide, e.g., such polypeptide sequences are disclosed in WO01/45746. In another embodiment, the half-life of a Fab according to this invention is increased by methods disclosed in for example Dennis, M. S., et al., (2002) JBC 277(38):35035-35043 for serum albumin binding peptide sequences.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 2 under the heading of “preferred substitutions”. If such substitutions result in a change in biological activity, then more substantial changes, denominated “exemplary substitutions” in the table below, or as further described below in reference to amino acid classes, may be introduced and the products screened.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (1), Pro (P), Phe (F), Trp (W), Met (M)
(2) uncharged polar: Gly (O), Ser (5), Thr (T), Cys (C), Tyr (Y), Asn (N), Gln (O)
(3) acidic: Asp (D), Glu (E)
(4) basic: Lys (K), Arg (R), H is (H)

Alternatively, naturally occurring residues may be divided into groups based on common side-chain properties:

(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: H is, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites.

One type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of the immunoglobulin polypeptides of the invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In accordance with this description and the teachings of the art, it is contemplated that in some embodiments, an antibody used in methods of the invention may comprise one or more alterations as compared to the wild type counterpart antibody, for example in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), for example, as described in WO99/51642. See also Duncan & Winter Nature 322:738-40 (1988); U.S. Pat. No. 5,648,260; U.S. Pat. No. 5,624,821; and WO94/29351 concerning other examples of Fc region variants.

### Antibody Derivatives

The antibodies of the present invention can be further modified to contain additional nonproteinaceous moieties that are known in the art and readily available. In certain embodiments, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### Uses

An antibody of the present invention may be used in, for example, in vitro, ex vivo and in vivo therapeutic methods. Variant antibodies of the invention can be used as an antagonist to partially or fully block the specific antigen activity in vitro, ex vivo and/or in vivo. Moreover, at least some of the antibodies diversified in accordance to the methods disclosed herein can neutralize antigen activity from other species. Accordingly, the antibodies of the invention can be used to inhibit a specific antigen activity, e.g., in a cell culture containing the antigen, in human subjects or in other mammalian subjects having the antigen with which an antibody of the invention cross-reacts (e.g. chimpanzee, baboon, marmoset, cynomolgus and rhesus, pig or mouse). Thus, in an exemplary embodiment, a diversified antibody of the invention can be used for inhibiting antigen activities by contacting the antibody with the antigen such that antigen activity is inhibited. In certain embodiments, the antigen is a human protein molecule.

In one embodiment, a diversified antibody in accordance to the methods of the invention can be used in a method for inhibiting an antigen in a subject suffering from a disorder in which the antigen activity is detrimental, comprising administering to the subject an antibody of the invention such that the antigen activity in the subject is inhibited. In certain embodiments, the antigen is a human protein molecule and the subject is a human subject. Alternatively, the subject can be a mammal expressing the antigen with which an antibody of the invention binds. Still further the subject can be a mammal into which the antigen has been introduced (e.g., by administration of the antigen or by expression of an antigen transgene). An antibody of the invention can be administered to a human subject for therapeutic purposes. Moreover, an antibody of the invention can be administered to a non-human mammal expressing an antigen with which the immunoglobulin cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

Having generally described the invention, the same will be more readily understood by reference to the following examples, which are provided by way of illustration and are not intended as limiting.

### Example 1

### Generation of an Antibody Discovery Library

It has been described previously the methods for generating an antibody library from lead sequences (U.S. Pat. No. 7,117,096) for affinity maturation and optimization. Such an approach was expanded to generate a nave discovery library without prior knowledge of lead sequences. The purpose of such a library design was to incorporate a large population of sequences that can produce productive and functional interactions with a wide array of antigens. To design such a “discovery” library, a knowledge-based, rational approach, utilizing existing data, is preferable to a purely randomization approach. A knowledge-based library design utilized structural database as well as sequence database and any combination of data, such as statistical distribution, probabilistic models, and structural motifs, to identify “good” sequences.

From a structural point of view, the scaffolds of antibodies, or the immunoglobulin fold, is one of the most abundant observed in nature and is highly conserved among various antibodies and related molecules. For the purposes of library design, we focused primarily on the structure of the Fv domain, the variable fragment of the antibody. Structurally, various functions of an antibody are confined to a discrete set of protein segments. The sites that recognize and bind antigen consist of three hyper-variable or complementarity-determining regions (CDRs) that lie within the variable (VH and VL) regions at the N-terminal ends of the two H and two L chains. The constant domains are not directly involved in binding to an antigen, but are involved in various structural support and effector functions.

Natural VH and VL chains have the same general structures, and each domain comprises four framework regions, whose sequences are somewhat conserved, connected by three CDRs. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen binding site.

The VL and VH domains interact via the five-stranded beta sheets to form a nine-stranded beta barrel of about 8.4 Angstroms radius, with the strands at the domain interface inclined at approximately 50 degree to one another. The domain pairing brings the CDR loops into close proximity. The CDRs themselves form some 25% of the VL-VH domain interface.

The six CDRs, (CDR-L1, -L2 and -L3 for the light chain, and CDR-H1, -H2 and -H3 for the heavy chain), are supported on the beta barrel framework, forming the antigen binding site. While their sequences are hypervariable in comparison with the rest of the immunoglobulin structure, some of the loops show a relatively high degree of both sequence and structural conservation. In particular, CDR-L2 and CDR-H1 are highly conserved in conformation.

A library for antibody discovery can be typically described generally by an amino acid sequence of a select number of FRs and variant mixtures of CDR sequences. Variants are introduced primarily in the CDR regions since these directly interact with antigens.

Computer-implemented analysis and modeling of antibody combining site (or antigen binding site), based on homology analysis comparing the target antibody sequence with those of antibodies with known structures or structural motifs in existing data bases (e.g. the Brookhaven Protein Data Bank), were utilized in PDL1 design. By using such homology-based modeling methods approximate three-dimensional structure of the target antibody was constructed.

The inventors believe that properly folded structures are essential for binding and function of an antibody. Identifying such sequences is still a challenge in modern protein science. By analyzing available high resolution crystal structures, we observed that the canonical structures can be seen in majority of the CDR as well as FR structures. These can be categorized into length groups and within each length group, e.g., VH_CDR3, canonical structures can be identified that provide compatible structures for a set of sequence variants,

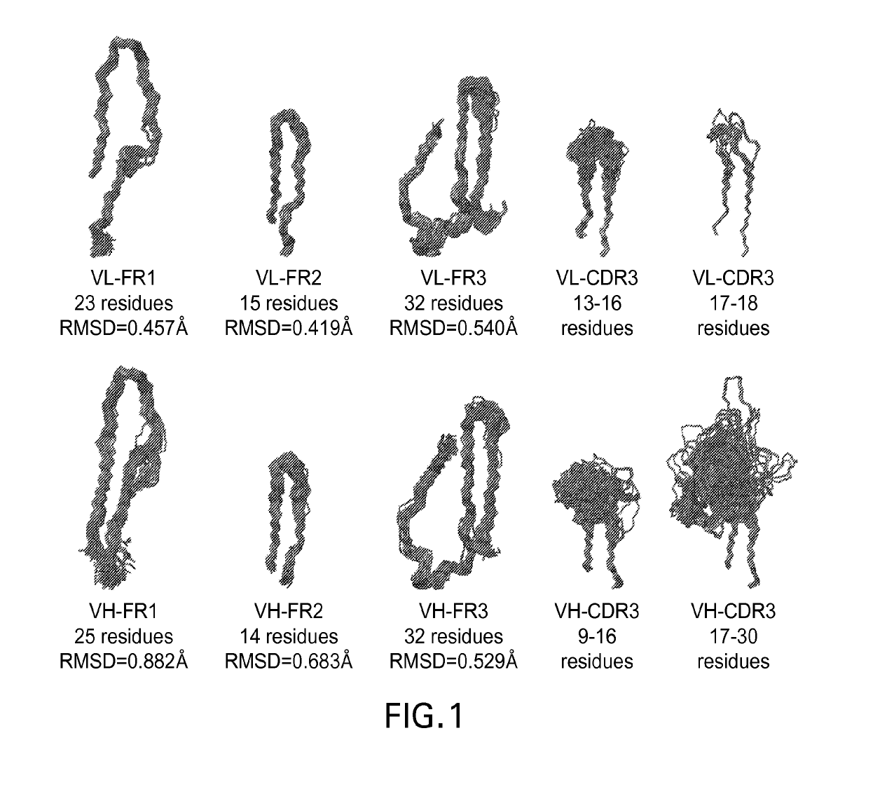

For some representative FRs, the root mean square deviation (RMSD) for alpha carbon is <1 Angstrom whereas CDR3 from both VH and VL show great backbone diversity among many lengths. FIG. 1 shows the consistency of FR backbones among several hundred known crystal structures. The figure also shows the diversity of structural folds for various lengths.

### Example 2

### Frameworks for Antibody Discovery Library PDL1

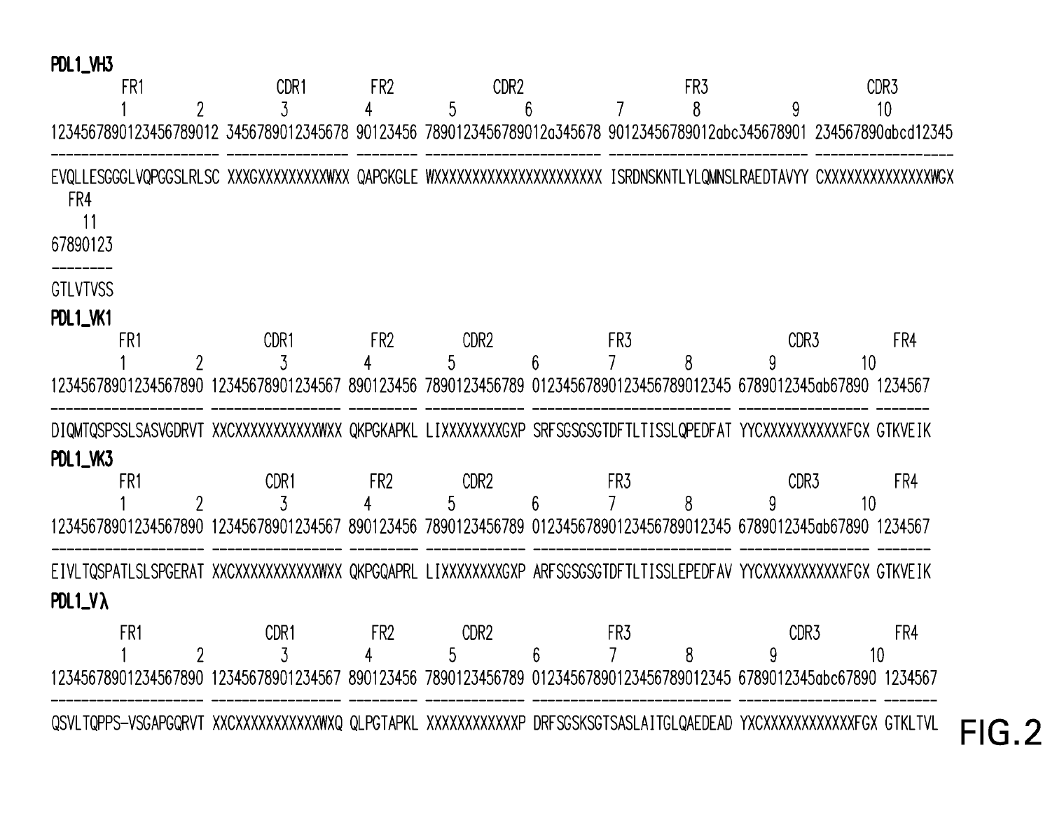

PDL1 library (production discovery library 1) is a fully human and synthetic Fab library. One VH framework from human germline VH3 family and three light chains from human germline VK1, VK3 and Vlambda 1 families were chosen, based on their highly frequent usage and canonical structures, to accommodate the designed CDR libraries. FIG. 2 illustrates those VH and VL framework sequences. X presents the amino acid in CDR region based on Abmaxis' definition.

### Example 3

### PDL1 Library CDR Length Selection

Sequence and structure analysis were carried out on SCOP 1.69 database (Murzin A. G., Brenner S. E., Hubbard T., Chathia C. (1995). SCOP: a structural classification of proteins database for the investigation of sequences and structures. J. Mol. Biol. 247, 536-540). 702 unique antibody heavy chain variable regions (VH) and 774 unique antibody light chain variable regions (VL) were analyzed. Each unique chain was parsed into 4 frameworks and 3 CDR segments based on Kabat definition except VH CDR1 which was defined as Chothia definition.

The CDR length distribution of the antibody sequences was obtained through analysis. The percentage for the certain CDR length over the total number of available sequence for that CDR was calculated and summarized in the table 1:

TABLE 1
CDR length distribution of human antibody in SCOP 1.69

| | CDR length | # of sequence | % |
|---|---|---|---|
| VH_CDR1 | 2 | 1 | <1 |
| | 5 | 1 | <1 |
| | 7 | 6 | 1 |
| | 8 | 5 | 1 |
| | 9 | 3 | <1 |
| | 10 | 622 | 89 |
| | 11 | 43 | 6 |
| | 12 | 19 | 3 |
| | 13 | 1 | <1 |
| VH_CDR2 | 13 | 1 | <1 |
| | 14 | 2 | <1 |
| | 15 | 3 | <1 |
| | 16 | 172 | 25 |
| | 17 | 454 | 65 |
| | 19 | 67 | 10 |
| | 22 | 3 | <1 |
| VH_CDR3 | 2 | 1 | <1 |
| | 3 | 16 | 2 |
| | 4 | 7 | 1 |
| | 5 | 65 | 9 |
| | 6 | 16 | 2 |
| | 7 | 51 | 7 |
| | 8 | 70 | 10 |
| | 9 | 55 | 8 |
| | 10 | 129 | 19 |
| | 11 | 98 | 14 |
| | 12 | 51 | 7 |
| | 13 | 41 | 6 |
| | 14 | 38 | 5 |
| | 15 | 9 | 1 |
| | 16 | 9 | 1 |
| | 17 | 9 | 1 |
| | 18 | 4 | 1 |
| | 19 | 10 | 1 |
| | 20 | 5 | 1 |
| | 22 | 4 | 1 |
| | 24 | 8 | 1 |
| VL_CDR1 | 8 | 1 | <1 |
| | 9 | 1 | <1 |
| | 10 | 37 | 5 |
| | 11 | 311 | 40 |
| | 12 | 27 | 3 |
| | 13 | 24 | 3 |
| | 14 | 106 | 14 |
| | 15 | 35 | 5 |
| | 16 | 168 | 22 |
| | 17 | 64 | 8 |
| VL_CDR2 | 2 | 1 | <1 |
| | 3 | 1 | <1 |
| | 5 | 2 | <1 |
| | 7 | 769 | 99 |
| | 8 | 1 | <1 |
| VL_CDR3 | 2 | 1 | <1 |
| | 7 | 2 | <1 |
| | 8 | 45 | 6 |
| | 9 | 642 | 83 |
| | 10 | 50 | 6 |
| | 11 | 28 | 4 |
| | 12 | 2 | <1 |

PDL1 library captured the majority of the CDR length and structural diversity as shown in table 2.

TABLE 2
PDL1 captured CDR length distribution

| | PDL1 captured CDR Length | % |
|---|---|---|
| VH_CDR1 | 10, 11, 12 | 98 |
| VH_CDR2 | 16, 17, 19 | 99 |
| VH_CDR3 | 5, 7, 8, 9, 10, 11, 12, 13, 14 | 86 |
| VL_CDR1 | 10, 11, 14, 15, 16, 17 | 93 |
| VL_CDR2 | 7 | 99 |
| VL_CDR3 | 8, 9, 10, 11 | 99 |

### Example 4

### PDL1 CDR Structural Scaffold Selection

For the CDRs with the length listed in the tablet, the corresponded CDR structures in SCOP 1.69 structure database were clustered according to the CA atoms with a RMSD 2.5 Å cutoff. The number of the structural clusters were captured in PDL1 are summarized in table 3.

TABLE 3
The number of CDR structure scaffold and captured by PDL1

| | | Number of PDL1 CDR |
|---|---|---|
| | CDR Length | structural cluster |
| VH_CDR1 | 10 | 6 |
| | 11 | 1 |
| | 12 | 2 |
| VH_CDR2 | 16 | 2 |
| | 17 | 5 |
| | 19 | 2 |
| VH_CDR3 | 5 | 2 |
| | 7 | 2 |
| | 8 | 4 |
| | 9 | 5 |
| | 10 | 9 |
| | 11 | 9 |
| | 12 | 7 |
| | 13 | 8 |
| | 14 | 8 |
| VL_CDR1 | 10 | 2 |
| | 11 | 1 |
| | 14 | 6 |
| | 15 | 2 |
| | 16 | 2 |
| | 17 | 1 |
| VL_CDR2 | 7 | 1 |
| VL_CDR3 | 8 | 1 |
| | 9 | 2 |
| | 10 | 2 |
| | 11 | 2 |
| Total | | 94 |

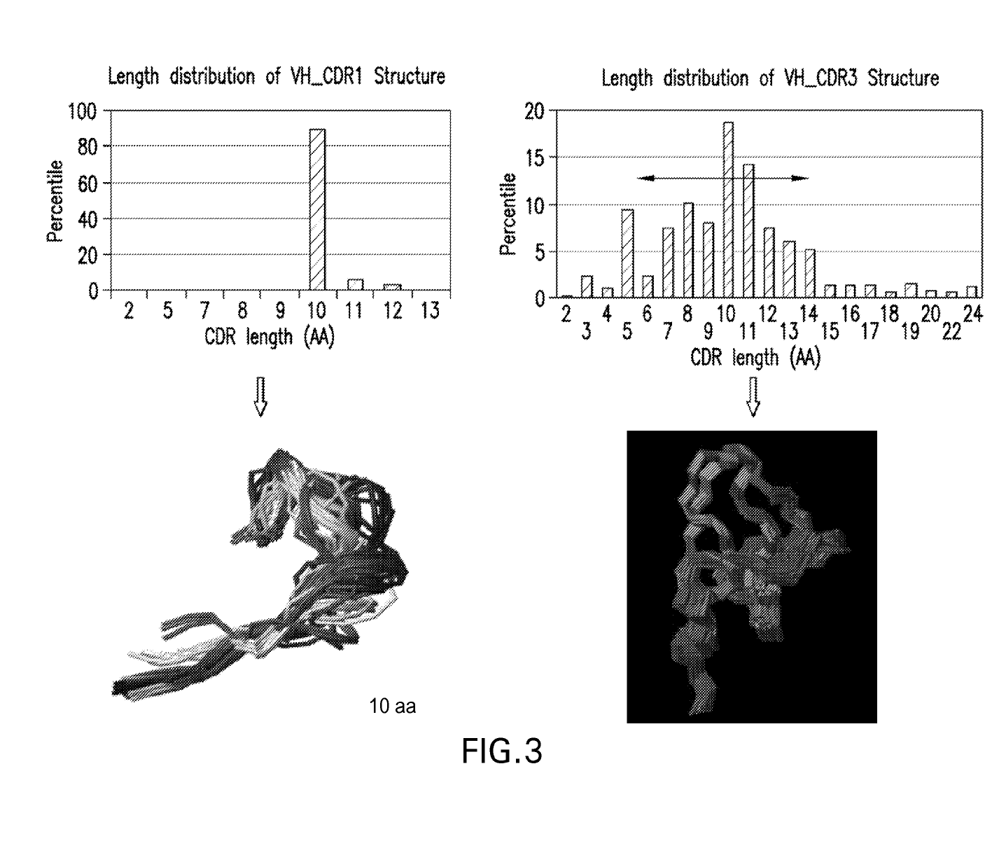

FIG. 3 further illustrate heavy chain CDR length distributions and CDR loop structure clusters.

### Example 5

### PDL1 CDR Library Design

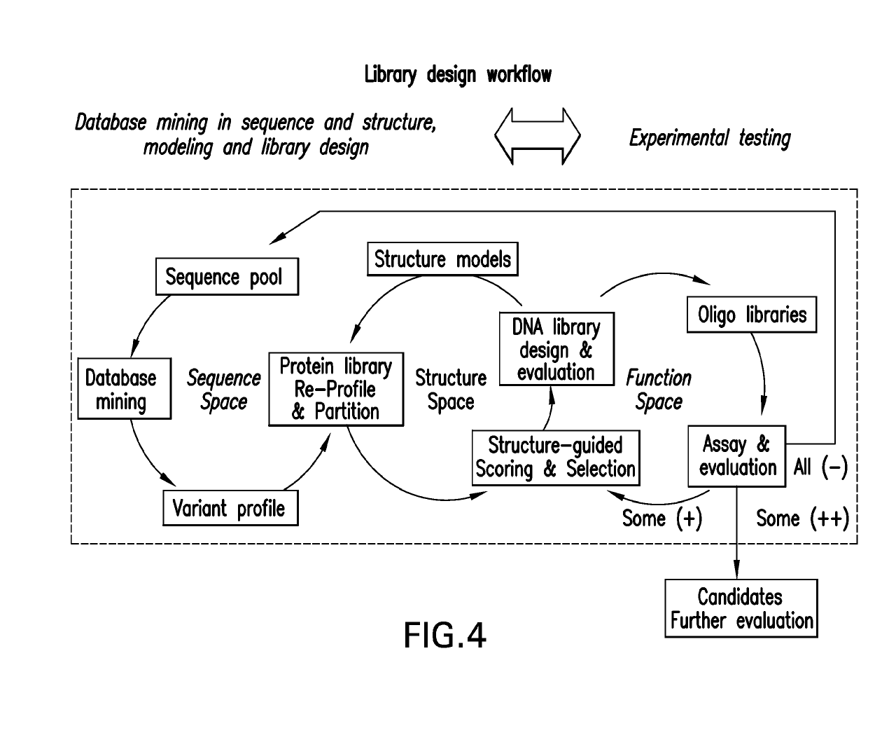

For each CDR structural cluster listed in table 3, the corresponded CDR sequences were used as lead sequences to generate the variant profile using both sequence and structure-based design as described in U.S. Pat. No. 7,117,096 B2. The design process is illustrated in FIG. 4

As for the sequence-based design, the antibody sequence database profiling was carried out for each leading sequence. The antibody sequence database used here are the combination of the antibody sequences from Kabat database, IMGT, PDB and Merck internal antibody database. The variant profiles were identified by the sequence alignment, statistical and probabilistic analysis using HMM and filtered with certain cutoff value to make the size of the variant profile within the computational or experimental limit.

As for the structure-based design, the variants were ranked against the corresponded CDR structural cluster by the van der Waals energy for the packing stabilities. In addition, the variants were also assessed on the chain and Fv structures. The chain and Fv structures were built by the Abmaxis antibody structure modeling package which was described in U.S. Pat. No. 117,096 B2.

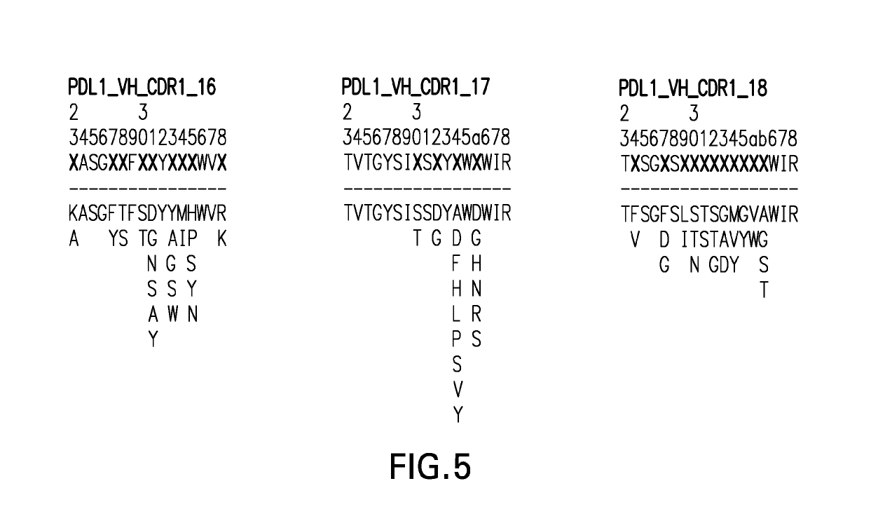

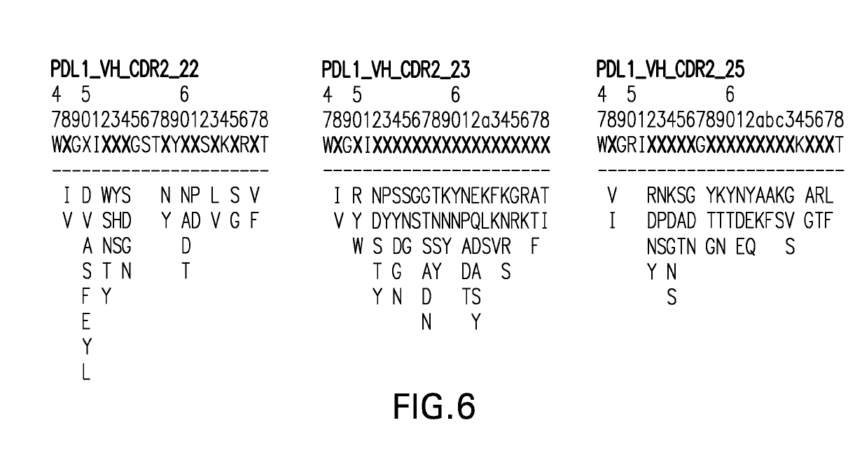

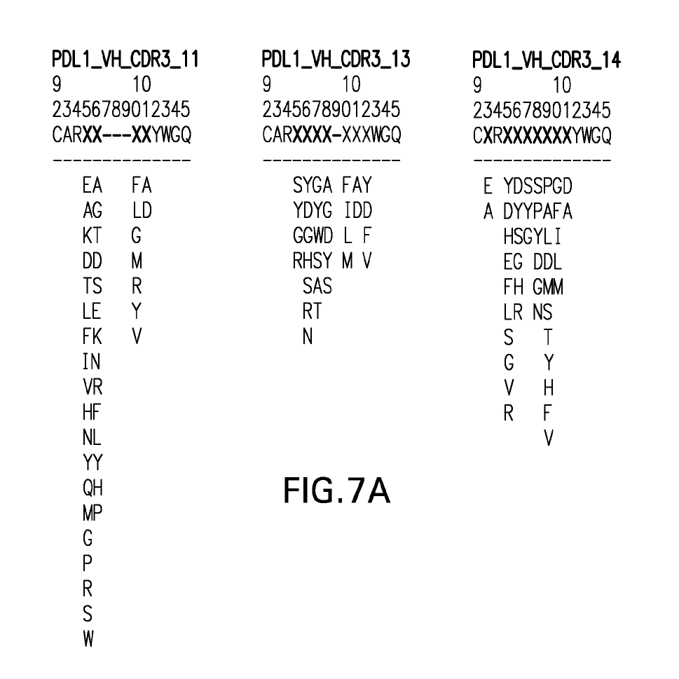

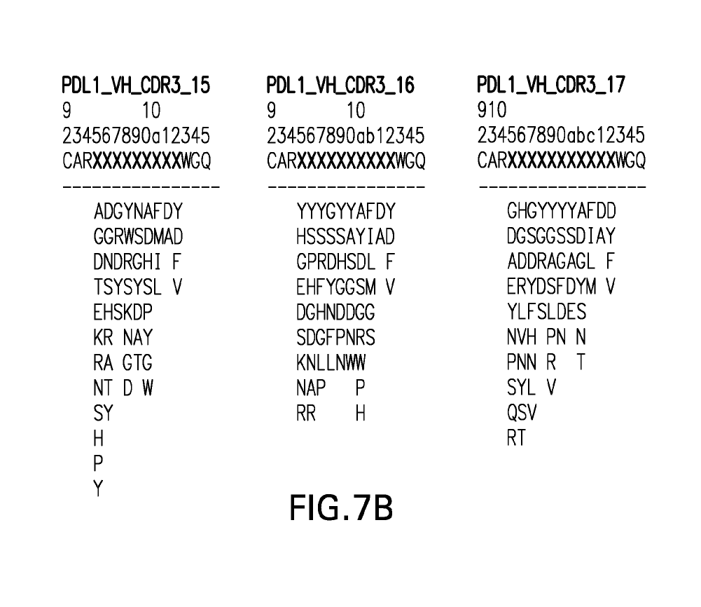

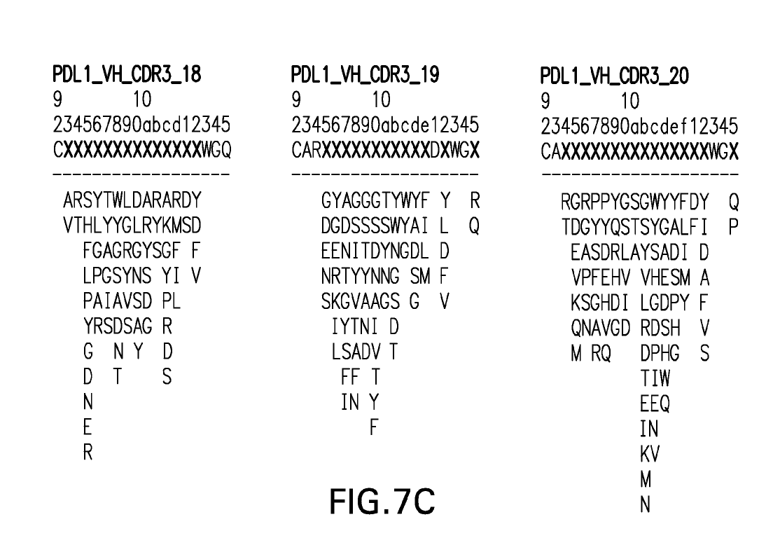

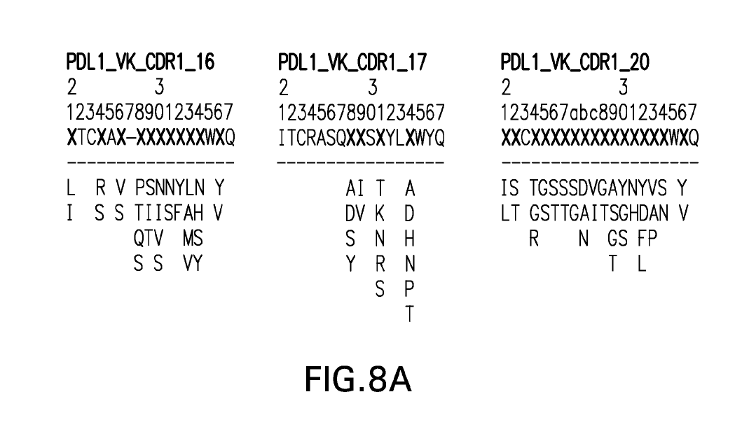

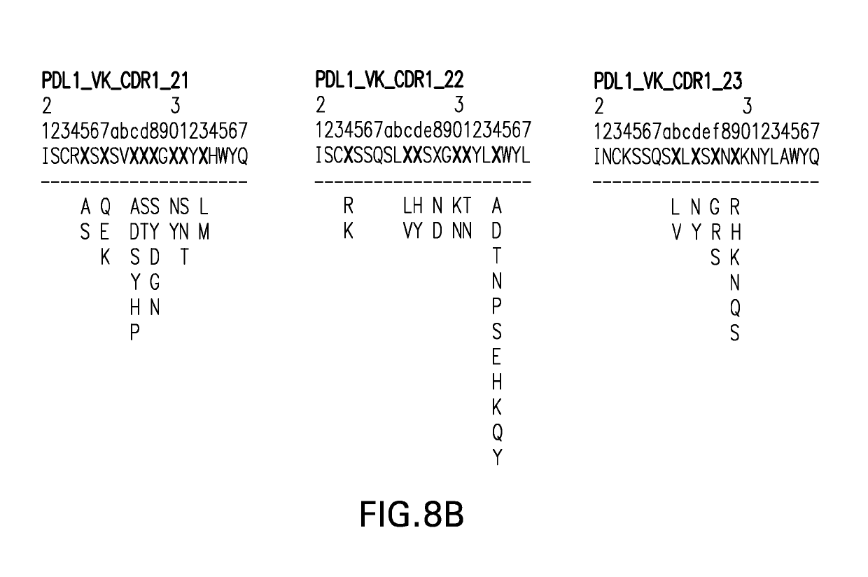

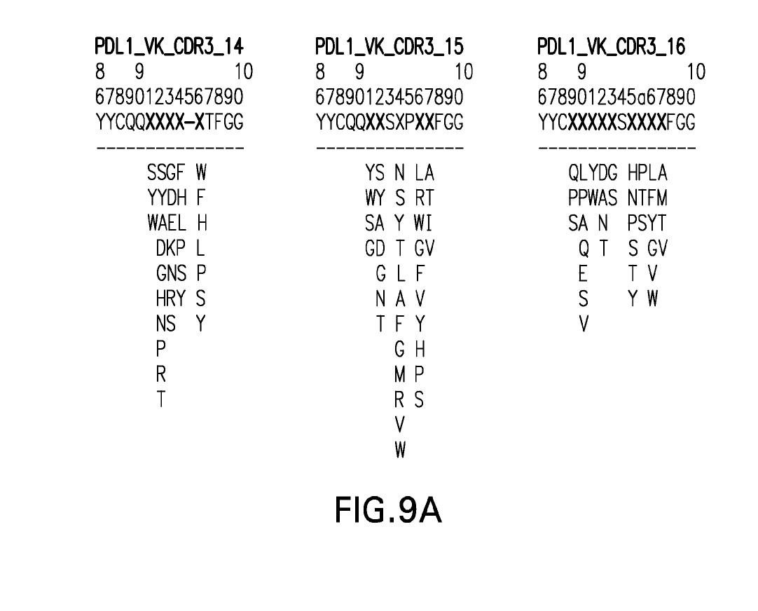

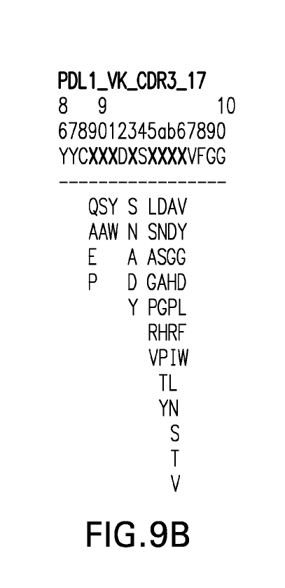

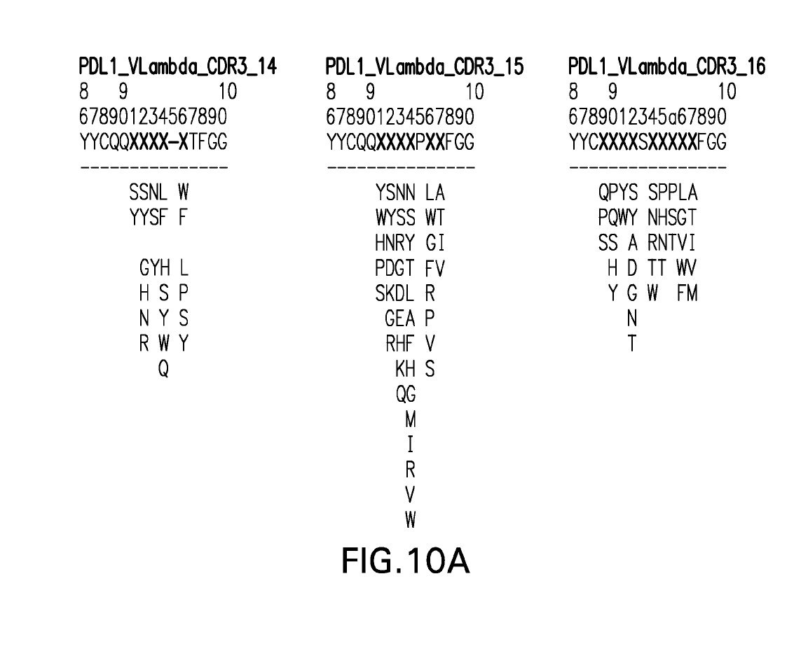

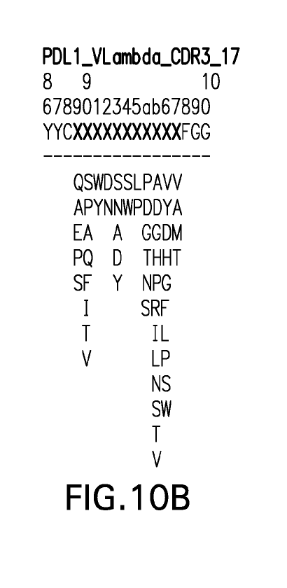

The final CDR libraries were obtained by consolidating both sequence and structure based design, as shown in FIG. 5-10.

The CDR libraries shown in FIG. 5-10 were split into multiple small libraries to avoid undesired amino acids and stop codons from radon DNA codon combinations, such as cyteins, glycosylation sites, amber stop codon, etc, and to reduce the final library sizes. Table 4 illustrates the changes of number of CDR libraries before and after library splitting.

TABLE 4
CDR libraries before and after splitting to avoid undesired features

Number of libraries

| | fully codon | limited codon |
|---|---|---|
| | degeneracy | degeneracy |
| VH_CDR1 | 9 | 24 |
| VH_CDR2 | 9 | 13 |
| VH_CDR3 | 54 | 523 |
| VL_CDR1 | 14 | 14 |
| VL_CDR2 | 1 | 1 |
| VL_CDR3 | 7 | 44 |
| VLambda_CDR3 | 7 | 33 |
| total | 101 | 642 |

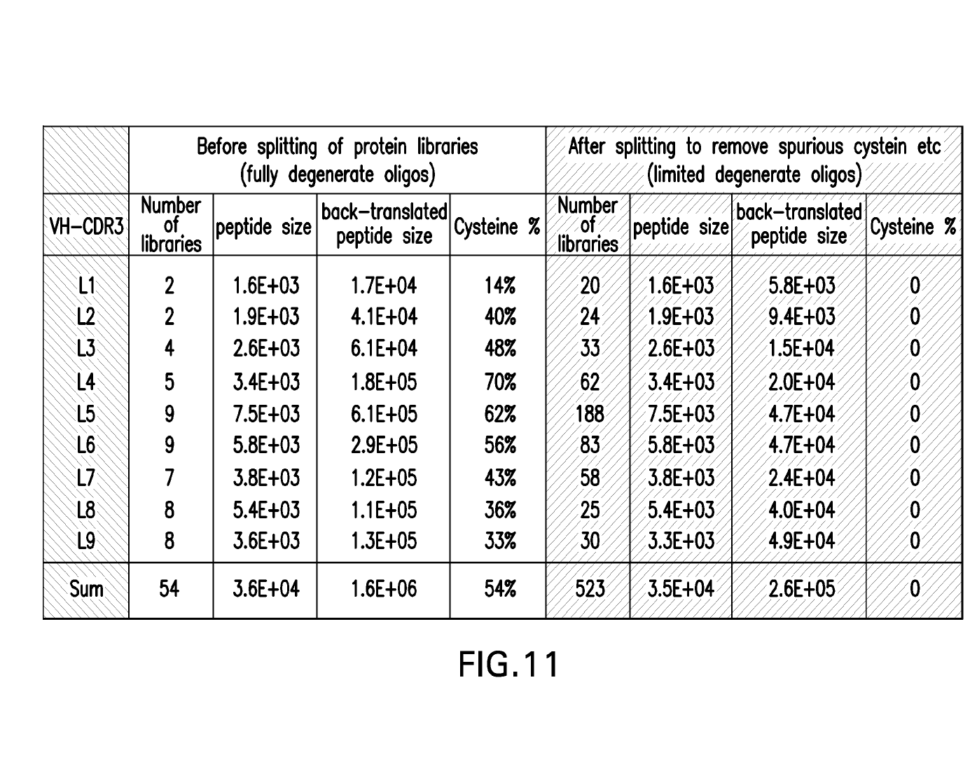

FIG. 11 shows examples to reduce the possibility of cyteins from 54% (before split) to 0% (post split), as well as the size of libraries.
The split final CDR libraries of PDL1 library are listed below.

PDL1_VH_CDR1.1
KASGX1TFX2X3YYMHWVR
X1: F Y
X2: S T
X3: D G N S
PDL1_VH_CDR1.2
KASGX1TFX2X3YX41VIHWVR
X1: F Y
X2: S T
X3: D G N S
X4: A G S W
PDL1_VH_CDR1.3
KASGX1TFX2X3YYMX4WVR
X1: F Y
X2: S T
X3: N S
X4: H P S Y
PDL1_VH_CDR1.4
KASGX1TFX2X3YX4MX5WVR
X1: F Y
X2: S T
X3: N S
X4: A G S W
X5: H P S Y
PDL1_VH_CDR1.5
KASGX1TFX2X3YYMX4WVR
X1: F Y
X2: S T
X3: A D S Y
X4: H P S Y
PDL1_VH_CDR1.6
KASGX1TFX2X3YX4MX5WVR
X1: F Y
X2: S T
X3: A D S Y
X4: A G
X5: H P S Y
PDL1_VH_CDR1.7
AASGX1TFX2X3YYMHWVR
X1: F Y
X2: S T
X3: D G N S
PDL1_VH_CDR1.8
AASGX1TFX2X3YX4MHWVR
X1: F Y
X2: S T
X3: D G N S
X4: A G S W
PDL1_VH_CDR1.9
KASGX1TFX2X3YYMX4WVR
X1: F Y
X2: S T
X3: N S
X4: H N
PDL1_VH_CDR1.10
KASGX1TFX2X3YX4MX5WVR
X1: F Y
X2: S T
X3: N S
X4: A G S W
X5: H N
PDL1_VH_CDR1.11
KASGYX1FTX2YX3X4X5WVK
X1: S T
X2: D G N S
X3: G W
X4: I M
X5: H N
PDL1_VH_CDR1.12
TVTGYSIX1SX2YX3WX4WIR
X1: S T
X2: D G
X3: A D F H L P S V Y
X4: D G H N R S
PDL1_VH_CDR1.13
TFSGFSLX1TX2GX3GVX4WIR
X1: S T
X2: S T
X3: M V
X4: A G S T
PDL1_VH_CDR1.14
TVSGX1SIX2SGX3YYWX4WIR
X1: D G
X2: N S
X3: A D G N S T
X4: G S
PDL1_VH_CDR2.1
WVGYISX1X2GSTYYX3X4SX5KX6RFT
X1: S Y
X2: A D S Y
X3: A D N T
X4: A D H P
X5: L V
X6: G S
PDL1_VH_CDR2.2
WIGX1IX2X3SGSTX4YNPSLKSRVT
X1: A S V D E F L Y
X2: N S T Y
X3: H Y
X4: N Y
PDL1_VH_CDR2.3
WIGX1IWX2SGSTX3YNPSLKSRVT
X1: A S V D E F L Y
X2: H Y
X3: N Y
PDL1_VH_CDR2.4
WVGRIX1PX2GGX3TYYADSX4KGRFT
X1: N S
X2: D G N S
X3: D G N S
X4: F V
PDL1_VH_CDR2.5
WIGX1IX2PX3X4GGTX5YNX6KFKGKAT
X1: R W
X2: D N
X3: D G N S
X4: N S
X5: K N
X6: E Q
PDL1_VH_CDR2.6
WMGYIX1YSGX2TX3YNPX4LKX5RISI
X1: N S T Y
X2: G S
X3: N S T Y
X4: A D S Y
X5: K N R S
PDL1_VH_CDR2.7
WIGRIX1PX2X3GGTX4YNX5KFKX6KAT
X1: D N
X2: N Y
X3: N S
X4: K N
X5: E Q
X6: G S
PDL1_VH_CDR2.8
WIGRIX1PX2X3GGTX4YNX5KFKGKAT
X1: D N
X2: S Y
X3: N S
X4: K N
X5: E Q
PDL1_VH_CDR2.9
WIGRIX1PX2X3GGTX4YNX5KFKGKAT
X1: D N
X2: D G N S
X3: N S
X4: K N
X5: E Q
PDL1_VH_CDR2.10
WVGRIRX1KX2X3GYTTX4YAASVKGRFT
X1: N S
X2: A S T
X3: D N
X4: D E
PDL1_VH_CDR2.11
WVGRIRX1KX2X3GGTTX4YAASVKGRFT
X1: N S
X2: A S T
X3: D N
X4: D E
PDL1_VH_CDR2.12
WIGX1IYPGX2GGTX3YNX4KFKGKATLT
X1: D Y
X2: N S
X3: K N R S
X4: E Q
PDL1_VH_CDR2.13
WIGX1IYPGX2GGTX3YNX4KFKGKATLT
X1: R W
X2: N S
X3: K N R S
X4: E Q
PDL1_VH_CDR3.1
CARX1X2X3X4YWGQ
X1: L I V D E F F I K N Q Y
X2: F L Y
X3: F L Y
X4: A D
PDL1_VH_CDR3.2
CARX1X2X3X4YWGQ
X1: A G P R S W
X2: F L Y
X3: F L Y
X4: A D
PDL1_VH_CDR3.3
CARX1X2X3X4YWGQ
X1: L Q V D E F H I K M N Y
X2: A G T D E K N R S
X3: F L Y
X4: A D
PDL1_VH_CDR3.4
CARX1X2X3X4YWGQ
X1: A G P R S W
X2: A G T D E K N R S
X3: F L Y
X4: A D
PDL1_VH_CDR3.5
CARX1X2X3X4YWGQ
X1: L I V D E F H K N Q Y
X2: F L Y
X3: G M R V
X4: A D
PDL1_VH_CDR3.6
CARX1X2X3X4YWGQ
X1: A G P R S W
X2: F L Y
X3: G M R V
X4: A D
PDL1_VH_CDR3.7
CARX1X2X3X4YWGQ
X1: L I V D E F H K N Q Y
X2: A G T D E K N R S
X3: G M R V
X4: A D
PDL1_VH_CDR3.8
CARX1X2X3X4YWGQ
X1: A G P R S W
X2: A G T D E K N R S
X3: G M R V
X4: A D
PDL1_VH_CDR3.9
CARX1X2X3X4YWGQ
X1: A E K T
X2: A G T D E K N R S
X3: F L
X4: A D
PDL1_VH_CDR3.10
CARX1X2X3X4YWGQ
X1: A T D E K N
X2: F H L P S Y
X3: G M R V
X4: A D
PDL1_VH_CDR3.11
CARX1X2X3X4YWGQ
X1: A E K T
X2: A G T D E K N R S
X3: G M R V
X4: A D
PDL1_VH_CDR3.12
CARX1X2X3X4YWGQ
X1: D F H I L N V Y
X2: F H L P S Y
X3: F L
X4: A D
PDL1_VH_CDR3.13
CARX1X2X3X4YWGQ
X1: G R W
X2: F H L P S Y
X3: F L
X4: A D
PDL1_VH_CDR3.14
CARX1X2X3X4YWGQ
X1: D F H I L N V Y
X2: A G T D E K N R S
X3: F L
X4: A D
PDL1_VH_CDR3.15
CARX1X2X3X4YWGQ
X1: G R W
X2: A G T D E K N R S
X3: F L
X4: A D
PDL1_VH_CDR3.16
CARX1X2X3X4YWGQ
X1: D F H I L N V Y
X2: F H L P S Y
X3: G M R V
X4: A D
PDL1_VH_CDR3.17
CARX1X2X3X4YWGQ
X1: G R W
X2: F H L P S Y
X3: G M R V
X4: A D
PDL1_VH_CDR3.18
CARX1X2X3X4YWGQ
X1: D F H I L N V Y
X2: A G T D E K N R S
X3: G M R V
X4: A D
PDL1_VH_CDR3.19
CARX1X2X3X4YWGQ
X1: G R W
X2: A G T D E K N R S
X3: G M R V
X4: A D
PDL1_VH_CDR3.20
CARX1X2X3X4YWGQ
X1: A T D E K N
X2: F H L P S Y
X3: F L
X4: A D
PDL1_VH_CDR3.21
CARX1X2X3YX4X5X6WGQ
X1: G R
X2: S Y
X3: S Y
X4: F I L M
X5: A D
X6: D F V Y
PDL1_VH_CDR3.22
CARX1X2X3YX4X5X6WGQ
X1: G R
X2: D G H R
X3: S Y
X4: F I L M
X5: A D
X6: D F V Y
PDL1_VH_CDR3.23
CARX1X2X3YX4X5X6WGQ
X1: G R
X2: S Y
X3: A G S T
X4: F I L M
X5: A D
X6: D F V Y
PDL1_VH_CDR3.24
CARX1X2X3YX4X5X6WGQ
X1: G R
X2: D G H R
X3: A G S T
X4: F I L M
X5: A D
X6: D F V Y
PDL1_VH_CDR3.25
CARX1X2X3X4X5X6X7WGQ
X1: G R
X2: S Y
X3: S Y
X4: A G
X5: F I L M
X6: A D
X7: D F V Y
PDL1_VH_CDR3.26
CARX1X2X3X4X5X6X7WGQ
X1: G R
X2: D G H R
X3: S Y
X4: A G
X5: F I L M
X6: A D
X7: D F V Y
PDL1_VH_CDR3.27
CARX1X2X3X4X5X6X7WGQ
X1: G R
X2: S Y
X3: A G S T
X4: A G
X5: F I L M
X6: A D
X7: D F V Y
PDL1_VH_CDR3.28
CARX1X2X3X4X5X6X7WGQ
X1: G R
X2: D G H R
X3: A G S T
X4: A G
X5: F I L M
X6: A D
X7: D F V Y
PDL1_VH_CDR3.29
CARGYYX1X2X3YWGQ
X1: A D G
X2: F I L M
X3: A D
PDL1_VH_CDR3.30
CARX1X2YX3X4X5YWGQ
X1: S Y
X2: D G H N R S
X3: A D G
X4: F I L M
X5: A D
PDL1_VH_CDR3.31
CARGX1YX2X3X4YWGQ
X1: D G H N R S
X2: A D G
X3: F I L M
X4: A D
PDL1_VH_CDR3.32
CARX1YX2X3X4X5YWGQ
X1: S Y
X2: G W
X3: A D G
X4: F I L M
X5: A D
PDL1_VH_CDR3.33
CARGYX1X2X3X4YWGQ
X1: G W
X2: A D G
X3: F I L M
X4: A D
PDL1_VH_CDR3.34
CARX1X2X3X4X5X6YWGQ
X1: S Y
X2: D G H N R S
X3: G W
X4: A D G
X5: F I L M
X6: A D
PDL1_VH_CDR3.35
CARGX1X2X3X4X5YWGQ
X1: D G H N R S
X2: G W
X3: A D G
X4: F I L M
X5: A D
PDL1_VH_CDR3.36
CARX1YYX2X3X4YWGQ
X1: S Y
X2: S Y
X3: F I L M
X4: A D
PDL1_VH_CDR3.37
CARGYYX1X2X3YWGQ
X1: S Y
X2: F I L M
X3: A D
PDL1_VH_CDR3.38
CARX1X2YX3X4X5YWGQ
X1: S Y
X2: D G H N R S
X3: S Y
X4: F I L M
X5: A D
PDL1_VH_CDR3.39
CARGX1YX2X3X4YWGQ
X1: D G H N R S
X2: S Y
X3: F I L M
X4: A D
PDL1_VH_CDR3.40
CARX1YX2X3X4X5YWGQ
X1: S Y
X2: G W
X3: S Y
X4: F I L M
X5: A D
PDL1_VH_CDR3.41
CARGYX1X2X3X4YWGQ
X1: G W
X2: S Y
X3: F I L M
X4: A D
PDL1_VH_CDR3.42
CARX1X2X3X4X5X6YWGQ
X1: S Y
X2: D G H N R S
X3: G W
X4: S Y
X5: F I L M
X6: A D
PDL1_VH_CDR3.43
CARGX1X2X3X4X5YWGQ
X1: D G H N R S
X2: G W
X3: S Y
X4: F I L M
X5: A D
PDL1_VH_CDR3.44
CARX1YYX2X3X4YWGQ
X1: S Y
X2: A D G
X3: F I L M
X4: A D
PDL1_VH_CDR3.45
CARX1X2X3X4X5X6DYWGQ
X1: D G H R
X2: S Y
X3: S Y
X4: D G
X5: A D S Y
X6: F I L M
PDL1_VH_CDR3.46
CARX1X2X3X4X5X6DYWGQ
X1: S Y
X2: D G H R
X3: S Y
X4: D G
X5: A D S Y
X6: F I L M
PDL1_VH_CDR3.47
CARX1X2X3X4X5X6DYWGQ
X1: D G H R
X2: D G H R
X3: S Y
X4: D G
X5: A D S Y
X6: F I L M
PDL1_VH_CDR3.48
CARX1X2GX3X4X5DYWGQ
X1: S Y
X2: S Y
X3: D G
X4: A D S Y
X5: F I L M
PDL1_VH_CDR3.49
CARX1X2GX3X4X5DYWGQ
X1: D G H R
X2: S Y
X3: D G
X4: A D S Y
X5: F I L M
PDL1_VH_CDR3.50
CARX1X2GX3X4X5DYWGQ
X1: S Y
X2: D G H R
X3: D G
X4: A D S Y
X5: F I L M
PDL1_VH_CDR3.51
CARX1X2GX3X4X5DYWGQ
X1: D G H R
X2: D G H R
X3: D G
X4: A D S Y
X5: F I L M
PDL1_VH_CDR3.52
CARX1X2X3YX4X5DYWGQ
X1: S Y
X2: S Y
X3: S Y
X4: A D S Y
X5: F I L M
PDL1_VH_CDR3.53
CARX1X2X3YX4X5DYWGQ
X1: D G H R
X2: S Y
X3: S Y
X4: A D S Y
X5: F I L M
PDL1_VH_CDR3.54
CARX1X2X3YX4X5DYWGQ
X1: S Y
X2: D G H R
X3: S Y
X4: A D S Y
X5: F I L M
PDL1_VH_CDR3.55
CARX1X2X3YX4X5DYWGQ
X1: D G H R
X2: D G H R
X3: S Y
X4: A D S Y
X5: F I L M
PDL1_VH_CDR3.56
CARX1X2GYX3X4DYWGQ
X1: S Y
X2: S Y
X3: A D S Y
X4: F I L M
PDL1_VH_CDR3.57
CARX1X2GYX3X4DYWGQ
X1: D G H R
X2: S Y
X3: A D S Y
X4: F I L M
PDL1_VH_CDR3.58
CARX1X2GYX3X4DYWGQ
X1: S Y
X2: D G H R
X3: A D S Y
X4: F I L M
PDL1_VH_CDR3.59
CARX1X2GYX3X4DYWGQ
X1: D G H R
X2: D G H R
X3: A D S Y
X4: F I L M
PDL1_VH_CDR3.60
CARX1X2X3X4X5X6DYWGQ
X1: S Y
X2: S Y
X3: S Y
X4: D G
X5: A D S Y
X6: F I L M
PDL1_VH_CDR3.61
CARX1YX2X3X4FX5YWGQ
X1: D E Y
X2: S Y
X3: S Y
X4: A D H P S Y
X5: A D
PDL1_VH_CDR3.62
CARX1X2X3X4X5FX6YWGQ
X1: D E Y
X2: D G
X3: S Y
X4: S Y
X5: A D H P S Y
X6: A D
PDL1_VH_CDR3.63
CARX1YGX2X3FX4YWGQ
X1: D E Y
X2: S Y
X3: A D H P S Y
X4: A D
PDL1_VH_CDR3.64
CARX1X2GX3X4FX5YWGQ
X1: D E Y
X2: D G
X3: S Y
X4: A D H P S Y
X5: A D
PDL1_VH_CDR3.65
CARX1YX2X3X4FX5YWGQ
X1: D E Y
X2: S Y
X3: D G N S
X4: A D H P S Y
X5: A D
PDL1_VH_CDR3.66
CARX1X2X3X4X5FX6YWGQ
X1: D E Y
X2: D G
X3: S Y
X4: D G N S
X5: A D H P S Y
X6: A D
PDL1_VH_CDR3.67
CARX1YGX2X3FX4YWGQ
X1: D E Y
X2: D G N S
X3: A D H P S Y
X4: A D
PDL1_VH_CDR3.68
CARX1X2GX3X4FX5YWGQ
X1: D E Y
X2: D G
X3: D G N S
X4: A D H P S Y
X5: A D
PDL1_VH_CDR3.69
CARX1YX2X3X4X5DYWGQ
X1: D F H L V Y
X2: S Y
X3: S Y
X4: A D F S V Y
X5: F I L M
PDL1_VH_CDR3.70
CARX1X2X3X4X5X6DYWGQ
X1: D F H L V Y
X2: G R
X3: S Y
X4: S Y
X5: A D F S V Y
X6: F I L M
PDL1_VH_CDR3.71
CARX1YGX2X3X4DYWGQ
X1: D F H L V Y
X2: S Y
X3: A D F S V Y
X4: F I L M
PDL1_VH_CDR3.72
CARX1X2GX3X4X5DYWGQ
X1: D F H L V Y
X2: G R
X3: S Y
X4: A D F S V Y
X5: F I L M
PDL1_VH_CDR3.73
CARX1YX2X3X4X5DYWGQ
X1: D F H L V Y
X2: S Y
X3: D G N S
X4: A D F S V Y
X5: F I L M
PDL1_VH_CDR3.74
CARX1X2X3X4X5X6DYWGQ
X1: D F H L V Y
X2: G R
X3: S Y
X4: D G N S
X5: A D F S V Y
X6: F I L M
PDL1_VH_CDR3.75
CARX1YGX2X3X4DYWGQ
X1: D F H L V Y
X2: D G N S
X3: A D F S V Y
X4: F I L M
PDL1_VH_CDR3.76
CARX1X2GX3X4X5DYWGQ
X1: D F H L V Y
X2: G R
X3: D G N S
X4: A D F S V Y
X5: F I L M
PDL1_VH_CDR3.77
CERYDSPX1GDYWGQ
X1: L M P T
PDL1_VH_CDR3.78
CARX1YYGYX2X3X4X5WGQ
X1: D G
X2: A D S Y
X3: F I L M
X4: A D
X5: D F V Y
PDL1_VH_CDR3.79
CARYGYGYX1X2X3X4WGQ
X1: A D S Y
X2: F I L M
X3: A D
X4: D F V Y
PDL1_VH_CDR3.80
CARX1GYGYX2X3X4X5WGQ
X1: D G
X2: A D S Y
X3: F I L M
X4: A D
X5: D F V Y
PDL1_VH_CDR3.81
CARYYGGYX1X2X3X4WGQ
X1: A D S Y
X2: F I L M
X3: A D
X4: D F V Y
PDL1_VH_CDR3.82
CARX1YGGYX2X3X4X5WGQ
X1: D G
X2: A D S Y
X3: F I L M
X4: A D
X5: D F V Y
PDL1_VH_CDR3.83
CARYGGGYX1X2X3X4WGQ
X1: A D S Y
X2: F I L M
X3: A D
X4: D F V Y
PDL1_VH_CDR3.84
CARX1GGGYX2X3X4X5WGQ
X1: D G
X2: A D S Y
X3: F I L M
X4: A D
X5: D F V Y
PDL1_VH_CDR3.85
CARYYYYGX1X2X3X4WGQ
X1: A D S Y
X2: F I L M
X3: A D
X4: D F V Y
PDL1_VH_CDR3.86
CARX1YYYGX2X3X4X5WGQ
X1: D G
X2: A D S Y
X3: F I L M
X4: A D
X5: D F V Y
PDL1_VH_CDR3.87
CARYGYYGX1X2X3X4WGQ
X1: A D S Y
X2: F I L M
X3: A D
X4: D F V Y
PDL1_VH_CDR3.88
CARYYYYYX1X2X3X4WGQ
X1: A D S Y
X2: F I L M
X3: A D
X4: D F V Y
PDL1_VH_CDR3.89
CARX1GYYGX2X3X4X5WGQ
X1: D G
X2: A D S Y
X3: F I L M
X4: A D
X5: D F V Y
PDL1_VH_CDR3.90
CARYYGYGX1X2X3X4WGQ
X1: A D S Y
X2: F I L M
X3: A D
X4: D F V Y
PDL1_VH_CDR3.91
CARX1YGYGX2X3X4X5WGQ
X1: D G
X2: A D S Y
X3: F I L M
X4: A D
X5: D F V Y
PDL1_VH_CDR3.92
CARYGGYGX1X2X3X4WGQ
X1: A D S Y
X2: F I L M
X3: A D
X4: D F V Y
PDL1_VH_CDR3.93
CARX1GGYGX2X3X4X5WGQ
X1: D G
X2: A D S Y
X3: F I L M
X4: A D
X5: D F V Y
PDL1_VH_CDR3.94
CARYYYGGX1X2X3X4WGQ
X1: A D S Y
X2: F I L M
X3: A D
X4: D F V Y
PDL1_VH_CDR3.95
CARX1YYGGX2X3X4X5WGQ
X1: D G
X2: A D S Y
X3: F I L M
X4: A D
X5: D F V Y
PDL1_VH_CDR3.96
CARYGYGGX1X2X3X4WGQ
X1: A D S Y
X2: F I L M
X3: A D
X4: D F V Y
PDL1_VH_CDR3.97
CARX1GYGGX2X3X4X5WGQ
X1: D G
X2: A D S Y
X3: F I L M
X4: A D
X5: D F V Y
PDL1_VH_CDR3.98
CARYYGGGX1X2X3X4WGQ
X1: A D S Y
X2: F I L M
X3: A D
X4: D F V Y
PDL1_VH_CDR3.99
CARX1YYYYX2X3X4X5WGQ
X1: D G
X2: A D S Y
X3: F I L M
X4: A D
X5: D F V Y
PDL1_VH_CDR3.100
CARX1YGGGX2X3X4X5WGQ
X1: D G
X2: A D S Y
X3: F I L M
X4: A D
X5: D F V Y
PDL1_VH_CDR3.101
CARYGGGGX1X2X3X4WGQ
X1: A D S Y
X2: F I L M
X3: A D
X4: D F V Y
PDL1_VH_CDR3.102
CARX1GGGGX2X3X4X5WGQ
X1: D G
X2: A D S Y
X3: F I L M
X4: A D
X5: D F V Y
PDL1_VH_CDR3.103
CARYGYYYX1X2X3X4WGQ
X1: A D S Y
X2: F I L M
X3: A D
X4: D F V Y
PDL1_VH_CDR3.104
CARX1GYYYX2X3X4X5WGQ
X1: D G
X2: A D S Y
X3: F I L M
X4: A D
X5: D F V Y
PDL1_VH_CDR3.105
CARYYGYYX1X2X3X4WGQ
X1: A D S Y
X2: F I L M
X3: A D
X4: D F V Y
PDL1_VH_CDR3.106
CARX1YGYYX2X3X4X5WGQ
X1: D G
X2: A D S Y
X3: F I L M
X4: A D
X5: D F V Y
PDL1_VH_CDR3.107
CARYGGYYX1X2X3X4WGQ
X1: A D S Y
X2: F I L M
X3: A D
X4: D F V Y
PDL1_VH_CDR3.108
CARX1GGYYX2X3X4X5WGQ
X1: D G
X2: A D S Y
X3: F I L M
X4: A D
X5: D F V Y
PDL1_VH_CDR3.109
CARYYYGYX1X2X3X4WGQ
X1: A D S Y
X2: F I L M
X3: A D
X4: D F V Y
PDL1_VH_CDR3.110
CARX1X2X3YX4X5X6DYWGQ
X1: D E Y
X2: D G H R
X3: S Y
X4: S Y
X5: A G
X6: F I L M
PDL1_VH_CDR3.111
CARX1YGYX2X3X4DYWGQ
X1: D E Y
X2: S Y
X3: A G
X4: F I L M
PDL1_VH_CDR3.112
CARX1X2GYX3X4X5DYWGQ
X1: D E Y
X2: D G H R
X3: S Y
X4: A G
X5: F I L M
PDL1_VH_CDR3.113
CARX1YX2X3X4X5X6DYWGQ
X1: D E Y
X2: S Y
X3: D G
X4: S Y
X5: A G
X6: F I L M
PDL1_VH_CDR3.114
CARX1X2X3X4X5X6X7DYWGQ
X1: D E Y
X2: D G H R
X3: S Y
X4: D G
X5: S Y
X6: A G
X7: F I L M
PDL1_VH_CDR3.115
CARX1YGX2X3X4X5DYWGQ
X1: D E Y
X2: D G
X3: S Y
X4: A G
X5: F I L M
PDL1_VH_CDR3.116
CARX1X2GX3X4X5X6DYWGQ
X1: D E Y
X2: D G H R
X3: D G
X4: S Y
X5: A G
X6: F I L M
PDL1_VH_CDR3.117
CARX1YX2YX3YX4DYWGQ
X1: D E Y
X2: S Y
X3: S Y
X4: F I L M
PDL1_VH_CDR3.118
CARX1X2X3YX4YX5DYWGQ
X1: D E Y
X2: D G H R
X3: S Y
X4: S Y
X5: F I L M
PDL1_VH_CDR3.119
CARX1YGYX2YX3DYWGQ
X1: D E Y
X2: S Y
X3: F I L M
PDL1_VH_CDR3.120
CARX1X2GYX3YX4DYWGQ
X1: D E Y
X2: D G H R
X3: S Y
X4: F I L M
PDL1_VH_CDR3.121
CARX1YX2X3X4YX5DYWGQ
X1: D E Y
X2: S Y
X3: D G
X4: S Y
X5: F I L M
PDL1_VH_CDR3.122
CARX1X2X3X4X5YX6DYWGQ
X1: D E Y
X2: D G H R
X3: S Y
X4: D G
X5: S Y
X6: F I L M
PDL1_VH_CDR3.123
CARX1YGX2X3YX4DYWGQ
X1: D E Y
X2: D G
X3: S Y
X4: F I L M
PDL1_VH_CDR3.124
CARX1X2GX3X4YX5DYWGQ
X1: D E Y
X2: D G H R
X3: D G
X4: S Y
X5: F I L M
PDL1_VH_CDR3.125
CARX1YX2YX3X4X5DYWGQ
X1: D E Y
X2: S Y
X3: S Y
X4: A G
X5: F I L M
PDL1_VH_CDR3.126
CARX1X2X3YX4YFX5YWGQ
X1: H P S Y
X2: A D G N S T
X3: D G
X4: A G S T
X5: A D
PDL1_VH_CDR3.127
CARRX1X2YX3YFX4YWGQ
X1: A D G N S T
X2: D G
X3: A G S T
X4: A D
PDL1_VH_CDR3.128
CARX1X2X3RX4YFX5YWGQ
X1: H P S Y
X2: A D G N S T
X3: D G
X4: A G S T
X5: A D
PDL1_VH_CDR3.129
CARRX1X2RX3YFX4YWGQ
X1: A D G N S T
X2: D G
X3: A G S T
X4: A D
PDL1_VH_CDR3.130
CARX1X2X3YX4WFX5YWGQ
X1: H P S Y
X2: A D G N S T
X3: D G
X4: A G S T
X5: A D
PDL1_VH_CDR3.131
CARRX1X2YX3WFX4YWGQ
X1: A D G N S T
X2: D G
X3: A G S T
X4: A D
PDL1_VH_CDR3.132
CARX1X2X3RX4WFX5YWGQ
X1: H P S Y
X2: A D G N S T
X3: D G
X4: A G S T
X5: A D
PDL1_VH_CDR3.133
CARRX1X2RX3WFX4YWGQ
X1: A D G N S T
X2: D G
X3: A G S T
X4: A D
PDL1_VH_CDR3.134
CARX1X2GX3X4X5MDX6WGQ
X1: A D H P S Y
X2: D G H R
X3: S Y
X4: N Y
X5: A D G
X6: D F V Y
PDL1_VH_CDR3.135
CARX1X2GX3X4X5MDX6WGQ
X1: A D H P S Y
X2: D G H R
X3: K N R S
X4: N Y
X5: A D G
X6: D F V Y
PDL1_VH_CDR3.136
CARX1X2X3YX4X5FDYWGQ
X1: A G T D E K N R S
X2: D G N S
X3: G R
X4: S Y
X5: A D H P S Y
PDL1_VH_CDR3.137
CARX1X2X3WX4X5FDYWGQ
X1: A G T D E K N R S
X2: D G N S
X3: G R
X4: S Y
X5: A D H P S Y
PDL1_VH_CDR3.138
CARX1X2X3YX4X5FDYWGQ
X1: A G T D E K N R S
X2: D G N S
X3: G R
X4: D G N S
X5: A D H P S Y
PDL1_VH_CDR3.139
CARX1X2X3WX4X5FDYWGQ
X1: A G T D E K N R S
X2: D G N S
X3: G R
X4: D G N S
X5: A D H P S Y
PDL1_VH_CDR3.140
CARX1YYGX2YYX3DX4WGQ
X1: D G
X2: S Y
X3: F I L M
X4: D F V Y
PDL1_VH_CDR3.141
CARYGYGX1YYX2DX3WGQ
X1: S Y
X2: F I L M
X3: D F V Y
PDL1_VH_CDR3.142
CARX1GYGX2YYX3DX4WGQ
X1: D G
X2: S Y
X3: F I L M
X4: D F V Y
PDL1_VH_CDR3.143
CARYYGGX1YYX2DX3WGQ
X1: S Y
X2: F I L M
X3: D F V Y
PDL1_VH_CDR3.144
CARX1YGGX2YYX3DX4WGQ
X1: D G
X2: S Y
X3: F I L M
X4: D F V Y
PDL1_VH_CDR3.145
CARYGGGX1YYX2DX3WGQ
X1: S Y
X2: F I L M
X3: D F V Y
PDL1_VH_CDR3.146
CARX1GGGX2YYX3DX4WGQ
X1: D G
X2: S Y
X3: F I L M
X4: D F V Y
PDL1_VH_CDR3.147
CARYYYYGYYX1DX2WGQ
X1: F I L M
X2: D F V Y
PDL1_VH_CDR3.148
CARX1YYYGYYX2DX3WGQ
X1: D G
X2: F I L M
X3: D F V Y
PDL1_VH_CDR3.149
CARYGYYGYYX1DX2WGQ
X1: F I L M
X2: D F V Y
PDL1_VH_CDR3.150
CARYYYYX1YYX2DX3WGQ
X1: S Y
X2: F I L M
X3: D F V Y
PDL1_VH_CDR3.151
CARX1GYYGYYX2DX3WGQ
X1: D G
X2: F I L M
X3: D F V Y
PDL1_VH_CDR3.152
CARYYGYGYYX1DX2WGQ
X1: F I L M
X2: D F V Y
PDL1_VH_CDR3.153
CARX1YGYGYYX2DX3WGQ
X1: D G
X2: F I L M
X3: D F V Y
PDL1_VH_CDR3.154
CARYGGYGYYX1DX2WGQ
X1: F I L M
X2: D F V Y
PDL1_VH_CDR3.155
CARX1GGYGYYX2DX3WGQ
X1: D G
X2: F I L M
X3: D F V Y
PDL1_VH_CDR3.156
CARYYYGGYYX1DX2WGQ
X1: F I L M
X2: D F V Y
PDL1_VH_CDR3.157
CARX1YYGGYYX2DX3WGQ
X1: D G
X2: F I L M
X3: D F V Y
PDL1_VH_CDR3.158
CARYGYGGYYX1DX2WGQ
X1: F I L M
X2: D F V Y
PDL1_VH_CDR3.159
CARX1GYGGYYX2DX3WGQ
X1: D G
X2: F I L M
X3: D F V Y
PDL1_VH_CDR3.160
CARYYGGGYYX1DX2WGQ
X1: F I L M
X2: D F V Y
PDL1_VH_CDR3.161
CARX1YYYX2YYX3DX4WGQ
X1: D G
X2: S Y
X3: F I L M
X4: D F V Y
PDL1_VH_CDR3.162
CARX1YGGGYYX2DX3WGQ
X1: D G
X2: F I L M
X3: D F V Y
PDL1_VH_CDR3.163
CARYGGGGYYX1DX2WGQ
X1: F I L M
X2: D F V Y
PDL1_VH_CDR3.164
CARX1GGGGYYX2DX3WGQ
X1: D G
X2: F I L M
X3: D F V Y
PDL1_VH_CDR3.165
CARYYYYX1YX2X3DX4WGQ
X1: S Y
X2: A G
X3: F I L M
X4: D F V Y
PDL1_VH_CDR3.166
CARX1YYYX2YX3X4DX5WGQ
X1: D G
X2: S Y
X3: A G
X4: F I L M
X5: D F V Y
PDL1_VH_CDR3.167
CARYGYYX1YX2X3DX4WGQ
X1: S Y
X2: A G
X3: F I L M
X4: D F V Y
PDL1_VH_CDR3.168
CARX1GYYX2YX3X4DX5WGQ
X1: D G
X2: S Y
X3: A G
X4: F I L M
X5: D F V Y
PDL1_VH_CDR3.169
CARYYGYX1YX2X3DX4WGQ
X1: S Y
X2: A G
X3: F I L M
X4: D F V Y
PDL1_VH_CDR3.170
CARX1YGYX2YX3X4DX5WGQ
X1: D G
X2: S Y
X3: A G
X4: F I L M
X5: D F V Y
PDL1_VH_CDR3.171
CARYGGYX1YX2X3DX4WGQ
X1: S Y
X2: A G
X3: F I L M
X4: D F V Y
PDL1_VH_CDR3.172
CARYGYYX1YYX2DX3WGQ
X1: S Y
X2: F I L M
X3: D F V Y
PDL1_VH_CDR3.173
CARX1GGYX2YX3X4DX5WGQ
X1: D G
X2: S Y
X3: A G
X4: F I L M
X5: D F V Y
PDL1_VH_CDR3.174
CARYYYGX1YX2X3DX4WGQ
X1: S Y
X2: A G
X3: F I L M
X4: D F V Y
PDL1_VH_CDR3.175
CARX1YYGX2YX3X4DX5WGQ
X1: D G
X2: S Y
X3: A G
X4: F I L M
X5: D F V Y
PDL1_VH_CDR3.176
CARYGYGX1YX2X3DX4WGQ
X1: S Y
X2: A G
X3: F I L M
X4: D F V Y
PDL1_VH_CDR3.177
CARX1GYGX2YX3X4DX5WGQ
X1: D G
X2: S Y
X3: A G
X4: F I L M
X5: D F V Y
PDL1_VH_CDR3.178
CARYYGGX I YX2X3DX4WGQ
X1: S Y
X2: A G
X3: F I L M
X4: D F V Y
PDL1_VH_CDR3.179
CARX1YGGX2YX3X4DX5WGQ
X1: D G
X2: S Y
X3: A G
X4: F I L M
X5: D F V Y
PDL1_VH_CDR3.180
CARYGGGX1YX2X3DX4WGQ
X1: S Y
X2: A G
X3: F I L M
X4: D F V Y
PDL1_VH_CDR3.181
CARX1GGGX2YX3X4DX5WGQ
X1: D G
X2: S Y
X3: A G
X4: F I L M
X5: D F V Y
PDL1_VH_CDR3.182
CARYYYYGYX1X2DX3WGQ
X1: A G
X2: F I L M
X3: D F V Y
PDL1_VH_CDR3.183
CARX1GYYX2YYX3DX4WGQ
X1: D G
X2: S Y
X3: F I L M
X4: D F V Y
PDL1_VH_CDR3.184
CARX1YYYGYX2X3DX4WGQ
X1: D G
X2: A G
X3: F I L M
X4: D F V Y
PDL1_VH_CDR3.185
CARYGYYGYX1X2DX3WGQ
X1: A G
X2: F I L M
X3: D F V Y
PDL1_VH_CDR3.186
CARX1GYYGYX2X3DX4WGQ
X1: D G
X2: A G
X3: F I L M
X4: D F V Y
PDL1_VH_CDR3.187
CARYYGYGYX1X2DX3WGQ
X1: A G
X2: F I L M
X3: D F V Y
PDL1_VH_CDR3.188
CARX1YGYGYX2X3DX4WGQ
X1: D G
X2: A G
X3: F I L M
X4: D F V Y
PDL1_VH_CDR3.189
CARYGGYGYX1X2DX3WGQ
X1: A G
X2: F I L M
X3: D F V Y
PDL1_VH_CDR3.190
CARX1GGYGYX2X3DX4WGQ
X1: D G
X2: A G
X3: F I L M
X4: D F V Y
PDL1_VH_CDR3.191
CARYYYGGYX1X2DX3WGQ
X1: A G
X2: F I L M
X3: D F V Y
PDL1_VH_CDR3.192
CARX1YYGGYX2X3DX4WGQ
X1: D G
X2: A G
X3: F I L M
X4: D F V Y
PDL1_VH_CDR3.193
CARYGYGGYX1X2DX3WGQ
X1: A G
X2: F I L M
X3: D F V Y
PDL1_VH_CDR3.194
CARYYGYX1YYX2DX3WGQ
X1: S Y
X2: F I L M
X3: D F V Y
PDL1_VH_CDR3.195
CARX1GYGGYX2X3DX4WGQ
X1: D G
X2: A G
X3: F I L M
X4: D F V Y
PDL1_VH_CDR3.196
CARYYGGGYX1X2DX3WGQ
X1: A G
X2: F I L M
X3: D F V Y
PDL1_VH_CDR3.197
CARX1YGGGYX2X3DX4WGQ
X1: D G
X2: A G
X3: F I L M
X4: D F V Y
PDL1_VH_CDR3.198
CARYGGGGYX1X2DX3WGQ
X1: A G
X2: F I L M
X3: D F V Y
PDL1_VH_CDR3.199
CARX1GGGGYX2X3DX4WGQ
X1: D G
X2: A G
X3: F I L M
X4: D F V Y
PDL1_VH_CDR3.200
CARX1YGYX2YYX3DX4WGQ
X1: D G
X2: S Y
X3: F I L M
X4: D F V Y
PDL1_VH_CDR3.201
CARYGGYX1YYX2DX3WGQ
X1: S Y
X2: F I L M
X3: D F V Y
PDL1_VH_CDR3.202
CARX1GGYX2YYX3DX4WGQ
X1: D G
X2: S Y
X3: F I L M
X4: D F V Y
PDL1_VH_CDR3.203
CARYYYGX1YYX2DX3WGQ
X1: S Y
X2: F I L M
X3: D F V Y
PDL1_VH_CDR3.204
CARX1X2YYGYYX3DX4 WGQ
X1: D G
X2: G R
X3: F I L M
X4: D F V Y
PDL1_VH_CDR3.205
CARX1YGYGYYX2DX3WGQ
X1: D G
X2: F I L M
X3: D F V Y
PDL1_VH_CDR3.206
CARX1X2GYGYYX3DX4WGQ
X1: D G
X2: G R
X3: F I L M
X4: D F V Y
PDL1_VH_CDR3.207
CARX1YYGGYYX2DX3WGQ
X1: D G
X2: F I L M
X3: D F V Y
PDL1_VH_CDR3.208
CARX1X2YGGYYX3DX4WGQ
X1: D G
X2: G R
X3: F I L M
X4: D F V Y
PDL1_VH_CDR3.209
CARX1YGGGYYX2DX3WGQ
X1: D G
X2: F I L M
X3: D F V Y
PDL1_VH_CDR3.210
CARX1X2GGGYYX3DX4WGQ
X1: D G
X2: G R
X3: F I L M
X4: D F V Y
PDL1_VH_CDR3.211
CARX1YYYX2YX3X4DX5WGQ
X1: D G
X2: S Y
X3: A G
X4: F I L M
X5: D F V Y
PDL1_VH_CDR3.212
CARX1X2YYX3YX4X5DX6WGQ
X1: D G
X2: G R
X3: S Y
X4: A G
X5: F I L M
X6: D F V Y
PDL1_VH_CDR3.213
CARX1YGYX2YX3X4DX5WGQ
X1: D G
X2: S Y
X3: A G
X4: F I L M
X5: D F V Y
PDL1_VH_CDR3.214
CARX1YYYX2YYX3DX4WGQ
X1: D G
X2: S Y
X3: F I L M
X4: D F V Y
PDL1_VH_CDR3.215
CARX1X2GYX3YX4X5DX6WGQ
X1: D G
X2: G R
X3: S Y
X4: A G
X5: F I L M
X6: D F V Y
PDL1_VH_CDR3.216
CARX1YYGX2YX3X4DX5WGQ
X1: D G
X2: S Y
X3: A G
X4: F I L M
X5: D F V Y
PDL1_VH_CDR3.217
CARX1X2YGX3YX4X5DX6WGQ
X1: D G
X2: G R
X3: S Y
X4: A G
X5: F I L M
X6: D F V Y
PDL1_VH_CDR3.218
CARX1YGGX2YX3X4DX5WGQ
X1: D G
X2: S Y
X3: A G
X4: F I L M
X5: D F V Y
PDL1_VH_CDR3.219
CARX1X2GGX3YX4X5DX6WGQ
X1: D G
X2: G R
X3: S Y
X4: A G
X5: F I L M
X6: D F V Y
PDL1_VH_CDR3.220
CARX1YYYGYX2X3DX4WGQ
X1: D G
X2: A G
X3: F I L M
X4: D F V Y
PDL1_VH_CDR3.221
CARX1X2YYGYX3X4DX5WGQ
X1: D G
X2: G R
X3: A G
X4: F I L M
X5: D F V Y
PDL_VH_CDR3.222
CARX1YGYGYX2X3DX4WGQ
X1: D G
X2: A G
X3: F I L M
X4: D F V Y
PDL1_VH_CDR3.223
CARX1X2GYGYX3X4DX5WGQ
X1: D G
X2: G R
X3: A G
X4: F I L M
X5: D F V Y
PDL1_VH_CDR3.224
CARX1YYGGYX2X3DX4WGQ
X1: D G
X2: A G
X3: F I L M
X4: D F V Y
PDL1_VH_CDR3.225
CARX1X2YYX3YYX4DX5WGQ
X1: D G
X2: G R
X3: S Y
X4: F I L M
X5: D F V Y
PDL1_VH_CDR3.226
CARX1X2YGGYX3X4DX5WGQ
X1: D G
X2: G R
X3: A G
X4: F I L M
X5: D F V Y
PDL1_VH_CDR3.227
CARX1YGGGYX2X3DX4WGQ
X1: D G
X2: A G
X3: F I L M
X4: D F V Y
PDL1_VH_CDR3.228
CARX1X2GGGYX3X4DX5WGQ
X1: D G
X2: G R
X3: A G
X4: F I L M
X5: D F V Y
PDL1_VH_CDR3.229
CARX1YGYX2YYX3DX4WGQ
X1: D G
X2: S Y
X3: F I L M
X4: D F V Y
PDL1_VH_CDR3.230
CARX1X2GYX3YYX4DX5WGQ
X1: D G
X2: G R
X3: S Y
X4: F I L M
X5: D F V Y
PDL1_VH_CDR3.231
CARX1YYGX2YYX3DX4WGQ
X1: D G
X2: S Y
X3: F I L M
X4: D F V Y
PDL1_VH_CDR3.232
CARX1X2YGX3YYX4DX5WGQ
X1: D G
X2: G R
X3: S Y
X4: F I L M
X5: D F V Y
PDL1_VH_CDR3.233
CARX1YGGX2YYX3DX4WGQ
X1: D G
X2: S Y
X3: F I L M
X4: D F V Y
PDL1_VH_CDR3.234
CARX1X2GGX3YYX4DX5WGQ
X1: D G
X2: G R
X3: S Y
X4: F I L M
X5: D F V Y
PDL1_VH_CDR3.235
CARX1YYYGYYX2DX3WGQ
X1: D G
X2: F I L M
X3: D F V Y
PDL1_VHSDR3.236
CARGX1X2X3X4YX5X6DX7WGQ
X1: D G H R
X2: S Y
X3: F L Y
X4: S Y
X5: A D S Y
X6: F I L M
X7: D F V Y
PDL1_VH_CDR3.237
CARGX1RX2X3YX4X5DX6WGQ
X1: D G H R
X2: F L Y
X3: S Y
X4: A D S Y
X5: F I L M
X6: D F V Y
PDL1_VH_CDR3.238
CARGX1X2GX3YX4X5DX6WGQ
X1: D G H R
X2: S Y
X3: S Y
X4: A D S Y
X5: F I L M
X6: D F V Y
PDL1_VH_CDR3.239
CARGX1RGX2YX3X4DX5WGQ
X1: D G H R
X2: S Y
X3: A D S Y
X4: F I L M
X5: D F V Y
PDL1_VH_CDR3.240
CARGX1X2X3X4WX5X6DX7WGQ
X1: D G H R
X2: S Y
X3: F L Y
X4: S Y
X5: A D S Y
X6: F I L M
X7: D F V Y
PDL1_VH_CDR3.241
CARGX1RX2X3WX4X5DX6WGQ
X1: D G H R
X2: F L Y
X3: S Y
X4: A D S Y
X5: F I L M
X6: D F V Y
PDL1_VH_CDR3.242
CARGX1X2GX3WX4X5DX6WGQ
X1: D G H R
X2: S Y
X3: S Y
X4: A D S Y
X5: F I L M
X6: D F V Y
PDL1_VH_CDR3.243
CARGX1RGX2WX3X4DX5WGQ
X1: D G H R
X2: S Y
X3: A D S Y
X4: F I L M
X5: D F V Y
PDL1_VH_CDR3.244
CARX1X2YYX3X4X5FX6YWGQ
X1: S Y
X2: D Y
X3: D G N S
X4: G S
X5: H P S Y
X6: A D
PDL1_VH_CDR3.245
CARGX1YYX2X3X4FX5YWGQ
X1: D Y
X2: D G N S
X3: G S
X4: H P S Y
X5: A D
PDL1_VH_CDR3.246
CARX1X2YGX3X4X5FX6YWGQ
X1: S Y
X2: D Y
X3: D G N S
X4: G S
X5: F I P S Y
X6: A D
PDL1_VH_CDR3.247
CARGX1YGX2X3X4FX5YWGQ
X1: D Y
X2: D G N S
X3: G S
X4: H P S Y
X5: A D
PDL1_VH_CDR3.248
CARX1X2YYX3X4GFX5YWGQ
X1: S Y
X2: D Y
X3: D G N S
X4: G S
X5: A D
PDL1_VH_CDR3.249
CARGX1YYX2X3GFX4YWGQ
X1: D Y
X2: D G N S
X3: G S
X4: A D
PDL1_VH_CDR3.250
CARX1X2YGX3X4GFX5YWGQ
X1: S Y
X2: D Y
X3: D G N S
X4: G S
X5: A D
PDL1_VH_CDR3.251
CARGX1YGX2X3GFX4YWGQ
X1: D Y
X2: D G N S
X3: G S
X4: A D
PDL1_VH_CDR3.252
CARX1X2YYX3X4X5X6DYWGQ
X1: D G
X2: A G P R
X3: S Y
X4: D G N S
X5: A G P R
X6: F I L M
PDL1_VH_CDR3.253
CARYX1YGX2X3X4X5DYWGQ
X1: A G P R
X2: S Y
X3: D G N S
X4: A G P R
X5: F I L M
PDL1_VH_CDR3.254
CARX1X2YGX3X4X5X6DYWGQ
X1: D G
X2: A G P R
X3: S Y
X4: D G N S
X5: A G P R
X6: F I L M
PDL1_VH_CDR3.255
CARYX1YYGX2X3X4DYWGQ
X1: A G P R
X2: D G N S
X3: A G P R
X4: F I L M
PDL1_VH_CDR3.256
CARX1X2YYGX3X4X5DYWGQ
X1: D G
X2: A G P R
X3: D G N S
X4: A G P R
X5: F I L M
PDL1_VH_CDR3.257
CARYX1YGGX2X3X4DYWGQ
X1: A G P R
X2: D G N S
X3: A G P R
X4: F I L M
PDL1_VH_CDR3.258
CARX1X2YGGX3X4X5DYWGQ
X1: D G
X2: A G P R
X3: D G N S
X4: A G P R
X5: F I L M
PDL1_VH_CDR3.259
CARYX1YYX2X3X4X5DYWGQ
X1: A G P R
X2: S Y
X3: D G N S
X4: H Y
X5: F I L M
PDL1_VH_CDR3.260
CARX1X2YYX3X4X5X6DYWGQ
X1: D G
X2: A G P R
X3: S Y
X4: D G N S
X5: H Y
X6: F I L M
PDL1_VH_CDR3.261
CARYX1YGX2X3 X4X5DYWGQ
X1: A G P R
X2: S Y
X3: D G N S
X4: H Y
X5: F I L M
PDL1_VH_CDR3.262
CARX1X2YGX3X4X5X6DYWGQ
X1: D G
X2: A G P R
X3: S Y
X4: D G N S
X5: H Y
X6: F I L M
PDL1_VH_CDR3.263
CARYX1YYGX2X3X4DYWGQ
X1: A G P R
X2: D G N S
X3: H Y
X4: F I L M
PDL1_VH_CDR3.264
CARX1X2YYGX3X4X5DYWGQ
X1: D G
X2: A G P R
X3: D G N S
X4: H Y
XS: F I L M
PDL1_VH_CDR3.265
CARYX1YGGX2X3X4DYWGQ
X1: A G P R
X2: D G N S
X3: H Y
X4: F I L M
PDL1_VH_CDR3.266
CARX1X2YGGX3X4X5DYWGQ
X1: D G
X2: A G P R
X3: D G N S
X4: H Y
X5: F I L M
PDL1_VH_CDR3.267
CARYX1YYX2X3X4X5DYWGQ
X1: A G P R
X2: S Y
X3: D G N S
X4: A G P R
X5: F I L M
PDL1_VH_CDR3.268
CARX1YYYGX2X3GX4YWGQ
X1: E G
X2: A S
X3: S Y
X4: A D
PDL1_VH_CDR3.269
CARX1X2YYGX3X4GX5YWGQ
X1: S Y
X2: D G N S
X3: A S
X4: S Y
X5: A D
PDL1_VH_CDR3.270
CARX1X2YYGX3X4GX5YWGQ
X1: E G
X2: D G N S
X3: A S
X4: S Y
X5: A D
PDL1_VH_SDR3.271
CARX1YYYGX2X3GX4YWGQ
X1: S Y
X2: A S
X3: G R W
X4: A D
PDL1_VH_CDR3.272
CARX1YYYGX2X3GX4YWGQ
X1: E G
X2: A S
X3: G R W
X4: A D
PDL1_VH_CDR3.273
CARX1X2YYGX3X4GX5YWGQ
X1: S Y
X2: D G N S
X3: A S
X4: G R W
X5: A D
PDL1_VH_CDR3.274
CARX I X2YYGX3X4GX5YWGQ
X1: E G
X2: D G N S
X3: A S
X4: G R W
X5: A D
PDL1_VH_CDR3.275
CARX1YYYGX2X3X4X5YWGQ
X1: S Y
X2: A S
X3: S Y
X4: F S
X5: A D
PDL1_VH_CDR3.276
CARX1YYYGX2X3X4X5YWGQ
X1: E G
X2: A S
X3: S Y
X4: F S
X5: A D
PDL1_VH_CDR3.277
CARX1X2YYGX3X4X5X6YWGQ
X1: S Y
X2: D G N S
X3: A S
X4: S Y
X5: F S
X6: A D
PDL1_VH_CDR3.278
CARX1X2YYGX3X4X5X6YWGQ
X1: E G
X2: D G N S
X3: A S
X4: S Y
X5: F S
X6: A D
PDL1_VH_CDR3.279
CARX1YYYGX2X3X4X5YWGQ
X1: S Y
X2: A S
X3: G R W
X4: F S
X5: A D
PDL1_VH_CDR3.280
CARX1YYYGX2X3X4X5YWGQ
X1: E G
X2: A S
X3: G R W
X4: F S
X5: A D
PDL1_VH_CDR3.281
CARX1X2YYGX3X4X5X6YWGQ
X1: S Y
X2: D G N S
X3: A S
X4: G R W
X5: F S
X6: A D
PDL1_VH_CDR3.282
CARX1X2YYGX3X4X5X6YWGQ
X1: E G
X2: D G N S
X3: A S
X4: G R W
X5: F S
X6: A D
PDL1_VH_CDR3.283
CARX1YYYGX2X3GX4YWGQ
X1: S Y
X2: A S
X3: S Y
X4: A D
PDL1_VH_CDR3.284
CARX1X2X3X4YYX5X6DYWGQ
X1: G D E K N R S
X2: H P S Y
X3: F H L P S Y
X4: S Y
X5: A G
X6: F I L M
PDL1_VH_CDR3.285
CARX1GX2X3YYX4X5DYWGQ
X1: G D E K N R S
X2: F H L P S Y
X3: S Y
X4: A G
X5: F I L M
PDL1_VH_CDR3.286
CARX1X2X3X4YX5X6X7DYWGQ
X1: G D E K N R S
X2: H P S Y
X3: F H L P S Y
X4: S Y
X5: D G
X6: A G
X7: F I L M
PDL1_VH_CDR3.287
CARX1GX2X3YX4X5X6DYWGQ
X1: G D E K N R S
X2: F H L P S Y
X3: S Y
X4: D G
X5: A G
X6: F I L M
PDL1_VH_CDR3.288
CARGYX1YX2WYX3DX4WGQ
X1: S Y
X2: S Y
X3: F I L M
X4: D F V Y
PDL1_VH_CDR3.289
CARYRX1YX2WYX3DX4WGQ
X1: S Y
X2: S Y
X3: F I L M
X4: D F V Y
PDL1_VH_CDR3.290
CARGRX1YX2WYX3DX4WGQ
X1: S Y
X2: S Y
X3: F I L M
X4: D F V Y
PDL1_VH_CDR3.291
CARYYX1GX2WYX3DX4WGQ
X1: S Y
X2: S Y
X3: F I L M
X4: D F V Y
PDL1_VH_CDR3.292
CARGYX1GX2WYX3DX4WGQ
X1: S Y
X2: S Y
X3: F I L M
X4: D F V Y
PDL1_VH_CDR3.293
CARYRX1GX2WYX3DX4WGQ
X1: S Y
X2: S Y
X3: F I L M
X4: D F V Y
PDL1_VH_CDR3.294
CARGRX1GX2WYX3DX4WGQ
X1: S Y
X2: S Y
X3: F I L M
X4: D F V Y
PDL1_VH_CDR3.295
CARYYX I YX2YX3X4DX5WGQ
X1: S Y
X2: S Y
X3: A G
X4: F I L M
X5: D F V Y
PDL1_VH_CDR3.296
CARGYX1YX2YX3X4DX5WGQ
X1: S Y
X2: S Y
X3: A G
X4: F I L M
X5: D F V Y
PDL1_VH_CDR3.297
CARYRX1YX2YX3X4DX5WGQ
X1: S Y
X2: S Y
X3: A G
X4: F I L M
X5: D F V Y
PDL1_VH_CDR3.298
CARYYX1YX2YYX3DX4WGQ
X1: S Y
X2: S Y
X3: F I L M
X4: D F V Y
PDL1_VH_CDR3.299
CARGRX1YX2YX3X4DX5WGQ
X1: S Y
X2: S Y
X3: A G
X4: F I L M
X5: D F V Y
PDL1_VH_CDR3.300
CARYYX1GX2YX3X4DX5WGQ
X1: S Y
X2: S Y
X3: A G
X4: F I L M
X5: D F V Y
PDL1_VH_CDR3.301
CARGYX1GX2YX3X4DX5WGQ
X1: S Y
X2: S Y
X3: A G
X4: F I L M
X5: D F V Y
PDL1_VH_CDR3.302
CARYRX1GX2YX3X4DX5WGQ
X1: S Y
X2: S Y
X3: A G
X4: F I L M
X5: D F V Y
PDL1_VH_CDR3.303
CARGRX1GX2YX3X4DX5WGQ
X1: S Y
X2: S Y
X3: A G
X4: F I L M
X5: D F V Y
PDL1_VH_CDR3.304
CARYYX1YX2WX3X4DX5WGQ
X1: S Y
X2: S Y
X3: A G
X4: F I L M
X5: D F V Y
PDL1_VH_CDR3.305
CARGYX1YX2WX3X4DX5WGQ
X1: S Y
X2: S Y
X3: A G
X4: F I L M
X5: D F V Y
PDL1_VH_CDR3.306
CARYRX1YX2WX3X4DX5WGQ
X1: S Y
X2: S Y
X3: A G
X4: F I L M
X5: D F V Y
PDL1_VH_CDR3.307
CARGRX1YX2WX3X4DX5WGQ
X1: S Y
X2: S Y
X3: A G
X4: F I L M
X5: D F V Y
PDL1_VH_CDR3.308
CARYYX1GX2WX3X4DX5WGQ
X1: S Y
X2: S Y
X3: A G
X4: F I L M
X5: D F V Y
PDL1_VH_CDR3.309
CARGYX I YX2YYX3DX4WGQ
X1: S Y
X2: S Y
X3: F I L M
X4: D F V Y
PDL1_VH_CDR3.310
CARGYX1GX2WX3X4DX5WGQ
X1: S Y
X2: S Y
X3: A G
X4: F I L M
X5: D F V Y
PDL1_VH_CDR3.311
CARYRX1GX2WX3X4DX5WGQ
X1: S Y
X2: S Y
X3: A G
X4: F I L M
X5: D F V Y
PDL1_VH_CDR3.312
CARGRX1GX2WX3X4DX5WGQ
X1: S Y
X2: S Y
X3: A G
X4: F I L M
X5: D F V Y
PDL1_VH_CDR3.313
CARYRX 1YX2YYX3DX4WGQ
X1: S Y
X2: S Y
X3: F I L M
X4: D F V Y
PDL1_VH_CDR3.314
CARGRX1YX2YYX3DX4WGQ
X1: S Y
X2: S Y
X3: F I L M
X4: D F V Y
PDL1_VH_CDR3.315
CARYYX1GX2YYX3DX4WGQ
X1: S Y
X2: S Y
X3: F I L M
X4: D F V Y
PDL1_VH_CDR3.316
CARGYX1GX2YYX3DX4WGQ
X1: S Y
X2: S Y
X3: F I L M
X4: D F V Y
PDL1_VH_CDR3.317
CARYRX1GX2YYX3DX4WGQ
X1: S Y
X2: S Y
X3: F I L M
X4: D F V Y
PDL1_VH_CDR3.318
CARGRX1GX2YYX3DX4WGQ
X1: S Y
X2: S Y
X3: F I L M
X4: D F V Y
PDL1_VH_CDR3.319
CARYYX1YX2WYX3DX4WGQ
X1: S Y
X2: S Y
X3: F I L M
X4: D F V Y
PDL1_VH_CDR3.320
CARX 1X2X3X4X5YX6X7DYWGQ
X1: H Y
X2: S Y
X3: S Y
X4: D G N S
X5: H P S Y
X6: A D S Y
X7: F I L M
PDL1_VH_CDR3.321
CARX1GX2X3X4YX5X6DYWGQ
X1: H Y
X2: S Y
X3: D G N S
X4: H P S Y
X5: A D S Y
X6: F I L M
PDL1_VH_CDR3.322
CARX1X2GX3X4YX5X6DYWGQ
X1: H Y
X2: S Y
X3: D G N S
X4: H P S Y
X5: A D S Y
X6: F I L M
PDL1_VH_CDR3.323
CARX1GGX2X3YX4X5DYWGQ
X1: H Y
X2: D G N S
X3: H P S Y
X4: A D S Y
X5: F I L M
PDL1_VH_CDR3.324
CARX1X2X3X4GYX5X6DYWGQ
X1: H Y
X2: S Y
X3: S Y
X4: D G N S
X5: A D S Y
X6: F I L M
PDL1_VH_CDR3.325
CARX1GX2X3GYX4X5DYWGQ
X1: H Y
X2: S Y
X3: D G N S
X4: A D S Y
X5: F I L M
PDL1_VH_CDR3.326
CARX1X2GX3GYX4X5DYWGQ
X1: H Y
X2: S Y
X3: D G N S
X4: A D S Y
X5: F I L M
PDL1_VH_CDR3.327
CARX1GGX2GYX3X4DYWGQ
X1: H Y
X2: D G N S
X3: A D S Y
X4: F I L M
PDL1_VH_CDR3.328
CARX1GGYX2GYYX3DX4WGQ
X1: D G
X2: S Y
X3: F I L M
X4: D F V Y
PDL1_VH_CDR3.329
CARX1GYGX2GYYX3DX4WGQ
X1: D G
X2: S Y
X3: F I L M
X4: D F V Y
PDL1_VH_CDR3.330
CARX1GGGX2GYYX3DX4WGQ
X1: D G
X2: S Y
X3: F I L M
X4: D F V Y
PDL1_VH_CDR3.331
CARX1GYYGGYYX2DX3WGQ
X1: D G
X2: F I L M
X3: D F V Y
PDL1_VH_CDR3.332
CARX1GGYGGYYX2DX3WGQ
X1: D G
X2: F I L M
X3: D F V Y
PDL1_VH_CDR3.333
CARX1GYGGGYYX2DX3WGQ
X1: D G
X2: F I L M
X3: D F V Y
PDL1_VH_CDR3.334
CARX1GGGGGYYX2DX3WGQ
X1: D G
X2: F I L M
X3: D F V Y
PDL1_VH_CDR3.335
CARX1GYYX2X3YX4X5DX6WGQ
X1: D G
X2: S Y
X3: S Y
X4: A G
X5: F I L M
X6: D F V Y
PDL1_VH_CDR3.336
CARX1GGYX2X3YX4X5DX6WGQ
X1: D G
X2: S Y
X3: S Y
X4: A G
X5: F I L M
X6: D F V Y
PDL1_VH_CDR3.337
CARX1GYGX2X3YX4X5DX6WGQ
X1: D G
X2: S Y
X3: S Y
X4: A G
X5: F I L M
X6: D F V Y
PDL1_VH_CDR3.338
CARX1GYYX2X3YYX4DX5WGQ
X1: D G
X2: S Y
X3: S Y
X4: F I L M
X5: D F V Y
PDL1_VH_CDR3.339
CARX1GGGX2X3YX4X5DX6WGQ
X1: D G
X2: S Y
X3: S Y
X4: A G
X5: F I L M
X6: D F V Y
PDL1_VH_CDR3.340
CARX1GYYGX2YX3X4DX5WGQ
X1: D G
X2: S Y
X3: A G
X4: F I L M
X5: D F V Y
PDL1_VH_CDR3.341
CARX1GGYGX2YX3X4DX5WGQ
X1: D G
X2: S Y
X3: A G
X4: F I L M
X5: D F V Y
PDL1_VH_CDR3.342
CARX1GYGGX2YX3X4DX5WGQ
X1: D G
X2: S Y
X3: A G
X4: F I L M
X5: D F V Y
PDL1_VH_CDR3.343
CARX1GGGGX2YX3X4DX5WGQ
X1: D G
X2: S Y
X3: A G
X4: F I L M
X5: D F V Y
PDL1_VH_CDR3.344
CARX1GYYX2GYX3X4DX5WGQ
X1: D G
X2: S Y
X3: A G
X4: F I L M
X5: D F V Y
PDL1_VH_CDR3.345
CARX1GGYX2GYX3X4DX5WGQ
X1: D G
X2: S Y
X3: A G
X4: F I L M
X5: D F V Y
PDL1_VH_CDR3.346
CARX1GYGX2GYX3X4DX5WGQ
X1: D G
X2: S Y
X3: A G
X4: F I L M
X5: D F V Y
PDL1_VH_CDR3.347
CARX1GGGX2GYX3X4DX5WGQ
X1: D G
X2: S Y
X3: A G
X4: F I L M
X5: D F V Y
PDL1_VH_CDR3.348
CARX1GYYGGYX2X3DX4WGQ
X1: D G
X2: A G
X3: F I L M
X4: D F V Y
PDL1_VH_CDR3.349
CARX1GGYX2X3YYX4DX5WGQ
X1: D G
X2: S Y
X3: S Y
X4: F I L M
X5: D F V Y
PDL1_VH_CDR3.350
CARX1GGYGGYX2X3DX4WGQ
X1: D G
X2: A G
X3: F I L M
X4: D F V Y
PDL1_VH_CDR3.351
CARX1GYGGGYX2X3DX4WGQ
X1: D G
X2: A G
X3: F I L M
X4: D F V Y
PDL1_VH_CDR3.352
CARX1GGGGGYX2X3DX4WGQ
X1: D G
X2: A G
X3: F I L M
X4: D F V Y
PDL1_VH_CDR3.353
CARX1GYGX2X3YYX4DX5WGQ
X1: D G
X2: S Y
X3: S Y
X4: F I L M
X5: D F V Y
PDL1_VH_CDR3.354
CARX1GGGX2X3YYX4DX5WGQ
X1: D G
X2: S Y
X3: S Y
X4: F I L M
X5: D F V Y
PDL1_VH_CDR3.355
CARX1GYYGX2YYX3DX4WGQ
X1: D G
X2: S Y
X3: F I L M
X4: D F V Y
PDL1_VH_CDR3.356
CARX1GGYGX2YYX3DX4WGQ
X1: D G
X2: S Y
X3: F I L M
X4: D F V Y
PDL1_VH_CDR3.357
CARX1GYGGX2YYX3DX4WGQ
X1: D G
X2: S Y
X3: F I L M
X4: D F V Y
PDL1_VH_CDR3.358
CARX1GGGGX2YYX3DX4WGQ
X1: D G
X2: S Y
X3: F I L M
X4: D F V Y
PDL1_VH_CDR3.359
CARX1GYYX2GYYX3DX4WGQ
X1: D G
X2: S Y
X3: F I L M
X4: D F V Y
PDL1_VH_CDR3.360
CARX1GYX2YGX3X4X5DYWGQ
X1: D G
X2: S Y
X3: S Y
X4: A D S Y
X5: F I L M
PDL1_VH_CDR3.361
CARX1YGX2YGX3X4X5DYWGQ
X1: D G
X2: S Y
X3: S Y
X4: A D S Y
X5: F I L M
PDL1_VH_CDR3.362
CARX1GGX2YGX3X4X5DYWGQ
X1: D G
X2: S Y
X3: S Y
X4: A D S Y
X5: F I L M
PDL1_VH_CDR3.363
CARX1YYX2GGX3X4X5DYWGQ
X1: D G
X2: S Y
X3: S Y
X4: A D S Y
X5: F I L M
PDL1_VH_CDR3.364
CARX1GYX2GGX3X4X5DYWGQ
X1: D G
X2: S Y
X3: S Y
X4: A D S Y
X5: F I L M
PDL1_VH_CDR3.365
CARX1YGX2GGX3X4X5DYWGQ
X1: D G
X2: S Y
X3: S Y
X4: A D S Y
X5: F I L M
PDL1_VH_CDR3.366
CARX1GGX2GGX3X4X5DYWGQ
X1: D G
X2: S Y
X3: S Y
X4: A D S Y
X5: F I L M
PDL1_VH_CDR3.367
CARX1YYX2YX3X4X5X6DYWGQ
X1: D G
X2: S Y
X3: S Y
X4: S Y
X5: A D S Y
X6: F I L M
PDL1_VH_CDR3.368
CARX1GYX2YX3X4X5X6DYWGQ
X1: D G
X2: S Y
X3: S Y
X4: S Y
X5: A D S Y
X6: F I L M
PDL1_VH_CDR3.369
CARX1YGX2YX3X4X5X6DYWGQ
X1: D G
X2: S Y
X3: S Y
X4: S Y
X5: A D S Y
X6: F I L M
PDL1_VH_CDR3.370
CARX1GGX2YX3X4X5X6DYWGQ
X1: D G
X2: S Y
X3: S Y
X4: S Y
X5: A D S Y
X6: F I L M
PDL1_VH_CDR3.371
CARX1YYX2GX3X4X5X6DYWGQ
X1: D G
X2: S Y
X3: S Y
X4: S Y
X5: A D S Y
X6: F I L M
PDL1_VH_CDR3.372
CARX1GYX2GX3X4X5X6DYWGQ
X1: D G
X2: S Y
X3: S Y
X4: S Y
X5: A D S Y
X6: F I L M
PDL1_VH_CDR3.373
CARX1YGX2GX3X4X5X6DYWGQ
X1: D G
X2: S Y
X3: S Y
X4: S Y
X5: A D S Y
X6: F I L M
PDL1_VH_CDR3.374
CARX1GGX2GX3X4X5X6DYWGQ
X1: D G
X2: S Y
X3: S Y
X4: S Y
X5: A D S Y
X6: F I L M
PDL1_VH_CDR3.375
CARX1YYX2YGX3X4X5DYWGQ
X1: D G
X2: S Y
X3: S Y
X4: A D S Y
X5: F I L M
PDL1_VH_CDR3.376
CARX1GYX2YX3YX4X5DX6WGQ
X1: D G
X2: S Y
X3: S Y
X4: A G
X5: F I L M
X6: D F V Y
PDL1_VH_CDR3.377
CARX1YGX2YX3YX4X5DX6WGQ
X1: D G
X2: S Y
X3: S Y
X4: A G
X5: F I L M
X6: D F V Y
PDL1_VH_CDR3.378
CARX1GGX2YX3YX4X5DX6WGQ
X1: D G
X2: S Y
X3: S Y
X4: A G
X5: F I L M
X6: D F V Y
PDL1_VH_CDR3.379
CARX1YYX2GX3YX4X5DX6WGQ
X1: D G
X2: S Y
X3: S Y
X4: A G
X5: F I L M
X6: D F V Y
PDL1_VH_CDR3.380
CARX1GYX2GX3YX4X5DX6WGQ
X1: D G
X2: S Y
X3: S Y
X4: A G
X5: F I L M
X6: D F V Y
PDL1_VH_CDR3.381
CARX1YGX2GX3YX4X5DX6WGQ
X1: D G
X2: S Y
X3: S Y
X4: A G
X5: F I L M
X6: D F V Y
PDL1_VH_CDR3.382
CARX1GGX2GX3YX4X5DX6WGQ
X1: D G
X2: S Y
X3: S Y
X4: A G
X5: F I L M
X6: D F V Y
PDL1_VH_CDR3.383
CARX1YYX2YX3YYX4DX5WGQ
X1: D G
X2: S Y
X3: S Y
X4: F I L M
X5: D F V Y
PDL1_VH_CDR3.384
CARX1GYX2YX3YYX4DX5WGQ
X1: D G
X2: S Y
X3: S Y
X4: F I L M
X5: D F V Y
PDL1_PDL1CDR3.385
CARX1YGX2YX3YYX4DX5WGQ
X1: D G
X2: S Y
X3: S Y
X4: F I L M
X5: D F V Y
PDL1_VH_CDR3.386
CARX1GGX2YX3YYX4DX5WGQ
X1: D G
X2: S Y
X3: S Y
X4: F I L M
X5: D F V Y
PDL1_VH_CDR3.387
CARX1YYX2GX3YYX4DX5WGQ
X1: D G
X2: S Y
X3: S Y
X4: F I L M
X5: D F V Y
PDL1_VH_CDR3.388
CARX1GYX2GX3YYX4DX5WGQ
X1: D G
X2: S Y
X3: S Y
X4: F I L M
X5: D F V Y
PDL1_VH_CDR3.389
CARX1YGX2GX3YYX4DX5WGQ
X1: D G
X2: S Y
X3: S Y
X4: F I L M
X5: D F V Y
PDL1_VH_CDR3.390
CARX1GGX2GX3YYX4DX5WGQ
X1: D G
X2: S Y
X3: S Y
X4: F I L M
X5: D F V Y
PDL1_VH_CDR3.391
CARX1YYX2YX3YX4X5DX6WGQ
X1: D G
X2: S Y
X3: S Y
X4: A G
X5: F I L M
X6: D F V Y
PDL1_VH_CDR3.392
CARDX1X2YYX3X4X5X6DX7WGQ
X1: D G
X2: S Y
X3: S Y
X4: S Y
X5: A D S Y
X6: F I L M
X7: D F V Y
PDL1_VH_CDR3.393
CARDX1X2GYX3X4X5X6DX7WGQ
X1: D G
X2: S Y
X3: S Y
X4: S Y
X5: A D S Y
X6: F I L M
X7: D F V Y
PDL1_VH_CDR3.394
CARDX1X2YGX3X4X5X6DX7WGQ
X1: D G
X2: S Y
X3: S Y
X4: S Y
X5: A D S Y
X6: F I L M
X7: D F V Y
PDL1_VH_CDR3.395
CARDX1X2GGX3X4X5X6DX7WGQ
X1: D G
X2: S Y
X3: S Y
X4: S Y
X5: A D S Y
X6: F I L M
X7: D F V Y
PDL1_VH_CDR3.396
CARX1X2X3YX4X5X6X7X8DYWGQ
X1: D G N S
X2: H Y
X3: S Y
X4: S Y
X5: S Y
X6: S Y
X7: A D S Y
X8: F I L M
PDL1_VH_CDR3.397
CARX1X2X3YGX4X5X6X7DYWGQ
X1: D G N S
X2: H Y
X3: S Y
X4: S Y
X5: S Y
X6: A D S Y
X7: F I L M
PDL1_VH_CDR3.398
CARX1X2X3YX4GX5X6X7DYWGQ
X1: D G N S
X2: H Y
X3: S Y
X4: S Y
X5: S Y
X6: A D S Y
X7: F I L M
PDL1_VH_CDR3.399
CARX1X2X3YGGX4X5X6DYWGQ
X1: D G N S
X2: H Y
X3: S Y
X4: S Y
X5: A D S Y
X6: F I L M
PDL1_VH_CDR3.400
CARYX1YX2X3X4X5YX6DYWGQ
X1: N S T Y
X2: S Y
X3: S Y
X4: D G N S
X5: S Y
X6: F I L M
PDL1_VH_CDR3.401
CARGX1YX2X3 X4X5YX6DYWGQ
X1: N S T Y
X2: S Y
X3: S Y
X4: D G N S
X5: S Y
X6: F I L M
PDL1_VH_CDR3.402
CARYX1YX2X3X4X5YX6DYWGQ
X1: N S T Y
X2: D G
X3: S Y
X4: D G N S
X5: S Y
X6: F I L M
PDL1_VH_CDR3.403
CARGX1YX2X3X4X5YX6DYWGQ
X1: N S T Y
X2: D G
X3: S Y
X4: D G N S
X5: S Y
X6: F I L M
PDL1_VH_CDR3.404
CARYX1YX2GX3X4YX5DYWGQ
X1: N S T Y
X2: S Y
X3: D G N S
X4: S Y
X5: F I L M
PDL1_VH_CDR3.405
CARGX1YX2GX3X4YX5DYWGQ
X1: N S T Y
X2: S Y
X3: D G N S
X4: S Y
X5: F I L M
PDL1_VH_CDR3.406
CARYX1YX2GX3X4YX5DYWGQ
X1: N S T Y
X2: D G
X3: D G N S
X4: S Y
X5: F I L M
PDL1_VH_CDR3.407
CARGX1YX2GX3X4YX5DYWGQ
X1: N S T Y
X2: D G
X3: D G N S
X4: S Y
X5: F I L M
PDL1_VH_CDR3.408
CARDX1X2GYYX3X4X5X6YWGQ
X1: D G H R
X2: D G N S
X3: A S D E Y
X4: A D S Y
X5: F I L M
X6: A D
PDL1_VH_CDR3.409
CARX1X2X3X4X5GYX6X7DYWGQ
X1: A E G P Q R
X2: L V
X3: D F H L V Y
X4: G R
X5: A G L P R V
X6: A D N S T Y
X7: F I L M
PDL1_VH_CDR3.410
CARX1X2X3YYX4YX5X6DX7WGQ
X1: D G
X2: D G H R
X3: G S
X4: F S Y
X5: A D G
X6: F I L M
X7: D F V Y
PDL1_VH_CDR3.411
CARDX1YGX2YX3YX4X5DX6WGQ
X1: G R
X2: S Y
X3: S Y
X4: A G
X5: F I L M
X6: D F V Y
PDL1_VH_CDR3.412
CARDX1YYGYX2YX3X4DX5WGQ
X1: G R
X2: S Y
X3: A G
X4: F I L M
X5: D F V Y
PDL1_VH_CDR3.413
CARDX1YGGYX2YX3X4DX5WGQ
X1: G R
X2: S Y
X3: A G
X4: F I L M
X5: D F V Y
PDL1_VH_CDR3.414
CARDX1YYX2GX3YX4X5DX6WGQ
X1: G R
X2: S Y
X3: S Y
X4: A G
X5: F I L M
X6: D F V Y
PDL1_VH_CDR3.415
CARDX1YGX2GX3YX4X5DX6WGQ
X1: G R
X2: S Y
X3: S Y
X4: A G
X5: F I L M
X6: D F V Y
PDL1_VH_CDR3.416
CARDX1YYGGX2YX3X4DX5WGQ
X1: G R
X2: S Y
X3: A G
X4: F I L M
X5: D F V Y
PDL1_VH_CDR3.417
CARDX1YGGGX2YX3X4DX5WGQ
X1: G R
X2: S Y
X3: A G
X4: F I L M
X5: D F V Y
PDL1_VH_CDR3.418
CARDX1YYX2YX3YYX4DX5WGQ
X1: G R
X2: S Y
X3: S Y
X4: F I L M
X5: D F V Y
PDL1_VH_CDR3.419
CARDX1YGX2YX3YYX4DX5WGQ
X1: G R
X2: S Y
X3: S Y
X4: F I L M
X5: D F V Y
PDL1_VH_CDR3.420
CARDX1YYGYX2YYX3DX4WGQ
X1: G R
X2: S Y
X3: F I L M
X4: D F V Y
PDL1_VH_CDR3.421
CARDX1YGGYX2YYX3DX4WGQ
X1: G R
X2: S Y
X3: F I L M
X4: D F V Y
PDL1_VH_CDR3.422
CARDX1YYX2GX3YYX4DX5WGQ
X1: G R
X2: S Y
X3: S Y
X4: F I L M
X5: D F V Y
PDL1_VH_CDR3.423
CARDX1YGX2GX3YYX4DX5WGQ
X1: G R
X2: S Y
X3: S Y
X4: F I L M
X5: D F V Y
PDL1_VH_CDR3.424
CARDX1YYGGX2YYX3DX4WGQ
X1: G R
X2: S Y
X3: F I L M
X4: D F V Y
PDL1_VH_CDR3.425
CARDX1YGGGX2YYX3DX4WGQ
X1: G R
X2: S Y
X3: F I L M
X4: D F V Y
PDL1_VH_CDR3.426
CARDX1YYX2YX3YX4X5DX6WGQ
X1: G R
X2: S Y
X3: S Y
X4: A G
X5: F I L M
X6: D F V Y
PDL1_VH_CDR3.427
CARX1X2YX3YX4X5X6YFDYWGQ
X1: D G N S
X2: P R
X3: S Y
X4: G S
X5: G S
X6: S Y
PDL1_VH_CDR3.428
CARX1X2YGYX3X4X5YFDYWGQ
X1: D G N S
X2: P R
X3: G S
X4: G S
X5: S Y
PDL1_VH_CDR3.429
CARX1X2YX3GX4X5X6YFDYWGQ
X1: D G N S
X2: P R
X3: S Y
X4: G S
X5: G S
X6: S Y
PDL1_VHCDR3.430
CARX1X2YGGX3X4X5YFDYWGQ
X1: D G N S
X2: P R
X3: G S
X4: G S
X5: S Y
PDL1_VH_CDR3.431
CARX1X2YX3YX4X5X6X7FDYWGQ
X1: D G N S
X2: P R
X3: S Y
X4: G S
X5: G S
X6: S Y
X7: A G
PDL1_VH_CDR3.432
CARX1X2YGYX3X4X5X6FDYWGQ
X1: D G N S
X2: P R
X3: G S
X4: G S
X5: S Y
X6: A G
PDL1_VH_CDR3.433
CARX1X2YX3GX4X5X6X7FDYWGQ
X1: D G N S
X2: P R
X3: S Y
X4: G S
X5: G S
X6: S Y
X7: A G
PDL1_VH_CDR3.434
CARX1X2YGGX3X4X5X6FDYWGQ
X1: D G N S
X2: P R
X3: G S
X4: G S
X5: S Y
X6: A G
PDL1_VH_CDR3.435
CARX1X2YYX3X4X5YX6X7DYWGQ
X1: G D E
X2: A G P R
X3: S Y
X4: S Y
X5: S Y
X6: A D S Y
X7: F I L M
PDL1_VH_CDR3.436
CARX1X2YYGX3X4YX5X6DYWGQ
X1: G D E
X2: A G P R
X3: S Y
X4: S Y
X5: A D S Y
XS: F I L M
PDL1_VH_CDR3.437
CARX1X2YYX3GX4YX5X6DYWGQ
X1: G D E
X2: A G P R
X3: S Y
X4: S Y
X5: A D S Y
X6: F I L M
PDL1_VH_CDR3.438
CARX1X2YYGGX3YX4X5DYWGQ
X1: G D E
X2: A G P R
X3: S Y
X4: A D S Y
X5: F I L M
PDL1_VH_CDR3.439
CARX1X2YYX3X4GYX5X6DYWGQ
X1: G D E
X2: A G P R
X3: S Y
X4: S Y
X5: A D S Y
X6: F I L M
PDL1_VH_CDR3.440
CARX1X2YYGX3GYX4X5DYWGQ
X1: G D E
X2: A G P R
X3: S Y
X4: A D S Y
X5: F I L M
PDL1_VH_CDR3.441
CARX1X2YYX3GGYX4X5DYWGQ
X1: G D E
X2: A G P R
X3: S Y
X4: A D S Y
X5: F I L M
PDL1_VH_CDR3.442
CARX1X2YYGGGYX3X4DYWGQ
X1: G D E
X2: A G P R
X3: A D S Y
X4: F I L M
PDL1_VH_CDR3.443
CARX1X2IYX3X4YX5X6X7DX8WGQ
X1: D G H N R S
X2: A G P R
X3: S Y
X4: D Y
X5: S Y
X6: A G P R
X7: F I L M
X8: D F V Y
PDL1_VH_CDR3.444
CARSGYYX1X2YYAMDYWGQ
X1: G R
X2: A D S Y
PDL1_VH_CDR3.445
CARSYX1YX2X3YYAMDYWGQ
X1: A G S T
X2: G R
X3: A D S Y
PDL1_VH_CDR3.446
CARSGX1YX2X3YYAMDYWGQ
X1: A G S T
X2: G R
X3: A D S Y
PDL1_VH_CDR3.447
CARSYYX1X2X3YYAMDYWGQ
X1: A D G N S T
X2: G R
X3: A D S Y
PDL1_VH_CDR3.448
CARSGYX1X2X3YYAMDYWGQ
X1: A D G N S T
X2: G R
X3: A D S Y
PDL1_VH_CDR3.449
CARSYX1X2X3X4YYAMDYWGQ
X1: A G S T
X2: A D G N S T
X3: G R
X4: A D S Y
PDL1_VH_CDR3.450
CARSGX1X2X3X4YYAMDYWGQ
X1: A G S T
X2: A D G N S T
X3: G R
X4: A D S Y
PDL1_VH_CDR3.451
CARSYYYX1X2YYAMDYWGQ
X1: S Y
X2: A D S Y
PDL1_VH_CDR3.452
CARSGYYX1X2YYAMDYWGQ
X1: S Y
X2: A D S Y
PDL1_VH_CDR3.453
CARSYX1YX2X3YYAMDYWGQ
X1: A G S T
X2: S Y
X3: A D S Y
PDL1_VH_CDR3.454
CARSGX1YX2X3YYAMDYWGQ
X1: A G S T
X2: S Y
X3: A D S Y
PDL1_VH_CDR3.455
CARSYYX1X2X3YYAMDYWGQ
X1: A D G N S T
X2: S Y
X3: A D S Y
PDL1_VH_CDR3.456
CARSGYX1X2X3YYAMDYWGQ
X1: A D G N S T
X2: S Y
X3: A D S Y
PDL1_VH_CDR3.457
CARSYX1X2X3X4YYAMDYWGQ
X1: A G S T
X2: A D G N S T
X3: S Y
X4: A D S Y
PDL1_VH_CDR3.458
CARSGX1X2X3X4YYAMDYWGQ
X1: A G S T
X2: A D G N S T
X3: S Y
X4: A D S Y
PDL1_VH_CDR3.459
CARSYYYX1X2YYAMDYWGQ
X1: G R
X2: A D S Y
PDL1_VH_CDR3.460
CARX1YYYX2X3X4YAMDYWGQ
X1: F H L P S Y
X2: S Y
X3: D G N S
X4: A D S Y
PDL1_VH_CDR3.461
CARX1YYYX2X3X4YAMDYWGQ
X1: D G
X2: S Y
X3: D G N S
X4: A D S Y
PDL1_VH_CDR3.462
CARX1X2YYX3X4X5YAMDYWGQ
X1: F H L P S Y
X2: A G
X3: S Y
X4: D G N S
X5: A D S Y
PDL1_VH_CDR3.463
CARX1X2YYX3X4X5YAMDYWGQ
X1: D G
X2: A G
X3: S Y
X4: D G N S
X5: A D S Y
PDL1_VH_CDR3.464
CARX1YYYX2X3X4YAMDYWGQ
X1: F H L P S Y
X2: G L R V
X3: D G N S
X4: A D S Y
PDL1_VH_CDR3.465
CARX1YYYX2X3X4YAMDYWGQ
X1: D G
X2: G L R V
X3: D G N S
X4: A D S Y
PDL1_VH_CDR3.466
CARX1X2YYX3X4X5YAMDYWGQ
X1: F H L P S Y
X2: A G
X3: G L R V
X4: D G N S
X5: A D S Y
PDL1_VH_CDR3.467
CARX1X2YYX3X4X5YAMDYWGQ
X1: D G
X2: A G
X3: G L R V
X4: D G N S
X5: A D S Y
PDL1_VH_CDR3.468
CVTSLTWLLRRKRSYWGQ
PDL1_VH_CDR3.469
CARX1GYYX2X3X4YYX5X6DX7WGQ
X1: D G
X2: S Y
X3: S Y
X4: G S
X5: A D S Y
X6: F I L M
X7: D F V Y
PDL1_VH_CDR3.470
CARX1GYGX2X3X4YYX5X6DX7WGQ
X1: D G
X2: S Y
X3: S Y
X4: G S
X5: A D S Y
X6: F I L M
X7: D F V Y
PDL1_VH_CDR3.471
CARX1GYYGX2X3YYX4X5DX6WGQ
X1: D G
X2: S Y
X3: G S
X4: A D S Y
X5: F I L M
X6: D F V Y
PDL1_VH_CDR3.472
CARX1GYGGX2X3YYX4X5DX6WGQ
X1: D G
X2: S Y
X3: G S
X4: A D S Y
X5: F I L M
X6: D F V Y
PDL1_VH_CDR3.473
CARX1GYYX2X3X4YGX5X6DX7WGQ
X1: D G
X2: S Y
X3: S Y
X4: G S
X5: A D S Y
X6: F I L M
X7: D F V Y
PDL1_VH_CDR3.474
CARX1GYGX2X3X4YGX5X6DX7WGQ
X1: D G
X2: S Y
X3: S Y
X4: G S
X5: A D S Y
X6: F I L M
X7: D F V Y
PDL1_VH_CDR3.475
CARX1GYYGX2X3YGX4X5DX6WGQ
X1: D G
X2: S Y
X3: G S
X4: A D S Y
X5: F I L M
X6: D F V Y
PDL1_VH_CDR3.476
CARX1GYGGX2X3YGX4X5DX6WGQ
X1: D G
X2: S Y
X3: G S
X4: A D S Y
X5: F I L M
X6: D F V Y
PDL1_VH_CDR3.477
CARSX1X2YX3X4YX5YX6MDX7WGQ
X1: G R
X2: F I N Y
X3: S Y
X4: D F V Y
X5: D Y
X6: A G
X7: D F V Y
PDL1_VH_CDR3.478
CARSX1X2X3X4X5YX6YX7MDX8WGQ
X1: G R
X2: F I N Y
X3: A G S T
X4: S Y
X5: D F V Y
X6: D Y
X7: A G
X8: D F V Y
PDL1_VH_CDR3.479
CARDGX1X2YX3YYYYX4DX5WGQ
X1: A G S T
X2: A S
X3: S Y
X4: F I L M
X5: D F V Y
PDL1_VH_CDR3.480
CARDGX1X2YX3YYX4YX5DX6WGQ
X1: A G S T
X2: A S
X3: S Y
X4: G W
X5: F I L M
X6: D F V Y
PDL1_VH_CDR3.481
CARDGX1X2YX3YYYGX4DX5WGQ
X1: A G S T
X2: A S
X3: S Y
X4: F I L M
X5: D F V Y
PDL1_VH_CDR3.482
CARDGX1X2YX3YYX4GX5DX6WGQ
X1: A G S T
X2: A S
X3: S Y
X4: G W
X5: F I L M
X6: D F V Y
PDL1_VH_CDR3.483
CARX1X2YYX3X4X5YYAX6DYWGQ
X1: D G N S
X2: G R
X3: S Y
X4: G I S V
X5: N S T Y
X6: F I L M
PDL1_VH_CDR3.484
CARX1X2YYX3X4X5YYAX6DYWGQ
X1: D G N S
X2: G R
X3: D G
X4: G I S V
X5: N S T Y
X6: F I L M
PDL1_VH_CDR3.485
CARX1X2YYX3X4X5GYAX6DYWGQ
X1: D G N S
X2: G R
X3: S Y
X4: G I S V
X5: N S T Y
X6: F I L M
PDL1_VH_CDR3.486
CARX1X2YYX3X4X5GYAX6DYWGQ
X1: D G N S
X2: G R
X3: D G
X4: G I S V
X5: N S T Y
X6: F I L M
PDL1_VH_CDR3.487
CARDX1YX2X3X4X5YYX6X7DX8WGQ
X1: I L
X2: F I N S T Y
X3: N S T Y
X4: A G S T
X5: S Y
X6: A G
X7: F I L M
X8: D F V Y
PDL1_VH_CDR3.488
CARGX1X2YX3X4X5YYX6X7DYWGQ
X1: E G K R
X2: D G
X3: S Y
X4: D G N S
X5: S Y
X6: A D S Y
X7: F I L M
PDL1_VH_CDR3.489
CARGX1X2YX3X4X5WYX6X7DYWGQ
X1: E G K R
X2: D G
X3: S Y
X4: D G N S
X5: S Y
X6: A D S Y
X7: F I L M
PDL1_VH_CDR3.490
CARX1YX2X3X4X5TX6WYPDLWGR
X1: G D E
X2: A D N T
X3: G I S V
X4: A G S T
X5: G S
X6: N S T Y
PDL1_VH_CDR3.491
CARX1GX2X3X4X5TX6WYPDLWGR
X1: G D E
X2: A D N T
X3: G I S V
X4: A G S T
X5: G S
X6: N S T Y
PDL1_VH_CDR3.492
CARGX1X2YX3X4X5YYX6X7DYWGQ
X1: E G K R
X2: D G
X3: S Y
X4: D G N S
X5: S Y
X6: A D S Y
X7: F I L M
PDL1_VH_CDR3.493
CARGX1X2YX3X4X5WYX6X7DYWGQ
X1: E G K R
X2: D G
X3: S Y
X4: D G N S
X5: S Y
X6: A D S Y
X7: F I L M
PDL1_VH_CDR3.494
CARVX1YYYX2SSYYX3X4DX5WGQ
X1: G S
X2: G I S V
X3: A D S Y
X4: F I L M
X5: D F V Y
PDL1_VH_CDR3.495
CARVX1YYX2X3SSYYX4X5DX6WGQ
X1: G S
X2: D 0
X3: G I S V
X4: A D S Y
X5: F I L M
X6: D F V Y
PDL1_VH_CDR3.496
CARVX1YYYX2S SX3YX4X5DX6WGQ
X1: G S
X2: G I S V
X3: D G
X4: A D S Y
X5: F I L M
X6: D F V Y
PDL1_VH_CDR3.497
CARVX1YYX2X3SSX4YX5X6DX7WGQ
X1: G S
X2: D G
X3: G I S V
X4: D G
X5: A D S Y
X6: F I L M
X7: D F V Y
PDL1_VH_CDR3.498
CARVX1YYYX2SSYWX3X4DX5WGQ
X1: G S
X2: G I S V
X3: A D S Y
X4: F I L M
X5: D F V Y
PDL1_VH_CDR3.499
CARVX1YYX2X3SSYWX4X5DX6WGQ
X1: G S
X2: D G
X3: G I S V
X4: A D S Y
X5: F I L M
X6: D F V Y
PDL1_VH_CDR3.500
CARVX1YYYX2SSX3WX4X5DX6WGQ
X1: G S
X2: G I S V
X3: D G
X4: A D S Y
X5: F I L M
X6: D F V Y
PDL1_VH_CDR3.501
CARVX1YYX2X3SSX4WX5X6DX7WGQ
X1: G S
X2: D G
X3: G I S V
X4: D G
X5: A D S Y
X6: F I L M
X7: D F V Y
PDL1_VH_CDR3.502
CARX1X2YX3YX4SX5YYYX6DX7WGQ
X1: D E
X2: A G P R
X3: D Y
X4: D G
X5: S Y
X6: F I L M
X7: D F V Y
PDL1_VH_CDR3.503
CARX1X2YX3RX4SX5YYYX6DX7WGQ
X1: D E
X2: A G P R
X3: D Y
X4: D G
X5: S Y
X6: F I L M
X7: D F V Y
PDL1_VH_CDR3.504
CARX1X2YX3YX4SX5YYX6X7DX8WGQ
X1: D E
X2: A G P R
X3: D Y
X4: D G
X5: S Y
X6: A G
X7: F I L M
X8: D F V Y
PDL1_VH_CDR3.505
CARX1X2YX3RX4SX5YYX6X7DX8WGQ
X1: D E
X2: A G P R
X3: D Y
X4: D G
X5: S Y
X6: A G
X7: F I L M
X8: D F V Y
PDL1_VH_CDR3.506
CATGNSVRLASWEGYFYWGQ
PDL1_VH_CDR3.507
CARQGRVRLATESGLDYWGQ
PDL1_VH_CDR3.508
CARX1GX2DX3X4X5X6X7X8PFDX9WGP
X1: E K M V
X2: A S
X3: H R
X4: I L
X5: S T
X6: V D E I K M N
X7: D I N V
X8: D E H Q
X9: A D S Y
PDL1_VH_CDR3.509
CARX1X2X3YYX4X5X6X7YX8FDYWGQ
X1: D G
X2: G R
X3: G S
X4: G V
X5: A S
X6: G S
X7: G S
X8: H P S Y
PDL1_VH_CDR3.510
CARX1X2X3YRX4X5X6X7YX8FDYWGQ
X1: D G
X2: G R
X3: G S
X4: G V
X5: A S
X6: G S
X7: G S
X8: H P S Y
PDL1_VH_CDR3.511
CARX1SFX2QLTX3YAX4FDIWGQ
X1: E V
X2: D E H Q
X3: G L R V
X4: A P
PDL1_VH_CDR3.512
CARX1X2YYYGSX3X4YX5X6DYWGQ
X1: D G
X2: A G P R
X3: S Y
X4: H P S Y
X5: A D S Y
X6: F I L M
PDL1_VH_CDR3.513
CARX1X2GYYGSX3X4YX5X6DYWGQ
X1: D G
X2: A G P R
X3: S Y
X4: H P S Y
X5: A D S Y
X6: F I L M
PDL1_VH_CDR3.514
CARX1X2YYYGSGX3YX4X5DYWGQ
X1: D G
X2: A G P R
X3: H P S Y
X4: A D S Y
X5: F I L M
PDL1_VH_CDR3.515
CARX1X2GYYGSGX3YX4X5DYWGQ
X1: D G
X2: A G P R
X3: H P S Y
X4: A D S Y
X5: F I L M
PDL1_VH_CDR3.516
CARX1X2YYYGSX3GYX4X5DYWGQ
X1: D G
X2: A G P R
X3: S Y
X4: A D S Y
X5: F I L M
PDL1_VH_CDR3.517
CARX1X2GYYGSX3GYX4X5DYWGQ
X1: D G
X2: A G P R
X3: S Y
X4: A D S Y
X5: F I L M
PDL1_VH_CDR3.518
CARX1X2YYYGSGGYX3X4DYWGQ
X1: D G
X2: A G P R
X3: A D S Y
X4: F I L M
PDL1_VH_CDR3.519
CARX1X2GYYGSGGYX3X4DYWGQ
X1: D G
X2: A G P R
X3: A D S Y
X4: F I L M
PDL1_VH_CDR3.520
CARGRPPYX1SX2WYYX3DYWGQ
X1: G S
X2: G S
X3: F L
PDL1_VH_CDR3.521
CARGRPPYX1SX2WGYX3DYWGQ
X1: G S
X2: G S
X3: F L
PDL1_VH_CDR3.522
CARGRPPYX1SX2WYX3X4DYWGQ
X1: G S
X2: G S
X3: A G
X4: F L
PDL1_VH_CDR3.523
CARGRPPYX1SX2WGX3X4DYWGQ
X1: G S
X2: G S
X3: A G
X4: F L
PDL1_VL_CDR1.1
MTCX1ASSSX2X3YX4X5WYQ
X1: R S
X2: I V
X3: N S
X4: I M
X5: H N Y
PDL1_VL_CDR1.2
LTCRAVTTX1NX2ANWVQ
X1: N S
X2: F Y
PDL1_VL_CDR1.3
ITCRASQX1X2X3X4YLX5WYQ
X1: A D S Y
X2: I V
X3: G S
X4: N S T
X5: A D N T
PDL1_VL_CDR1.4
LTCX1SS TGAVTX2X3X4YX5NWVQ
X1: G R
X2: S T
X3: G S
X4: H N
X5: A P
PDL1_VL_CDR1.5
ISCTGX1SSX2X3GX4YNX5VSWYQ
X1: S T
X2: D N
X3: I V
X4: A G S T
X5: D Y
PDL1_VL_CDR1.6
ISCTGX1SSDX2GX3YNX4VSWYQ
X1: S T
X2: I V
X3: A G S T
X4: F L Y
PDL1_VL_CDR1.7
ISCTGX1SSDX2GX3YNX4VSWYQ
X1: S T
X2: I V
X3: A G S T
X4: F L Y
PDL1_VL_CDR1.8
ISCTGTSSDX1GX2YX3X4VSWYQ
X1: I V
X2: A D G N S T
X3: D N
X4: F L Y
PDL1_VL_CDR1.9
ISCTGX1SSDX2GX3YNX4VSWYQ
X1: S T
X2: I V
X3: A G S T
X4: F L Y
PDL1_VL_CDR1.10
ISCRASX1SVX2X3X4GX5SX6MHWYQ
X1: E Q
X2: A D S Y
X3: S T
X4: S Y
X5: N Y
X6: F Y
PDL1_VL_CDR1.11
ISCRASX1SVX2X3X4GYSX5MHWYQ
X1: E K Q
X2: A D S Y
X3: S T
X4: S Y
X5: F Y
PDL1_VL_CDR1.12
ISCRSSQSLX1HSX2GX3TYLX4WYL
X1: L V
X2: D N
X3: K N
X4: A P S T D E H K N Q Y
PDL1_VL_CDR1.13
ISCRSSQSLX1HSX2GX3TYLX4WYL
X1: L V
X2: D N
X3: K N
X4: A P S T D E H K N Q Y
PDL1_VL_CDR1.14
INCKSSQSX1LX2SX3NX4KNYLAWYQ
X1: L V
X2: N Y
X3: G R S
X4: R H K N Q S
PDL1_VL_CDR2.1
LIYX1ASNX2X3SGVP
X1: A G T D E K N R S
X2: L R
X3: A E P Q
PDL1_VL_CDR3.1
YYCQQX1X2X3X4X5TFGG
X1: S Y
X2: S Y
X3: G D E K N R S
X4: F H L P S Y
X5: F H L P S Y
PDL1_VL_CDR3.2
YYCQQWX1X2X3X4TFGG
X1: S Y
X2: G D E K N R S
X3: F H L P S Y
X4: F H L P S Y
PDL1_VL_CDR33
YYCQQX1X2X3X4X5TFGG
X1: S Y
X2: A D G H N P R S T
X3: G D E K N R S
X4: F H L P S Y
X5: F H L P S Y
PDL1_VL_CDR3.4
YYCQQWX1X2X3X4TFGG
X1: A D G H N P R S T
X2: G D E K N R S
X3: F H L P S Y
X4: F H L P S Y
PDL1_VL_CDR3.5
YYCQQX1X2X3X4WTFGG
X1: S Y
X2: S Y
X3: G D E K N R S
X4: F H L P S Y
PDL1_VL_CDR3.6
YYCQQWX1X2X3 WTFGG
X1: S Y
X2: G D E K N R S
X3: F H L P S Y
PDL1_VI_CDR3.7
YYCQQX1X2X3X4WTFGG
X1: S Y
X2: A D G H N P R S T
X3: G D E K N R S
X4: F H L P S Y
PDL1_VL_CDR3.8
YYCQQWX1X2X3WTFGG
X1: A D G H N P R S T
X2: G D E K N R S
X3: F H L P S Y
PDL1_VL_CDR3.9
YYCQQGX1SX2PX3TFGG
X1: S Y
X2: S F L Y
X3: R W
PDL1_VL_CDR3.10
YYCQQX1X2SX3PX4TFGG
X1: S Y
X2: A D G N S T
X3: S F L Y
X4: R W
PDL1_VL_CDR3.11
YYCQQGX1SX2PX3TFGG
X1: A D G N S T
X2: S F L Y
X3: R W
PDL1_VL_CDR3.12
YYCQQX1X2SX3PX4TFGG
X1: S Y
X2: S Y
X3: A G L M R S T V W
X4: R W
PDL1_VL_CDR3.13
YYCQQGX1SX2PX3TFGG
X1: S Y
X2: A G L M R S T V W
X3: R W
PDL1_VL_CDR3.14
YYCQQX1X2SX3PX4TFGG
X1: S Y
X2: A D G N S T
X3: A G L M R S T V W
X4: R W
PDL1_VL_CDR3.15
YYCQQGX1SX2PX3TFGG
X1: A D G N S T
X2: A G L M R S T V W
X3: R W
PDL1_VL_CDR3.16
YYCQQX1X2SX3PX4TFGG
X1: S Y
X2: S Y
X3: S F L Y
X4: F H L P S Y
PDL1_VL_CDR3.17
YYCQQGX1SX2PX3TFGG
X1: S Y
X2: S F L Y
X3: F H L P S Y
PDL1_VL_CDR3.18
YYCQQX1X2SX3PX4TFGG
X1: S Y
X2: A D G N S T
X3: S F L Y
X4: F H L P S Y
PDL1_VL_CDR3.19
YYCQQGX1SX2PX3TFGG
X1: A D G N S T
X2: S F L Y
X3: F H L P S Y
PDL1_VL_CDR3.20
YYCQQX1X2SX3PX4TFGG
X1: S Y
X2: S Y
X3: A G L M R S T V W
X4: F H L P S Y
PDL1_VL_CDR3.21
YYCQQGX1SX2PX3TFGG
X1: S Y
X2: A G L M R S T V W
X3: F H L P S Y
PDL1_VL_CDR3.22
YYCQQX1X2SX3PX4TFGG
X1: S Y
X2: A D G N S T
X3: A G L M R S T V W
X4: F H L P S Y
PDL1_VLCDR3.23
YYCQQGX1SX2PX3TFGG
X1: A D G N S T
X2: A G L M R S T V W
X3: F H L P S Y
PDL1_VL_CDR3.24
YYCQQX1X2SX3PX4TFGG
X1: S Y
X2: S Y
X3: S F L Y
X4: R W
PDL1_VL_CDR3.25
YYCQQYX1SX2PX3X4FGG
X1: S Y
X2: N S T Y
X3: F L Y
X4: A I T V
PDL1_VL_CDR3.26
YYCQQWX1SX2PX3X4FGG
X1: S Y
X2: N S T Y
X3: F L Y
X4: A I T V
PDL1_VL_CDR3.27
YYCQQYX1SX2PX3X4FGG
X1: D G N S
X2: N S T Y
X3: F L Y
X4: A I T V
PDL1_VL_CDR3.28
YYCQQWX1SX2PX3X4FGG
X1: D G N S
X2: N S T Y
X3: F L Y
X4: A I T V
PDL1_VL_CDR3.29
YYCQQYX1SX2PX3X4FGG
X1: S Y
X2: N S T Y
X3: L G R V W
X4: A I T V
PDL1_VL_CDR3.30
YYCQQWX1SX2PX3X4FGG
X1: S Y
X2: N S T Y
X3: L G R V W
X4: A I T V
PDL1_VL_CDR3.31
YYCQQYX1SX2PX3X4FGG
X1: D G N S
X2: N S T Y
X3: L G R V W
X4: A I T V
PDL1_VL_CDR3.32
YYCQQWX1SX2PX3X4FGG
X1: D G N S
X2: N S T Y
X3: L G R V W
X4: A I T V
PDL1_VL_CDR3.33
YYCX1X2YX3SSX4X5X6X7FGG
X1: P Q S
X2: P Q S
X3: A D N T
X4: H N P T
X5: P S T
X6: F L Y
X7: A M T V
PDL1_VL_CDR3.34
YYCX1X2YX3SSX4X5X6X7FGG
X1: P Q S
X2: P Q S
X3: A D N T
X4: H N P T
X5: P S T
X6: G L V W
X7: A M T V
PDL1_VL_CDR3.35
YYCQX1YDX2SX3X4X5X6FGG
X1: L A E P Q S V
X2: G S
X3: H N P S T Y
X4: P T
X5: F L Y
X6: A M T V
PDL1_VL_CDR3.36
YYCQX1WDX2SX3X4X5X6FGG
X1: L A E P Q S V
X2: G S
X3: H N P S T Y
X4: P T
X5: F L Y
X6: A M T V
PDL1_VL_CDR337
YYCQX1YDX2SX3X4X5X6FGG
X1: L A E P Q S V
X2: G S
X3: H N P S T Y
X4: P T
X5: G L V W
X6: A M T V
PDL1_VL_CDR3.38
YYCQX1WDX2SX3X4X5X6FGG
X1: L A E P Q S V
X2: G S
X3: H N P S T Y
X4: P T
X5: G L V W
X6: A M T V
PDL1_VL_CDR3.39
YYCX1X2YDX3SX4X5X6YVFGG
X1: A E P Q
X2: A S
X3: A D S Y
X4: L S
X5: D G N S
X6: A D G H P R
PDL1_VL_CDR3.40
YYCX1X2WDX3SX4X5X6YVFGG
X1: A E P Q
X2: A S
X3: A D S Y
X4: L S
X5: D G N S
X6: A D G H P R
PDL1_VL_CDR3.41
YYCX1X2YDX3SX4X5X6X7VFGG
X1: A E P Q
X2: A S
X3: A D S Y
X4: L S
X5: D G N S
X6: A D G H P R
X7: G L V W
PDL1_VL_CDR3.42
YYCX1X2WDX3SX4X5X6X7VFGG
X1: A E P Q
X2: A S
X3: A D S Y
X4: L S
X5: D G N S
X6: A D G H P R
X7: G L V W
PDL1_VL_CDR3.43
YYCQSYDX1SX2X3X4X5VFGG
X1: N S
X2: L A G P R S V
X3: A D H N P S T Y
X4: A D G H I L N P R S T V
X5: D F V Y
PDL1_VL_CDR3.44
YYCQSWDX1SX2X3X4X5VFGG
X1: N S
X2: L A G P R S V
X3: A D H N P S T Y
X4: A D G H I L N P R S T V
X5: D F V Y
PDL1_VLambda_CDR3.1
YYCQQX1X2X3X4X5TFGG
X1: S Y
X2: S Y
X3: N S
X4: F H L P S Y
X5: F H L P S Y
PDL1_VLambda_CDR3.2
YYCQQX1X2X3X4X5TFGG
X1: S Y
X2: D G H N R S
X3: N S T Y
X4: F H L P S Y
X5: F H L P S Y
PDL1_VLambda_CDR3.3
YYCQQX1X2X3WX4TFGG
X1: S Y
X2: S Y
X3: N S
X4: F H L P S Y
PDL1_VLambda_CDR3.4
YYCQQX1X2X3WX4TFGG
X1: S Y
X2: D G H N R S
X3: N S
X4: F H L P S Y
PDL1_VLambda_CDR3.5
YYCQQX1X2X3X4WTFGG
X1: S Y
X2: S Y
X3: N S
X4: L P S F H Q Y
PDL1_VLambda_CDR3.6
YYCQQX1X2X3X4WTFGG
X1: S Y
X2: D G H N R S
X3: N S
X4: F H L P S Y
PDL1_VLambda_CDR3.7
YYCQQX1X2X3WWTFGG
X1: S Y
X2: S Y
X3: N S
PDL1_VLambda_CDR3.8
YYCQQX1X2X3WWTFGG
X1: S Y
X2: D G H N R S
X3: N S
PDL1_VLambda_CDR3.9
YYCQQX1X2X3X4PX5TFGG
X1: H P S Y
X2: K N R S
X3: R G D E H K N Q S
X4: F H I L N Y
X5: L W
PDL1_VLambda_CDR3.10
YYCQQWX1X2X3PX4TFGG
X1: K N R S
X2: R G D E H K N Q S
X3: F H I L N Y
X4: L W
PDL1_VLambda_CDR3.11
YYCQQX1X2X3X4PX5TFGG
X1: H P S Y
X2: K N R S
X3: R G D E H K N Q S
X4: A G L M R S T V W
X5: L W
PDL1_VLambda_CDR3.12
YYCQQWX1X2X3PX4TFGG
X1: K N R S
X2: R G D E H K N Q S
X3: A G L M R S T V W
X4: L W
PDL1_VLambda_CDR3.13
YYCQQYX1X2X3PX4X5FGG
X1: S Y
X2: N S
X3: N S T Y
X4: F L P S
X5: A I T V
PDL1_VLambda_CDR3.14
YYCQQWX1X2X3PX4X5FGG
X1: S Y
X2: N S
X3: N S T Y
X4: F L P S
X5: A I T V
PDL1_VLambda_CDR3.15
YYCQQYX1X2X3PX4X5FGG
X1: D G N S
X2: N S
X3: N S T Y
X4: F L P S
X5: A I T V
PDL1_VLambda_CDR3.16
YYCQQWX1X2X3PX4X5FGG
X1: D G N S
X2: N S
X3: N S T Y
X4: F L P S
X5: A I T V
PDL1_VLambda_CDR3.17
YYCQQYX1X2X3PX4X5FGG
X1: S Y
X2: N S
X3: N S T Y
X4: L G R V W
X5: A I T V
PDL1_VLambda_CDR3.18
YYCQQWX1X2X3PX4X5FGG
X1: S Y
X2: N S
X3: N S T Y
X4: L G R V W
X5: A I T V
PDL1_VLambda_CDR3.19
YYCQQYX1X2X3PX4X5FGG
X1: D G N S
X2: N S
X3: N S T Y
X4: L G R V W
X5: A I T V
PDL1_VLambda_CDR3.20
YYCQQWX1X2X3PX4X5FGG
X1: D G N S
X2: N S
X3: N S T Y
X4: L G R V W
X5: A I T V
PDL1_VLambda_CDR3.21
YYCX1X2YX3SSX4X5X6X7FGG
X1: P Q S
X2: P Q S
X3: A D G N S T
X4: H N P T
X5: P S T
X6: F L
X7: A M T V
PDL1_VLambda_CDR3.22
YYCX1X2YX3SSX4X5X6X7FGG
X1: P Q S
X2: P Q S
X3: A D G N S T
X4: H N P T
X5: P S T
X6: G L V W
X7: A M T V
PDL1_VLambda_CDR3.23
YYCQX1YX2SX3PPX4X5FGG
X1: P Q S
X2: S Y
X3: N S
X4: G L V W
X5: A I T V
PDL1_VLambda_CDR3.24
YYCQX1WX2SX3PPX4X5FGG
X1: P Q S H Y
X2: S Y
X3: N S
X4: G L V W
X5: A I T V
PDL1_VLambda_CDR3.25
YYCQX1YX2SX3PPX4X5FGG
X1: P Q S
X2: D G N S
X3: N S
X4: G L V W
X5: A I T V
PDL1_VLambda_CDR3.26
YYCQX1WX2SX3PPX4X5FGG
X1: P Q S
X2: D G N S
X3: N S
X4: G L V W
X5: A I T V
PDL1_VLambda_CDR3.27
YYCQX1YX2SX3PPX4X5FGG
X1: P Q S
X2: S Y
X3: R S T W
X4: G L V W
X5: A I T V
PDL1_VLambda_CDR3.28
YYCQX1WX2SX3PPX4X5FGG
X1: P Q S B Y
X2: S Y
X3: R S T W
X4: G L V W
X5: A I T V
PDL1_VLambda_CDR3.29
YYCQX1YX2SX3PPX4X5FGG
X1: P Q S
X2: D G N S
X3: R S T W
X4: G L V W
X5: A I T V
PDL1_VLambda_CDR3.30
YYCQX1WX2SX3PPX4X5FGG
X1: P Q S
X2: D G N S
X3: R S T W
X4: G L V W
X5: A I T V
PDL1_VLambda_CDR3.31
YYCX1X2WDX3SLX4X5X6VFGG
X1: A E P Q S
X2: A F I S T V
X3: A D S Y
X4: D G N S
X5: A D G H P R
X6: H P S Y
PDL1_VLambda_CDR3.32
YYCX1X2WDX3SLX4X5X6VFGG
X1: A E P Q S
X2: A F I S T V
X3: A D S Y
X4: D G N S
X5: A D G H P R
X6: G L V W
PDL1_VLambda_CDR3.33
YYCQX1YX2X3X4PX5X6X7X8PGG
X1: P Q S
X2: D N
X3: N S
X4: S W
X5: P T
X6: A D G H I L N P R S T V

### Example 6

### Adapter-Directed Antibody Phage Display

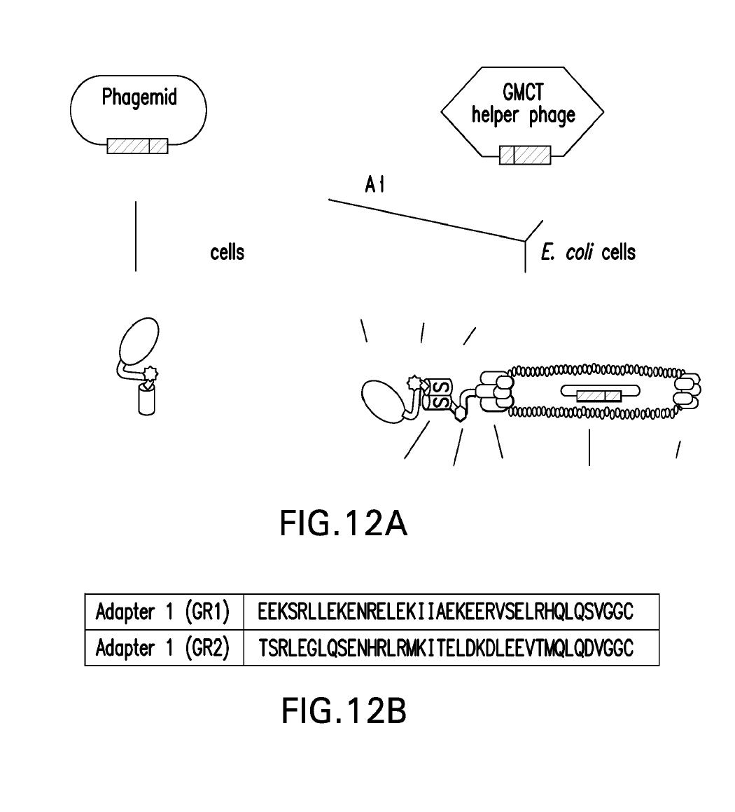

The adapter-directed phage display system is illustrated in FIG. 12. This system was comprised of two components as described in U.S. Pat. No. 7,175,983 B2: a phagemid vector avoiding any sequence encoding for phage coat protein, and a helper phage GMCT with one copy of recombinant coat protein. In the phagemid vector, the target gene was fused in-frame with the HA-tag (for detection) and adapter 1 sequence. The protein of interest was expressed as a secretory protein with adapter 2 as a tag (left panel in FIG. 12A). Diverse DNA sequences were inserted into this vector to construct an expression library. In the engineered helper phage genome, the adapter 2, which is able to form a pairwise heterodimer with the adapter 1, was fused in-frame with one of the phage coat proteins. For detection purposes, the Myc-tag sequence was placed in-between adapter 2 and coat protein. Both adapter fusions are transported into the periplasm, where the adapter association occurs followed by the stabilization of the heterodimer by a disulfide bond. The incorporation of the heterodimer into the phage particle thus leads to the display of target protein on the phage surface (right panel in FIG. 12A).

We chose two coiled-coil sequences, derived from the intracellular C-terminus of gamma-aminobutyric acid (B) receptors GABA_{B}-R1 and GABA_{B}-R2GR1, as the adapters in this study. The adapter sequences, termed GR1 and GR2, are shown in FIG. 12B. A previous study proved that these sequences preferentially formed parallel coiled-coil heterodimers under physiological buffer and temperature conditions (Kammerer, R. A., Frank, S., Schulthess, T., Landwehr, R., Lustig, A. & Engel, J. (1999). Heterodimerization of a functional GABAB receptor is mediated by parallel coiled-coil alpha-helices. Biochemistry. 38, 13263-13269.). The coiled-coil peptides from GABA_{B}-R1 only folded into relatively unstable homodimers, whereas the coiled-coil peptides from GABA_{B}-R2 were largely unstructured and could not form any homodimers. To further stabilize the heterodimer on the phage surface, we have introduced a spacer sequence (ValGlyGlyCys) after each coiled-coil sequence to lock the heterodimeric coiled-coil pair via a disulfide bond between the cysteine residues.

### Example 7

### Construction of GMCT Helper Phage Vector

A unique KpnI site was introduced into the gene III leader sequence of M13KO7 helper phage vector (Amersham Pharmacia) by PCR-based site-directed silent mutagenesis.

The KO7 genome was amplified by PCR using the following primers containing KpnI sites: p3KN1:5′-TTTAGTGGTA CCTTTCTATTCTCACTCCGCTG-3′ and p3KN2:5′-TAGAAAGGTACCACTAAAGGAATTGCGAATAA-3′. The PCR products were gel purified, cut with KpnI and ligated, then transformed into TG1 cells by electroporation. The kanamycin-resistant colonies were grown overnight in 96-well microtiter plates in 207 medium with 70 μg/ml kanamycin. The supernatants were directly employed to phage ELISA assay for phage production screening to eliminate the loss-of-function mutants caused by PCR errors. The presence of KpnI site in phage-positive clones was further confirmed by KpnI digestion and sequencing.

GMCT helper phage vector was constructed from KpnI-introduced M13KO7 helper phage vector by replacing the KpnI-BamHI fragment of WT gene III with a synthetic DNA fragment, which encodes two cistrons. The first is for a partial pIII leader (amino acid residues 11-19), the pIII C-terminus domain (amino acids 217 to 405) sequence fused with the adaptor GR2 and myc-tag sequence; the second for a ribosome binding sequence (S/D), the bacterial protein OmpA leader sequence and the wild-type gene III fragment before BamHI site.

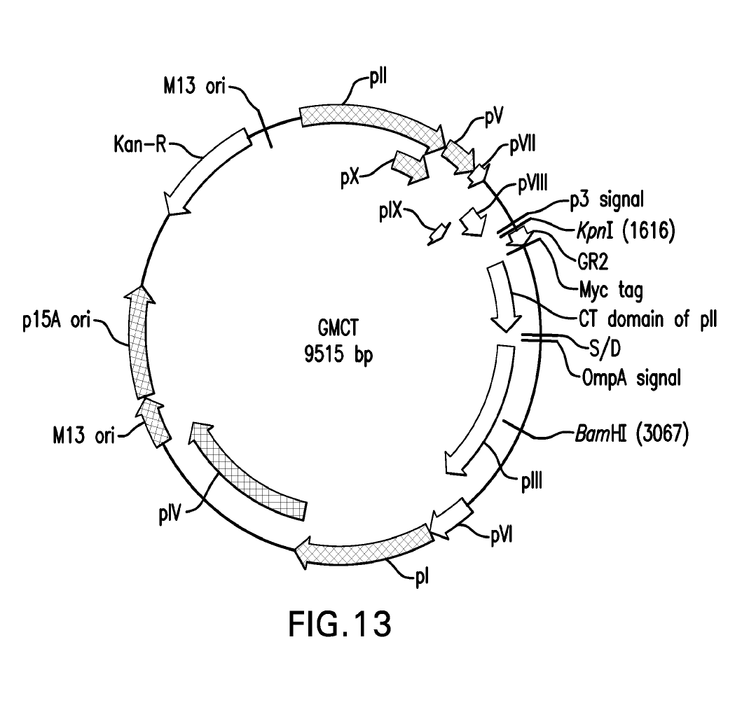

Both the engineered and wild-type gene III were under the control of the original gene III promoter (shown in FIG. 13).

### Example 8

### Helper Phage Generation

Escherichia coli TG1 (supE Δ(hsdM-mcrB)5(rₖ⁻mₖ⁻McrB⁻)thi Δ(lac-proAB/F′ traD36), LacI^{q} Δ(lacZ)M15) was used for phage production; The TG1 culture supernatant containing helper phage virus was streaked on a 2×YT agar plate. 4 ml of soft agar mixed with 0.5 ml of TG1 culture (OD600=0.5) was poured on to the plate. Phage plaques were formed after an overnight incubation at 37° C. A single phage plaque was picked and used to inoculate 10 ml 2×YT culture with 70 μg/ml kanamycin. After incubation at 37° C. for 2 hours with constant shaking at 250 rpm, the culture was transferred to a 2 liter flask containing 500 ml 2×YT with 70 μg/ml kanamycin, and incubated overnight with constant shaking at 250 rpm. The phage particles in the supernatants were then precipitated using polyethylene glycol (PEG)/NaCl followed by 10000 rpm centrifugation, and re-suspended in phosphate-buffed saline (PBS). PEG-precipitation was repeated once. The phage concentration was determined by OD₂₆₈ measurement, assuming 1 unit at OD₂₆₈ is approximately 5×10¹² phage particles/ml. The phage yield of GMCT helper phage was approximately 8×10¹¹ phage/ml in the culture supernatant, which was similar to that of wild type helper phage M13KO7, indicating that assembly of recombinant pill into phage particles did not significantly affect phage production.

### Example 9

### Phagemid Fab-Display Vector Construction

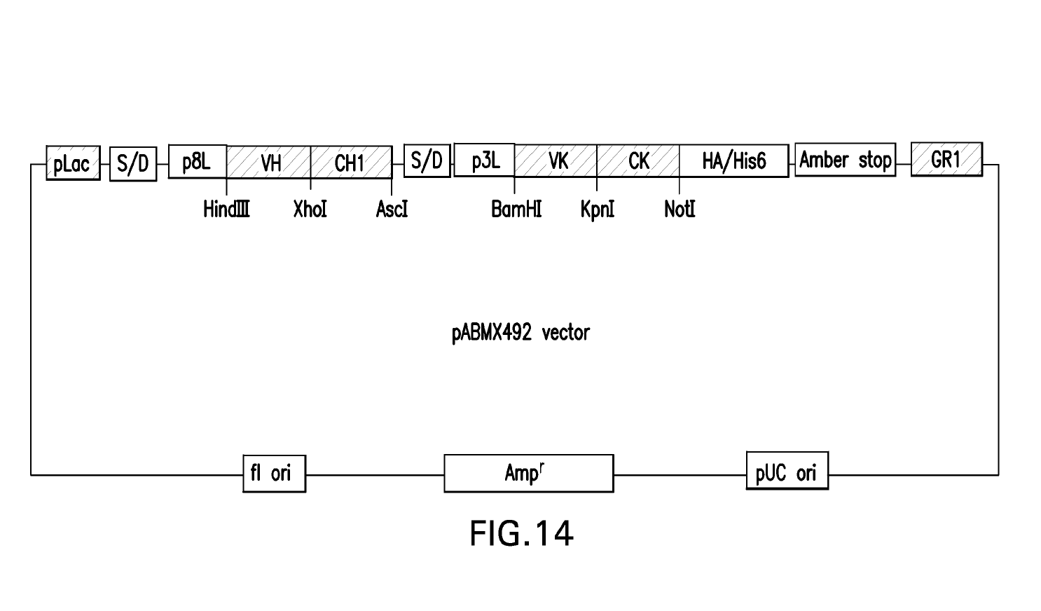

The Fab-display phagemid vector was derived from pBluescript SK(+) (Stratagene). A unique AgeI restriction site was introduced immediately after the lac promoter by PCR-based site-directed mutagenesis, with a set of primers (pBS-Ska: 5′-GGAATTGTGAGCGGATAACAATTTACCGGTCACACAGGAAACAGCTATGACCATG-3′ and pBS-SKb: 5′ CATGGTCATAGCTGTTTCCTGTGTGACCGGTAAATTGTTATCCGCTCACAAT TCC-3′). The Xho I and Kpn I sites were then deleted by digestion and consequent blunt-end ligation. The synthetic DNA fragment flanked by AgeI site at 5′ and San site at 3′, containing two cistrons for both light chain and heavy chain of Fab molecule, and coding sequences for adaptor GR1 and HA-(His)₆-tag (referred to as DH tag), was cloned into the engineered pBluescript SK(+). Both light and heavy chains are driven by a single lac promoter, with ribosome-binding sequence S/D and secretion leader sequence in front of each chain. The VH and VK from an anti-IL13 receptor (IL-13R) antibody, and the constant regions of CH1 and Ck of human IgG1 antibody were used for the vector construction. In the pABMX492 vector, the expression of antibody heavy chain and light chain is driven by a single lac promoter. Only light chain was fused with adapter GR1 sequence. An amber stop codon which reads as amino acid Glutamine (Q) in TG1 strain was placed in between HA/His-tag and GR1 adapter (vector map shown in FIG. 14).

### Example 10

### Adapter-Directed Phage Display of Fab Molecules

The Fab display experiments were carried out by infection of TG1 cells harboring pABMX492 vector with GMCT helper phage. The infected TG1 cells were grown in 2×YT/Amp/Kan at 30° C. overnight. The phagemid particles were precipitated by PEG/NaCl from culture supernatants, and resuspended in PBS. The PEG-precipitation was repeated once. The antibody displayed on phage surface was detected by its antigen binding activity via phage ELISA assay. Briefly, the antigens were first coated on ELISA plates. After 5% milk/PBS blocking, the phage solution in milk/PBS was added to ELISA plates. The phages bound to antigen on the plate were detected by incubation with HRP-conjugated anti-M13 antibody. The substrate ABTS [2,2′Azino-bis(3-ethylbenzthiazoline-6-sulfonic acid)] was used for measurement of HRP activity.

Phage ELISA data showed a dose-dependent binding activity of pABMX492 phages to antigen IL-13R, with a binding saturation at around 10¹³ phage/ml. This result confirmed the functional display of anti-IL13 receptor Fab on phage surface through adapter-directed display system.

### Example 11

### Construction of Display Vectors for Fab Antibody Libraries

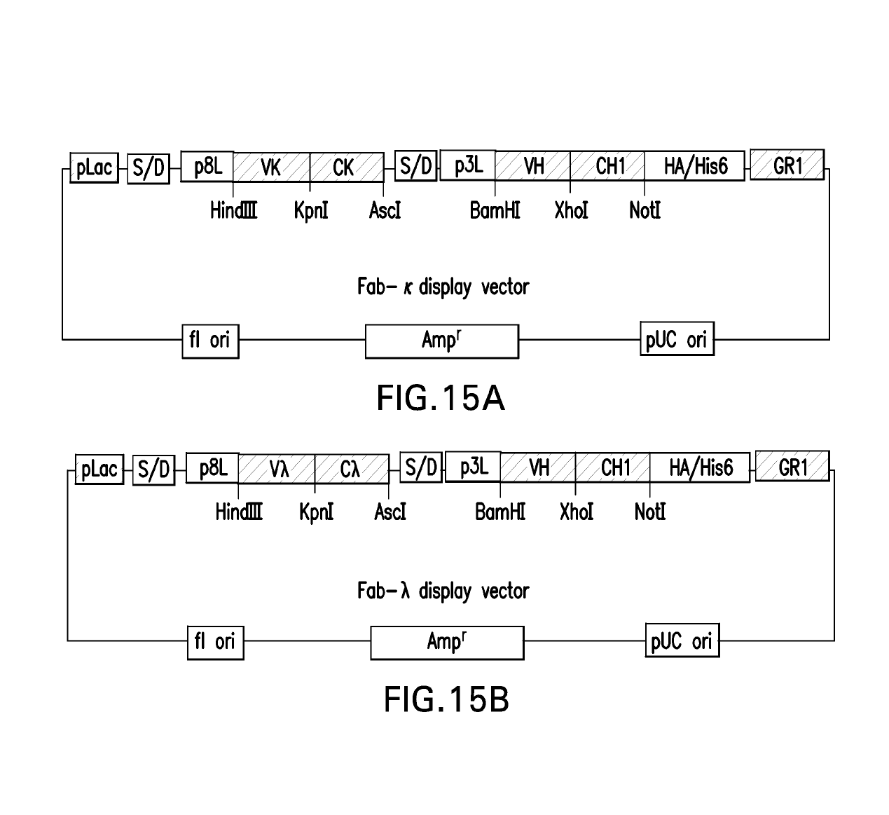

Two vectors were constructed for Fab library cloning from the engineered pBluescript SK(+) described in example 9. Briefly, the AgeI and BglII fragment in the engineered pBluescript SK(+) was replaced by a synthetic DNA fragment containing expression cassettes for both light and heavy chains. The vector map for Fab library with human k light chain is illustrated in FIG. 15A. The vector map for Fab library with human lambda light chain is illustrated in FIG. 15B. In both Fab display vectors, the cistron for light chain was placed in front of heavy chain cistron. Another difference from vector pABMX492 is that both k and lambda vectors didn't have an amber stop between HA/His tag and adapter GR1.

### Example 12

### PDL1 Library Construction Strategy

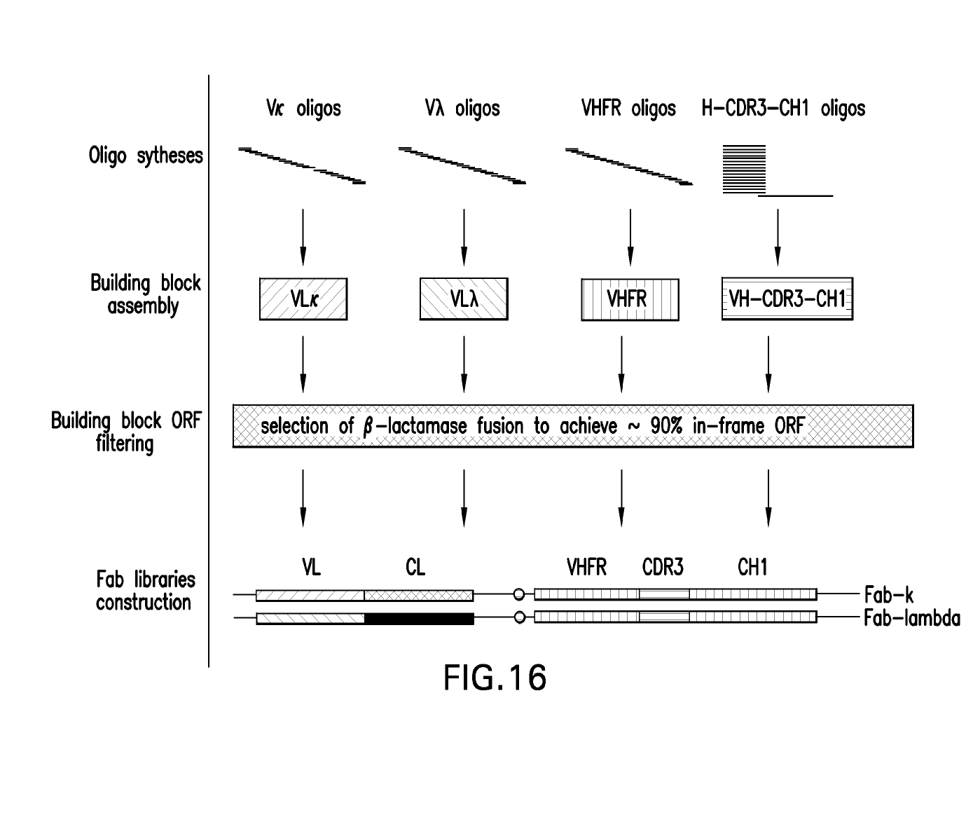

Highly diverse synthetic libraries were computationally designed to capture the most important structure diversity. Construction of these synthetic libraries consisted of the following four major consecutive steps: syntheses of library degenerated oligos (40 to 90mer), assembly of building blocks and chain libraries from 45 to 90mer oligos, ORF filtering of building blocks to remove open-reading frame shifts, and Fab library construction by cloning light chain and heavy chain into Fab display vectors (FIG. 16). In the PDL1 library construction, heavy chain library was assembled from two building blocks: VHFR building block and VH-CDR3-CH1 building block. VHFR building block contained VH-FR1, CDR1, FR2, CDR2 and FR3. The VH-CDR1 and CDR2 contributed the amino acid diversity of 10⁵ to 10⁶. The building block of VH-CDR3-CH1 had amino acid diversity of 10⁵ to 10⁶ in the CDR3 region. The chain libraries for light chains were assembled into full length of VLs including FR1,2,3 and CDR1,2,3. The amino acid diversity of K and lambda light chain libraries was 10⁵ to 10⁸ in the CDR regions.

### Example 13

### Library Oligo Synthesis and Quality Analysis

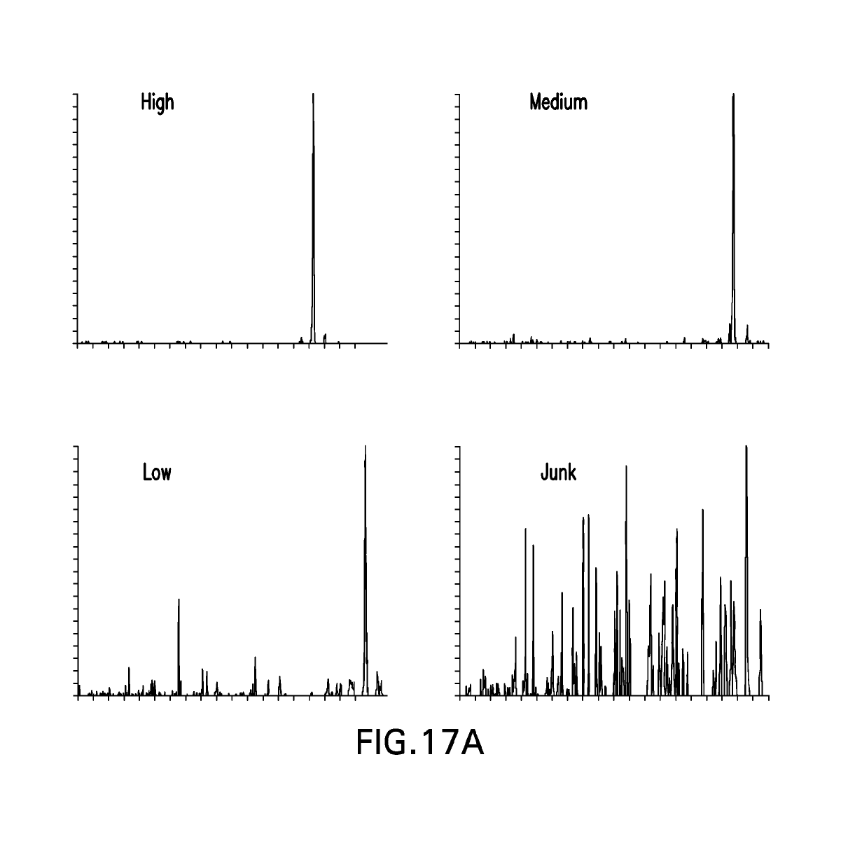

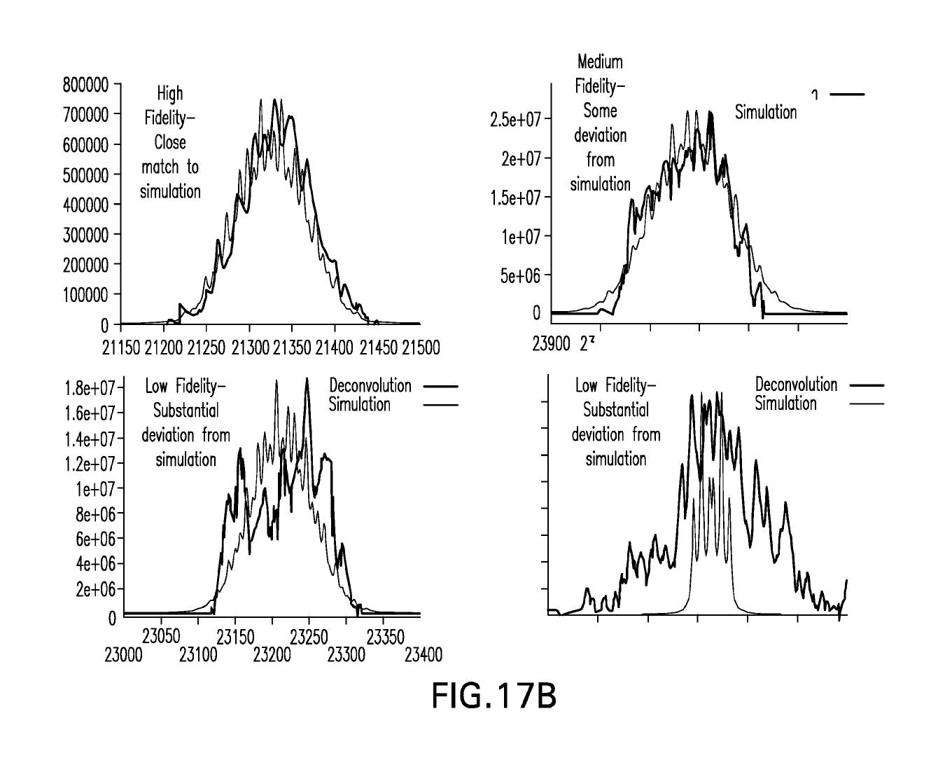

All library oligos were simultaneously sent to three vendors (Operon, Sigma, and IDT) for synthesis. All oligos including degenerate oligos were analyzed by Mass Spectrometry. The purity of oligos was assessed by level of target oligo molecular weight peak over side reaction products (FIG. 17A). The oligo fidelity was evaluated by MS profile comparison of actual oligo mass spectrometry (Main peak) with predicted MS masses from simulation based on design. We classified the oligos with close match between actual MS and simulation as high fidelity, oligos with some deviation from simulations as medium fidelity, and oligos with substantial deviation from simulation as low fidelity (FIG. 17B). All oligos were then divided into four quality groups according to their purity and fidelity: High, Medium, Low, and Junk. Those of low and junk purity were re-synthesized. From the triplicates of each oligo, the one with highest quality was used for building block assembly.

### Example 14

### Assembly of Building Block and Chain Libraries

Two rounds of PCRs using Pfu DNA polymerase (Stratagene #600602) were involved in construction of each building block or chain library: assembly PCR and amplification PCR. To ensure an equal representation of each designed sequence, 20 to 50 overlapping oligos including degenerate oligos with high quality were mixed proportionally according to their complexity and then assembled to form the template for PCR amplification.

### Example 14

### 1. Construction of VH-FR Building Blocks

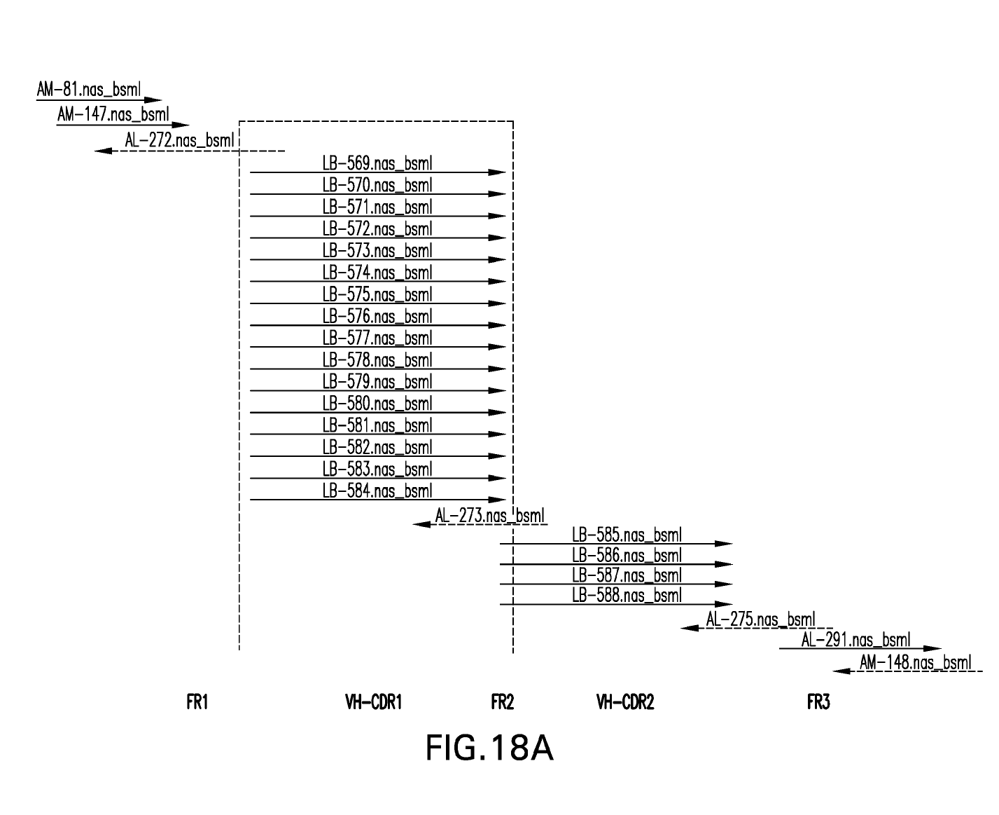

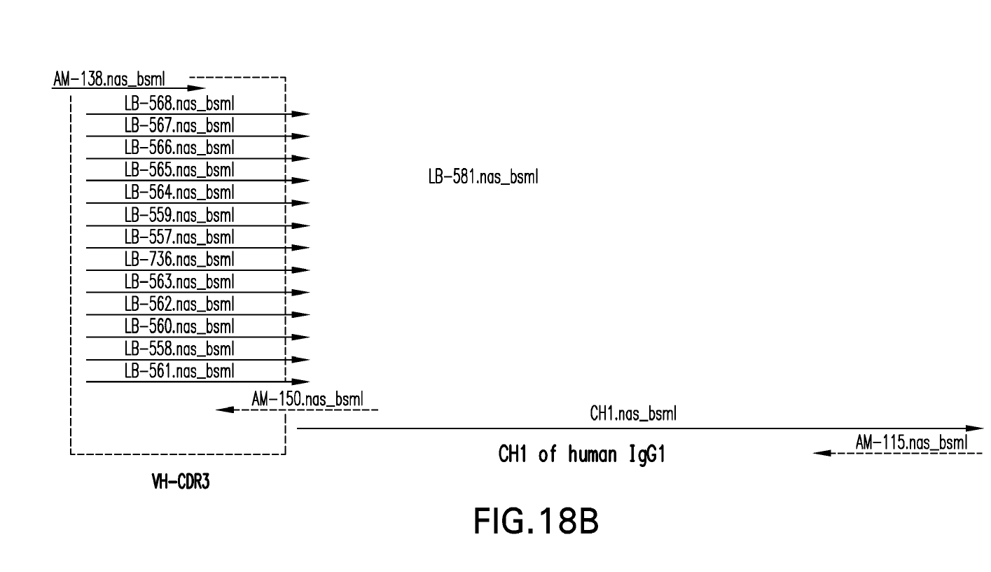

Total 12 of CDR2 oligos were divided into 6 groups based on the overlapping sequences at their 5′ and 3′ to assemble with 16 CDR1 oligos and 7 FR oligos to make VHFR building block. The FIG. 18A illustrated group 1 assembly of 28 overlapping oligos. The assembly PCR was carried as following. The oligo stocks of 100 uM were mixed together according to oligo diversity to make 0.5 uM oligo solution. 50 ul of PCR solutions were then prepared as described in the PCR assembly table 5. The PCR solutions were preheated at 94° C. for 2 minutes, then 25 thermal cycles of denature (94° C., 45 second), anneal (51° C., 45 second), and elongate (72° C., 1 min.) were performed, followed with a cycle of 72 C, 10 min for completion.

TABLE 5
PCR Assembly for VHFR

| Assembly reaction | #1 | #2 | #3 |
|---|---|---|---|
| oligo mix | 1 ul | 2 ul | 3 ul |
| 10 × Pfu buffer | 5 ul | 5 ul | 5 ul |
| dNTP (2.5 mM each) | 4 ul | 4 ul | 4 ul |
| Mg(Ac) 25 mM | 2 ul | 2 ul | 2 ul |
| H2O | 36 ul | 35 ul | 34 ul |
| Pfu turbo | 2 ul | 2 ul | 2 ul |
| Total | 50 ul | 50 ul | 50 ul |

The assembled building blocks were further amplified by PCR by using the primers AM-61 (5′-ACCCTCGTTCCGATGCTAAGCTTCGC-3′) and AM-148 (5′-ACGGGCGCAGTAGTACACTGC-3). The PCR solutions were prepared by following the table 6, then 35 cycles of PCR reactions were run as described above.

TABLE 6
PCR Amplification of VH-FR building block

| Amplification reaction | #1 | #2 | #3 |
|---|---|---|---|
| Assembly reaction | 10 ul | 10 ul | 10 ul |
| | assembly #1 | assembly #2 | assembly #3 |
| 5′ primer AM-61 (10 uM) | 5 ul | 5 ul | 5 ul |
| 3′ primer AM-148 (10 uM) | 5 ul | 5 ul | 5 ul |
| 10 × Pfu buffer | 10 ul | 10 ul | 10 ul |
| dNTP (2.5 mM each) | 8 ul | 8 ul | 8 ul |
| H2O | 60 ul | 60 ul | 60 ul |
| Pfu | 2 ul | 2 ul | 2 ul |
| Total | 100 ul | 100 ul | 100 ul |

The assembled building blocks were purified by electrophoresis in 1.5% agarose gel, were then combined from all 6 groups according to their diversity to make equal representation of each designed sequence in the final building block library.

### Example 14

### 2: Construction of VH-CDR3-CH1 Building Block

Total 516 oligos were used to assemble with CH1 DNA fragment to make VH-CDR3-CH1 building block. The human CH1 fragment was amplified from pABMX492 vector by PCR, using a set of primers: AM-58 (5′-GTCTCGAGCGCAAGCACCAAAGGC-3′) and AM-115 (5′-CATGTGTGAGTTTTGTCACAAGATTTGGGC-3′). The CDR3 oligos (total 516) were divided into 12 groups to assemble with CH₁ (FIG. 18B shows one subgroup assembly). The assembly PCRs were performed as described in Example 14.1. Briefly, the oligos were mixed with CH1 fragment as described in the table 7, then 25 cycles of assembly PCR was carried out with each cycle of 94° C., 45 second of denature, 51° C.; 45 second of anneal; and 72° C., 1 min of elongate.

TABLE 7
PCR Assembly for VH-CDR3-CH1

| Assembly reaction | #1 | #2 | #3 |
|---|---|---|---|
| oligo mix | 1 ul | 2 ul | 3 ul |
| CH1 | 5 ul | 5 ul | 5 ul |
| 10 x Pfu buffer | 5 ul | 5 ul | 5 ul |
| dNTP (2.5 mM each) | 4 ul | 4 ul | 4 ul |
| Mg(Ac) 25 mM | 2 ul | 2 ul | 2 ul |
| H2O | 31 ul | 30 ul | 29 ul |
| Pfu turbo | 2 ul | 2 ul | 2 ul |
| Total | 50 ul | 50 ul | 50 ul |

The assembled building blocks were further amplified by 35 cycles of PCR by using the primer AM-138 (5′-GACACTGCAGTGTACTACTGCGCC-3′) and AM-115. The PCR solutions were prepared by following the table 4, then 35 cycles of PCR reactions were run as described in example 14.1.

TABLE 8
PCR Amplification of VH-CDR3-CH1 building block

| Amplification reaction | #1 | #2 | #3 |
|---|---|---|---|
| Assembly reaction | 10 ul | 10 ul | 10 ul |
| | assembly #1 | assembly #2 | assembly #3 |
| 5′ primer AM-138 (10 uM) | 5 ul | 5 ul | 5 ul |
| 3′ primer AM-115 (10 uM) | 5 ul | 5 ul | 5 ul |
| 10 x Pfu buffer | 10 ul | 10 ul | 10 ul |
| dNTP (2.5 mM each) | 8 ul | 8 ul | 8 ul |
| H2O | 60 ul | 60 ul | 60 ul |
| Pfu | 2 ul | 2 ul | 2 ul |
| Total | 100 ul | 100 ul | 100 ul |

The PCR products from all 12 assembly were purified by electrophoresis in 1.5% agarose gel. The gel-purified VH-CDR3-CH1 building blocks from all 12 groups were then combined according to their diversity to make equal representation of each sequence in the building block library.

### Example 14

### Construction of Vk1 Chain Library

Two Vk chain libraries were assembled through overlapping oligos. One was Vk1 chain library with diversity of 2.7×10⁷, another was Vk3 chain library with diversity of 1.7×10⁷ in the CDR regions.

For Vk1 chain library assembly, 44 oligos were mixed together according to oligo diversity to make 0.5 uM oligo solution. 50 ul of PCR solutions were then prepared as described in the PCR assembly table 5. The PCR solutions were preheated at 94° C. for 2 minutes, then 25 thermal cycles of denature (94° C., 45 second), anneal (51° C., 45 second), and elongate (72° C., 1 min.) were performed, followed with a cycle of 72° C., 10 min for completion.

The assembled building blocks were further amplified by PCR by using the primers AM-141 (5′-ACCCTCGTTCCGATGCTAAGCTTCGCTGACATCCAGATGACCCAGTCT-3′) and AM-153 (5′-GAATACAGATGGTGCAGCCAC-3′). The PCR solutions were prepared by following the table 9, then 35 cycles of PCR reactions were run as described above. The PCR product of Vk1 chain library was purified by electrophoresis in 1.5% agarose gel.

TABLE 9
PCR Amplification of Vk1 chain library

| Amplification reaction | #1 | #2 | #3 |
|---|---|---|---|
| Assembly reaction | 10 ul | 10 ul | 10 ul |
| | assembly #1 | assembly #2 | assembly #3 |
| 5′ primer AM-141 (10 uM) | 5 ul | 5 ul | 5 ul |
| 3′ primer AM-153 (10 uM) | 5 ul | 5 ul | 5 ul |
| 10 x Pfu buffer | 10 ul | 10 ul | 10 ul |
| dNTP (2.5 mM each) | 8 ul | 8 ul | 8 ul |
| H2O | 60 ul | 60 ul | 60 ul |
| Pfu | 2 ul | 2 ul | 2 ul |
| Total | 100 ul | 100 ul | 100 ul |

### Example 14

### Construction of Vk3 Chain Library

For Vk3 chain library assembly, 44 oligos covering 1.7×10⁷ amino acid diversity were mixed together according to oligo diversity to make 0.5 uM oligo solution. The PCR solutions were prepared as described in the PCR assembly table 5. The PCR solutions were preheated at 94° C. for 2 minutes, then 25 thermal cycles of denature (94° C., 45 second), anneal (51° C., 45 second), and elongate (72° C., 1 min.) were performed, with a following cycle of 72° C., 10 min for completion.

The assembled building block was further amplified by PCR by using the primers AM-155 (5′-ACCCTCGTTCCGATGCTAAGCTTCGCTGAAATCGTGCTGACCCAG-3′) and AM-153 (5′-GAATACAGATGGTGCAGCCAC-3′). The PCR solutions were prepared by following the table 10, then 35 cycles of PCR reactions were run as described above. The PCR product of Vk3 chain library was purified by electrophoresis in 1.5% agarose gel.

TABLE 10
PCR Amplification of Vk3 chain library

| Amplification reaction | #1 | #2 | #3 |
|---|---|---|---|
| Assembly reaction | 10 ul | 10 ul | 10 ul |
| | assembly #1 | assembly #2 | assembly #3 |
| 5′ primer AM-155 (10 uM) | 5 ul | 5 ul | 5 ul |
| 3′ primer AM-153 (10 uM) | 5 ul | 5 ul | 5 ul |
| 10 x Pfu buffer | 10 ul | 10 ul | 10 ul |
| dNTP (2.5 mM each) | 8 ul | 8 ul | 8 ul |
| H2O | 60 ul | 60 ul | 60 ul |
| Pfu | 2 ul | 2 ul | 2 ul |
| Total | 100 ul | 100 ul | 100 ul |

### Example 14

### Construction of V-Lambda Chain Library

For V-lamda chain library assembly, 34 oligos covering 1×10⁵ amino acid diversity were mixed together according to oligo diversity. The PCR solutions were prepared as described in the PCR assembly table 5. The PCR solutions were preheated at 94° C. for 2 minutes, then 25 thermal cycles of denature (94° C., 45 second), anneal (51° C., 45 second), and elongate (72° C., 1 min.) were performed, with a following cycle of 72° C., 10 min for completion.

The assembled building blocks were further amplified by PCR by using the primers AM-163 (5′-CCTTTCTATTCTCACTCGCTGGATCC-3′) and AM-164 (5′-GGCAGCCTTGGCTGACCCAGCAC-3′). The PCR solutions were prepared by following the table 11, then 35 cycles of PCR reactions were run as described above. The PCR product of V-lambda chain library was purified by electrophoresis in 1.5% agarose gel.

TABLE 11
PCR Amplification of V-lambda chain library

| Amplification reaction | #1 | #2 | #3 |
|---|---|---|---|
| Assembly reaction | 10 ul | 10 ul | 10 ul |
| | assembly #1 | assembly #2 | assembly #3 |
| 5′ primer AM-163 (10 uM) | 5 ul | 5 ul | 5 ul |
| 3′ primer AM-164 (10 uM) | 5 ul | 5 ul | 5 ul |
| 10 x Pfu buffer | 10 ul | 10 ul | 10 ul |
| dNTP (2.5 mM each) | 8 ul | 8 ul | 8 ul |
| H2O | 60 ul | 60 ul | 60 ul |
| Pfu | 2 ul | 2 ul | 2 ul |
| Total | 100 ul | 100 ul | 100 ul |

### Example 15

### ORF Filtering for Assembled Building Blocks and Chain Libraries

The most common errors observed in the synthetic oligos were deletion and insertion, which caused gene open-reading shifts. Based on our observation, using high purity oligos alone could not guarantee a high level of ORFs in the assembled building blocks by normally giving around 50% correct open-reading frame, thereby a selective step to filter away the DNA fragments with OFR frameshifts and stop codons was setup for all building blocks and chain libraries.

### Example 15

### Construction of pMAS3 Vector for ORF Filtering

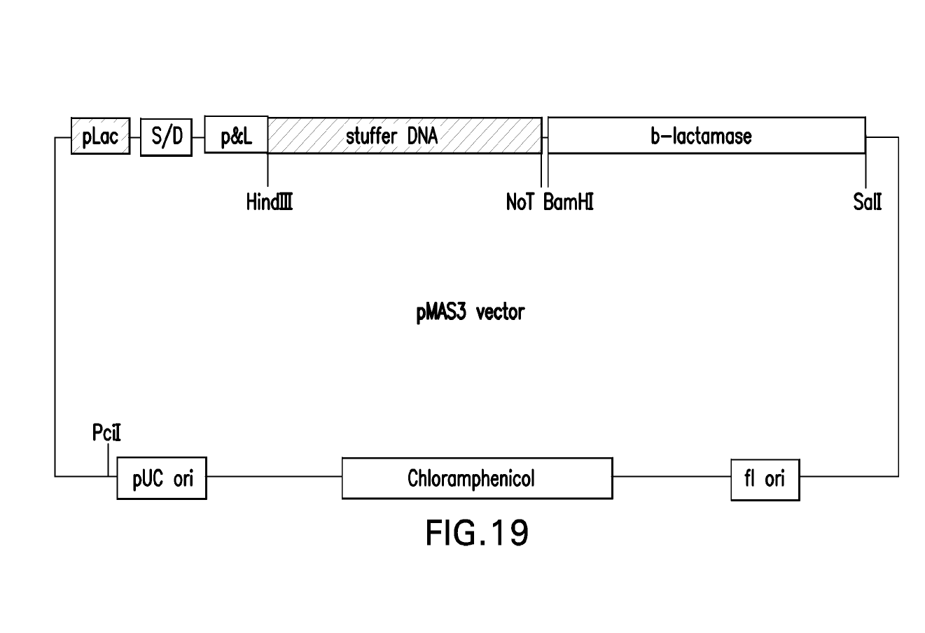

The vector pMAS3 was constructed from commercial vector pBC SK (+) (Strategen) by four steps. First step, the Xho I and Kpn I sites were deleted by digestion and subsequent blunt-end ligation; in the second step, the PciI to BamHI fragment in PBC SK(+) was replace with corresponding fragment from pABMX493 vector (Example 11); in the third step, a stuffer DNA fragment of neomycin was reversely cloned into modified PBC SK(+) vector by HindIII and BamHI; finally, a β-lactamase gene amplified from pBluescipt SK (+) was cloned into engineered pBC SK(+) vector by BamHI and SalI site. The amplification primers was Amp-411 (5′-GGTGGCGGATCCCACCCAGAAACGCTG-3′) and Amp-413 (5′-ATTAATGTCGACTTACCAATGCTTAATCAGTGAG-3′). The PMAS3 vector map is illustrated in FIG. 19.

### Example 15

### ORF Filtering of all Building Blocks and Chain Libraries

The assembled building blocks and chain libraries were first amplified by PCR as described in Example 14 to introduce HindIII and NotI at two ends. The amplification primers used for amplification are listed in the table 12.

TABLE 12
primers for amplification of building blocks and chain libraries

| Primer | Sequence | Amplification |
|---|---|---|
| AM-61 | ACCCTCGTTCCGATGCTAAGCTTCGC | VHFR building block |
| AM-149 | CGTACGGCTCGAGACAGCGGCCGCACGGGCGCAGTAGTACACTGCAGT | VHFR building block |
| AM-152 | CTCGTTCCGATGCTAAGCTTCGCTGACACTGCAGTGTACTACTGCG | VH-CDR3-CH building |
| | | blocks |
| AM-97 | CGTACGGCTCGAGACAGCGGCCGCTGTGTGAGTTTTGTCACAAGATTTGG | VH-CDR3-CH building |
| | | blocks |
| AM-141 | ACCCTCGTTCCGATGCTAAGCTTCGCTGACATCCAGATGACCCAGTCT | Vk1 chain library |
| AM-154 | CGTACGGCTCGAGACAGCGGCCGCGAATACAGATGGTGCAGCCAC | Vk1 chain library |
| AM-155 | ACCCTCGTTCCGATGCTAAGCTTCGCTGAAATCGTGCTGACCCAG | VK3 chain library |
| AM-154 | CGTACGGCTCGAGACAGCGGCCGCGAATACAGATGGTGCAGCCAC | VK3 chain library |
| AM-165 | ACCCTCGTTCCGATGCTAAGCTTCGCTCAGTCTGTGCTGACCCAGCCA | V-lambda chain library |
| AM-66 | CCGTACGGCTCGAGACAGCGGCCGCCAGCACGGTCAGCTTGGTACC | V-lambda chain library |

Each amplified building block or chain library was cloned into pMAS3, using Hind III and Not I to make β-lactamase fusions. The ligation products were purified by Qiaquick PCR Purification kit (Qiagen), then transform into XL1-Blue cells by electroporation. The transformed cells were plated out on 2YT agar plates with 2% glucose and 30 μg/ml chloramphenicol for an overnight incubation. To ensure a full coverage of library diversity, multiple electroporations were performed until the colonies number reached over at least 5 times of the actual diversity of each building block or chain library.

The cells on chloramphenicol plates were scraped off with 2YT broth/glucose (2%)/chloramphenicol (30 μg/ml) and combined. A portion of the combined cells, which covered at least 10 time of building block diversity, were plated out on 2YT agar plates with both 30 μg/ml chloramphenicol and 100 μg/ml carbenicillin for selection. A few dilution plates were also prepared to determine the output. After overnight incubation at 37° C., the survival cells on the double antibiotic selection plates were harvested. The DNA pool for each individual building block or chain library was prepared from above survival cells.

The bacterial colonies from pre-filtering and post-filtering plates were sent out for DNA sequencing. The results showed that most of the building blocks contained around 50% ORFs before chloramphenicol/carbenicillin double selection. After filtering, the percentage of antibody open-reading frames was significantly increased to over 95% (Table 13).

TABLE 13
sequence analysis for building blocks or chain libraries

| Building blocks | | | |
|---|---|---|---|
| or Chain libraies | Pre-filtering ORF | Post-filtering ORF | Complexity |
| Vk1 | 42% (57/137) | 96% (360/373) | 2.4 × 10\hat{ }7 |
| Vk3 | 28% (32/116) | 94% (456/485) | 1.6 × 10\hat{ }7 |
| Vλ | 46% (63/136) | 95% (150/157) | 10\hat{ }5 |
| VHFR (CDR1 & | 53% (300/576) | 95% (1090/1142) | 5.6 × 10\hat{ }5 |
| CDR2) | | | |
| VH-CDR3-CH1 | 83% (300/360) | 98% (1241/1259) | 2.7 × 10\hat{ }5 |

### Example 16

### Construction of Fab Libraries

The final Fab libraries were constructed by two steps. In the first step, the Vk and V-lambda light chain libraries were respectively cloned into Fab-display vectors (FIG. 15); the assembled heavy chain library from VHFR and CDR3-CH1 building blocks was then cloned into the step 1 vectors with eight k or lambda light chain libraries.

### Example 16

### Cloning of Light Chain Libraries into Fab Display Vectors

The K1, K3 and lambda chain libraries were amplified from post-filtering DNA pools by PCR as described in Example 9, using primers listed in table 14 with HindIII and KpnI sites at two ends.

TABLE 14
amplification primers for post-filtering chain libraries

| Primer | Sequence | Amplification |
|---|---|---|
| AM-141 | ACCCTCGTTCCGATGCTAAGCTTCGCTGACATCCAGATGACCCAGTCT | Vk1 chain library |
| AM-153 | GAATACAGATGGTGCAGCCAC | Vk1 chain library |
| AM-155 | ACCCTCGTTCCGATGCTAAGCTTCGCTGAAATCGTGCTGACCCAG | VK3 chain library |
| AM-153 | GAATACAGATGGTGCAGCCAC | VK3 chain library |
| AM-189 | CTCGTTCCGATGCTAAGCTTCGCTCAGTCTGTGCTGACCCAG | V-lambda chain |
| | | library |
| AM-164 | GGCAGCCTTGGCTGACCCAGCAC | V-lambda chain |
| | | library |

The PCR solutions were preheated at 94° C. for 2 minutes, then 35 thermal cycles of denature (94° C., 45 second), anneal (51° C., 45 second), and elongate (72° C., 1 min.) were performed, with a following cycle of 72 C, 10 min for completion. The PCR products of all three chain libraries were separately purified by Qiaquick PCR Purification kit (Qiagen). The purified DNA of K1, K3, and Lambda chain libraries were separately digested with HindIII and KpnI, and purified by electrophoresis in 1.5% agarose gel, thus cloned respectively into Fab-k or Fab-lambda display vectors precut with same restriction enzymes. The ligation products from all three light chain libraries were separately purified by Qiaquick PCR Purification kit (Qiagen), separately transform into XL1 blue cells by electroporation, then the transformed cells for all three libraries were separately plated out on ˜100 2YT agar plates with 2% glucose and 100 carbenicillin. The cloning processes were repeated multiple times until the colonies number reached over 5 to 10 times of the diversity of each chain library. The colonies for individual light chain library on the plates were harvested in 2YT with 2% glucose and 100 μg/ml carbenicillin. The bacterial glycerol stocks and plasmid DNA were made for each individual library.

### Example 16

### Cloning of Heavy Chain Library into Light Chain Library Vectors

The heavy chain building blocks VHFR and VH-CDR3-CH1 were amplified from post-filtering DNA pools by PCR as described in Example 14, using primers listed in table 15. The PCR solutions were preheated at 94° C. for 2 minutes, then 30 thermal cycles of denature (94° C., 45 second), anneal (55° C., 45 second), and elongate (72° C., 1 min.) were performed, with a following cycle of 72° C., 10 min for completion. The PCR products were purified by electrophoresis in 1.5% agarose gel.

TABLE 15
primers for amplification of building blocks and chain libraries

| Primer | Sequence | Amplification |
|---|---|---|
| AM-190 | TCTCACTCCGCTGGATCCGAAGTGCAGCTGCTGGAATC | VHFR building block |
| AM-148 | C-ACGGGCGCAGTAGTACACTGC | VHFR building block |
| AM-138 | GAcACTGCAGTGTACTACTGCGCC | VH-CDR3-CH building blocks |
| AM-124 | GTCGTATGGATAAGCGGCCGCTGTGTGAGTTTTGTCACAAGATTTG | VH-CDR3-CH building blocks |

The f [symbol] sec; 55 C, 45 sec; 72 C, 1 [symbol] min”; then 72 C, 10 min for completion. More than 100 PCR reactions were performed to make enough DNA for subsequent cloning into Fab display vectors.

TABLE 16
CR assembly for heavy chain library

| | |
|---|---|
| AM-190, 10 uM | 3 ul |
| AM-124, 10 uM | 3 ul |
| VHFR DNA | 2 ul |
| VH-CDR3-CH1 DNA | 2 ul |
| H2O | 40 ul |
| Pfu mastermix | 50 ul |
| Total (ul) | 100 ul |

The PCR products of heavy chain library were purified by Qiaquick PCR

Purification kit (Qiagen). The purified library DNA was digested with BamHI and NotI, and purified by electrophoresis in 1.5% agarose gel, thus cloned separately with three Fab display vectors with Vk1, VK3, V-lambda light chain library. The ligation products for Fab-K1, Fab-K3 and Fab-lambda were purified by Qiaquick PCR Purification kit (Qiagen), and separately transform into TG1 cells by electroporation. The transformed cells for all three libraries were separately plated out on more than 1000 2YT agar plates with 2% glucose and 100 μg/ml carbenicillin. The cloning processes were repeated multiple times to reach enough number of the transformants for coverage of the diversity of heavy chain library. The total complexity of PDL1 library size reached 9×10⁹, including PDL1-K1 sub-library with 3.6×10⁹,PDL1-K3 sub-library with 3.2×10⁹, and PDL1-lambda sub-library with 2.2×10⁹ (Table 17). The colonies for individual library on the plates were harvested in 2YT with 2% glucose and 100 μg/ml carbenicillin. The bacterial glycerol stocks were made for each individual library.

The bacterial colonies on 2YT plates for each individual library were sent out for DNA sequencing. The ORF % of each individual sub-libraries is listed in the table 17. The percentage of antibody open-reading frames for PDL1 library was 85%.

TABLE 17
PDL1 Fab Library

| Fab Libraries | Open Reading Frame | Complexity |
|---|---|---|
| PDL1-k1 | 84% (333/396) | 3.6 × 10⁹ |
| PDL1-k3 | 86% (596/694) | 3.2 × 10⁹ |
| PDL1-l1 | 83% (254/305) | 2.2 × 10⁹ |
| Total | 85% (1183/1395) | 9 × 10⁹ |

### Example 17

### Preparation of Phage Display Library of PDL1

For individual sub-library, 3 to 5 liters of library TG1 cells were inoculate in 2YT/2% glucose/100 μg/ml carbenicillin, with starting OD₆₀₀=0.1. The library cells were grew at 37° C. with 250 rpm shaking until OD₆₀₀ reached to ˜0.8-0.9. The library cells were then infected with GMCT helper phages (MOI=10) for 1 hour at 37° C. The infected TG1 cells were grown in 2×YT/carbenicillin/Kanamycin at 22° C. for overnight. The overnight culture supernatants were harvested by centrifugation at 8000×g for 60 minutes, and filtered though 45 um low binding membrane filter (Corning). The phagemid particles were precipitated by PEG/NaCl from culture supernatants, and resuspended in PBS. The PEG-precipitation was repeated once. The phage concentration was determined by OD₂₆₈ measurement, assuming 1 unit at OD₂₆₈ is approximately 5×10¹² phage particles/ml, and confirmed by colony formation assay on 2YT plate with 2% glucose/100 μg/ml carbenicillin. Library phage stocks were made with 15% glycerol, and stored at −80° C.

### Example 18

### PDL1 Phage Library Panning Against Multiple Antigen Proteins

Antigen protein was coated on Maxisorp well stripe (Nunc-Immuno Modules) at a concentration of 1-10 μg/ml for overnight at 4° C. Multiple wells of antigen were prepared for each library. 5% milk in PBS was used to block the coated wells at RT for 1-2 hour. After wash with PBS, 100 ul of phage library solution/well (usually 1-5×10¹² in 2% milk-PBSK) was added into 4 parallel wells, and incubate for designed length of time (usually 1-2 hours). After several washing with PBST and PBS, the bound phages was eluted from the wells with fresh-prepared 1.4% triethylamine in ddH2O (10 min incubation at room temperature), followed immediately with neutralization by adding 50 ul of 1M Tris-HCl (pH 6.8).

For some antigens, 1 ml of antigen protein was coated into immunotube at a concentration of 10 to 500 μg/ml for overnight at 4° C. The volume of block, wash and elution solutions were increased accordingly.

The eluted, enriched phage pool was further amplified through following steps. First, TG1 cells were infected with eluted phages at 37° C. for 1 hour, then plated out on 2YT agar plates with 2% glucose and 100 μg/ml carbenicillin for overnight culture. Thus TG1 cells harboring enriched phagemid library were harvested from the plates, and infected with helper phage GMCT for 1 hour. The Fab-display phages were then generated from those TG1 cells harboring both library phagemids and GMCT helper phage genome by overnight growth in 2×YT/carbenicillin/Kanamycin at 22° C. The phagemid particles were purified from overnight culture supernatants by precipitation with PEG/NaCl, and re-suspended in PBS. The PEG-precipitation was repeated once. The phage concentration was determined by OD₂₆₈ measurement.

With amplified first round phages, the panning process as described above was repeated twice for further enrichment of PCSK9-binding phages. The eluted phages from the third round panning were used to infect TG1 cells. The TG1 cells harboring phagemids from third round panning were picked from 2YT agar plates for Fab ELISA screening assay.

### Example 19

### Fab ELISA Screening for Binders

Over 10000 clones from third round panning were picked for each antigen by MegaPix Picking Robot (Genetix), and inoculated into 384-well plates with 60 ul of 2YT/2% Glucose/carbenicillin for overnight culture at 30° C. with 450 rpm shaking. The duplicated plates were made by transferring-1-3 ul overnight culture from each well into new plates with 50 ul/well of 2YT/0.1% Glucose/carbenicillin. The duplicated plates were incubated in a shaker at 30° C. for 6 hours, then 10 μl/well of IPTG was added for final concentration of 1 mM. After overnight culture at 22° C., the soluble Fab in IPTG-induction plates were released by adding lysozyme into each well.

To detect the antigen binding activity of soluble Fabs generated from above experiment, the antigen plates were generated by overnight coating of 5 μg/ml antigen. After blocking with milk-PBS and wash with PBST, 15-20 ul of Fab samples from IPTG-induction plates was transferred into antigen plates for 1-2 hours incubation at room temperature. The plates were washed 5 times with PBS-T, and added with 1:2000 diluted goat anti-human Kappa-HRP (SouthernBiotech Cat. No. 2060-05) or 1:10,000 diluted goat anti-human Fab-HRP in 5% MPBS for 1 hour incubation. After washing away unbound HRP-conjugates with PBST, the substrate solution QuantaBlu WS (Pierce 15169) was then added to each well and incubated for 5-15 mins. The relative fluorescence units (RFU) of each well was measured to determine the Fab binding activity by using excitation wavelength 330 nm and emission detection wavelength 410 nm.

### Example 20

### Isolation of Anti-Human PCSK9 Antibodies from PDL1 Library

The ELISA results showed 30 to 80% clones from third round panning of individual PDL1 sun-libraries bound to antigen PCSK9. The positive clones were then sent out for DNA sequencing. Total 128 unique Fab sequences were identified from those PDL1 library pannings.

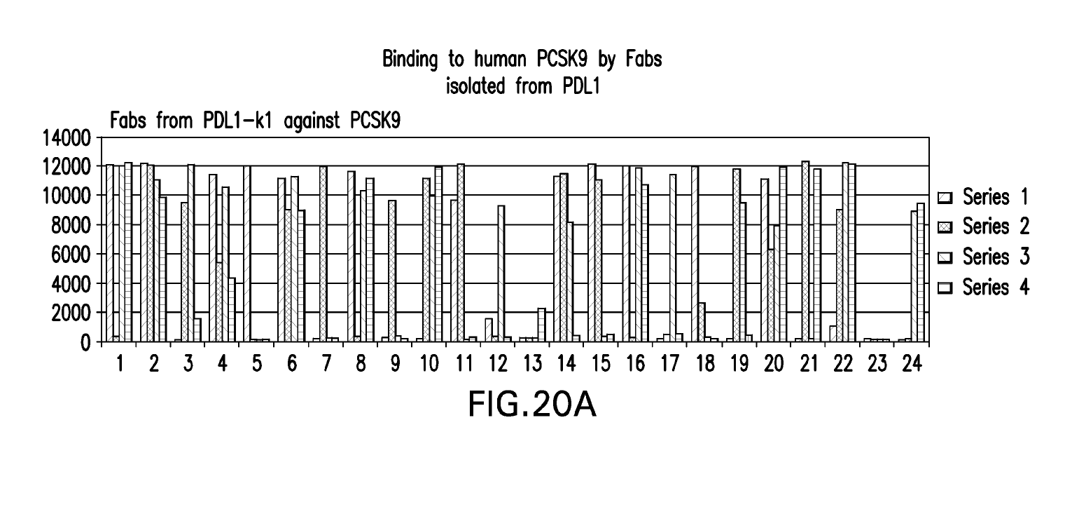

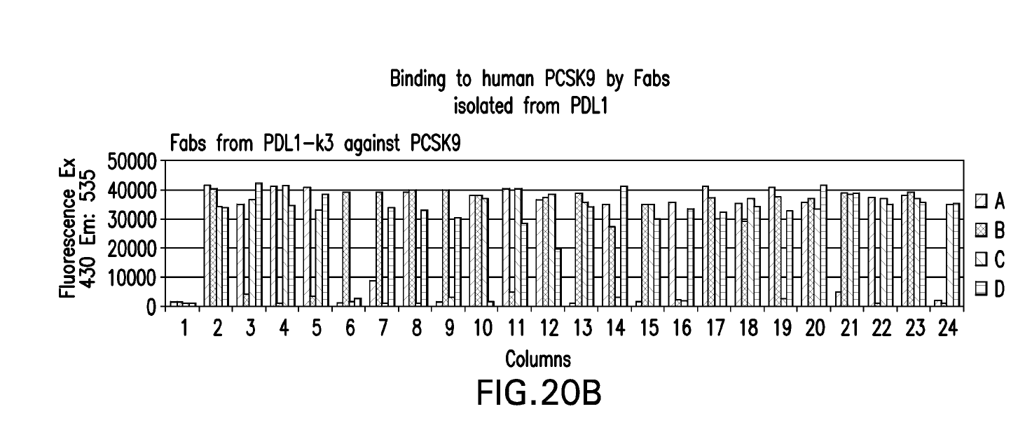

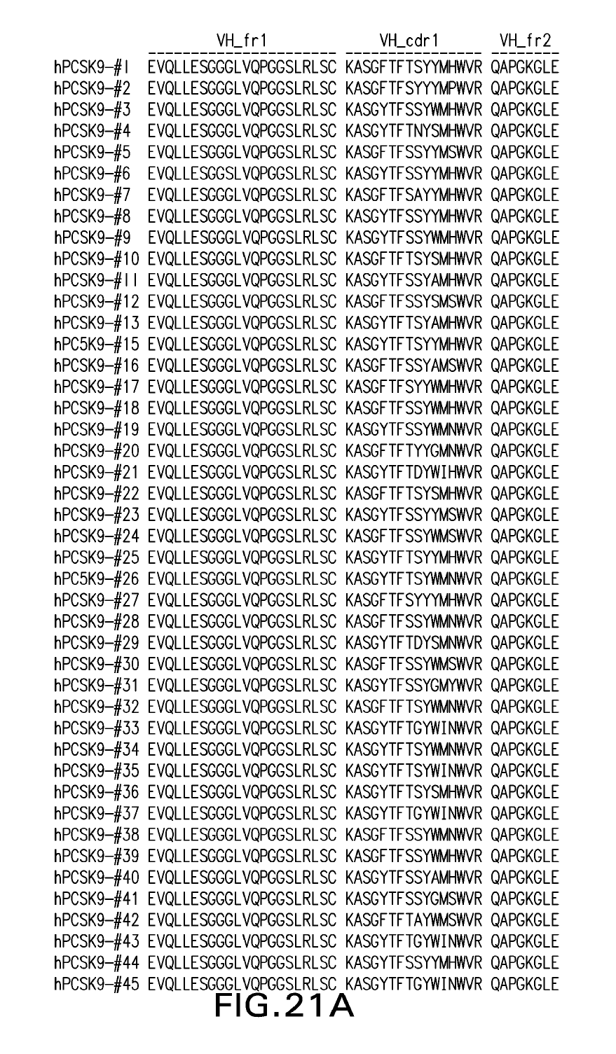

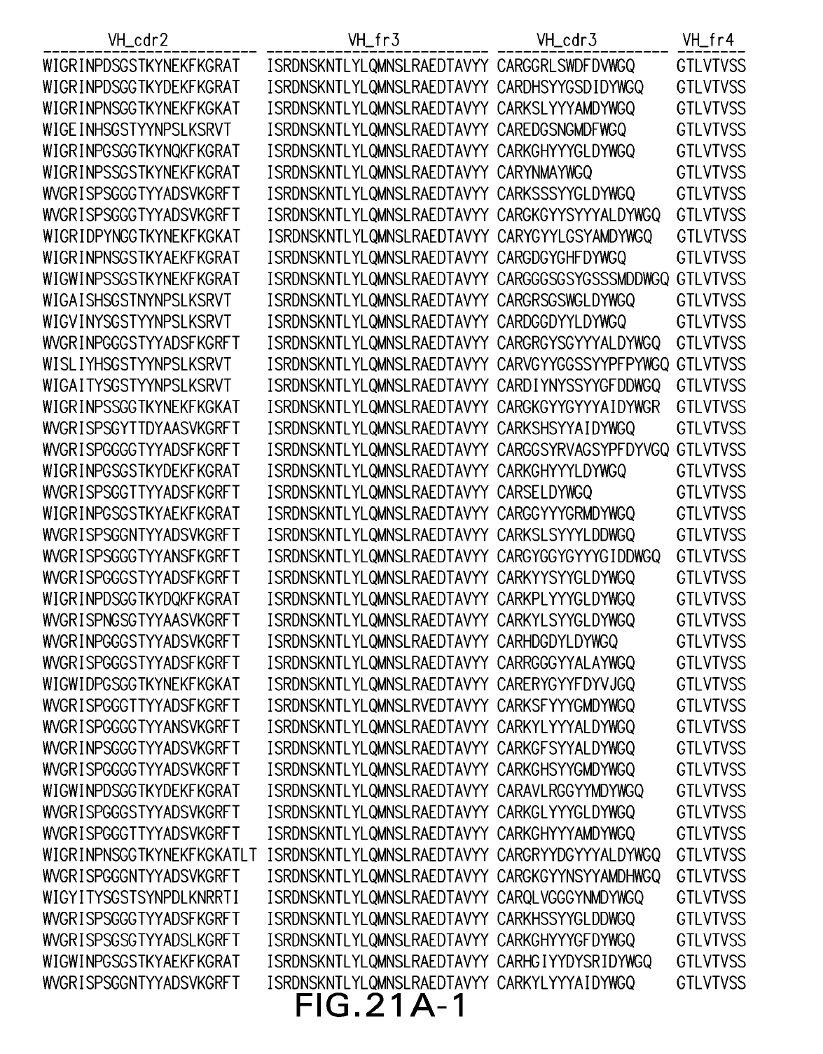

FIG. 20 shows the ELISA data that illustrates the binding of PDL1 Fabs to human PCSK9.

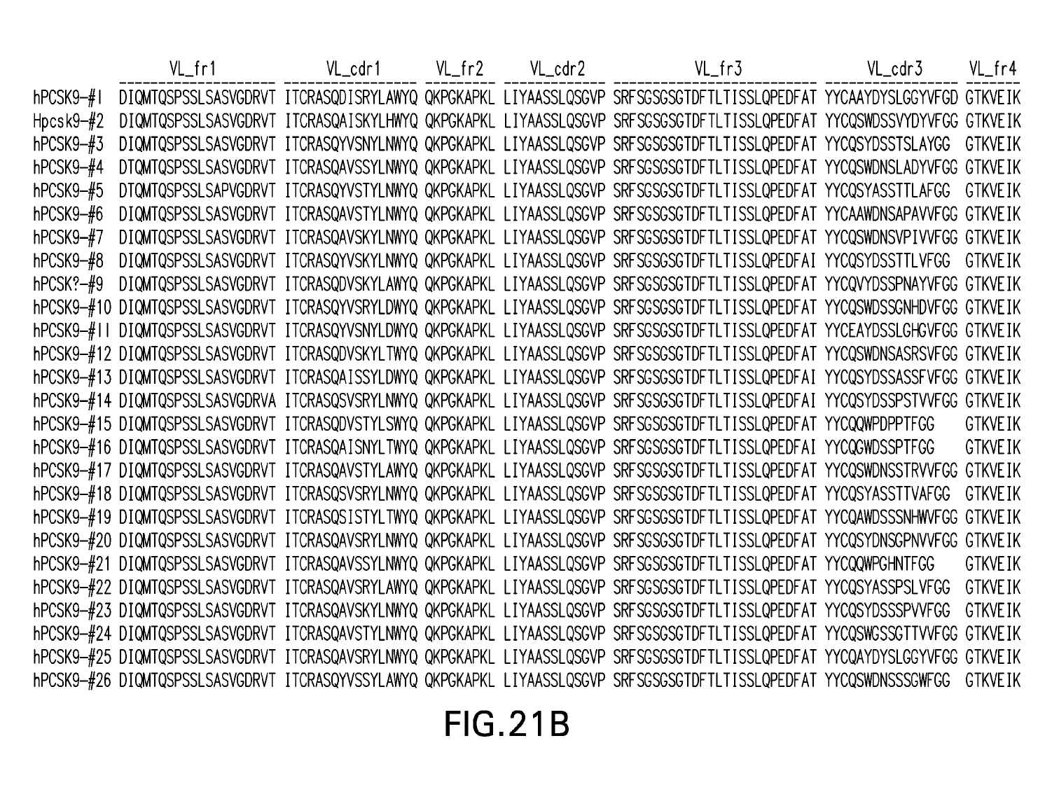

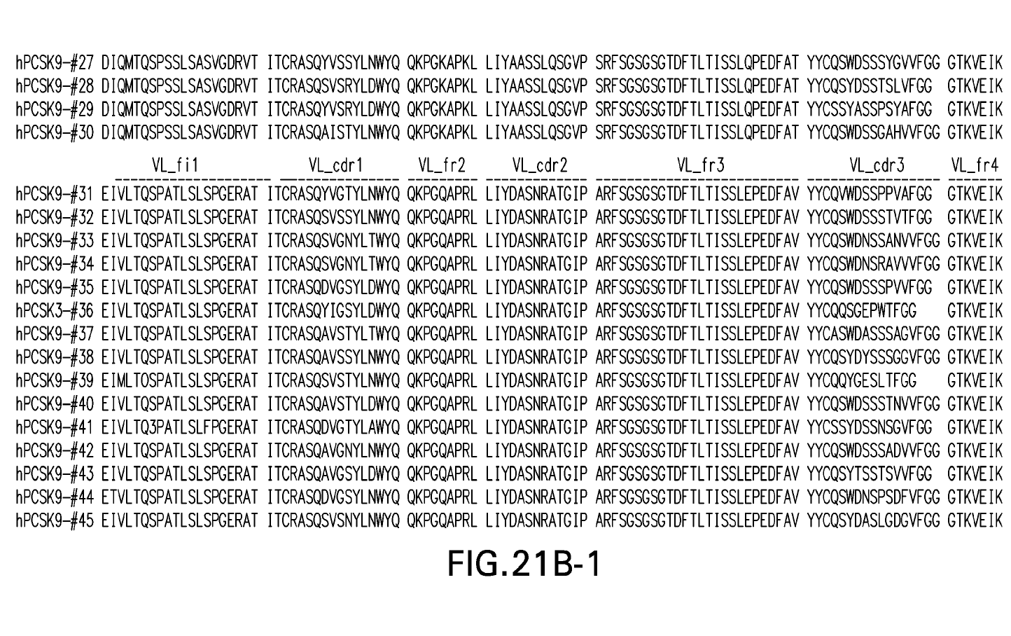

FIG. 21A shows the sequences of VH from a panel of anti-human PCSK9 antibodies isolated from PDL1 library panning.

FIG. 21B shows the sequences of VK from a panel of anti-human PCSK9 antibodies isolated from PDL1 library panning.

### Example 21

### Isolation of Anti-Mouse PCSK9 Antibodies from PDL1 Library

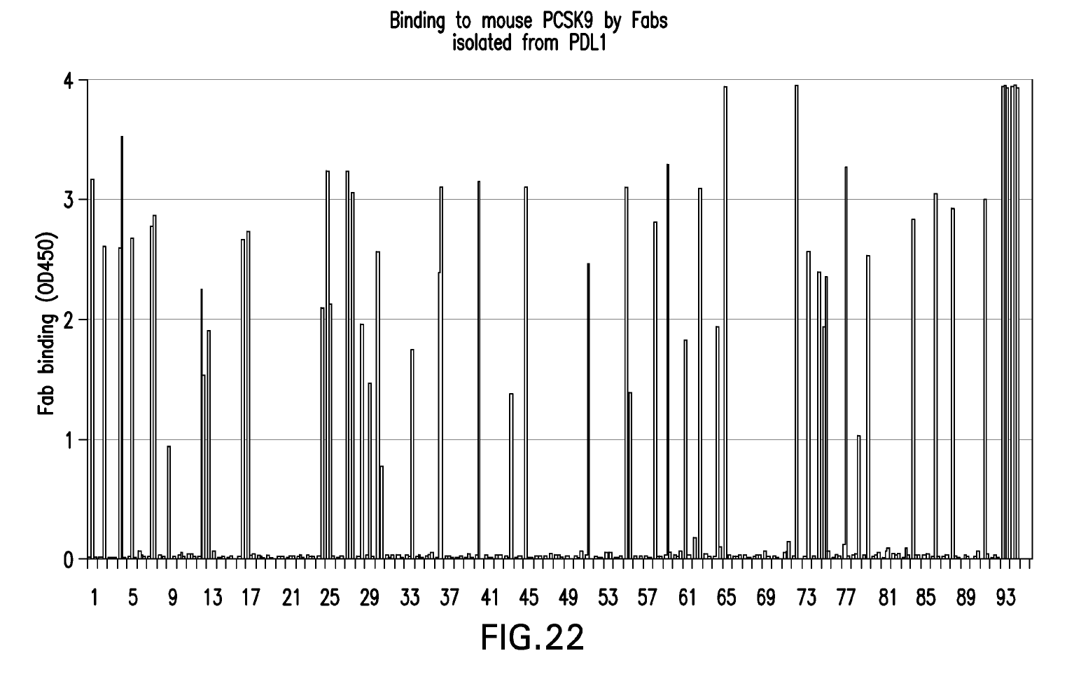

FIG. 22 shows the ELISA data that illustrated the binding of PDL1-Fabs to mouse PCSK9.

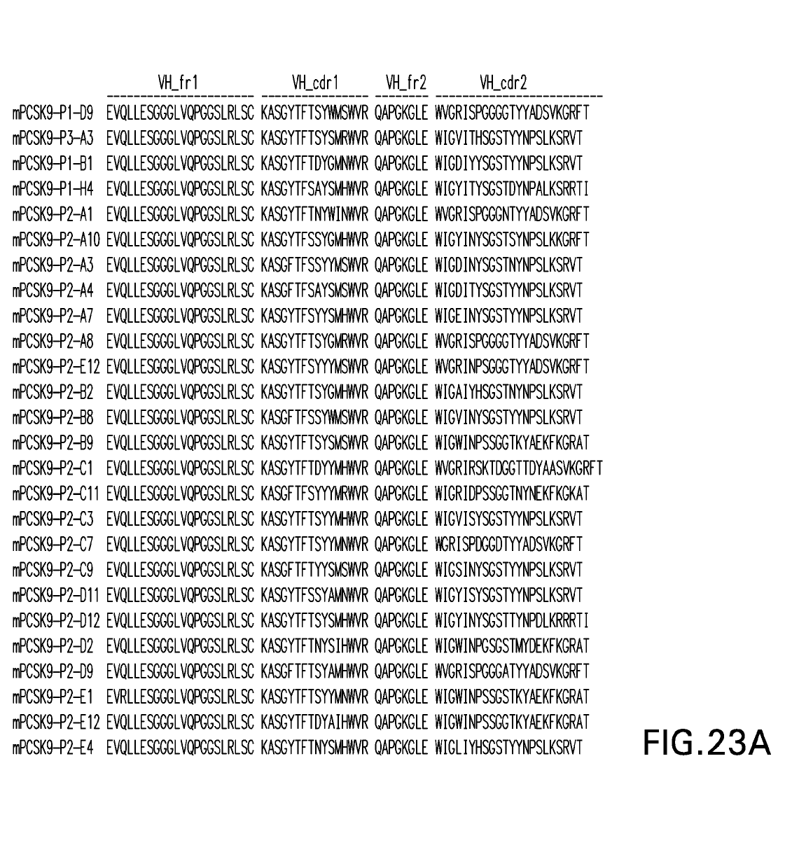

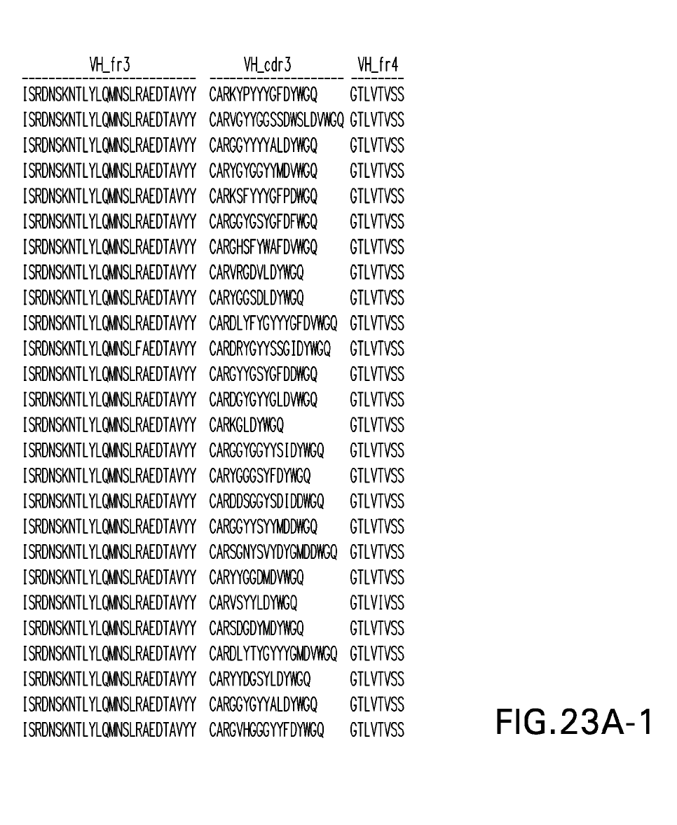

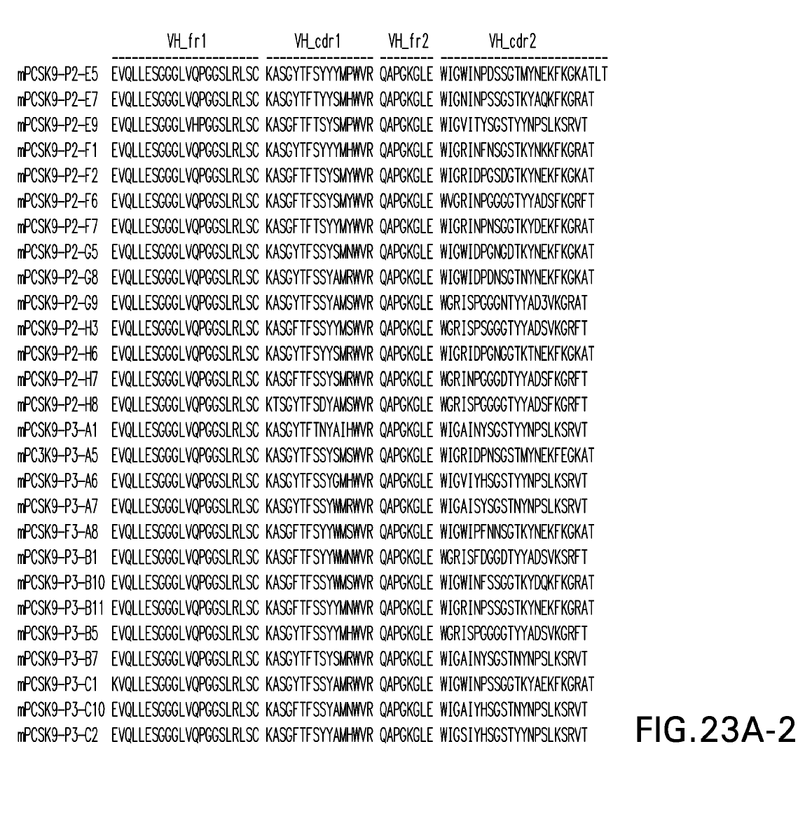

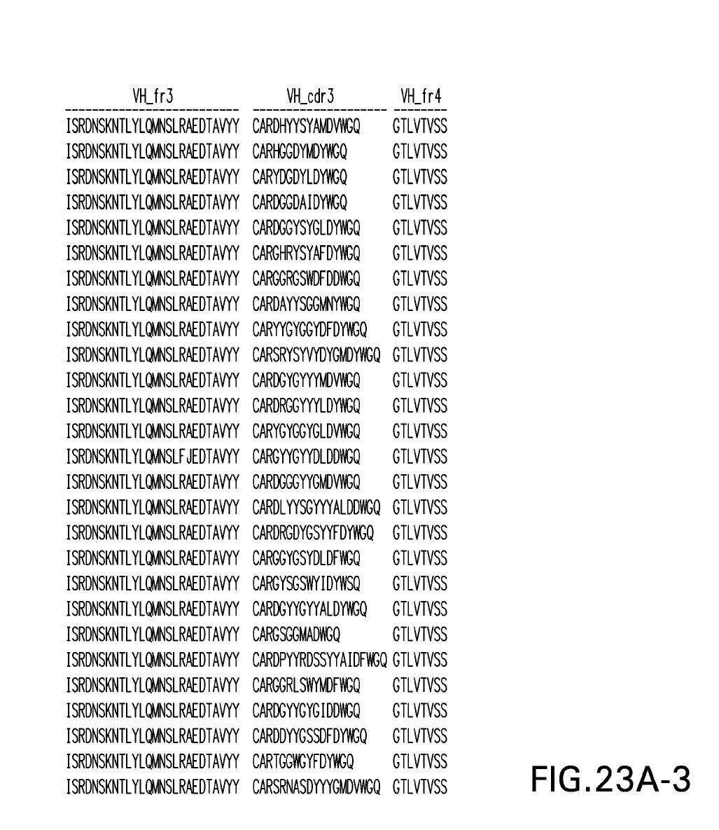

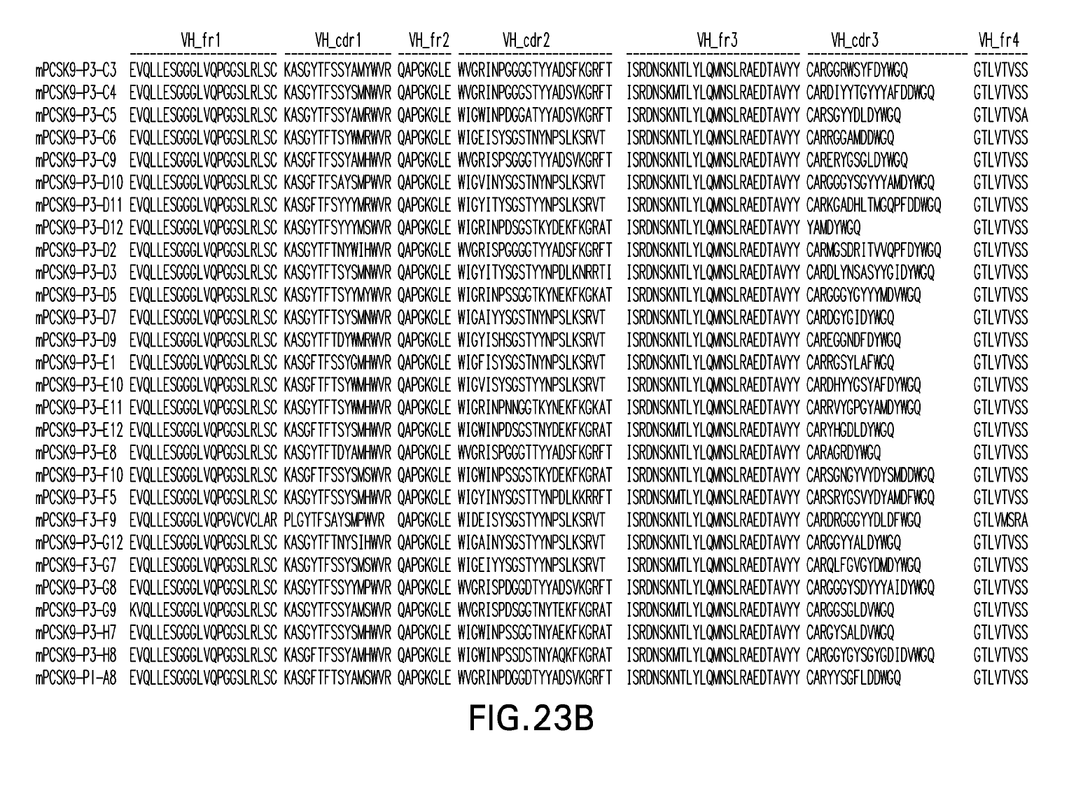

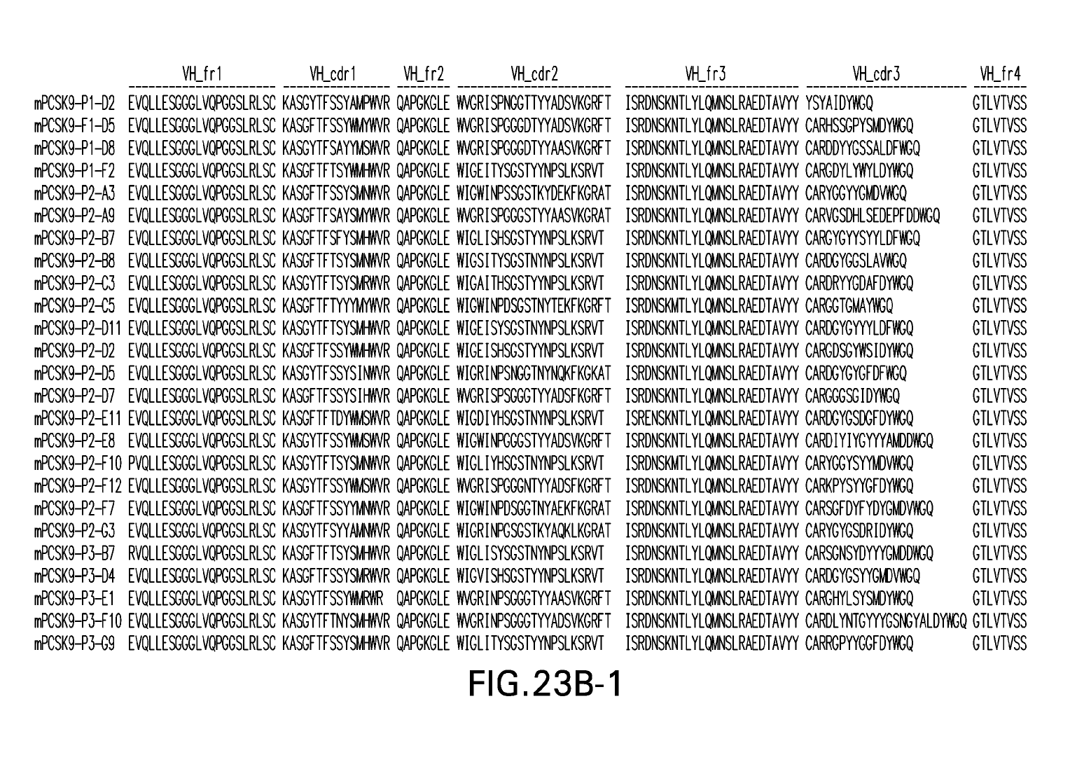

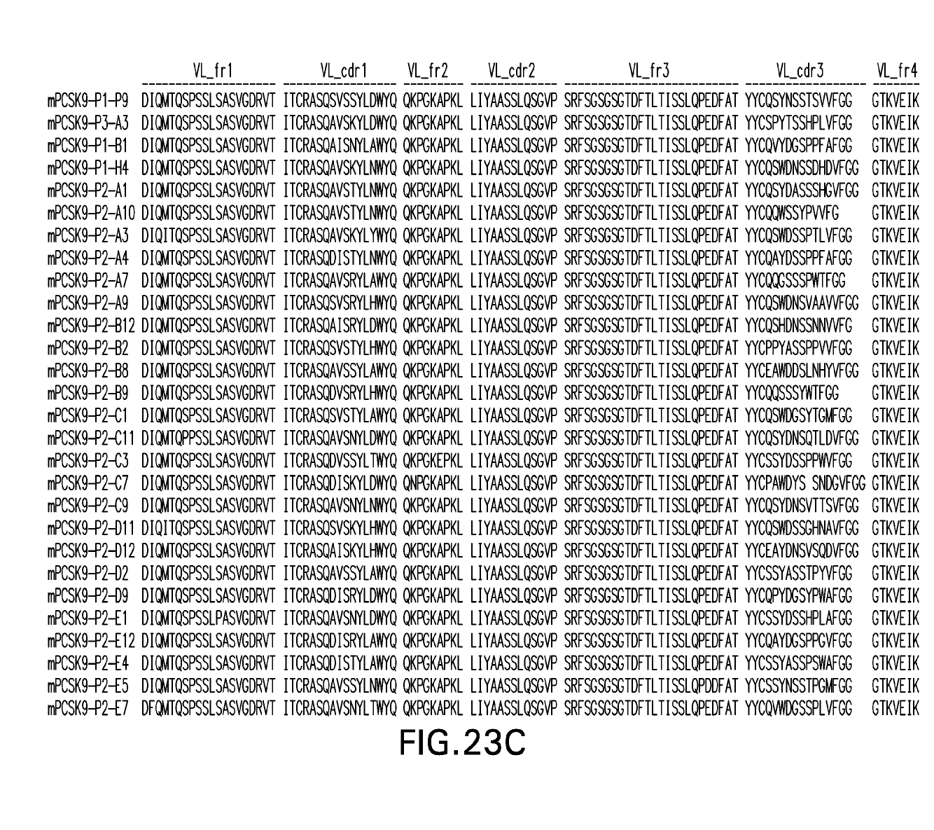

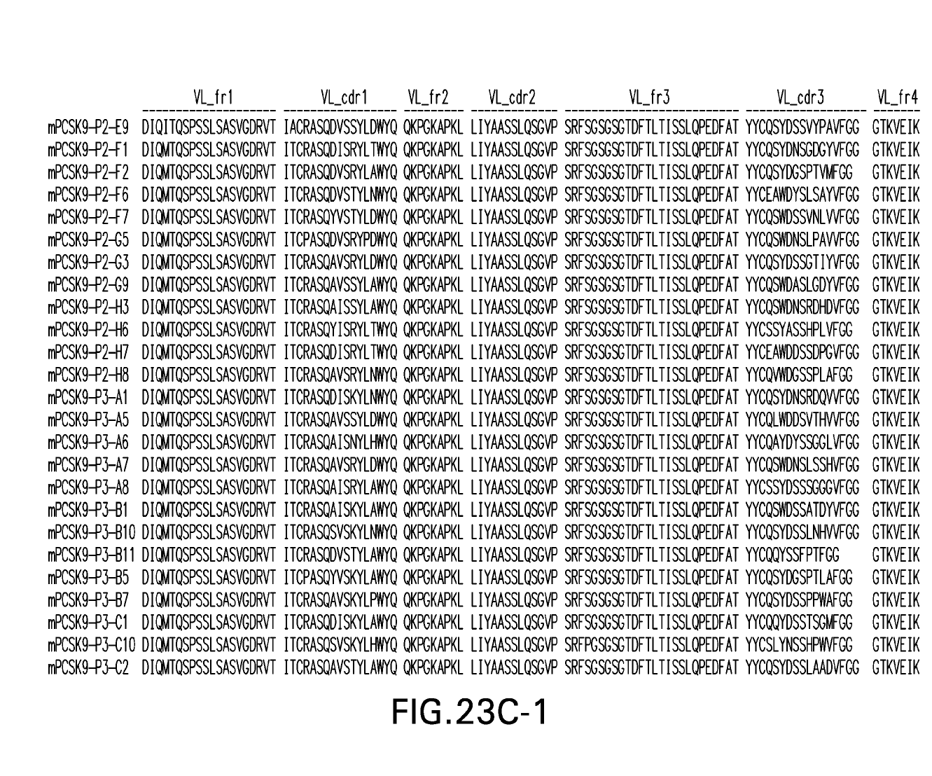

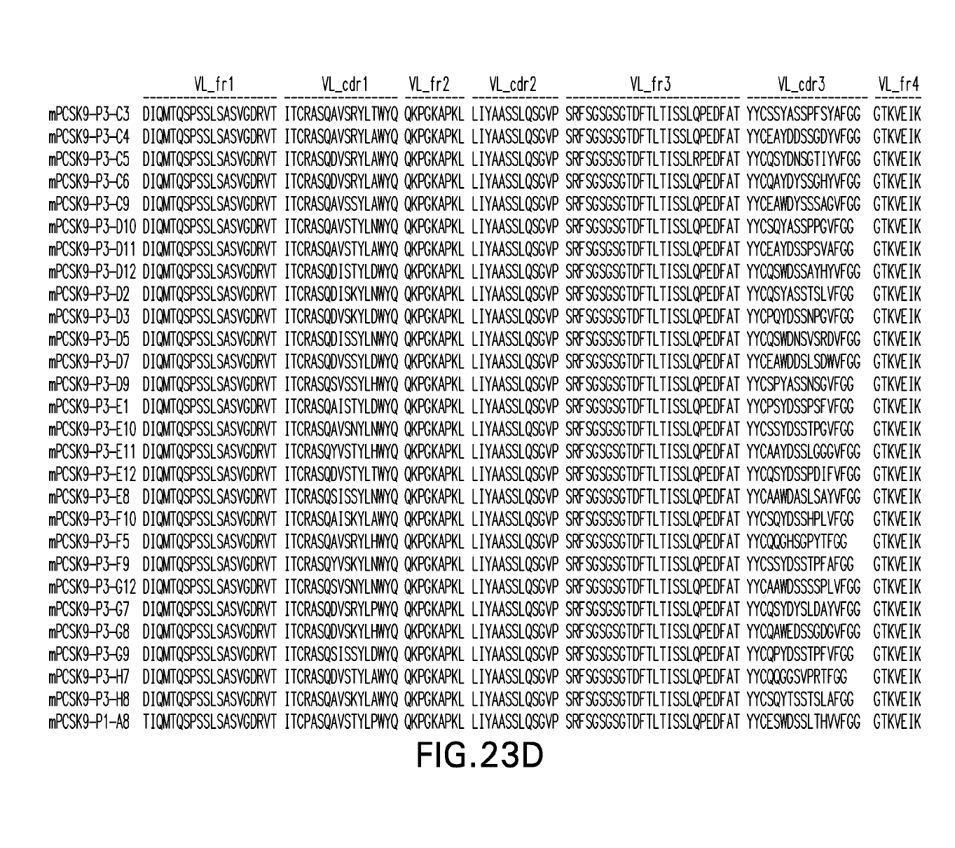

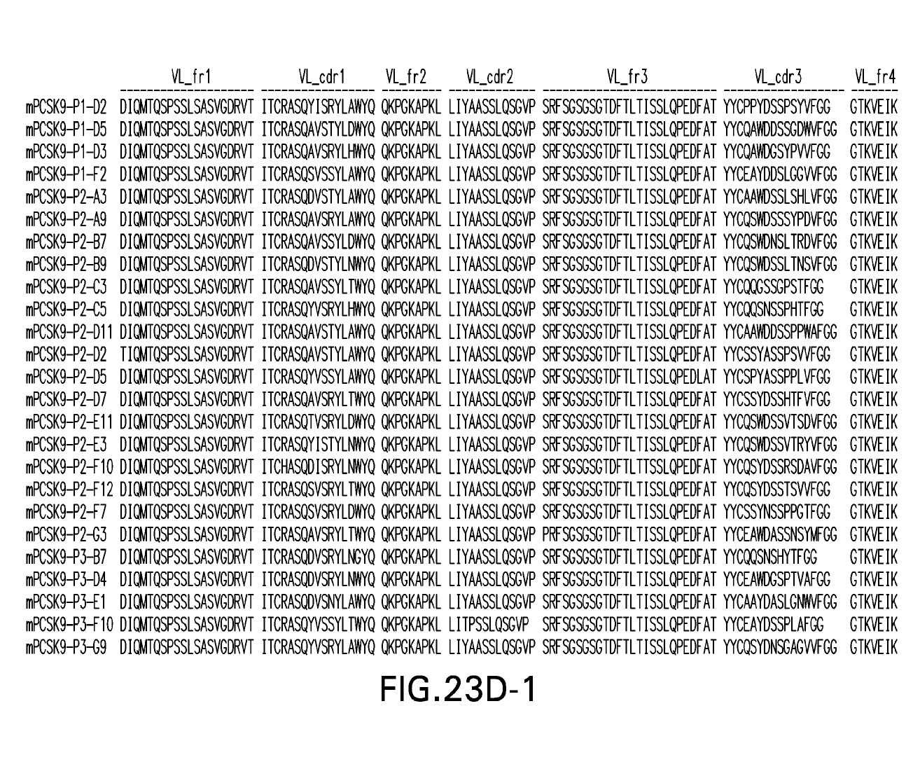

FIGS. 23A and 23B show the sequences of VH from a panel of anti-mouse PCSK9 antibodies isolated from PDL1 library panning.

FIGS. 23C and 23D show the sequences of VK from a panel of anti-mouse PCSK9 antibodies isolated from PDL1 library panning.

### Example 22

### Isolation of Anti-Human her3 Antibodies from PDL1 Library

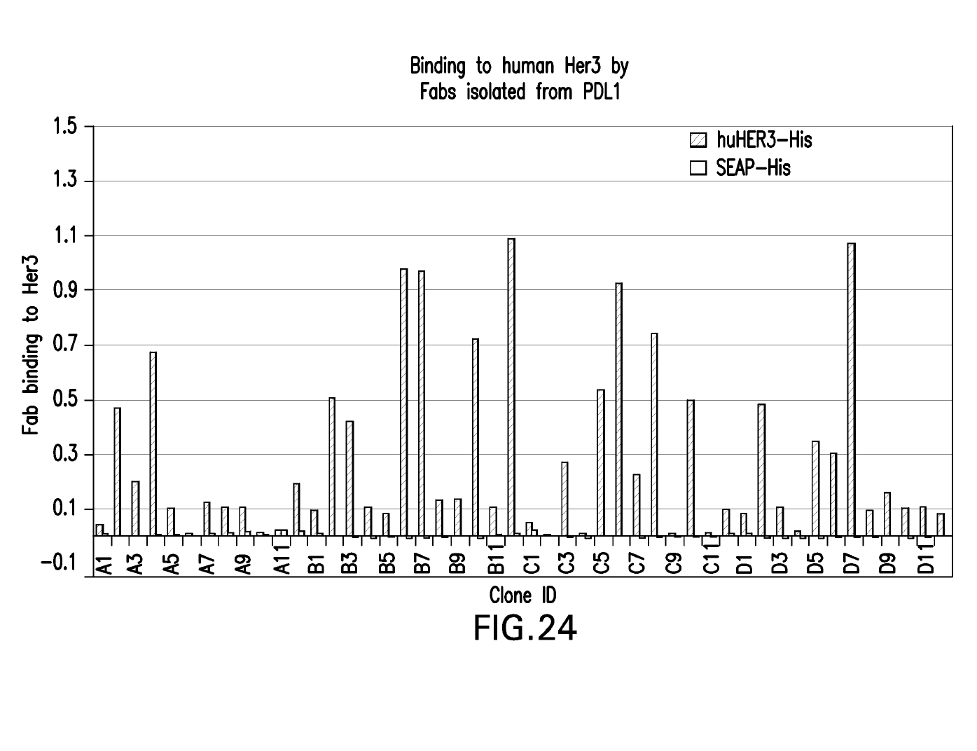

FIG. 24 shows the ELISA data that illustrated the binding of PDL1-Fabs to human Her3 protein.

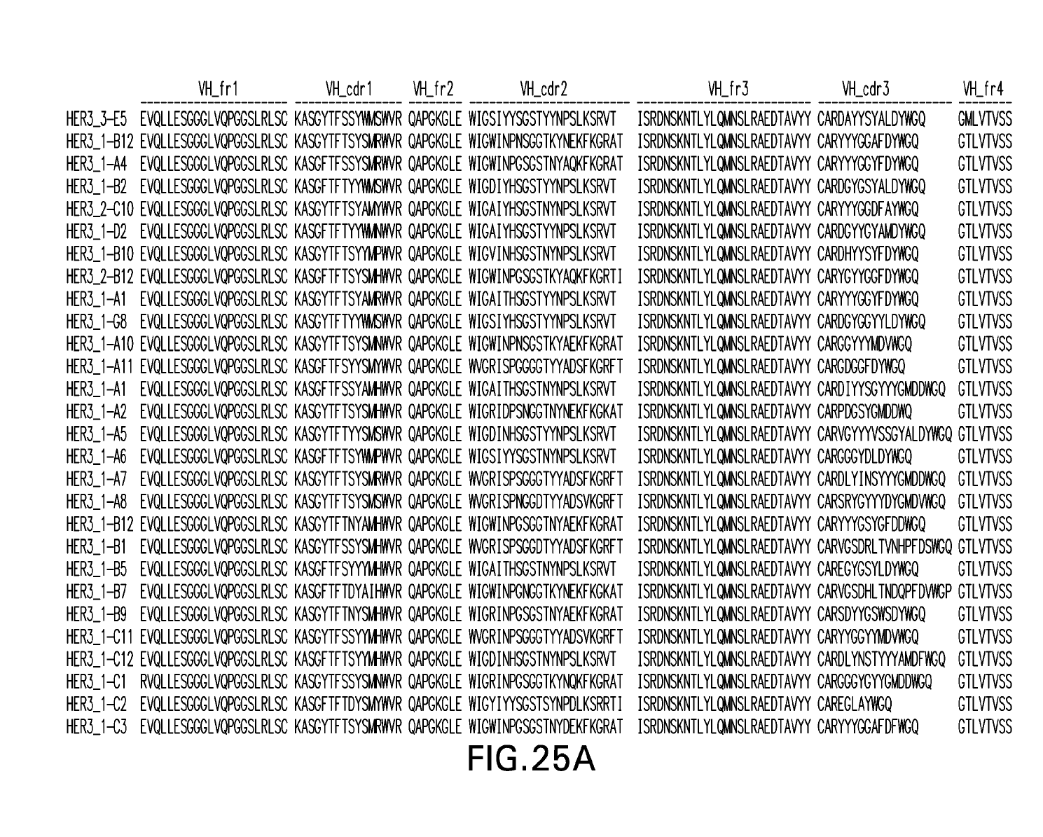

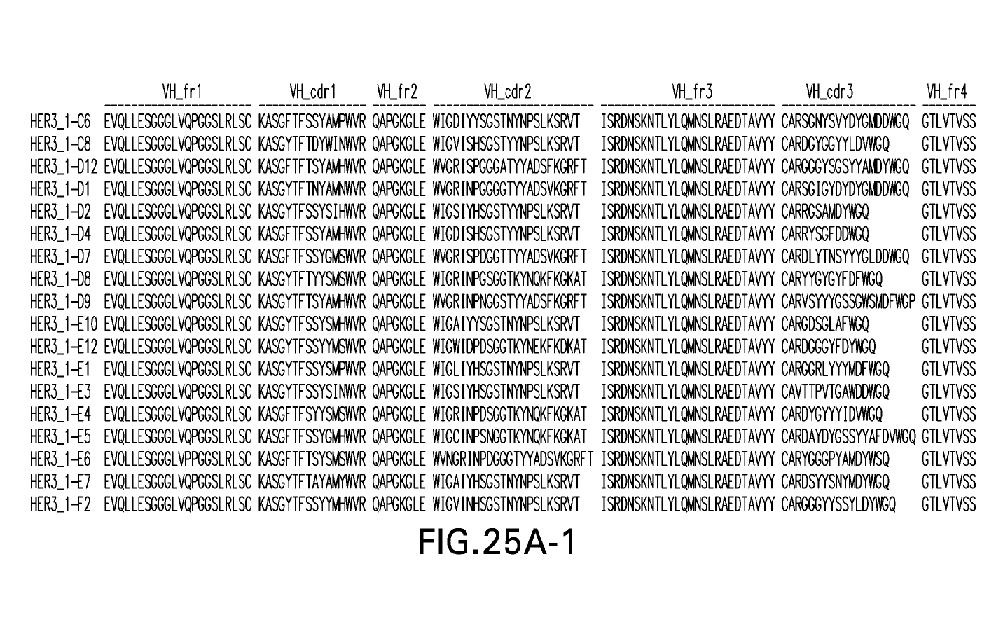

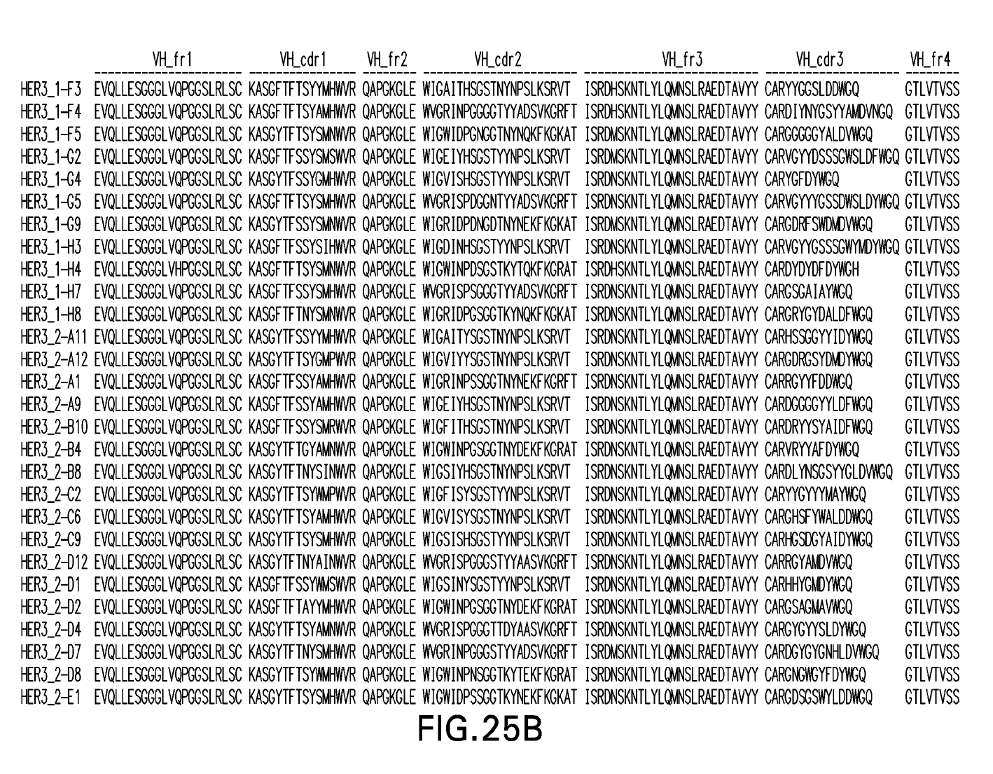

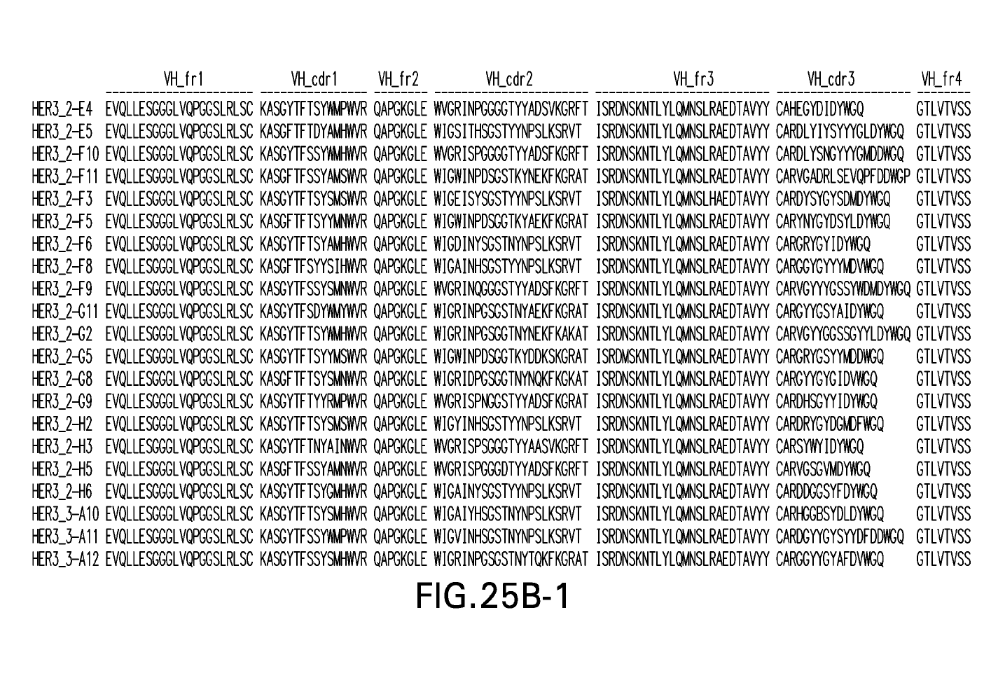

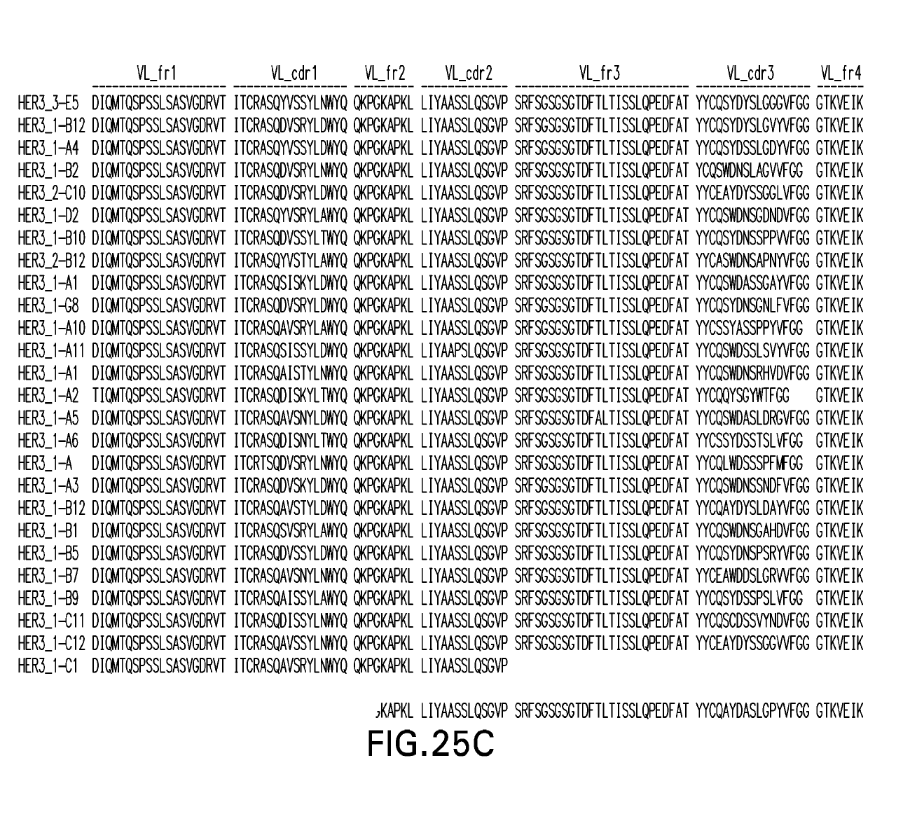

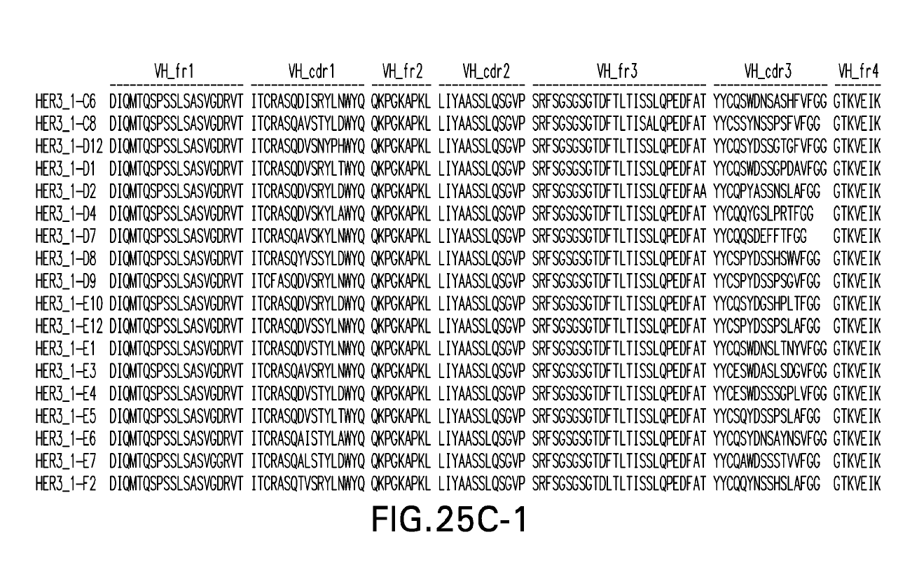

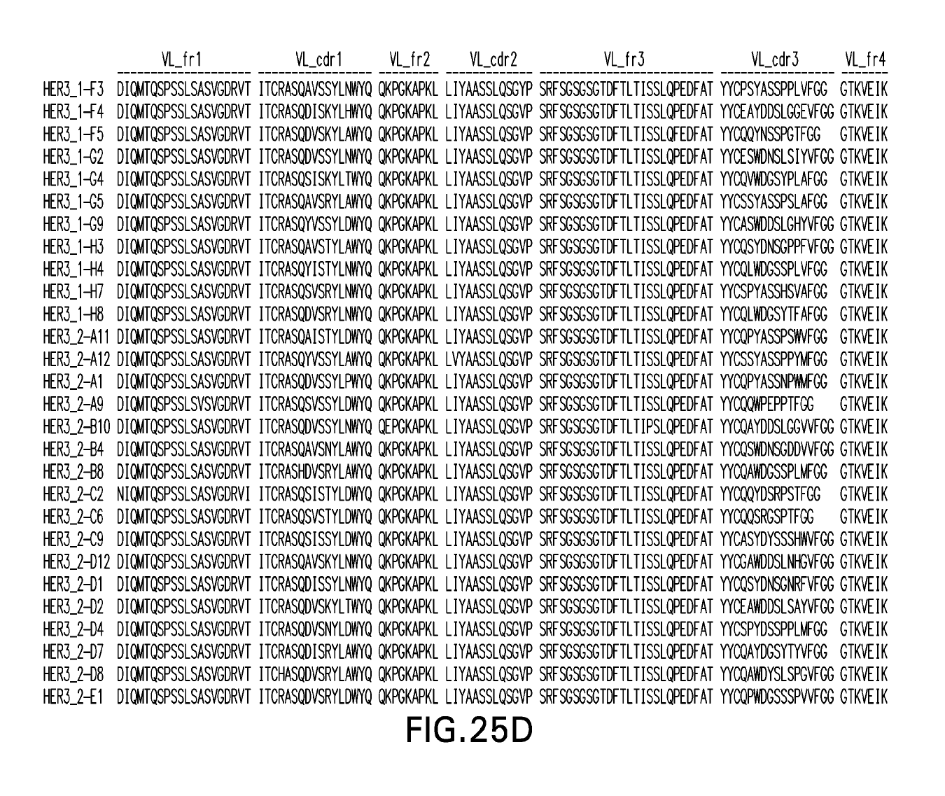

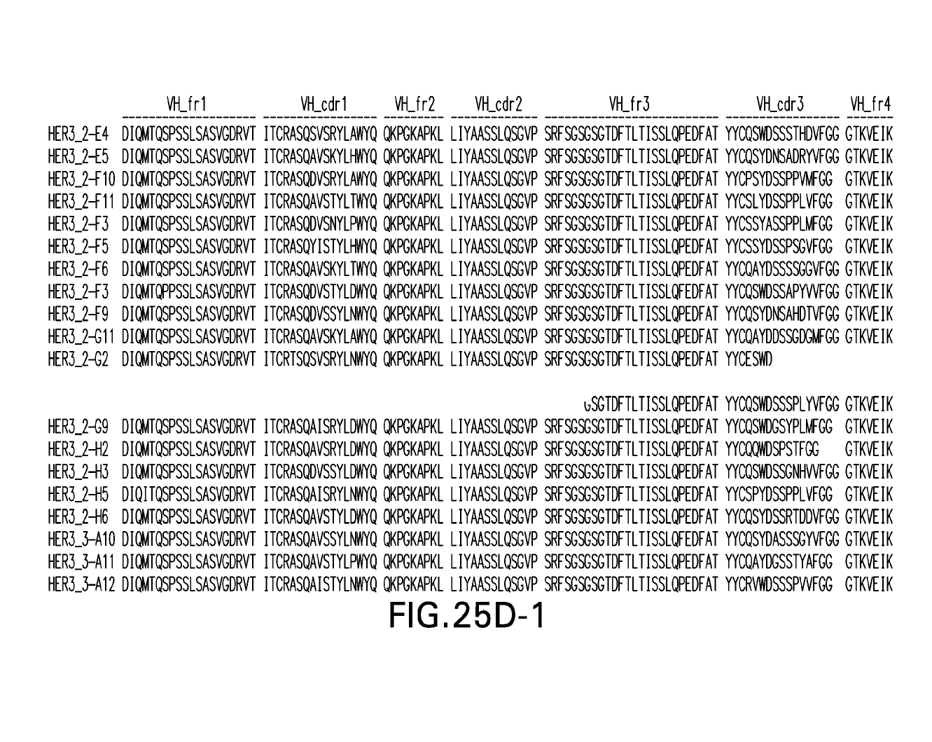

FIGS. 25A and 25B show the sequences of VH from a panel of anti-human Her3 antibodies isolated from PDL1 library panning.

FIGS. 25C and 25D show the sequences of VK from a panel of anti-human Her3 antibodies isolated from PDL1 library panning.

### Example 23

### Isolation of Anti-Mouse IL13R-Fc Antibodies from PDL1 Library

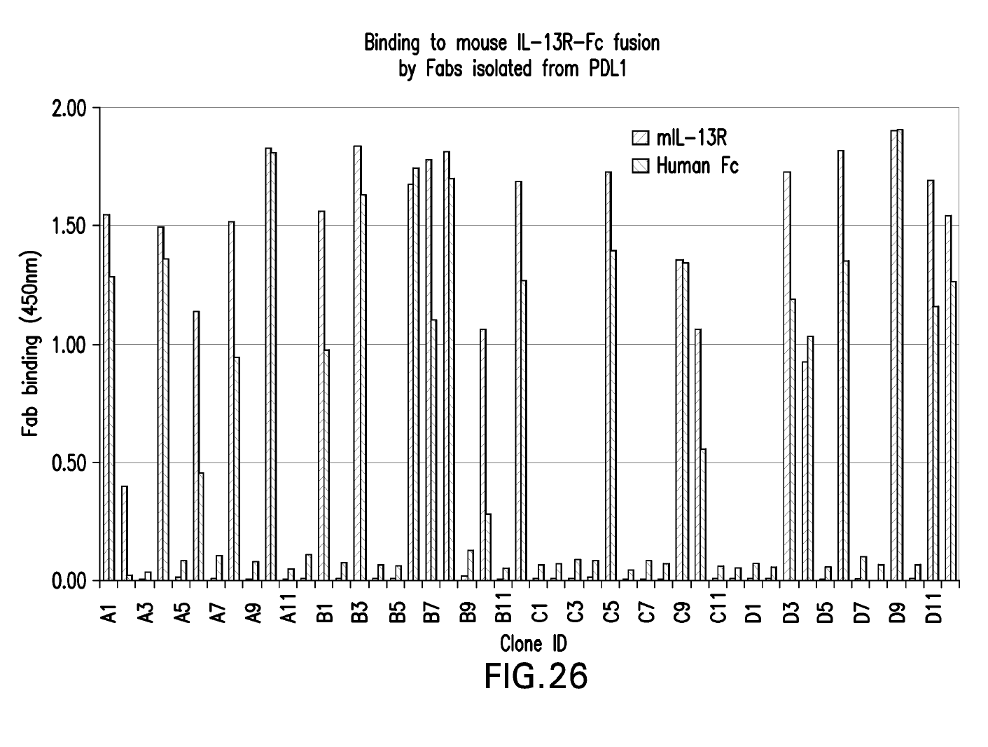

FIG. 26 shows the ELISA data that illustrated the binding of PDL1-Fabs to mouse IL13R-Fc protein.

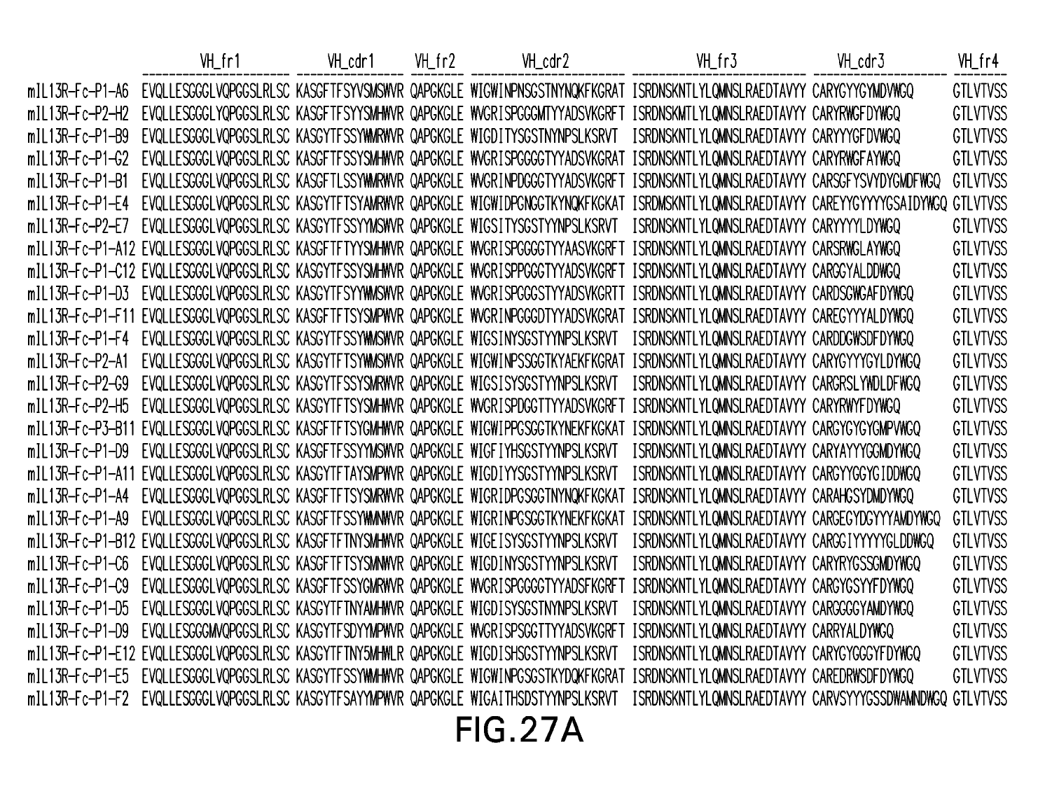

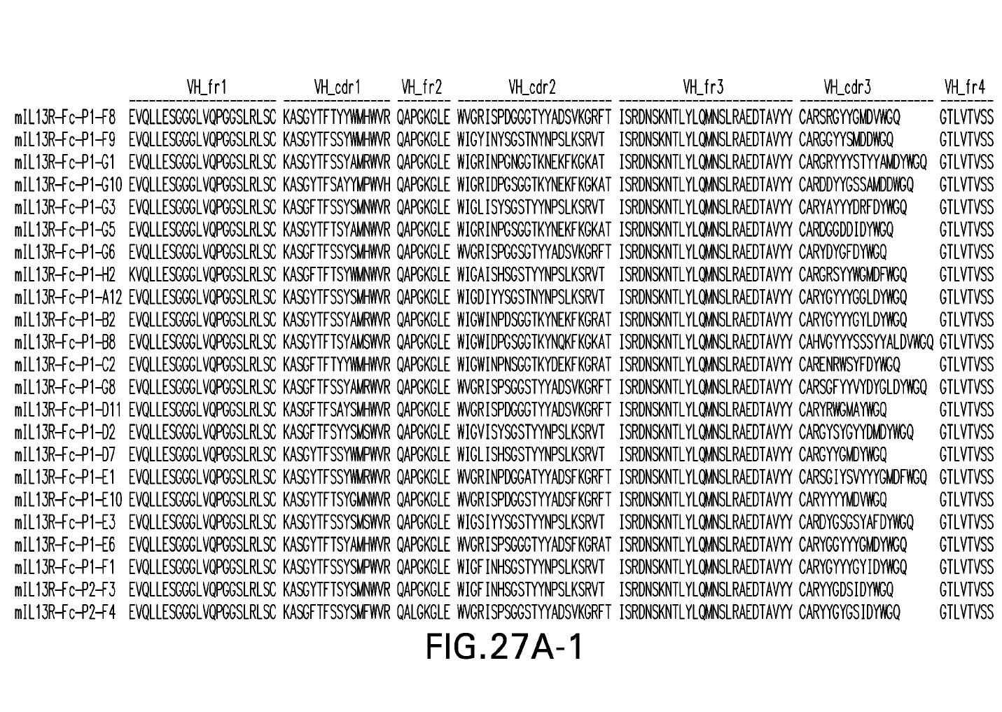

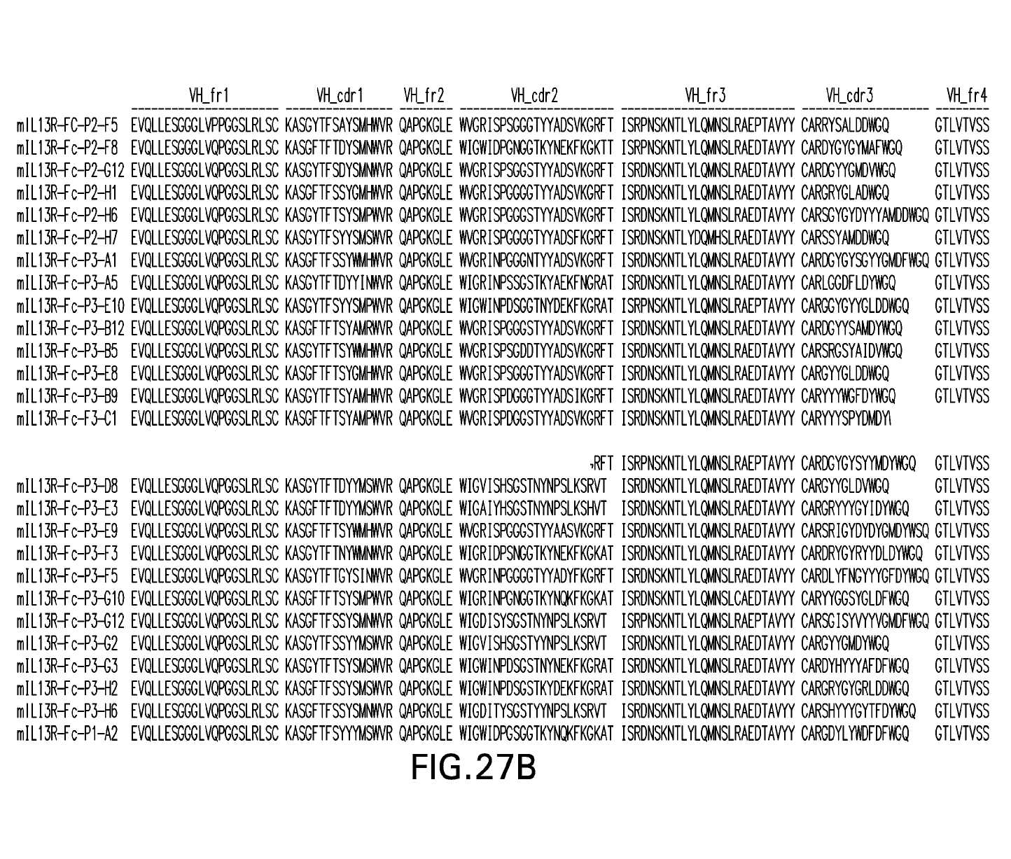

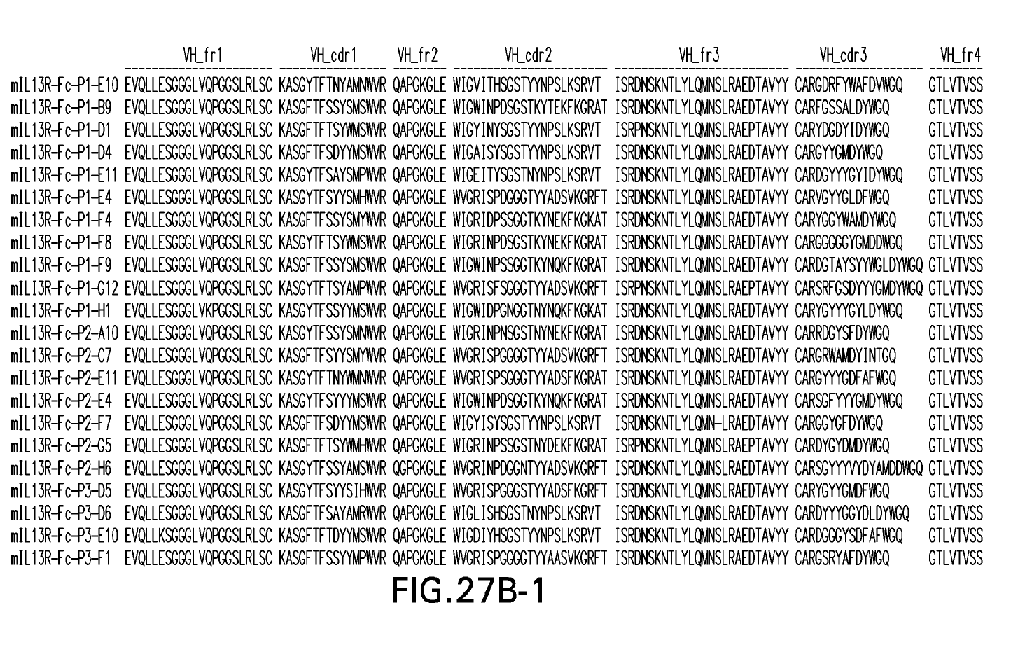

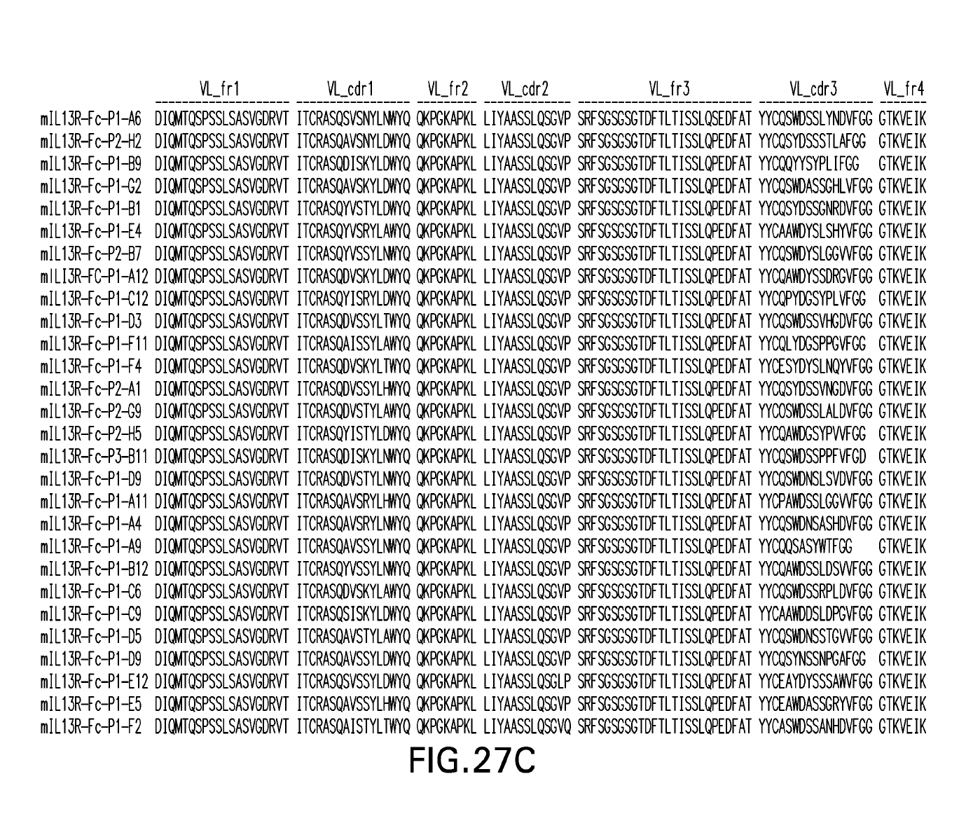

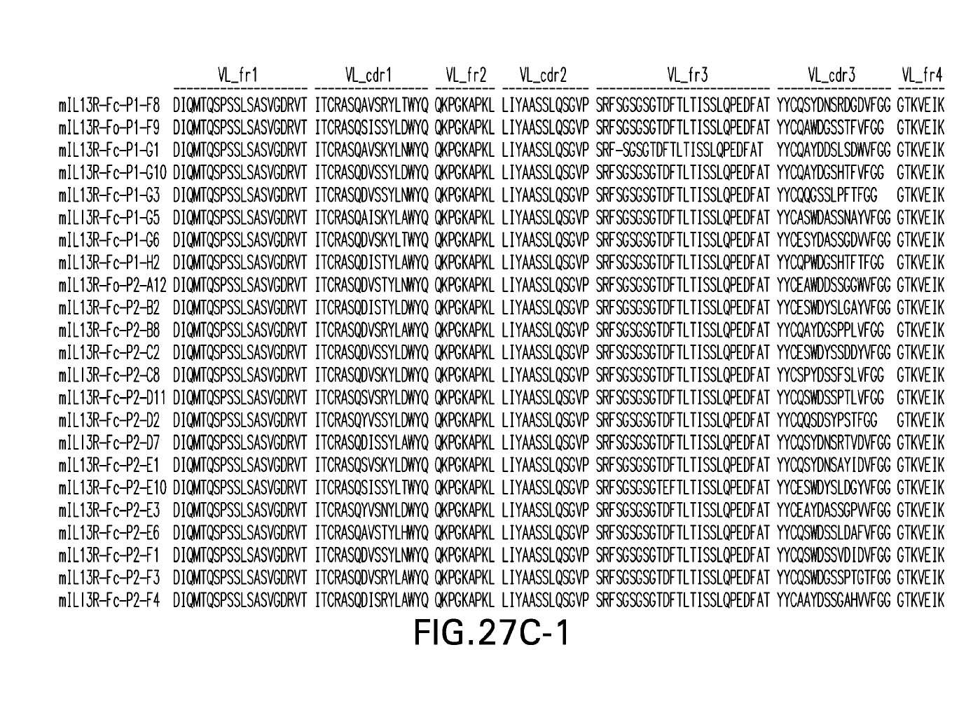

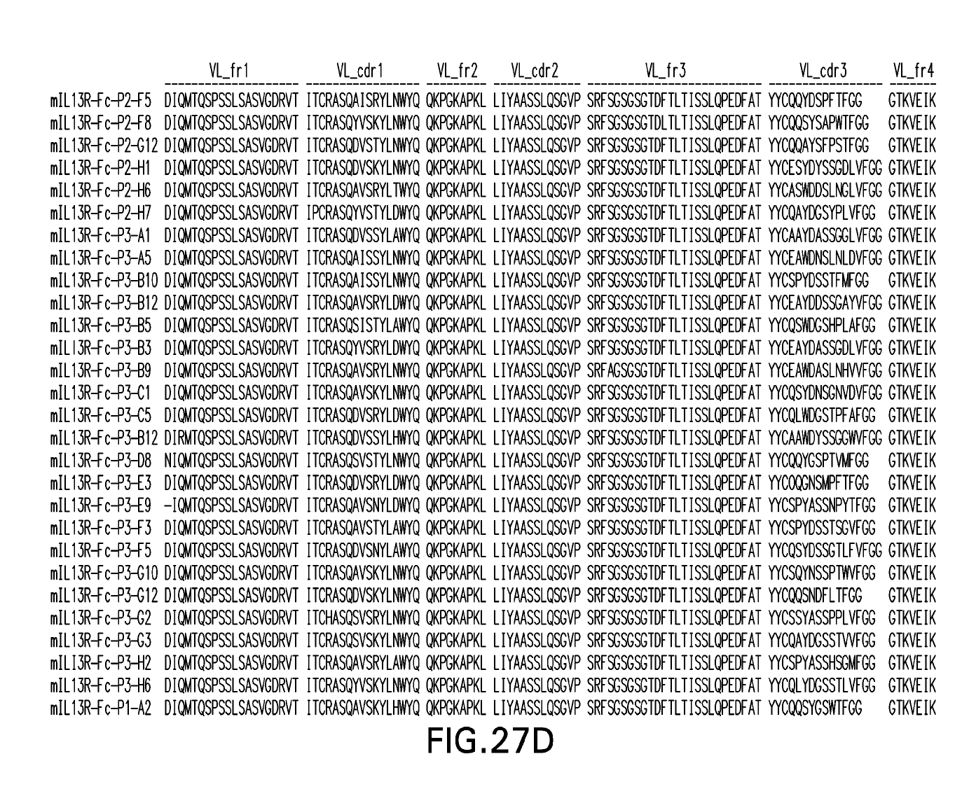

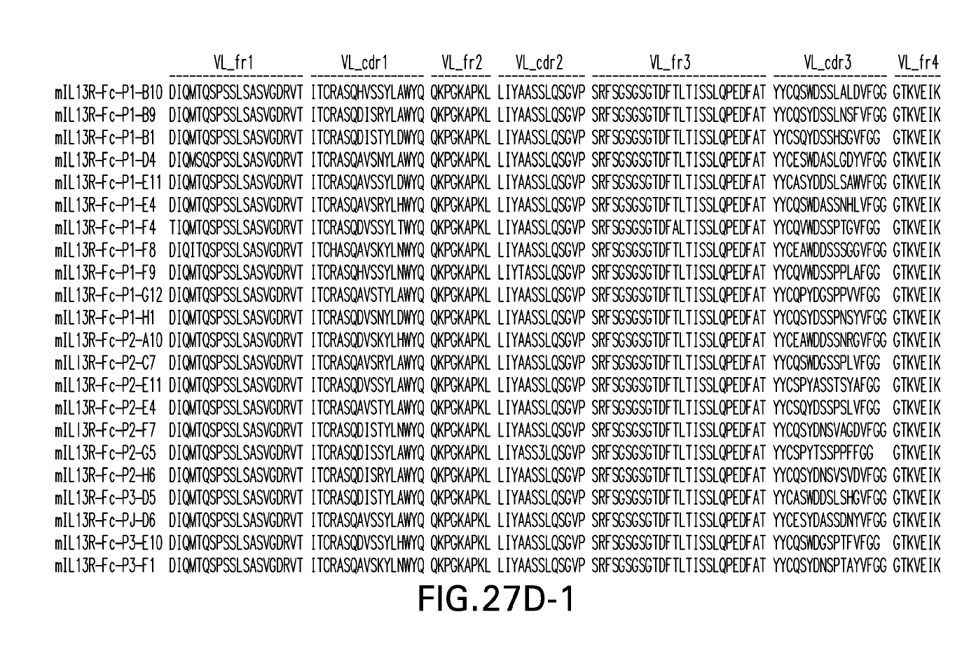

FIGS. 27A and 27B show the sequences of VH from a panel of anti-mouse IL13R-Fc antibodies isolated from PDL1 library panning.

FIGS. 27C and 27D show the sequences of VK from a panel of anti-mouse IL13R-Fc antibodies isolated from PDL1 library panning.

### Example 24

### Isolation of Anti-Mouse EPHA2 Antibodies from PDL1 Library

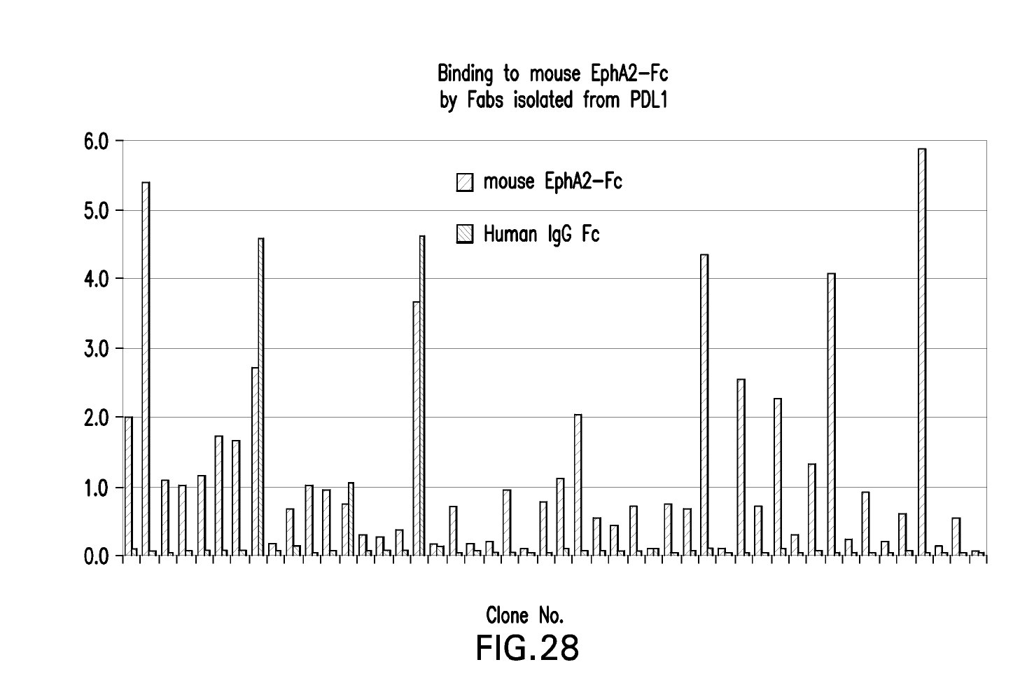

FIG. 28 shows the ELISA data that illustrated the binding of PDL1-Fabs to mouse EPHA2 protein.

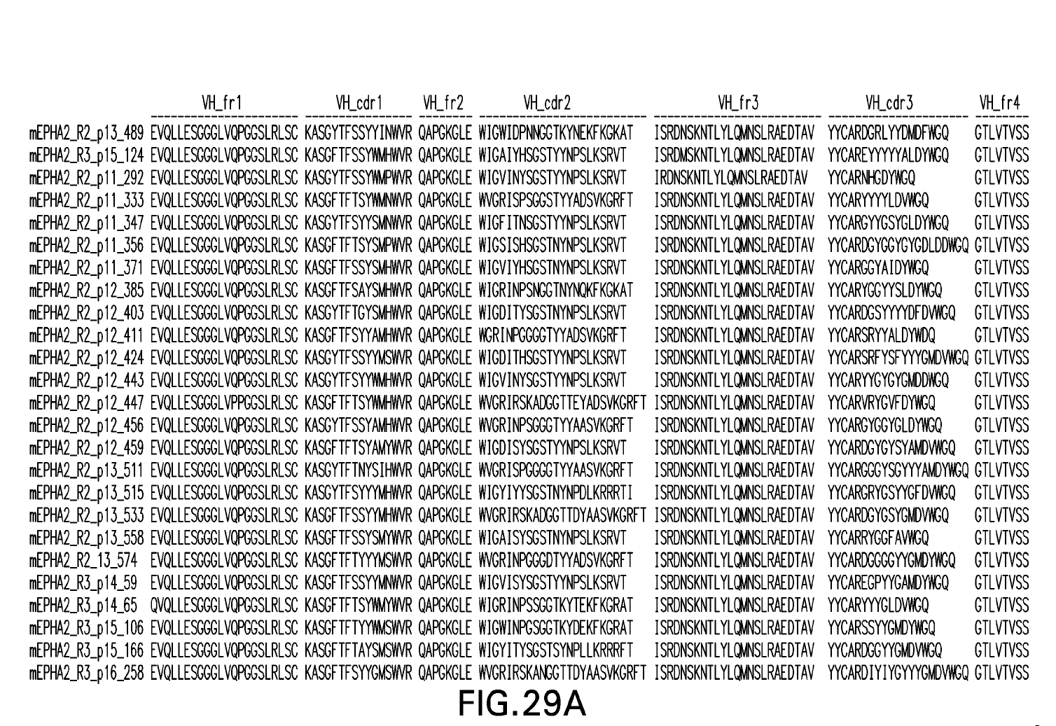

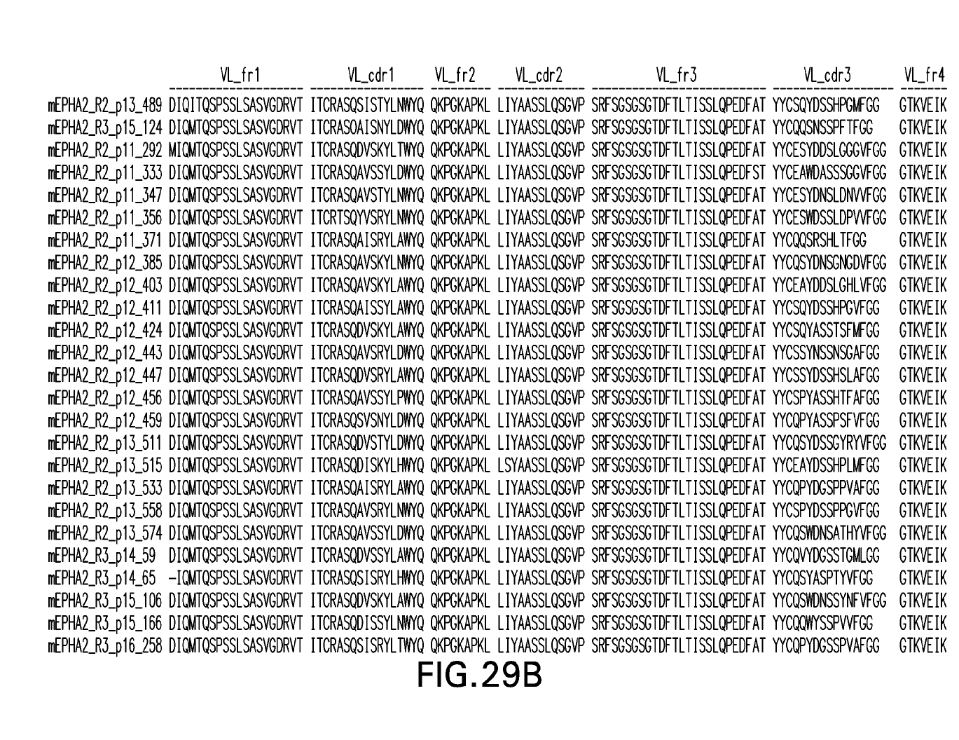

FIG. 29A shows the sequences of VH from a panel of anti-mouse EPHA2 antibodies isolated from PDL1 library panning.

FIG. 29B shows the sequences of VK from a panel of anti-mouse EPHA2 antibodies isolated from PDL1 library panning.

### Example 25

### Isolation of Anti-HSV Viral Protein gE Antibodies from PDL1 Library

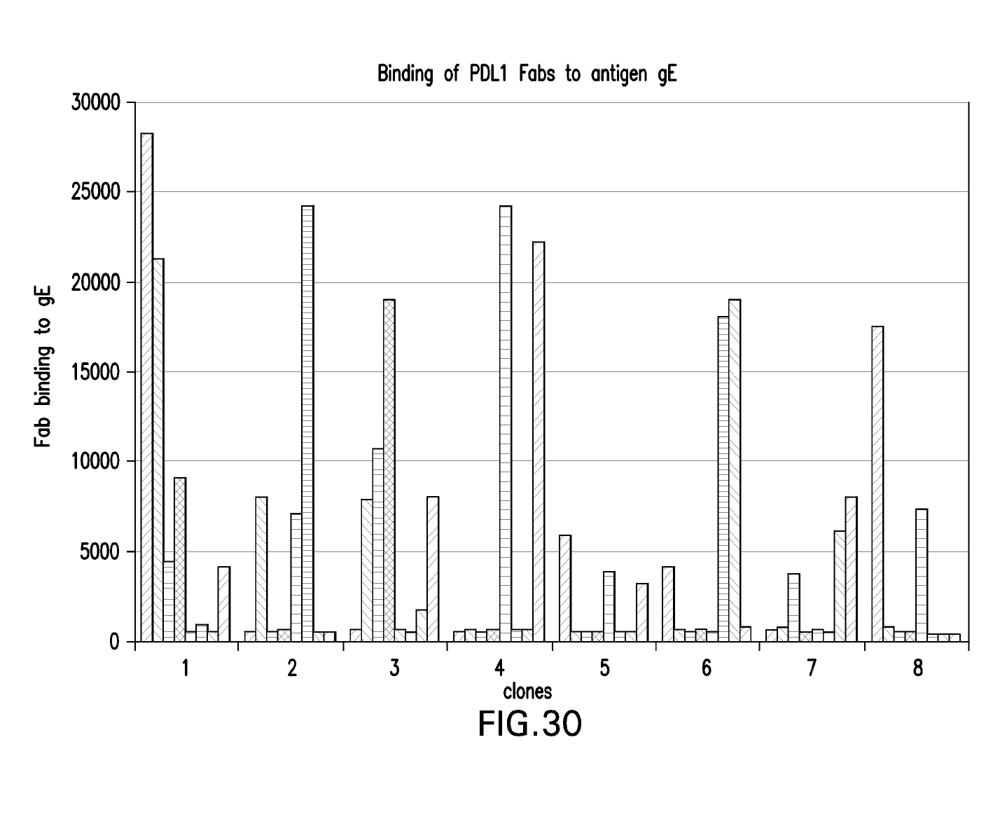

FIG. 30 shows the ELISA data that illustrated the binding of PDL1-Fabs to HSV viral protein gE protein.

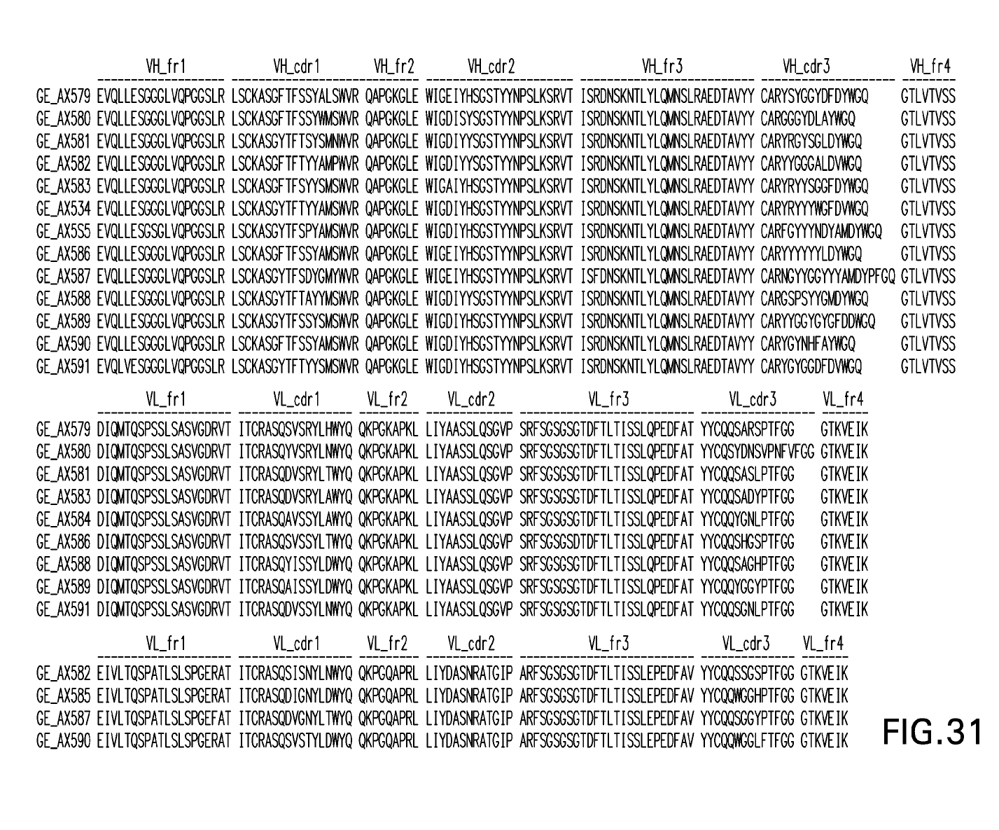

FIG. 31 shows the sequences of VH and Vk from a panel of anti-HSV viral protein gE antibodies isolated from PDL1 library panning.

### Example 26

### Isolation of Anti-Human TrkA Antibodies from PDL1 Library

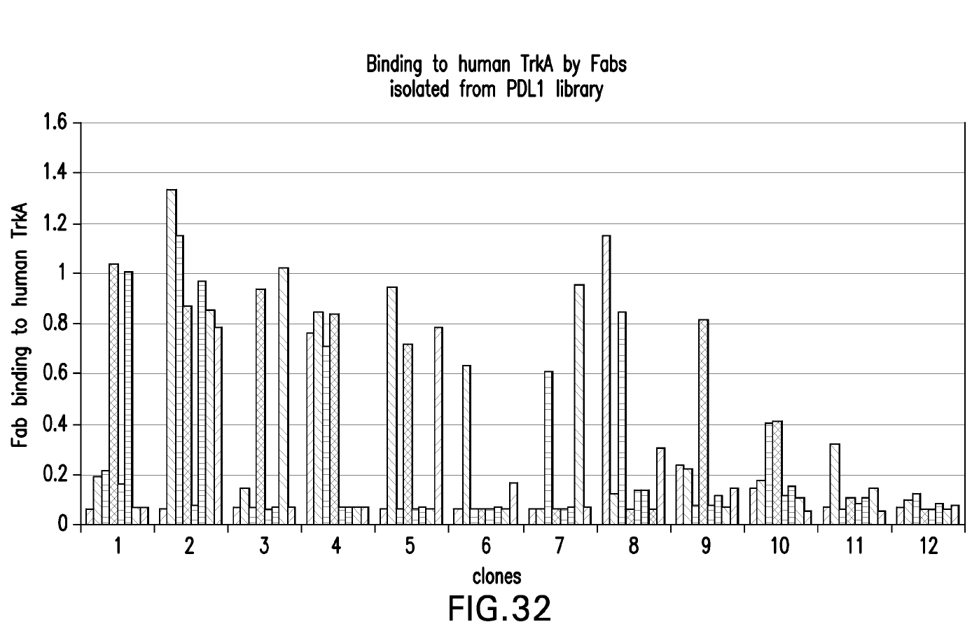

FIG. 32 shows the ELISA data that illustrated the binding of PDL1-Fabs to TrkA protein.

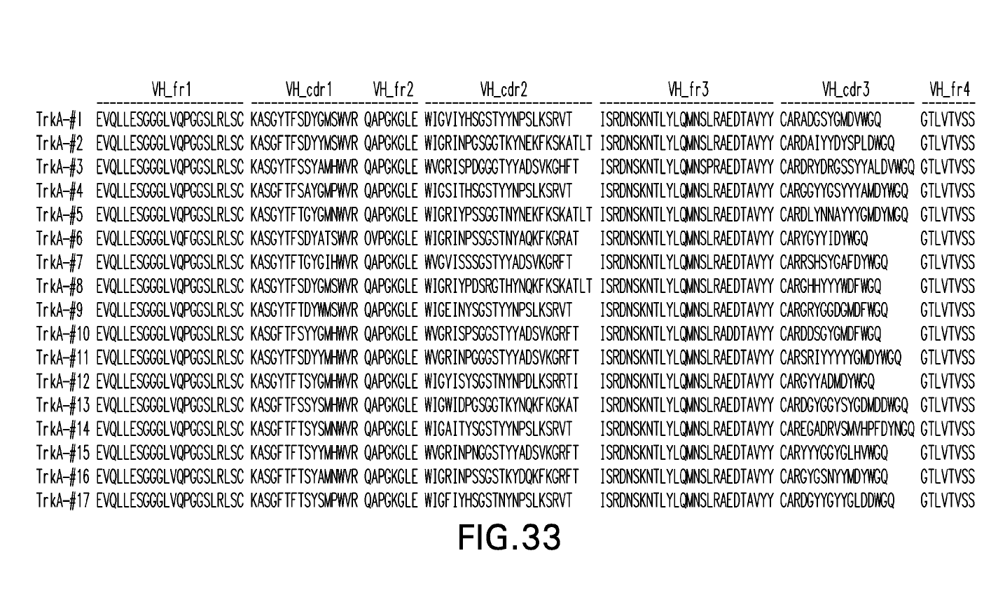

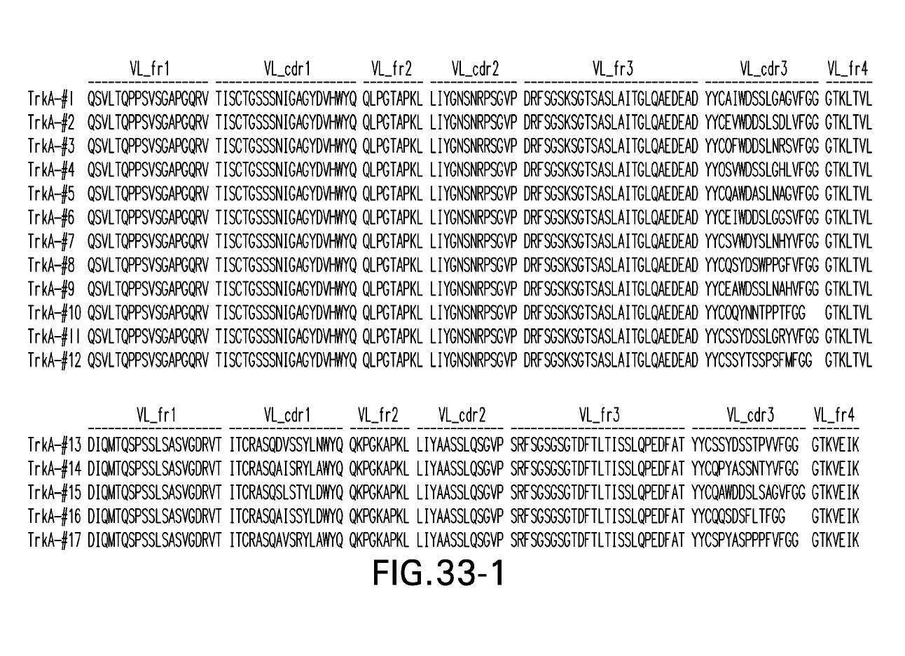

FIG. 33 shows the sequences of VH, Vk, and Vλ from a panel of anti-TrkA antibodies isolated from PDL1 library panning.

### Example 27

### Fab Protein Expression and Purification

50 ml of overnight cultures for individual clones in 2YT/2% glucose/Carbenicillin 100 μg/ml were grown in 37° C. shaker incubator. In the second day, 750 mL to IL of 2YT/0.1% glucose/100 ug/mL Carbenicillin was inoculated for each clone by transferring 5-10 ml of the overnight culture. The cultures were grown at 30° C. with shaking for approximately 3-4 hrs until OD₆₀₀˜1. IPTG was added to the culture to reach the final concentration of 0.1-0.5 mM. After overnight IPTG induction at 22° C., the cells pellets were collected by centrifugation at 10,000 rpm for 10-15 mins, to proceed for periplasmic preparation.

Soluble Fabs were extracted from cell periplasm. The periplasmic preparation was performed as following. The TG1 pellet was re-suspend in 20 mL pre-chilled PPB buffer (20% Sucrose+2 mM EDTA+30 mM Tris, pH=8), and incubated on ice for 1 hour. The supernatant with soluble Fab was collected by centrifugation. Subsequently, the cell pellet was further re-suspended in 20 mL pre-chilled 5 mM magnesium sulfate with 1 hour incubation on ice. Two supernatants were combined for further Fab purification.

The supernatant from periplasmic preparation was loaded into a 1 ml protein-G column (HiTrap, GE healthcare) for Fab purification. After column wash with 50 ml PBS, Fab protein was eluted with 0.3M acetic acid, pH3 buffer. The eluted fractions were collected, and neutralized with 0.5 volume of 1M Tris-HCl, pH9 buffer. The Fab samples were buffer-exchanged to PBS, and a small portion was injected into Size Exclusion HPLC to check protein purity, and used for Fab ELISA assay. We have completed the Fab expression and purification for all 128 PCSK9 hits. Overall summary of Fab yields is ˜2 mg/L with high degree of variability, from less than 1 mg/L to well over 10 mg/L. The ELISA assay results confirmed all 128 Fab bound to human PCSK9 antigen.

### Example 28

### Biacore-Based PCSP9-LDL Receptor Interaction Assay

The LDL-Receptor (LDLR) and EGF_AB domain of LDLR (this domain involves the interaction with PCSK9) were immobilized on two different flow cells in the same CM5 chips by coupling of amine groups of LDLR or EGF_AB domain onto carboxylated surfaces of sensor chips according to the instruction of Amine Coupling Kit (GE/Biacore). Briefly, LDLR and EGF_AB were diluted to 20 μg/ml in pH 4.5 10 mM Acetate buffer and injected to two flow cells on the same CM5 chip to achieve an immobilization level of ˜1500 RU. 100 nM human PCSK9 alone in running buffer (1×HBSP with 0.1 mM CaCl₂) was injected into the flow cells (at 20 ul/min for 2.5 min) to measure the interaction of PSK9 with LDLR and EGF_AB domain. After injection, the flow cells were regenerated by 10 mM HCl.

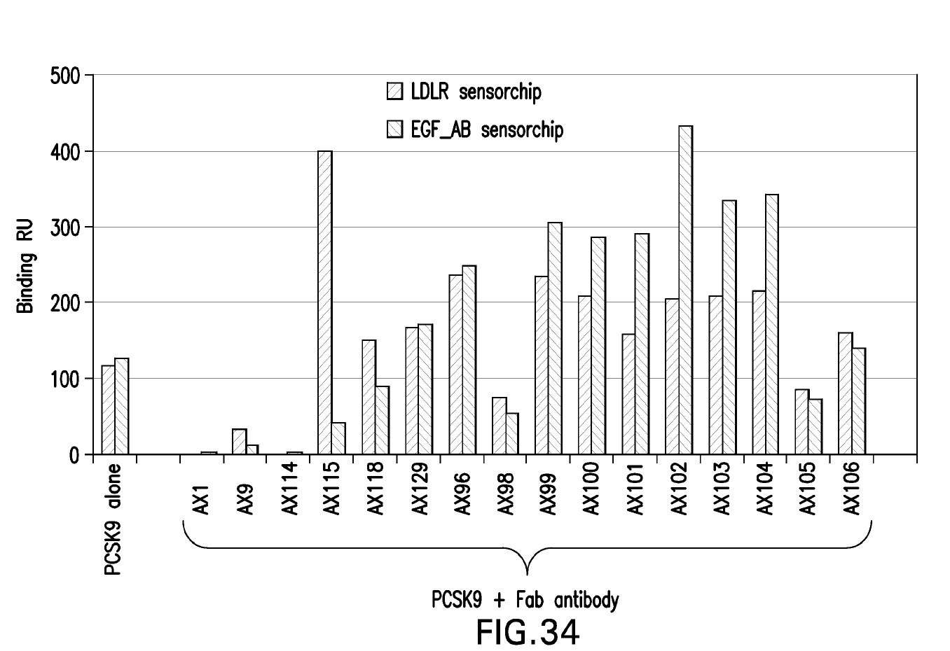

To determine the impact of the binding of Fab antibody to PCSK9, each purified Fab sample (1 uM in the running buffer) was incubated with human PCSK9 at the concentration of 100 nM for 30 min at room temperature. The prepared PCSK9/Fab samples were injected into the CM5 chip, and binding of PCS9/Fab complex was measured. As shown in FIG. 34, human PCSK9 alone bound to both LDLR and EGF_AB domain, When the binding of Fab antibody did not inhibit the PCSK9-LDLR interaction, the binding of PCS9/Fab complex to LDLR or EGF_AB resulted in higher binding RU then PCSK9 alone. Among the Fab antibodies tested, AX1, AX9 and AX114 Fabs showed significant inhibition on PCSK9 binding to LDLR or EGF_AB domain.

### Example 29

### Biacore-Based Competition Assay for Binding Epitope Binning

Human PCSK9 protein was immobilized on CM5 chip by coupling primary amine groups of PCSK9 onto carboxylated surfaces of sensor chips according to the instruction of Amine Coupling Kit (GE/Biacore). Briefly, hPCSK9 protein was diluted to 50 μg/ml in pH 5.5/10 mM Acetate solution, and was injected onto the NHS/EDC activated surface to achieve an immobilization level of 1000-2000 RU, followed with surface inactivation by injection of Ethanolamine. The Fab or IgG protein in FIBS-P buffer) was then injected for 3 minutes binding, followed with 5 minutes dissociation. In the binding epitope binning assay, two flow cells were immobilized with same amount of hPCSK9 protein to detect the binding competition between antibody 1 and antibody 2. On the flow cell 1, antibody 1 was injected twice to occupy its binding epitope, antibody 2 was then injected for binding. The flow cell 2 was setup as a reference, only antibody 2 was injected onto it for binding. To determine whether there was competition between antibody 1 and antibody 2, the sensorgrams of antibody 2 from both flow cells were overplayed. When two antibodies competed, pre-occupation of antibody 1 could significantly or totally inhibit the antibody 1 binding. Cross competition for 19 antibodies from PDL1 library was completed, and 3 independent epitope bins on human PCSK9 were identified, see table 18.

TABLE 18

| Bin 1 binder | Bin 2 binder | Bin 3 binder |
|---|---|---|
| AX114 | AX1 | AX116 |
| AX132 | AX9 | |
| AX139 | AX40 | |
| AX212 | AX56 | |
| AX213 | AX115 | |
| AX210 | AX118 | |
| AX211 | AX119 | |
| AX212 | AX188 | |
| | AX189 | |
| | AX191 | |

### Example 30

### Biacore Assay for Affinity Measurement

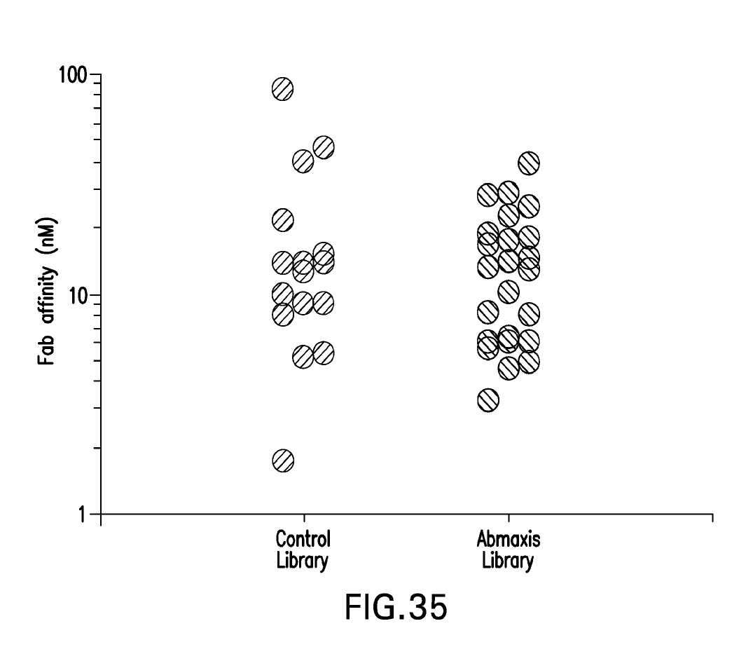

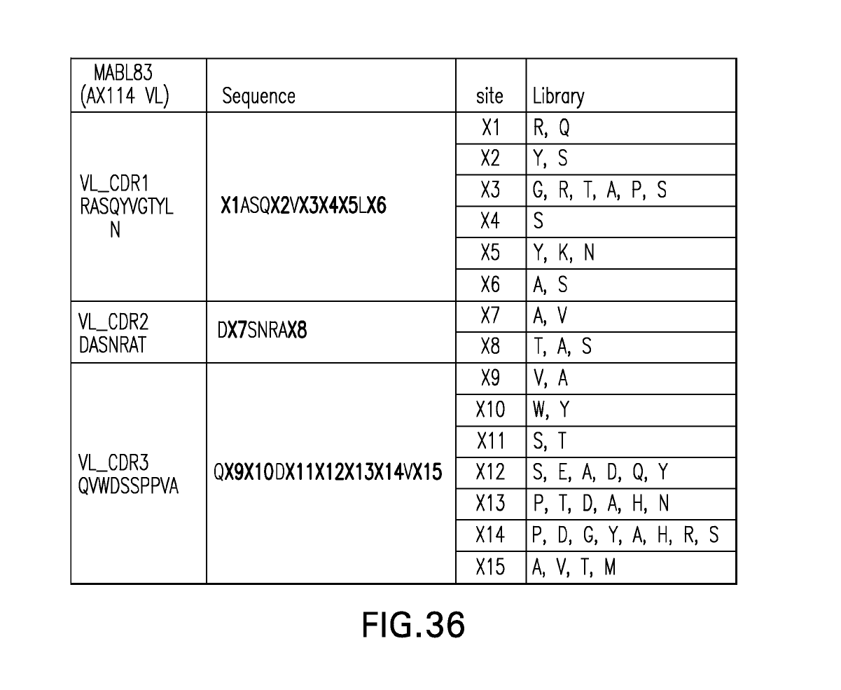

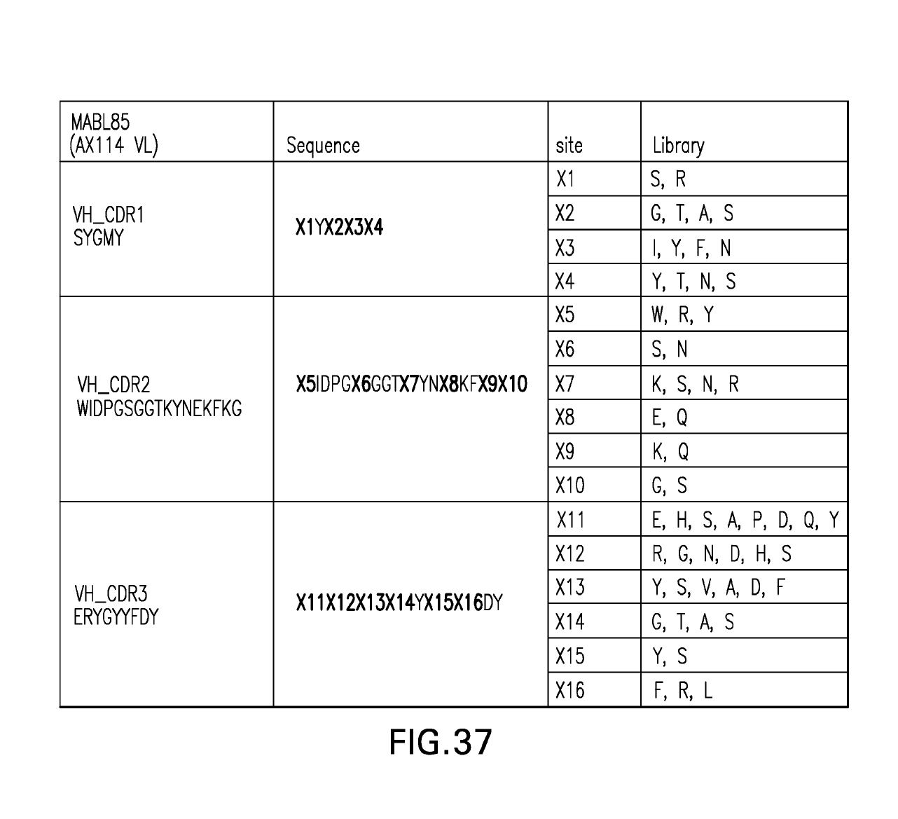

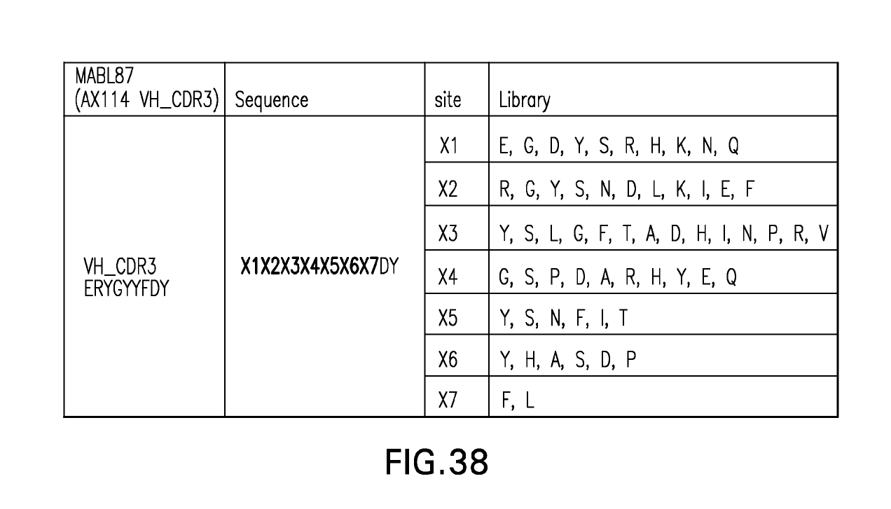

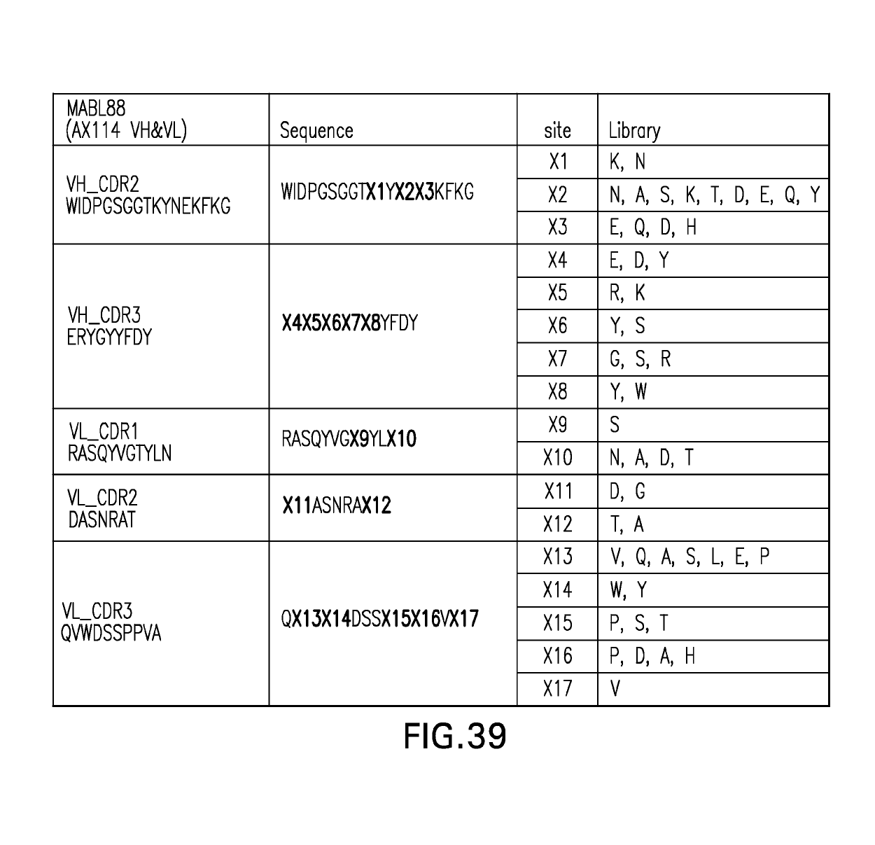

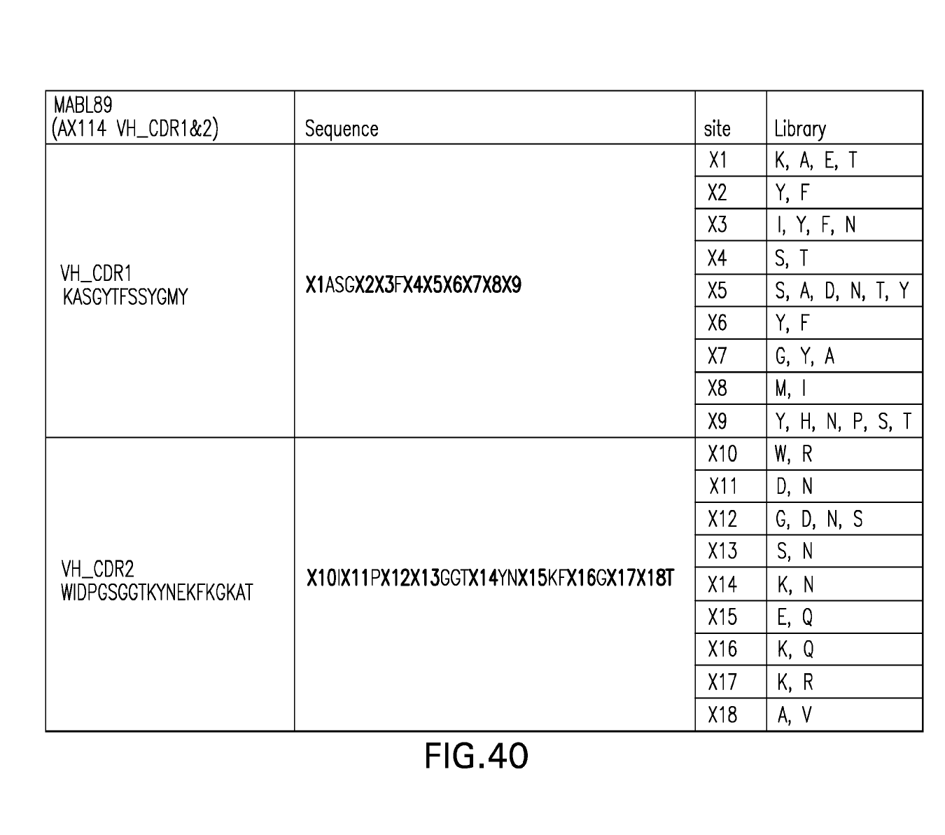

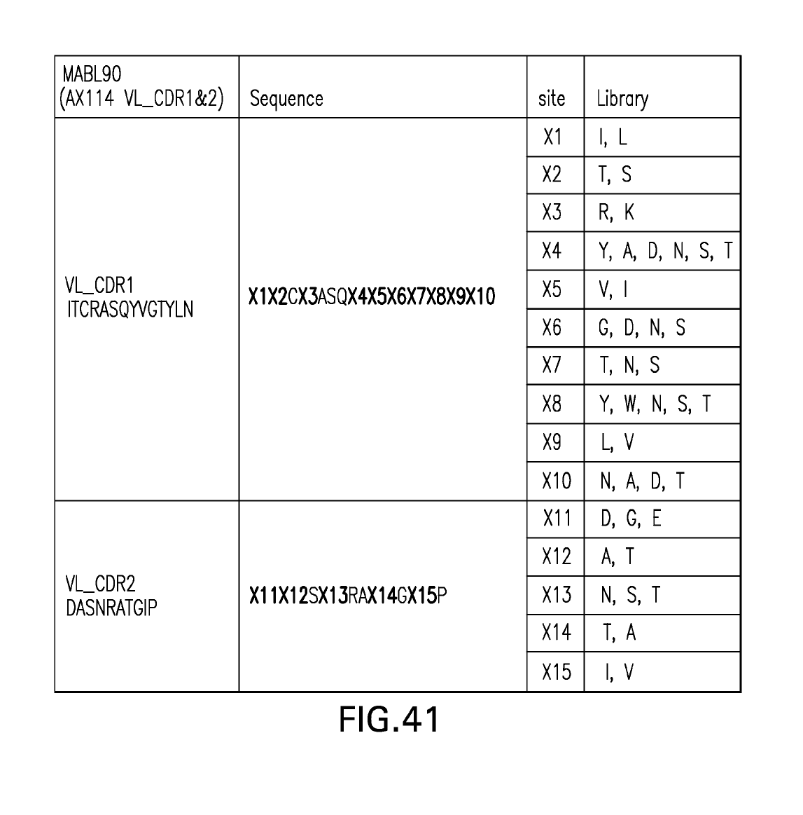

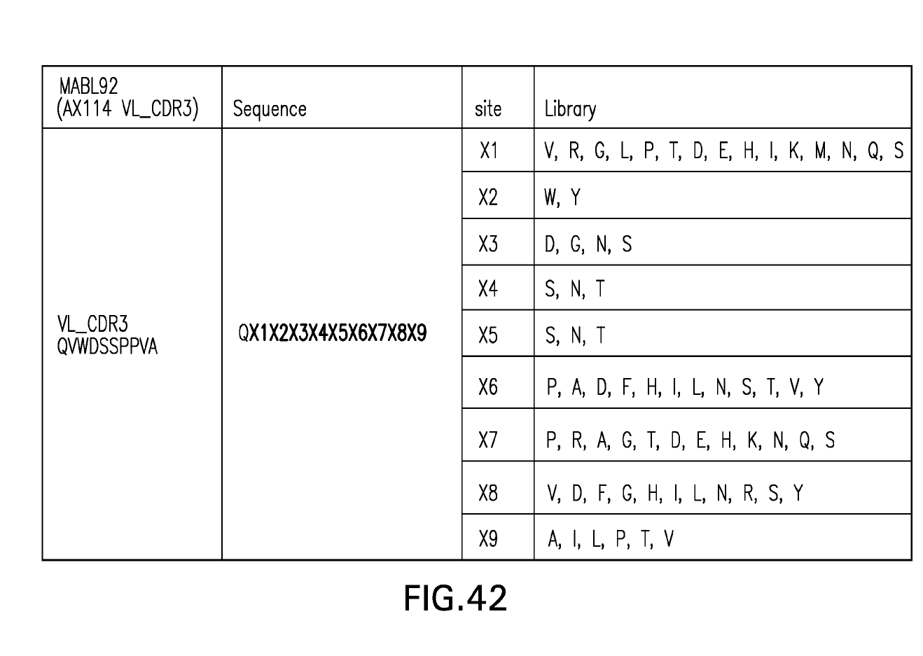

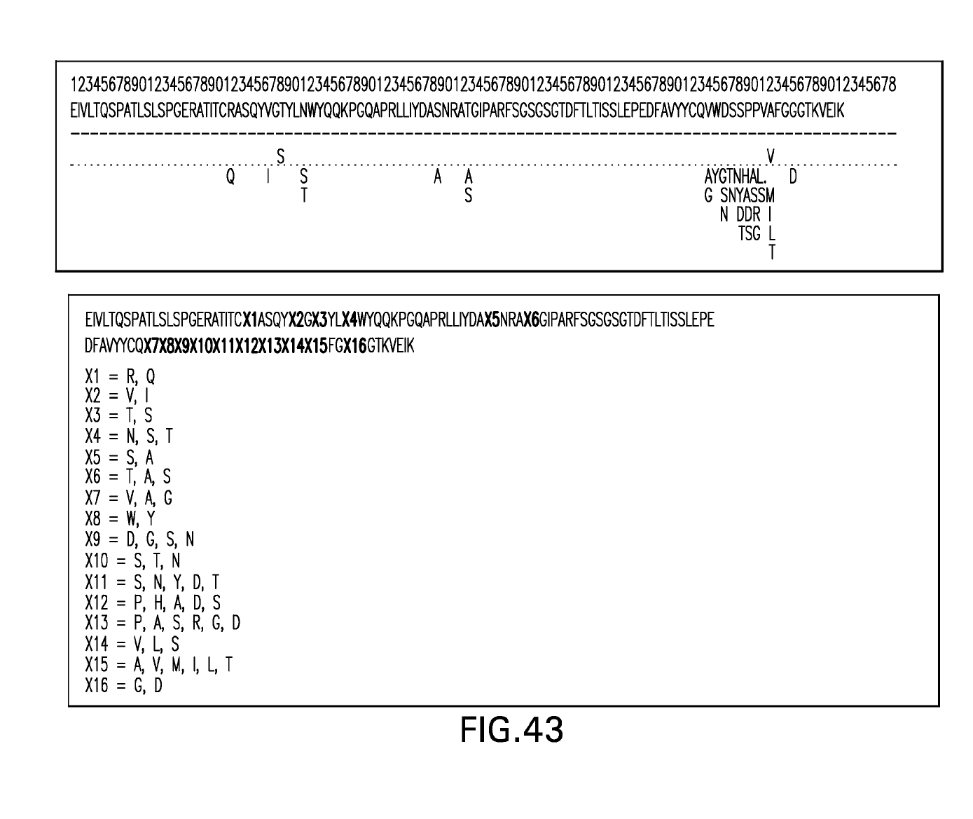

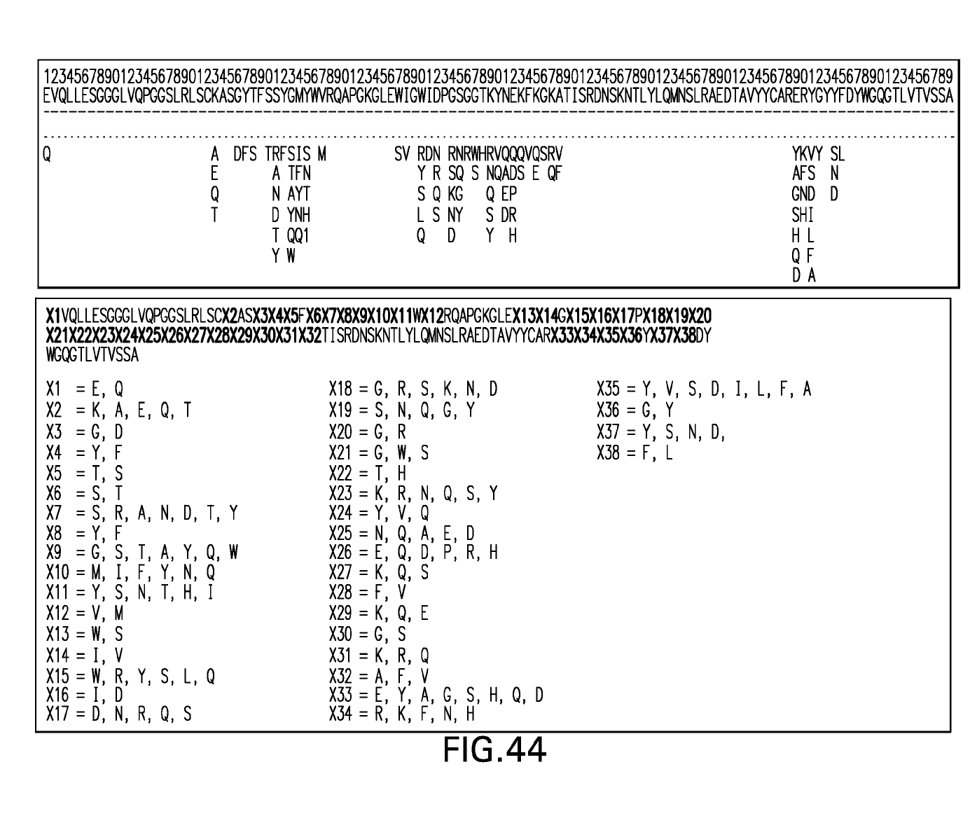

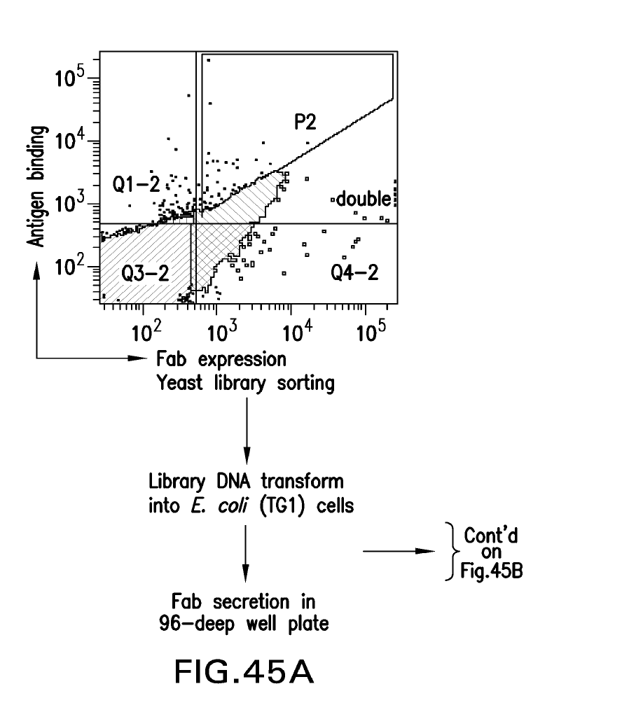

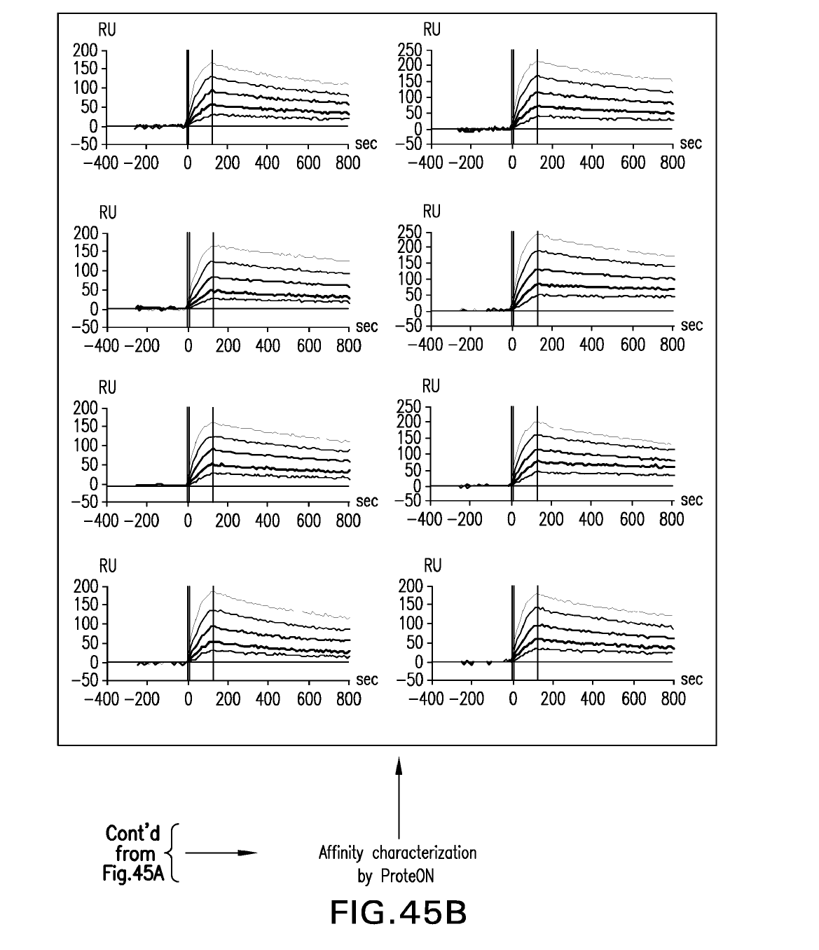

To determine the binding affinity of Fab to PCSK9, Fab capture-based bioacore assay was developed. First, goat anti-Fab IgGs were immobilized onto CM5 chip by amine coupling as described above. The anti-Fab IgGs were diluted to 200 μg/ml in pH 5/10 mM Acetate solution, and injected onto the NHS/EDC activated surface to achieve an immobilization level of ˜10000 RU, followed with surface inactivation by injection of Ethanolamine. Then Fab samples at concentration of 2 μg/ml in FIBS-P running buffer were injected for 3 mins at flow speed of 20 ul/min, followed with K-injection (3 min injection for association and 6 mins for dissociation) of PCSK9 at concentration of 10 to 100 nM. The sensorchip surface was regenerated by 30 second injection of 100 mM phosphoric acid. The binding sensorgrames were fitted with 1:1 Langmuir binding model to determine the binding affinity. We have randomly picked 34 Fabs panned from PDL1 library and 16 Fabs from a control library to compare the affinity distribution. The data in FIG. 35 shows that the Fabs from PDL1 library had affinity of a range of 2-50 nM to PCSK9 protein, which was comparable to that of the control library.

In the following examples, reference is made to various provisional applications each of which is incorporated by reference in its entirety. Specifically, each of Ser. No. 61/256,720 ('720), Filed Oct. 30, 2009 and 61/323,117 ('117), Filed Apr. 12, 2010 (Collectively Attorney Docket No, MRL-ACV-00028) is incorporated by reference in its entirety. Likewise, each of Ser. Nos. 61/256,732 ('732) filed Oct. 30, 2009 and 61/323,148 ('148) filed Apr. 12, 2010 (Collectively Attorney Docket No, MRL-ACV-00029) is incorporated by reference in its entirety

### Example 31

### AX114 Optimization Library Design

### Example 31-1

### Antibody Structure Modeling for AX114 Fv

The AX114 antibody structure was built by using Abmaxis antibody structure prediction tool which have been described in U.S. Pat. No. 117,096 B2. The structure templates for each fragment, chain and Fv were selected by remote homology modeling. The predicted AX114 Fv structures were sorted by the energy scoring function implemented in CONGEN. The top 300 Fv structures were clustered into 6 clusters based on backbone Ca with 1.5 A cutoff. The Fv confirmations with the lowest energy score in each cluster are selected to stand for that cluster.

### Example 31-2

### Mutation Effects on AX114 Fv Folding Stability

For each selected AX114 Fv structure described in EXAMPLE 31-1, the mutation effects on the Fv folding stability have been used to computationally screen the possible mutations at interested regions, such as CDRs. The antibody folding stability is evaluated by the unfolding free energy. The unfolding free energy is defined as the antibody free energy change between the unfolded and folded state. The higher the unfolding free energy, the more stable the antibody structure.

In the mutation process, the increase of the unfolding free energy indicates the mutation stabilizes the Fv structure. On the other hand, the decrease of the unfolding free energy indicates the mutation destabilizes the Fv structure. An outline of the calculation of the unfolding free energy change during the mutation process is as follow:

The hydrogen atoms were added onto the modeled Fv structure using VMD. The energy minimization on the hydrogen atoms were carried out using NAMD. The energy minimized structure is the folded state for the wild type antibody. The mutant was generated using “SCWRL” based on the modeled Fv structure. The hydrogen atoms were added using VMD. The energy minimization was carried out on the mutated residue side-chains and the hydrogen atoms using NAMD. The energy minimized structure is the folded state for the mutant antibody.

As for the unfolded state, first, it is assumed that the unfolded state devoid of any interaction between residues. The unfolded state is simply modeled as the residues under mutations exposed to the solvent. Then, the charge-charge interaction in the unfolded state could be added in using Gaussian-chain model

The unfolding free energy for wild type antibody or mutant was obtained by subtracting the free energy for folded state from the one for unfolded state.

The change of unfolding free energy during the mutation process was obtained by subtracting the unfolding free energy for wild type from the one for mutant. Positive change means the mutant stabilize the Fv folded structure.

According to the inventors' calculation, both polar and non-polar contributions to the free energy of each state are considered. The polar contributions are obtained by solving Poisson-Boltzmann equation using the finite difference method with a grid size ˜0.3 Å in APBS.

The non-polar part is calculated to use the Apolar module in APBS, which includes both cavity creation term from scaled particle theory and the dispersion term from weeks-chandler-Anderson framework. Amber force field is used to set the parameters for each antibody atom including charges and radii.

### Example 31-3

### Mutation Effect on AX114 Binding Affinity to hPCSK9

Selected AX114 Fv structures (from Example 31-1) were docked with X-ray structure of hPCSK9 using Rosetta_Dock to predict the complex confirmation. The sampled complex confirmations were sorted by the Rosetta score and clustered according to the antibody backbone Ca. The confirmation with the lowest score in the biggest cluster was selected as the complex confirmation to screen the mutation effect on the AX114 binding affinity to hPCSK9.

The binding free energy is defined as the difference of free energy between the complex and separate proteins. Both polar and non-polar contributions were calculated using APBS with Amber force field.

### Example 31-4

### Mutation Scanning on AX114 CDRs

Multiple runs of mutation scanning were carried out on all 6 AX114 CDRs. It started with the single-mutation scanning. Except Cys, all other 19 amino acid types were evaluated at each CDR residue site. Both folding (EXAMPLE 31-2) and binding effects (EXAMPLE 31-3) were calculated. The mutations with equal, better and slightly worse on AX114 Fv folding and binding stability than wild type residue were selected to go to the multiple-mutation scanning. The multiple-mutation scanning was carried out on the interested regions or the regions structurally interacting with each other. The mutation scanning provided the profile of the possible mutation at the interested target site.

### Example 31-5

### AX114 CDRs Sequence Database Profiling

Each AX114 CDR sequence and 6 amino acid residues from the adjacent framework regions (3 front and 3 after) was used as lead sequence to search the antibody database and select the hit amino acid sequence by the remote homology. Variant profile was built to list all variants at each position based on the hit library and filtered with certain cutoff value to reduce the size of the resulting hit variant library within computational or experimental limit.

### Example 31-6

### Mutation Sorting on AX114 CDRs Using Rosetta Design

Rosetta_design was also used to evaluate the mutation effect on the AX114 folding stability. Two kinds of calculations were carried out: one with the GB calculation (electrostatic interaction) and one without. The scanning started with each CDR alone, then each chain, then Fv. The profile from Example 31-4 and example 31-5 were used as initial input file to provide the choices for each interested position. The mutations were ranked by the popularity and Rosetta score.

### Example 31-7

### AX114 Optimization Libraries

By combining the possible mutation profiles from Example 31-4, -5 and -6 and filtered with certain cutoff value, 7 optimization libraries were designed to target the VH chain, VL chain, VH_CDR3, VH_CDR1&2, VL_CDR3, VL_CDR1&2, and both VH&VL, and showed in FIG. 4-10. Refer to one of the '720 and/or '117 applications.

### Example 32

### Optimization Libraries of Antibody AX114

Two types of libraries were constructed using the adapter-directed phage display technology as described by Wang et al., Journal of Molecular Biology 2010, 395:1088-1101, using either pre-existed building blocks libraries from PDL1 library, or specific designed libraries based on AX114 structure modeling. Total 10 optimization libraries were constructed for AX114 antibody identified from PDL1 library, as listed Table 19.

TABLE 19
AX114 optimization libraries

| library | VH | Vk | library size (clones) |
|---|---|---|---|
| MABL81 | AX114 WT | Vk1 library from PDL1 | 7.80E+08 |
| MABL82 | AX114 WT | Vk3 library from PDL1 | 2.00E+08 |
| MABL83 | AX114 WT | Structure-based Vk library | 2.10E+08 |
| MABL85 | Structure-based VH-CDRs | AX132 (AX114 variant) | 1.10E+09 |
| | library | | |
| MABL86 | VH-CDR1, 2 library from | AX132 | 5.80E+07 |
| | PDL1 | | |
| MABL87 | AX114-CDR3 library | AX139 (AX114 variant) | 4.10E+08 |
| MABL88 | Structure-based VH-CDR2,3 | Structure-based Vk-CDR1,2,3 | 5.20E+08 |
| | library | library | |
| MABL89 | VH-CDR 1, 2 library | AX139 | 3.80E+08 |
| MABL90 | AX114 WT | Structure-based AX139 Vk3- | 1.70E+08 |
| | | CDR1 and CDR2 library | |
| MABL92 | AX114 WT | Structure-based AX139 Vk3- | 2.40E+08 |
| | | CDR3 library | |

Library construction process was described as following. Briefly, the AX114 vector DNA was prepared from TG1 bacterial cells using HiPure Plasmid Maxiprep kit (Invitrogen). AX114 or its variant vector DNA was digested with restriction enzymes BamHI and KpnI for cloning full Vk libraries, BamHI and BbsI cut for cloning Vk CDR1 and CDR2 libraries, and BbsI and KpnI cut for cloning Vk CDR3 libraries. To make the heavy chain libraries, the AX114 and its variant vector DNA were digested with restriction enzymes HindIII and XhoI for cloning full VH libraries, with HindIII and PstI cut for cloning VH-CDR1 and CDR2 libraries, and cut with PstI and XhoI for cloning VH-CDR3 libraries. The digested AX114 and its variant vector DNA were purified by electophoresis in 1% Agarose gel for library ligation and construction.

PCR reaction was used to amplify the VH CDRs libraries from either pre-existed PDL1 library or structure-based new libraries. The PCR primers were listed in the Table 20.

TABLE 20
primers for amplification of VH-CDRs libraries

| Primer | Sequence | Amplification |
|---|---|---|
| AM-190 | TCTCACTCCGCTGGATCCGAAGTGCAGCTGCTGGAATC | VH-CDR1 and CDR2 library |
| AM-148 | ACGGGCGCAGTAGTACACTGC | VH-CDR1 and CDR2 library |
| AM-152 | CTCGTTCCGATGCTAAGCTTCGCTGACACTGCAGTGTACTACTGCG | VH-CDR3 libraries |
| AM-150 | GCCTTTGGTGCTTGCGCTCGAGACAGTCACCAGCGTACCCTGACC | VH-CDR3 libraries |
| AM-155 | ACCCTCGTTCCGATGCTAAGCTTCGCTGAAATCGTGCTGACCCAG | VK3 chain library |
| AM-153 | GAATACAGATGGTGCAGCCAC | VK3 chain library |
| AM-260 | CCAGCCACCCTGTCTCTGTCTC | Vk3 CDR1 and CDR2 |
| | | libraries |
| AM-261 | GAAGTCGGTGCCAGAACCAGATC | Vk3 CDR1 and CDR2 |
| | | libraries |
| AL-521 | TCTCTGGAACCAGAAGACTTCGCCGTGTACTACTGCCAG | Vk3 CDR3 libraries |
| AL-522 | ACGTTTGATCTCCACTTTGGTACCACCACCGAAA | Vk3 CDR3 libraries |

The PCR programs were as following: 94 C, 2 min preheat; 30 cycles of “94 C, 45 see; 55 C, 45 sec; 72 C, 1 min”; then 72 C, 10 min for completion. 20 to 60 PCR reactions were performed for each library to make enough DNA for subsequent cloning into AX114 and its variant vectors. The PCR-amplified DNA was purified using Qiaquick PCR purification Kit (Qiagen), then digested with corresponding restriction enzymes as described above. The ligation reaction was carried out for overnight at 16° C., with the following reaction mixture: 10-20 ug vector DNA, 2-4 ug insert library DNA, 4000-8000 U T4 DNA ligase in 1× ligation buffer (New England BioLabs). The ligation products were purified by Qiaquick PCR Purification kit (Qiagen), and transform into TG1 cells by electroporation with a mix of 1.2 ug DNA and 200 ul of electro-competent cells for each. The electroporations were repeated to achieve enough colony formation unit (cfu). The transformed cells plated out on 10 2YT agar plates with 2% glucose and 100 ug/ml carbenicillin. The complexity of each library is listed in the Table 18.

All AX114 libraries were panned against PCSK9 antigen for 3-6 rounds as described in the Example 2. Refer to one of the '720 and/or '117 applications. The clones were picked for Fab ELISA screening as described in the Example 3. Refer to one of the '720 and/or '117 applications. The PCSK9 binding clones were expressed in TG1 cells for Fab secretion, Purified Fab proteins (Example 4—Refer to one of the '720 and/or '117 applications) were run on Biacore for affinity measurements (see Example 15—Refer to one of the '720 and/or '117 applications). From these libraries, total of 135 AX114 variants (listed in Table 20) that bond to human PCSK9 were identified, including AX132.

TABLE 20
sequence ID for AX114 and its variants

| Sequences | SEQ ID |
|---|---|
| VH | 1-135 |
| VK | 136-174 |
| VH_CDR1 | 175-268 |
| VH_CDR2 | 269-370 |
| VH_CDR3 | 371-423 |
| VK_CDR1 | 424-431 |
| VK_CDR2 | 432-436 |
| VK_CDR3 | 437-467 |

> (AX132 VH)
EVQLLESGGGLVQPGGSLRLSCKASGYTFSSYGMYWVRQAPGKGLEWIGW
IDPGSGGTKYNEKFKGKATISRDNSKNTLYLQMNSLRAEDTAVYYCARER
YGYYFDYWGQGTLVTVSSAS
>(AX132 VK)
EIVLTQSPATLSLSPGERATITCRASQYVGSYLNWYQQKPGQAPRLLIYD
ASNRATGIPARFSGSGSGTDFTLTISSLEPEDFAVYYCQVWDSSPPVVF
GGGTKVEIK

The sequence changes in the CDR regions for all XA114 variants are illustrated in FIGS. 11 and 12 as set forth in the '720 and/or '117 application.

### Example 33

### Cross Phage/Yeast Display for Ax114 Engineering

The cross-species display using the same display vector provides the possibility to display a library that can shuttle between phage and yeast surfaces, therefore the large size library can be first selected in phage display format, and then the enriched library be directly processed to yeast display and FACS sorting without the need of molecular cloning. MABL85 VH-maturation library (with diversity in all three CDRs) was cloned into this cross-display vector, see Wang et al., Journal Molecular Biology 2010, 395: 1088-1101. This affinity maturation library was display on phage, and panned against PCSK9 for one round. Then the first round panning-enriched VH library was transformed into S. cerevisiae KQP600 cells for yeast display, see Wang et at., Journal of Immunological Methods 2010, 354:11-19. 18 unique VH sequences with 2-10 folds affinity improvements were isolated from 5 rounds of FACS shorting.

Taking the advantage that the cross-species display vector worked for Fab secretion in both yeast and E. coli cells, we extracted the maturation library DNA from the yeast cells post 5 rounds of sorting and transformed them into E. coli TG1 cells that have higher growth rate and shorter culture time. After overnight incubation on agar plates, single TG1 colonies were picked, grown in a 96-deep-well plate, and induced overnight with IPTG for Fab expression. The Fab-containing supernatants were subjected to affinity characterization. In this process, affinity measurements were conducted on ProteOn XPR36 (BioRad), a protein interaction array system that can generate simultaneous analysis of up to six ligands with up to six analytes. The Fab-containing supernatants were flown through a GLC sensorchip immobilized with anti-Fab antibody (Sigma). Once the Fab molecules were purified and captured on the sensorchip, the antigen protein solutions of 5 different concentrations were injected simultaneously to generate real time sensorgrams for the kinetic analysis of each Fab sample. With this process, we were able to complete affinity measurements for 96 Fabs in 1 day.

FIG. 13 illustrates the process of affinity measurement for yeast hits in E. coli system. The library DNA was extracted from post-sorting yeast cells, and directly transformed into bacterial TG1 cells. The isolated colonies were picked up into a 96-deep-well plate, grown and induced overnight with 0.5 mM IPTG. The resulting culture supernatants were directly injected into ProteON XPR36 (BioRad) for Fab affinity characterization. The Fab-containing supernatants were flown through a GLC sensorchip immobilized with anti-Fab antibody (Sigma). Once the Fab molecules were purified and captured on the sensorchip, the antigen protein solutions of 5 different concentrations were injected simultaneously to generate real time sensorgrams for the kinetic analysis.

### Example 34

### Fab Domain Thermostability

Thermostabilities of Fabs and Fab domains were determined from DSC experiments by analysis and deconvolution of excess heat capacity function in Origin 5.0. The melting transition temperatures (Tm) for Fabs or Fab domains are indicated in Table 21. The Tm of various Fabs and Fab domains range from 72 to 78° C. for PDL1 derived antibodies, which is consistent with well folded antibody Fab region.

TABLE 21
Thermostabilities of AX114 variants

| IgG | Fab domain (Tm, ° C.) |
|---|---|
| Ax114-IgG1 | 76.7 |
| AX114-IgG2 | 76.5 |
| AX132-IgG2 | 77.4 |

### Example 35

### Anti-Pcsk9 Monoclonal Antibodies Expressed and Purification from Mammalian Cells

The DNA sequence encoding the Vk1 or VK3 light chain variable region was amplified by polymerase chain reaction from plasmid template. The product of this amplification was cloned into plasmid pVUNSAGS-FB-LCK that had been previously digested with Fsp1 and Bmt1, using the InFusion cloning system (Clontech). The resulting plasmid was verified by DNA sequencing across the variable region. Endotoxin-free plasmid preparations were made using the Qiagen Endo-Free plasmid maxiprep kit. The DNA sequence encoding the heavy chain variable region of VH3 was amplified by polymerase chain reaction, and the amplified product was cloned into plasmid pV1 JNSA-BF-HCG2M4 that had been previously digested with Fsp1 and Bmt1. The resulting plasmid was verified by DNA sequencing across the variable region. Endotoxin-free plasmid preparations were made using the Qiagen Endo-Free plasmid maxiprep kit.

The plasmid DNA for heavy and light chain was mixed at 1:3, and co-tranfected into HEK293 cells. After 5-7 days culture, the supernatant was harvested and proceeded for Protein-A column purification. Briefly, the cell free supernatant was loaded on to protein-A column pre-equilibrated with three column volume of 20 mM Tris-HCl 047.0 at a flow rate of 5.0 mL/min. The column was washed with three column volumes of the 20 mM Tris-HCl pH7.0 followed by a five column volume wash with 20 mM Tris-HCl pH7.0 containing 1M NaCl to remove the host cell proteins. The anti-PCSK9 antibody was eluted with five column volume of 100 mM Glycine, 100 mM Arginine pH 3.0 and immediately neutralized with 1M Tris-HCl pH8.0.

### Example 36

### Anti-Pcsk9 Monoclonal Antibodies Expressed and Purification from Glycoengineered Pichia Pastoris

Anti-PCSK9 IgG2 monoclonal antibodies expressed in glyco-engineered Pichia pastoris GFI 5.0 host YGLY8316, which is capable of transferring terminal galactose at its complex N-linked glycan. Anti-PCSK9 heavy and light chains were codon optimized and expressed under methanol tightly inducible promoter AOX1 using Saccharomyces cerevisiae alpha mating factor presequence as secretion signal sequence. Anti-PCSK9 antibody from Pichia pastoris GFI 5.0 host YGLY8316 was captured from cell free supernatant media by affinity chromatography using MabSelect™ medium from GE Healthcare (Cat. #17-5199-01). The cell free supernatant was loaded on to Mabselect column (XK 16/20, 1.6 cm×10.0 cm) pre-equilibrated with three column volume of 20 mM Tris-HCl pH7.0 at a flow rate of 5.0 mL/min. The column was washed with three column volumes of the 20 mM Tris-HCl pH7.0 followed by a five column volume wash with 20 mM Tris-HCl pH7.0 containing 1M NaCl to remove the host cell proteins. The anti-PCSK9 antibody was eluted with five column volume of 100 mM Glycine, 100 mM Arginine pH 3.0 and immediately neutralized with 1M Tris-HCl pH8.0. AX213 antibody was well expressed in Pichia, yielding ca. 300-700 mg/L of protein in small scale fermentation process. The yield for AX114 was 5 mg/L in small scale.

Strong Cation Exchange Chromatography employing Source 30S resin from GE Healthcare (Cat #17-1273-02) was used as the second step purification to remove the clipped species and aggregates. Mabselect pool of the anti-PCSK9 antibody was 5× diluted with 25 mM Sodium acetate pH5.0 and loaded on to the Source 305 column pre-equilibrated with three column volume of 25 mM Sodium acetate pH5.0. After loading, the column was washed with three column volume of the 25 mM Sodium acetate pH5.0 and elution was performed by developing a linear gradient over ten column volume ranging from 100 mM to 150 mM Sodium chloride in 25 mM Sodium acetate pH5.0. The fractions containing good assembled anti-PCSK9 antibody was pooled together. The Source 30S pooled fractions that contained the anti-PCSK9 antibody was buffer exchanged into the formulation buffer containing 6% Surcose, 100 mM Arginine, 100 mM Histidine pH6.0 (HyClone® Cat # RR10804.02) and sterile filtered using PES (PolyEtherSulfone) membrane filter and stored @4° C. until release.

### Example 37

### Biacore Assay for Affinity Measurement

To determine the binding affinity of Fab to PCSK9, Fab capture-based Biacore assay was developed. First, goat anti-Fab IgGs were immobilized onto CM5 chip by amine coupling as described above. The anti-Fab IgGs were diluted to 200 μg/ml in pH 5/10 mM Acetate solution, and injected onto the NHS/EDC activated surface to achieve an immobilization level of ˜10,000 RU, followed with surface inactivation by injection of Ethanolamine. Then Fab samples at concentration of 2 μg/ml in HBS-P running buffer were injected for 3 mins at flow speed of 20 ul/min, followed with K-injection (3 minutes injection for association and 6 minutes for dissociation) of PCSK9 at concentration of 10 to 100 nM. The sensor chip surface was regenerated by 30 second injection of 100 mM phosphoric acid. The binding sensorgrams were fitted with 1:1 Langmuir binding model to determine the binding affinity. The Fab affinities of AX114, AX132 and other variants are shown in Table 22.

TABLE 22
Fab binding affinity

| Fabs | Binding affinity to human PCSK9 | | |
|---|---|---|---|
| Name | ka (1/Ms) | kd (1/s) | KD (M) |
| AX114 | 7.17E+04 | 3.48E−03 | 4.85E−08 |
| AX132 | 9.11E+04 | 1.08E−03 | 1.18E−08 |
| AX137 | 1.07E+05 | 2.08E−03 | 1.95E−08 |
| AX139 | 8.97E+04 | 1.18E−03 | 1.32E−08 |
| AX201 | 1.31E+05 | 1.02E−03 | 7.82E−09 |
| AX202 | 1.09E+05 | 1.02E−03 | 9.43E−09 |
| AX204 | 2.34E+05 | 9.87E−04 | 8.39E−09 |
| AX205 | 1.04E+05 | 9.72E−04 | 9.45E−09 |
| AX206 | 1.22E+05 | 1.03E−03 | 8.42E−09 |
| AX207 | 1.11E+05 | 1.00E−03 | 9.23E−09 |
| AX208 | 9.84E+04 | 2.59E−03 | 2.64E−08 |
| AX209 | 1.12E+05 | 1.07E−03 | 9.60E−09 |
| AX210 | 1.72E+05 | 2.87E−04 | 1.67E−09 |
| Ax211 | 1.97E+05 | 3.29E−04 | 1.67E−09 |
| AX212 | 1.59E+05 | 2.33E−04 | 1.47E−09 |
| AX213 | 2.43E+05 | 3.29E−04 | 1.37E−09 |
| AX214 | 1.92E+05 | 3.19E−04 | 1.60E−09 |
| AX215 | 1.16E+05 | 3.76E−04 | 3.47E−09 |
| AX216 | 1.15E+05 | 2.88E−04 | 2.51E−09 |
| AX217 | 1.38E+05 | 3.40E−04 | 2.52E−09 |
| AX239 | 3.03E+04 | 9.60E−04 | 3.16E−08 |
| AX240 | 1.60E+05 | 1.66E−04 | 1.04E−09 |
| AX241 | 1.97E+05 | 1.60E−04 | 8.13E−10 |
| AX242 | 1.62E+05 | 1.93E−04 | 1.19E−09 |
| AX243 | 1.31E+05 | 1.91E−04 | 1.46E−09 |
| AX244 | 1.89E+05 | 2.03E−04 | 1.07E−09 |
| AX245 | 1.12E+05 | 3.19E−04 | 2.84E−09 |
| AX246 | 2.49E+05 | 2.01E−04 | 8.06E−10 |
| AX247 | 1.86E+05 | 2.44E−04 | 1.31E−09 |
| AX248 | 1.89E+05 | 2.07E−04 | 1.09E−09 |
| AX249 | 1.90E+05 | 2.06E−04 | 1.08E−09 |
| AX250 | 3.07E+05 | 2.40E−04 | 7.81E−10 |
| AX251 | 2.54E+05 | 2.52E−04 | 9.93E−10 |
| AX252 | 1.37E+05 | 4.67E−04 | 3.42E−09 |
| AX253 | 1.61E+05 | 6.81E−04 | 4.23E−09 |
| AX254 | 9.24E+04 | 2.95E−04 | 3.19E−09 |
| AX255 | 9.61E+04 | 3.91E−04 | 4.07E−09 |
| AX256 | 1.26E+05 | 3.65E−04 | 2.90E−09 |
| AX257 | 2.53E+05 | 1.68E−04 | 6.64E−10 |
| AX258 | 1.12E+05 | 6.30E−04 | 5.63E−09 |
| AX259 | 6.92E+04 | 6.04E−04 | 8.73E−09 |
| AX260 | 4.19E+04 | 5.20E−04 | 1.24E−08 |
| AX260 | 2.64E+04 | 5.70E−04 | 2.16E−08 |
| AX263 | 2.78E+04 | 1.70E−04 | 6.11E−09 |
| AX267 | 1.29E+05 | 4.75E−04 | 3.70E−09 |
| AX268 | 2.77E+04 | 4.17E−04 | 1.50E−08 |
| AX269 | 1.13E+05 | 3.14E−04 | 2.77E−09 |
| AX269 | 9.49E+04 | 3.20E−04 | 3.37E−09 |
| AX299 | 1.03E+05 | 2.57E−04 | 2.51E−09 |
| AX300 | 1.15E+05 | 3.12E−04 | 2.72E−09 |
| AX301 | 1.28E+05 | 6.00E−04 | 4.67E−09 |
| AX302 | 1.14E+05 | 6.68E−04 | 5.89E−09 |
| AX303 | 7.37E+04 | 8.61E−04 | 1.17E−08 |
| AX306 | 9.04E+04 | 4.47E−04 | 4.94E−09 |
| AX307 | 8.88E+04 | 2.70E−03 | 3.03E−08 |
| AX308 | 5.23E+04 | 2.59E−03 | 4.96E−08 |
| AX310 | 1.09E+05 | 7.43E−04 | 6.79E−09 |
| AX311 | 1.47E+05 | 4.98E−04 | 3.39E−09 |
| AX312 | 1.58E+05 | 1.41E−03 | 8.94E−09 |
| AX313 | 2.13E+05 | 7.35E−04 | 3.46E−09 |
| AX314 | 1.09E+05 | 1.72E−03 | 1.58E−08 |
| AX315 | 4.65E+04 | 2.31E−04 | 4.97E−09 |
| AX316 | 1.57E+05 | 5.63E−04 | 3.59E−09 |
| AX318 | 1.67E+05 | 2.71E−05 | 1.62E−10 |
| AX319 | 2.59E+05 | 4.25E−05 | 1.64E−10 |
| AX320 | 1.51E+05 | 2.62E−05 | 1.74E−10 |
| AX322 | 1.19E+05 | 1.83E−04 | 1.54E−09 |
| AX323 | 1.79E+05 | 8.36E−05 | 4.66E−10 |
| AX325 | 1.39E+05 | 1.19E−04 | 8.53E−10 |
| AX326 | 1.68E+05 | 6.52E−05 | 3.87E−10 |
| AX329 | 1.33E+05 | 2.09E−04 | 1.57E−09 |

The Fabs which showed functional efficacy in the cell-base assays were converted into IgG molecules. The affinities of those IgG molecules were also measured by Biacore assay. Briefly, anti-human IgG monoclonal antibody form Human Antibody Capture Kit provided by Biacore was immobilized on CM5 chips at level of 8000 to 10000 RU. The IgG samples at concentration of ˜0.4 μg/ml was injected onto sensor chip for 2 minutes at a flow rate of 20 μl/min, then PCSK9 proteins at 5 concentrations (3.75 to 60 nM) were injected onto IgG captured flow cell for binding kinetic analysis. After each round injection, the sensor chip surface was regenerated by 30 second injection of 3M Magnesium Chloride. The affinities of AX114, AX1213 and other variants are shown in Table 22 and Table 23.

TABLE 22
Purified IgGs against human PCSK9

| Name | ka (1/Ms) | kd (1/s) | KD (M) |
|---|---|---|---|
| AX114 | 1.51E+05 | 3.61E−03 | 2.40E−08 |
| AX132 | 2.48E+05 | 1.52E−03 | 6.16E−09 |
| AX137 | 3.33E+05 | 3.32E−03 | 9.98E−09 |
| AX210 | 2.35E+05 | 6.21E−04 | 2.64E−09 |
| AX211 | 3.61E+05 | 5.89E−04 | 1.63E−09 |
| AX212 | 1.53E+05 | 3.24E−04 | 2.12E−09 |
| AX213 | 3.53E+05 | 7.30E−04 | 2.07E−09 |

TABLE 23
Purified IgGs against rhesus PCSK9

| Name | ka (1/Ms) | kd (1/s) | KD (M) |
|---|---|---|---|
| AX240 | 1.41E+06 | 3.09E−04 | 2.20E−10 |
| AX241 | 1.56E+06 | 3.46E−04 | 2.22E−10 |
| AX242 | 1.76E+06 | 3.25E−04 | 1.85E−10 |
| AX243 | 7.71E+05 | 8.70E−04 | 1.13E−09 |
| AX245 | 8.90E+05 | 1.05E−03 | 1.18E−09 |
| AX246 | 1.48E+06 | 6.14E−04 | 4.16E−10 |
| AX248 | 1.16E+06 | 4.58E−04 | 3.96E−10 |
| AX249 | 1.22E+06 | 6.53E−04 | 5.35E−10 |
| AX250 | 2.09E+06 | 6.23E−04 | 2.98E−10 |
| AX253 | 1.62E+06 | 1.76E−03 | 1.09E−09 |
| AX267 | 1.38E+06 | 1.26E−03 | 9.13E−10 |
| AX277 | 9.99E+05 | 1.67E−03 | 1.67E−09 |
| AX369 | 9.71E+05 | 2.30E−03 | 2.37E−09 |
| AX370 | 1.01E+06 | 2.31E−03 | 2.28E−09 |
| AX402 | 1.09E+06 | 2.32E−03 | 2.12E−09 |
| AX406 | 8.86E+05 | 2.01E−03 | 2.27E−09 |
| AX408 | 7.65E+05 | 1.50E−03 | 1.96E−09 |
| AX415 | 7.50E+05 | 3.12E−03 | 4.15E−09 |
| AX417 | 1.10E+06 | 9.04E−04 | 8.22E−10 |
| AX419 | 1.25E+06 | 9.41E−04 | 7.51E−10 |
| AX426 | 6.92E+05 | 6.28E−04 | 9.08E−10 |
| AX427 | 7.45E+05 | 5.60E−04 | 7.51E−10 |
| AX428 | 6.55E+05 | 4.43E−04 | 6.76E−10 |
| AX429 | 7.25E+05 | 5.88E−04 | 8.11E−10 |
| AX430 | 9.15E+05 | 8.52E−04 | 9.31E−10 |
| AX432 | 7.39E+05 | 1.25E−03 | 1.69E−09 |
| AX436 | 4.81E+05 | 7.93E−04 | 1.65E−09 |
| AX439 | 7.40E+05 | 7.36E−04 | 9.94E−10 |
| AX441 | 7.92E+05 | 8.22E−04 | 1.04E−09 |
| AX444 | 7.08E+05 | 4.99E−04 | 7.06E−10 |

LIST OF VH SEQUENCES
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARERYGYYFDYWGQGTLVT
VSSAS
>
QVQLLESGGGLVQPGGSLRLSCKASGYTFSSYSIYWVRQAPGKGLEWIGWIDPGNGGT
RYNQKFQSKATISRDNSKNTLYLQMNSLRAEDTAVYYCARARVGYSLDYWGQGTLVT
VSSAS
>
QVQLLESGGGLVQPGGSLRLSCKASGYTFSRQGFTWVRQAPGKGLEWIGWIDPGNGGT
RYNEKFKGKATISRDNSKNTLYLQMNSLRAEDTAVYYCARYRVGYSLDYWGQGTLVT
VSSAS
>
QVQLLESGGGLVQPGGSLRLSCKASGYTFSSYSFSWVRQAPGKGLEWIGYIDPGSGGTK
YNQKFQGKATISRDNSKNTLYLQMNSLRAEDTAVYYCARQRVGYSLDYWGQGTLVTV
SSAS
>
QVQLLESGGGLVQPGGSLRLSCKASGYTFSRYGINWVRQAPGKGLEWIGRIDPGNGGT
RYNEKFKGKATISRDNSKNTLYLQMNSLRAEDTAVYYCARANDGYSFDYWGQGTLVT
VSSAS
>
QVQLLESGGGLVQPGGSLRLSCKASGYTFSRYGISWVRQAPGKGLEQIGWIDPGSGGTR
YNQKFKSKATISRDNSKNTLYLQMNSLRAEDTAVYYCARARVGYSFDYWGQGTLVTV
SSAS
>
QVQLLESGGGLVQPGGSLRLSCKASGYTFSSYTNYWVRQAPGKGLEWIGYIDPGSGGT
RYNQKFQGKATISRDNSKNTLYLQMNSLRAEDTAVYYCARSRVGYSFDYWGQGTLVT
VSSAS
>
QVQLLESGGGLVQPGGSLRLSCKASGYTFSRYSYYWVRQAPGKGLEWIGYIDPGSGGT
KYNQKFQSKATISRDNSKNTLYLQMNSLRAEDTAVYYCARARVGYSFDYWGQGTLVT
VSSAS
>
QVQLLESGGGLVQPGGSLRLSCKASGYTFSSYANTWVRQAPGKGLEWIGWIDPGNGGT
SYNQKFKSKATISRDNSKNTLYLQMNSLRAEDTAVYYCARYRSGYSLDYWGQGTLVT
VSSAS
>
QVQLLESGGGLVQPGGSLRLSCKASGYTFSSYGYYWVRQAPGKGLEWIGWIDPGSGGT
RYNQKFKGKATISRDNSKNTLYLQMNSLRAEDTAVYYCARERVGYSLDYWGQGTLVT
VSSAS
>
QVQLLESGGGLVQPGGSLRLSCKASGYTFSRYAINWVRQAPGKGLEWIGWIDPGSGGT
RYNEKFKGQATISRDNSKNTLYLQMNSLRAEDTAVYYCARHRVGYSLDFWGQGTLVT
VSSAS
>
QVQLLESGGGLVQPGGSLRLSCKASGYTFSRYGIYWVRQAPGKGLEWIGYIDPGSGGTR
YNEKFKGKATISRDNSKNTLYLQMNSLRAEDTAVYYCARYHYGYSFDYWGQGTLVTV
SSAS
>
QVQLLESGGGLVQPGGSLRLSCKASGYTFSRYGYYWVRQVPGKGLEWIGYIDPGNGGT
SYNEKFKGKATISRDNSKNTLYLQMNSLRAEDTAVYYCARYHDGYSFDYWGQGTLVT
VSSAS
>
QVQLLESGGGLVQPGGSLRLSCKASGYTFSRYAYNWVRQAPGKGLEWIGWIDPGSGGT
RYNQKFKGKATISRDNSKNTLYLQMNSLRAEDTAVYYCARERFAYYLDYWGQGTLVT
VSSAS
>
QVQLLESGGGLVQPGGSLRLSCKASGYTFSRYGFSWVRQAPGKGLEWIGRIDPGSGGTR
YNQQFQGKATISRDNSKNTLYLQMNSLRAEDTAVYYCARSRDGYYFDYWGQGTLVTV
SSAS
>
QVQLLESGGGLVQPGGSLRLSCKASGYTFSRYAFNWVRQAPGKGLEWIGYIDPGSGGT
RYNEKFQGKATISRDNSKNTLYLQMNSLRAEDTAVYYCARARDGYSFDYWGQGTLVT
VSSAS
>QVQLLESGGGLVQPGGSLRLSCKASGYTFSSYGIYWVRQAPGKGLEWIGWIDPGSGGT
RYNEKFQSKATISRDNSKNTLYLQMNSLRAEDTAVYYCARERAGYYLDYWGQGTLVT
VSSAS
>
QVQLLESGGGLVQPGGSLRLSCKASGYTFSRYTITWVRQAPGKGLEWIGYIDPGSGGTK
YNEKFQGKATISRDNSKNTLYLQMNSLRAEDTAVYYCARHRVGYYFDYWGQGTLVTV
SSAS
>
QVQLLESGGGLVQPGGSLRLSCKASGYTFSRYSFIWVRQAPGKGLEWIGYIDPGSGGTK
YNQKFKGKATISRDNSKNTLYLQMNSLRTEDTAVYYCARYRSGYSLDYWGQGTLVTV
SSAS
>
QVQLLESGGGLVQPGGSLRLSCKASGYTFSRYTFYWVRQAPGKGLEWIGYIDPGSGGT
RYNQKFKGKATISRDNSKNTLYLQMNSLRAEDTAVYYCARARVGYSFDYWGQGTLVT
VSSAS
>
QVQLLESGGGLVQPGGSLRLSCKASGQTFSSYGFNWVRQAPGKGLEWIGWIDPGSGGT
RYNQKFKSKATISRDNSKNTLYLQMNSLRAEDTAVYYCARANVGYSFDYWGQGTLVT
VSSAS
>
QVQLLESGGGLVQPGGSLRLSCKASGYTFSRYGYYWVRQAPGKGLEWIGRIDPGSGGT
SYNEKFQSKATISRDNSKNTLYLQMNSLRAEDTAVYYCARARVG?SFDYWGQGTLVTV
SSAS
>
QVQLLESGGGLVQPGGSLRLSCKASGYTFSRYGIYWVRQAPGKGLEWIGRIDPGNGGT
RYNQKFQSKATISRDNSKNTLYLQMNSLRAEDTAVYYCARSRVGYSFDYWGQGTLVT
VSSAS
>
QVQLLESGGGLVQPGGSLRLSCKASGYTFSRYANYWVRQAPGKGLEWIGYIDPGSGGT
KYNEKFKGKATISRDNSKNTLYLQMNSLRAEDTAVYYCARSHVGYYFDYWGQGTLVT
VSSAS
>
QVQLLESGGGLVQPGGSLRLSCKASGYTFSRYSNSWVRQAPGKGLEWIGWIDPGNGGT
RYNQKFQSKATISRDNSKNTLYLQMNSLRAEDTAVYYCARARVGYSFDYWGQGTLVT
VSSAS
>
QVQLLESGGGLVQPGGSLRLSCKASGYTFSSYSITWVRQAPGKGLEWIGWIDPGSGGTR
YNEKFKSKATISRDNSKNTLYLQMNSLRAEDTAVYYCARHRVGYSLDYWGQGTLVTV
SSAS
>
QVQLLESGGGLVQPGGSLRLSCKASGYTFSRYGFSWVRQAPGKGLEWIGWIDPGNGGT
RYNQQFKGKATISRDNSKNTLYLQMNSLRAEDTAVYYCARSRVGYSLDYWGQGTLVT
VSSAS
>
QVQLLESGGGLVQPGGSLRLSCKASGYTFSSYTYTWVRQAPGKGLEWIGWIDPGSGGT
RYNEKFEGKATISRDNSKNTLYLQMNSLRAEDTAVYYCARSRVGYSLDYWGQGTLVT
VSSAS
>
QVQLLESGGGLVQPGGSLRLSCKASGYTFSSYSYYWVRQAPGKGLEWIGRIDPGSGGT
RYNQKFQGKATISRDNSKNTLYLQMNSLRAEDTAVYYCARYRDGYSFDYWGQGTLVT
VSSAS
>
QVQLLESGGGLVQPGGSLRLSCKASGYTFSSYGYSWVRQAPGKGLEWIGRIDPGSGGT
KYNEKFKGKATISRDNSKNTLYLQMNSLRAEDTAVYYCARDRVGYSLDYWGQGTLVT
VSSAS
>
QVQLLESGGGLVQPGGSLRLSCKASGYTFSRYTFSWVRQAPGKGLEWIGYIDPGNGGT
RYNEQFKGKATISRDNSKNTLYLQMNSLRAEDTAVYYCARQRVGYNLDYWGQGTLVT
VSSAS
>
QVQLLESGGGLVQPGGSLRLSCKASGYTFSSYGFSWVRQAPGKGLEWIGWIDPGNGGT
RYNEQFQGKATISRDNSKNTLYLQMNSLRAEDTAVYYCARQRVGYSLDYWGEGTLVT
VSSAS
>
QVQLLESGGGLVQPGGSLRLSCKASGYTFSRYSNSWVRQAPGKGLEWIGWIDPGNGGT
RYNEQFQGKATISRDNSKNTLYLQMNSLRAEDTAVYYCARQRVGYSLDYWGQGTLVT
VSSAS
>
QVQLLESGGGLVQPGGSLRLSCKASGYTFSSYGQSWVRQAPGKGLEWIGRIDPGSGGT
RYNEKFQGKATISRDNSKNTLYLQMNSLRAEDTAVYYCARSRDGYSLDYWGQGTLVT
VSSAS
>
QVQLLESGGGLVQPGGSLRLSCKASGYTFSSYAISWVRQAPGKGLEWIGRIDPGSGGTR
YNEKFQSKATISRDNSKNTLYLQMNSLRAEDTAVYYCARSRDGYSLDYWGQGTLVTV
SSAS
>
QVQLLESGGGLVQPGGSLRLSCKASGYTFSRYSYYWVRQAPGKGLEWIGYIDPGSGGT
KYNQKFKGKATISRDNSKNTLYLQMNSLRAEDTAVYYCARYRDGYSFDYWGQGTLVT
VSSAS
>
QVQLLESGGGLVQPGGSLRLSCKASGYTFSSYTYNWVRQAPGKGLEWIGYIDPGNGGT
NYNQKFQSKATISRDNSKNTLYLQMNSLRAEDTAVYYCARSRVGYSLDYWGQGTLVT
VSSAS
>
QVQLLESGGGLVQPGGSLRLSCKASGYTFSSYTISWVRQAPGKGLEWIGYIDPGNGGTR
YNQQFQGKATISRDNSKNTLYLQMNSLRVEDTAVYYCARSRVGYSFDYWGQGTLVTV
SSAS
>
QVQLLESGGGLLQPGGSLRLSCKASGYTFSRYTFNWVRQAPGKGLEWIGWIDPGSGGT
RYNQKFQSKATISRDNSKNTLYLQMNSLRAEDTAVYYCARARVGYSFDYWGQGTLVT
VSSAS
>
QVQLLESGGGLVQPGGSLRLSCKGQRLPPRYGYYWVRQAPGKGLEWIGYIDPGNGGTR
YNEKFQSKATISRDNSKNTLYLQMNSLRAEDTAVYYCARYRSGYYLDYWGQGTLVTV
SSAS
>
QVQLLESGGGLVQPGGSLRLSCKASGYTYSRYTFSWVRQAPGKGLEWIGYIDPGSGGT
KYNQKFKGKATISRDNSKNTLYLQMNSLRAEDTAVYYCARARVGYSFDYWGQGTLVT
VSSAS
>
QVQLLESGGGLVQPGGSLRLSCKASGYTFSSYGYYWVRQAPGKGLEWIGYIDPGSGGT
RYNQKFKSKATISRDNSKNTLYLQMNSLRAEDTAVYYCARYRVGYSFDYWGQGTLVT
VSSAS
>
QVQLLESGGGLVQPGGSLRLSCKASGYTFSRYGFSWVRQAPGKGLEWIGRIDPGSGGTR
YNEKFQSKATISWDNSKNTLYLQMNSLRAEDTAVYYCARSRDGYSFDYWGQGTLVTV
SSAS
>
QVQLLESGGGLVQPGGSLRLSCKASDYTFSSYGNNWVRQAPGKGLEWIGRIDPGNGGT
RYNEKFQGKATISRDNSKNTLYLQMNSLRAEDTAVYYCARARDGYSLDYWGQGTLVT
VSSAS
>
QVQLLESGGGLVQPGGSLRLSCKASGYTFSSYGISWVRQAPGKGLEWIGWIDPGNGGT
KYNQKFQSKATISRDNSKNTLYLQMNSLRAEDTAVYYCARSRVGYSLDYWGQGTLVT
VSSAS
>
QVQLLESGGGLVQPGGSLRLSCKASGYTFSSYTINWVRQAPGKGLEWIGWIDPGSGGTR
YNEKFQGKATISRDNSKNTLYLQMNSLRAEDTAVYYCARHRVGYSFDYWGQGTLVTV
SSAS
>
QVQLLESGGGLVQPGGSLRLSCKASGYTFSRYGISWVRQAPGKGLEWIGRIDPGSGGTR
YNEKFQSKATISRDNSKNTLYLQMNSLRAQDTAVYYCARANDGYSFDYWGQGTLVTV
SSAS
>
QVQLLESGGGLVQPGGSLRLSCKASGYTFSRYGINWVRQAPGKGLEWIGRIDPGNGGT
RYNEKFKGKATISRDNSKNTLYLQMNSLRAEDTAVYYCARADVGYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGFVYYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGFIYYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGFLYYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGRIYYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGRFYYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGRLYYSLDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGFIYYDFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGKIYYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGYLYYSFDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGFIYYSLDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGNLYYSLDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCSRGKVGYSLDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGSIGYNLDYWGQGTLVT
VSSAS
>
EVQLLESGGGLVQPGGSLRLSCKASGYTFSSYGMYWVRQAPGKGLEWIGWIDPGSGGT
KYNEKFKGKATISRDNSKNTLYLQMNSLRAEDTAVYYCARGRDGYSLDYWGQGTLVT
VSSAS
>
EVQLLESGGGLVQPGGSLRLSCKASGFTFSAYGMYWVRQAPGKGLEWIGRIDPSNGGT
KYNQKFKGKATISRDNSKNTLYLQMNSLRAEDAAVYYCARERYGYYFDYWGQGTLV
TVSSAS
>
EVQLLESGGGLVQPGGSLRLSCKASGFTFSDYGMYWVRQAPGKGLEWIGRINPNSGGT
KYNEKFKGKATISRDNSKNTLYLQMNSLRAEDTAVYYCARERYGYYFDYWGQGTLVT
VSSAS
>
EVQLLESGGGLVQPGGSLRLSCKASGFTFSSYQMSWVRQAPGKGLEWIGRIDPGSGGTK
YNQKFKGKATISRDNSKNTLYLQMNSLRAEDTAVYYCARERYGYYFDYWGQGTLVTV
SSAS
>
EVQLLESGGGLVQPGGSLRLSCKASGFTFSYYYMYWVRQAPGKGLEWVGRISPSGGST
YYADSVKGRFTISRDNSKNTLYLQMNSLRAEDTAVYYCARERYGYYFDYWGQGTLVT
VSSAS
>
EVQLLESGGGLVQPGGSLRLSCKASGFTFTAYGMYWVRQAPGKGLEWIGRIDPGSGGT
KYNQKFKGKATISRDNSKNTLYLQMNSLRAEDTAVYYCARERYGYYFDYWGQGTLVT
VSSAS
>
EVQLLESGGGLVQPGGSLRLSCKASGFTFTSYQMSWVRQAPGKGLEWIGRINPGSGGT
KYDEKFKGRATISRDNSKNTLYLQMNSLRAEDTAVYYCARERYGYYFDYWGQGTLVT
VSSAS
>
EVQLLESGGGLVQPGGSLRLSCKASGFTFTSYWMSWVRQAPGKGLEWIGRINPGSGGT
KYDEKFKGRATISRDNSKNTLYLQMNSLRAEDTAVYYCARERYGYYFDYWGQGTLVT
VSSAS
>
EVQLLESGGGLVQPGGSLRLSCKASGLTFTDYGMYWVRQAPGKGLEWIGWINPDSGST
KYAEKFKGRATISRDNSKNTLYLQMNSLRAEDTAVYYCARERYGYYFDYWGQGTLVT
VSSAS
>
EVQLLESGGGLVQPGGSLRLSCKASGYTFSYYGMYWVRQAPGKGLEWIGRIDPGSGGT
KYNEKFKGKATISRDNSKNTLYLQMNSLRAEDTAVYYCARERYGYYFDYWGQGTLVT
VSSAS
>
EVQLLESGGGLVQPGGSLRLSCKASGYTFTDYGMYWVRQAPGKGLEWIGWIDPGSGG
TKYNEKFKGKATISRDNSKNTLYLQMNSLRAEDTAVYYCARERYGYYFDYWGQGTLV
TVSSAS
>
EVQLLESGGGLVQPGGSLRLSCKASGYTFTNYGMNWVRQAPGKGLEWIGRIDPSSGGT
KYNQKFKGKATISRDNSKNTLYLQMNSLRAEDTAVYYCARERYGYYFDYWGQGTLVT
VSSAS
>
EVQLLESGGGLVQPGGSLRLSCKASGYTFTSYGMYWVRQAPGKGLEWIGRIDPSSGGT
KYNQKFKGKATISRDNSKNTLYLQMNSLRAEDTAVYYCARERYGYYFDYWGQGTLVT
VSSAS
>
EVQLLESGGGLVQPGGSLRLSCKASGYTFTSYWMSWVRQAPGKGLEWVGRISPGGGTT
YYADSVKGRFTISRDNSKNTLYLQMNSLRAEDTAVYYCARERYGYYFDYWGQGTLVT
VSSAS
>
EVQLLESGGGLVQPGGSLRLSCQASGYTFSSYGMYWVRQAPGKGLEWIGRINPGSGGT
NYDEKFKGRATISRDNSKNTLYLQMNSLRAEDTAVYYCARERYGYYFDYWGQGTLVT
VSSAS
>
EVQLLESGGGLVQPGGSLRLSCEASGFTFSSYGMYWVRQAPGKGLEWIGRINPKQRWH
KYNQKFKGKVTISRDNSKNTLYLQMNSLRAEDTAVYYCARERYGYYFDYWGQGTLVT
VSSAS
>
EVQLLESGGGLVQPGGSLRLSCEASGFSFSNYGMYWVRQAPGKOLEWIGLDRPRQRW
HQLNEKFQGRATISRDNSKNTLYLQMNSLRAEDTAVYYCARERYGYYFDYWGQGTLV
TVSSAS
>
EVQLLESGGGLVQPGGSLRLSCAASGYSFSAYYIHWVRQAPGKGLESIGRIDPGSGGTK
YNEKFQGRATISRDNSKNTLYLQMNSLRAEDIAVYYCARERYGYYFDYWGQGTLVTV
SSAS
>
EVQLLESGGGLVQPGGSLRLSCKASGFSFSSYGIYWVRQAPGKGLEWIGLDRPKQRVGT
KYNQKFQGRVTISRDNSKNTLYLQMNSLRAEDTAVYYCARERYGYYFDYWGQGTLVT
VSSAS
>
EVQLLESGGGLVQPGDSLRLSCAASGYSFSTFGIYWVRQAPGKGLEWIGLDRPRQRWP
KYNEKFKGRATISRDNSKNTLYLQMNSLRAEDTAVYYCARERYGYYFDYWGQGTLVT
VSSAS
>
EVQLLESGGGLVQPGGSLRLSCEASGFTFSDYGIHWVRQAPGKGLEWIGLDRPKQRWH
QYNEKFQGRATISRDNSKNTLYLQMNSLRAEDTAVYYCARERYGYYFDYWGQGTLVT
VSSAS
>
EVQLLESGGGLVQPGGSLRLSCAASGYTFSDFGIYWVRQAPGKGLEWIGSDRPRQRVAP
KYNQKFKGRATISRDNSKNTLYLQMNSLRAEDTAVYYCARERYGYYFDYWGQGTLVT
VSSAS
>
EVQLLESGGGLVQPGGSLRLSCQASGYSFTTYGMYWVRQAPGKGLEWIGRIDPSSGGT
KYNQKFQGRVTISRDNSKNTLYLQMNSLRAEDTAVYYCARERYGYYFDYWGQGTLVT
VSSAS
>
EVQLLESGGGLVQPGGSLRLSCAASGFTFTNYGMYWVRQAPGKGLEWIGLDRPKQRGT
KYNQKFQGRATISRDNSKNTLYLQMNSLRAEDTAVYYCARERYGYYFDYWGQGTLVT
VSSAS
>
EVQLLESGGGLVQPGGSLRLSCKASGYTFSYFGIYWVRQAPGKGLEWIGSDRPRQRWH
QVQRKFKGRATISRDNSKNTLYLQMNSLRAEDTAVYYCARERYGYYFDYWGQGTLVT
VSSAS
>
EVQLLESGGGLVQPGGSLRLSCTASGFTFSNYGMYWVRQAPGKGLEWIGRIDPNSGGT
KYNEKFQGRATISRDNSKNTLYLQMNSLRAEDTAVYYCARERYGYYFDYWGQGTLVT
VSSAS
>
EVQLLESGGGLVQPGGSLRLSCTASGYSFTAFGMYWVRQAPGKGLEWIGRINPSSGGT
NYNEQFQGRATISRDNSKNTLYLQMNSLRAEDTAVYYCARERYGYYFDYWGQGTLVT
VSSAS
>
EVQLLESGGGLVQPGGSLRLSCKASGFTFSSYGMYWVRQAPGKGLEWIGSDRPRQRW
HQVQPKVQGRATISRDNSKNTLYLQMNSLRAEDTAVYYCARERYGYYFDYWGQGTLV
TVSSAS
>
EVQLLESGGGLVQPGGSLRLSCKASGYSFSAYGMYWVRQAPGKGLEWIGRIDPNSGGT
KQNEKFQGRATISRDNSKNTLYLQMNSLRAEDTAVYYCARERYGYYFDYWGQGTLVT
VSSAS
>
EVQLLESGGGLVQPGGSLRLSCEASGYTFTSFQMHWVRQAPGKGLESIGRIDPGSGGTK
YNQKFQGRATISRDNSKNTLYLQMNSLRAEDTAVYYCARERYGYYFDYWGQGTLVTV
SSAS
>
EVQLLESGGGLVQPGGSLRLSCAASGYTFSSFSMYWVRQAPGKGLEWIGLDRPRQRWH
QVNEKFQGRATISRDNSKNTLYLQMNSLRAEDTAVYYCARERYGYYFDYWGQGTLVT
VSSAS
>
EVQLLESGGGLVQPGGSLRLSCQASGYTFTNYGMYWVRQAPGKGLEWIGRIDPGNGGT
KYNEKFKGKVTISRDNSKNTLYLQMNSLRAEDTAVYYCARERYGYYFDYWGQGTLVT
VSSAS
>
EVQLLESGGGLVQPGGSLRLSCQASGYTFTAFGMYWVRQAPGKGLEWIGRIDPGSGGT
KYNEKFQGKVTISRDNSKNTLYLQMNSLRAEDTAVYYCARERYGYYFDYWGQGTLVT
VSSAS
>
EVQLLESGGGLVQPGGSLRLSCKASGFSFTSYGMYWVRQAPGKGLEWIGQDRPKYGGT
KYNEKFKGRVTISRDNSKNTLYLQMNSLRAEDTAVYYCARERYGYYFDYWGQGTLVT
VSSAS
>
EVQLLESGGGLVQPGGSLRLSCQASGFTFSTYGIYWVRQAPGKGLEWIGRINPSNGGTK
YNEKFKGRATISRDNSKNTLYLQMNSLRAEDTAVYYCARERYGYYFDYWGQGTLVTV
SSAS
>
EVQLLESGGGLVQPGGSLRLSCKASGFSFSSYGMYWVRQAPGKGLEWIGSDRPRQRWG
TKYNEKFQGRVTISRDNSKNTLYLQMNSLRAEDTAVYYCARERYGYYFDYWGQGTLV
TVSSAS
>
EVQLLESGGGLVQPGGSLRLSCEASGYSFSNFGMYWVRQAPGKGLEWIGWINPSNGGT
KYNEKFKGRATISRDNSKNTLYLQMNSLRAEDTAVYYCARERYGYYFDYWGQGTLVT
VSSAS
>
EVQLLESGGGLVQPGGSLRLSCTASDFSFSTFSMYWVRQAPGKGLEWIGSDQPRQGGT
KYNEKFKGRATISRDNSKNTLYLQMNSLRAEDTAVYYCARERYGYYFDYWGQGTLVT
VSSAS
>
EVQLLESGGGLVQPGGSLRLSCAASGYTFSDFGIYWVRQAPGKGLEWIGSDRPRQGGT
KYNEKFKDRVTISRDNSKNTLYLQMNSLRAEDTAVYYCARERYGYYFDYWGQGTLVT
VSSAS
>
EVQLLESGGGLVQPGGSLRLSCAASGYSFTSFGIYWVRQAPGKGLEWIGSDRPKQRGTK
YNEKFKGKVTISRDNSKNTLYLQMNSLRAEDTAVYYCARERYGYYFDYWGQGTLVTV
SSAS
>
EVQLLESGGGLVQPGGSLRLSCQASGFSFSDYYMSWVRQAPGKGLESIGRINPGSGGTK
YNEKFQGRATISRDNSKNTLYLQMNSLRAEDTAVYYCARERYGYYFDYWGQGTLVTV
SSAS
>
EVQLLESGGGLVQPGGSLRLSCAASGYSFSDYGIYWVRQAPGKGLEWIGSDRPKQRWH
QVNEKFQGKVTISRDNSKNTLYLQMNSLRAEDTAVYYCARERYGYYFDYWGQGTLVT
VSSAS
>
EVQLLESGGGLVQPGGSLRLSCKASGYSFSTYYMYWVRQAPGKGLEWIGRIDPDSGGT
KYNEKFQGRATISRDNSKNTLYLQMNSLRAEDTAVYYCARERYGYYFDYWGQGTLVT
VSSAS
>
EVQLLESGGGLVQPGGSLRLSCEASGYTFSAFQIYWVRQAPGKGLEWIGRIDPGNGGTK
YNEKFKGKATISRDNSKNTLYLQMNSLRAEDTAVYYCARERYGYYFDYWGQGTLVTV
SSAS
>
EVQLLESGGGLVQPGGSLRLSCQASGFSFSNFYMYWVRQAPGKGLEWIGRIDPGSGGT
KYNEKFQGRATISRDNSKNTLYLQMNSLRAEDTAVYYCARERYGYYFDYWGQGTLVT
VSSAS
>
EVQLLESGGGLVQPGGSLRLSCKASGFTFSAFGIYWVRQAPGKGLEWIGRINPDNGGTK
YNEKFQGRVTISRDNSKNTLYLQMNSLRAEDTAVYYCARERYGYYFDYWGQGTLVTV
SSAS
>
EVQLLESGGGLVQPGGSLRLSCKASGYTFSTYGMYWVRQAPGKGLEWIGRDQPRQGG
TNYNEKFQGRATISRDNSKNTLYLQMNSLRAEDTAVYYCARERYGYYFDYWGQGTLV
TVSSAS
>
EVQLLESGGGLVQPGGSLRLSCAASGYSFSTYGIYWVRQAPGKGLEWIGRINPNNGGTK
YNEKFQGKATISRDNSKNTLYLQMNSLRAEDTAVYYCARERYGYYFDYWGQGTLVTV
SSAS
>
EVQLLESGGGLVQPGGSLRLSCTASGFSFSAYGMYWVRQAPGKGLEWIGSDRPRQGGT
KYNEKFKGRVTISRDNSKNTLYLQMNSLRAEDTAVYYCARERYGYYFDYWGQGTLVT
VSSAS
>
EVQLLESGGGLVQPGGSLRLSCAASGFSFSNYYMYWVRQAPGKGLEWIGRIDPGSGGT
KYNEKFQGRATISRDNSKNTLYLQMNSLRAEDTAVYYCARERYGYYFDYWGQGTLVT
VSSAS
>
EVQLLESGGGLVQPGGSLRLSCEASGYTFTSYGMYWMRQAPGKGLEWIGRIDPGSGGT
KYNEKFQGRVTISRDNSKNTLYLQMNSLRAEDTAVYYCARERYGYYFDYWGQGTLVT
VSSAS
>
EVQLLESGGGLVQPGGSLRLSCAASGFTFTNYGMYWVRQAPGKGLEWIGSDRPKQRW
APKYNEKFKGRATISRDNSKNTLYLQMNSLRAEDTAVYYCARERYGYYFDYWGQGTL
VTVSSAS
>
EVQLLESGGGLVQPGGSLRLSCKASGYTFSDFAMYWVRQAPGKGLEWIGSDQPRQRW
HQVQPKVQGRVTISRDNSKNTLYLQMNSLRAEDTAVYYCARERYGYYFDYWGQGTLV
TVSSAS
>
EVQLLESGGGLVQPGGSLRLSCKASGYSFTNFGMYWVRQAPGKGLEWIGRINPGNGGT
KQNEKFKGRATISRDNSKNTLYLQMNSLRAEDTAVYYCARERYGYYFDYWGQGTLVT
VSSAS
>
EVQLLESGGGLVQPGGSLRLSCAASGYSFSYYGMYWVRQAPGKGLEWIGRINPSSGGT
KYNEKFKGRATISRDNSKNTLYLQMNSLRAEDTAVYYCARERYGYYFDYWGQGTLVT
VSSAS
>
EVQLLESGGGLVQPGGSLRLSCEASGYSFSAFGIYWVRQAPGKGLEWIGRINPNSGGTK
YNEKFKGRVTISRDNSKNTLYLQMNSLRAEDTAVYYCARERYGYYFDYWGQGTLVTV
SSAS
>
EVQLLESGGGLVQPGGSLRLSCKTSGYTFSAFQIYWVRQAPGKGLEWIGRIDPGSGGTK
YNQKFKGRTTISRDNSKNTLYLQMNSLRAEDTAVYYCARERYGYYFDYWGQGTLVTV
SSAS
>
EVQLLESGGGLVQPGGSLRLSCKASGYTFSSYGMYWVRQAPGKGLEWIGWIDPGSGGT
KYQDKFKGKATISRDNSKNTLYLQMNSLRAEDTAVYYCAREKYGYYFDYWGQGTLVT
VSSAS
>
EVQLLESGGGLVQPGGSLRLSCKASGYTFSSYGMYWVRQAPGKGLEWIGWIDPGSGGT
KYQEKFKGKVTISRDNSKNTLYLQMNSLRAEDTAVYYCAREKYGYYFDYWGQGTLVT
VSSAS
>
EVQLLESGGGLVQPGGSLRLSCKASGYTFSSYGMYWVRQAPGKGLEWIGWIDPGSGGT
KYQEKFKGKATISRDNSKNTLYLQMNSLRAEDTAVYYCARYKSGWYFDYWGQGTLV
TVSSAS
>
EVQLLESGGGLVQPGGSLRLSCKASGYTFSSYGMYWVRQAPGKGLEWIGWIDPGSGGT
KYQEKFKGKATISRDNSKNTLYLQMNSLRAEDTALYYCAREKYGYYFDYWGQGMLV
TVSSAS
>
EVQLLESGGGLVQPGGSLRLSCKASGYTFSSYGMYWVRQAPGKGLEWIGWIDPGSGGT
NYQEKFKGKATISRDNSKNTLYLQMNSLRAEDTAVYYCARYRSGYYFDYWGQGTLVT
VSSAS
>
EVQLLESGGGLVQPGGSLRLSCKASGYTFSSYGMYWVRQAPGKGLEWIGWIDPGSGGT
KYQEKFKGKATISRDNSKNTLYLQMNSLRAEDTAVYYCARYRSGYYFDYWGQGTLVT
VSSAS
>
EVQLLESGGGLVQPGGSLRLSCKASGYTFSSYGMYWVRQAPGKGLEWIGWIDPGSGGT
NYQQKFKGKATISRDNSKNTLYLQMNSLRAEDTAVYYCARYKSGYYFDYWGQGTLVT
VSSAS
>
EVQLLESGGGLVQPGGSLRLSCKASGYTFSSYGMYWVRQAPGKGLEWIGWIDPGSGGT
KYADKFKGKATISRDNSKNTLYLQMNSLRAEDTAVYYCARYRSGYYFDYWGQGTLVT
VSSAS
>
EVQLLESGGGLVQPGGSLRLSCKASGYTFSSYGMYWVRQAPGKGLEWIGWIDPGSGGT
KYEEKFKGKATISRDNSKNTLYLQMNSLRAEDTAVYYCARERYGYYFDYWGQGTLVT
VSSAS
>
EVQLLESGGGLVQPGGSLRLSCKASGYTFSSYGMYWVRQAPGKGLEWIGWIDPGSGGT
KYQDKFKGKATISRDNSKNTLYLQMNSLRAEDTAVYYCAREKSGYYFDYWGQGTLVT
VSSAS
>
EVQLLESGGGLVQPGGSLRLSCKASGYTFSSYGMYWVRQAPGKGLEWIGWIDPGSGGT
KYQEKFKGKATISRDNSKNTLYLQMNSLRAEDTAVYYCAREKYGYYFDYWGQGTLVT
VSSAS
>
EVQLLESGGGLVQPGGSLRLSCKASGYTFSSYGMYWVRQAPGKGLEWIGWIDPGSGGT
KYNDKFKGKATISRDNSKNTLYLQMNSLRAEDTAVYYCARYRSGYYFDYWGQGTLVT
VSSAS
>
EVQLLESGGGLVQPGGSVRLSCKASGYTFSSYGMYWVRQAPGKGLEWIGWIDPGSGGT
KYYEKFKGKATISRDNSKNTLYLQMNSLRAEDTAVYYCARYKSGYYFDYWGQGTLVT
VSSAS
>
EVQLLESGGGLVQPGGSLRLSCKASGYTFSSYGMYWVRQAPGKGLEWIGWIDPGSGGT
KYEEKFKGKATISRDNSKNTLYLQMNSLRAEDTAVYYCARYKSGYYFDYWGQGTLVT
VSSAS
>
EVQLLESGGGLVQPGGSLRLSCKASGYTFSSYGMYWMRQAPGKGLEWIGWIDPGSGG
TKYEDKFEGKATISRDNSKNTLYLQMNSLRAEDTAVYYCARERSGYYFDYWGQGTLV
TVSSAS
>
EVQLLESGGGLVQPGGSLRLSCKASGYTFSSYGMYWVRQAPGKGLEWIGWIDPGSGGT
NYTQKFKGKATISRDNSKNTLYLQMNSLRAEDTAVYYCARYRSGYYFDYWGQGTLVT
VSSAS
>
EVQLLESGGGLVQPGGSLRLSCKASGYTFSSYGMYWVRQAPGKGLEWIGWIDPGSGGT
KYSHKFKGKATISRDNSKNTLYLQMNSLRAEDTAVYYCARERYGYYFDYWGQGTLVT
VSSAS
LIST OF VK SEQUENCES
>
EIVLTQSPATLSLSPGERATITCRASQYVGTYLNWYQQKPGQAPRLLIYDASNRATGIPA
RFSGSGSGTDFTLTISSLEPEDFAVYYCQVWDSSPPVAFGGGTKVEIK
>
EIVLTQSPATLSLSPGERATITCRASQYVGSYLNWYQQKPGQAPRLLIYDASNRATGIPA
RFSGSGSGTDFTLTISSLEPEDFAVYYCQVWDSSPPVVFGGGTKVEIK
>
EIVLTQSPATLSLSPGERATITCRASQYVGTYLNWYQQKPGQAPRLLIYDASNRATGIPA
RFSGSGSGTDFTLTISSLEPEDFAVYYCQVWDSSPPVVFGGGTKVEIK
>
EIVLTQSPATLSLSPGERATITCRASQYVGSYLNWYQQKPGQAPRLLIYDASNRATGIPA
RFSGSGSGTDFTLTISSLEPEDFAVYYCQVWDSSPPVAFGGGTKVEIK
>
EIVLTQSPATLSLSPGERATITCRASQYVGSYLNWYQQKPGQAPRLLIYDASNRATGIPA
RFSGSGSGTDFTLTISSLEPEDFAVYYCQVWDSSPPLVFGGGTKVEIK
>
EIVLTQSPATLSLSPGERATITCRASQYVGSYLNWYQQKPGQAPRLLIYDASNRATGIPA
RFSGSGSGTDFILTISSLEPEDFAVYYCQVWDSSPPLAFGGGTKVEIK
>
EIVLTQSPATLSLSPGERATITCRASQYIGSYLNWYQQKPGQAPRLLIYDASNRATGIPAR
FSGSGSGTDFTLTISSLEPEDFAVYYCQAWDSSPPVVFGGGTKVEIK
>
EIVLTQSPATLSLSPGERATITCRASQYVGSYLNWYQQKPGQAPRLLIYDASNRATGIPA
RFSGSGSGTDFTLTISSLEPEDFAVYYCQVWDSSPPVMFGGGTKVEIK
>
DIQMTQSPSSLSASVGDRVTITCRASQAISNYLTWYQQKPGKAPKLLIYAASSLQSGVPS
RFSGSGSGTDFTLTISSLQPEDFATYYCQGWDSSPTFGGGTKVEIK
>
EIVLTQSPATLSLSPGERATITCRASQYVGTYLNWYQQKPGQAPRLLIYDASNRATGIPA
RFSGSGSGTDFTLTISSLEPEDFAVYYCQVWDSSPPLVFGGGTKVEIK
>
EIVLTQSPATLSLSPGERATITCRASQYVGSYLTWYQQKPGQAPRLLIYDASNRATGIPA
RFSGSGSGTDFTLTISSLEPEDFAVYYCQVWDSSPPVVFGGGTKVEIK
>
EIVLTQSPATLSLSPGERATITCRASQYVGTYLNWYQQKPGQAPRLLIYDASNRATGIPA
RFSGSGSGTDFTLTISSLEPEDFAVYYCQVWDNSPPVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWDSSPPVVFGGGTKVEIK
>
EIVLTQSPATLSLSPGERATITCRASQYIGSYLNWYQQKPGQAPRLLIYDASNRATGIPAR
FSGSGSGTDFTLTISSLEPEDFAVYYCQAWDSSPPVTFGDGTKVEIK
>
DIQMTQSPSSLSASVGDRVTITCRASQDVSNYLNWYQQKPGKAPKLLIYAASSLQSGVP
SRFSGSGSGTDFTLTISSLQPEDFATYYCQQSGSYLTFGGGTKVEIK
>
EIVLTQSPATLSLSPGERATITCQASQYVGSYLSWYQQKPGQAPRLLIYDASNRATGIPA
RFSGSGSGTDFTLTISSLEPEDFAVYYCQVWDSYHAVVFGGGTKVEIK
>
EIVLTQSPATLSLSPGERATITCQASQYVGSYLSWYQQKPGQAPRLLIYDASNRAAGIPA
RFSGSGSGTDFTLTISSLEPEDFAVYYCQVWGSYHAVMFGGGTKVEIK
>
EIVLTQSPATLSLSPGERATITCRASQYVGSYLSWYQQKPGQAPRLLIYDASNRAAGIPA
RFSGSGSGTDFTLNISSLEPEDFAVYYCQVWGSYHSVMFGGGTKVEIK
>
EIVLTQSPATLSLSPGERATITCQASQYVGSYLSWYQQKPGQAPRLLIYDASNRAAGIPA
RFSGSGSGTDFTLTISSLEPEDFAVYYCQVWDTDHSVVFGGGTKVEIK
>
EIVLTQSPATLSLSPGERATITCQASQYVGSYLSWYQQKPGQAPRLLIYDASNRATGIPA
RFSGSGSGTDFTLTISSLEPEDFAVYYCQVWDTDHAVAFGGGTKVEIK
>
EIVLTQSPATLSLSPGERATITCRASQYVGSYLSWYQQKPGQAPRLLIYDASNRASGIPA
RFSGSGSGTDFTLTISSLEPEDFAVYYCQVWDSSHSVIFGGGTKVEIK
>
EIVLTQSPATLSLSPGERATITCRASQYVGSYLSWYQQKPGQAPRLLIYDASNRATGIPA
RFSGSGSGTDFTLTISSLEPEDFAVYYCQVWDSYPPVVFGGGTKVEIK
>
EIVLTQSPATLSLSPGERATITCRASQYVGSYLSWYQQKPGQAPRLLIYDASNRATGIPA
RFSGSGSGTDFTLTISSLEPEDFAVYYCQVWDSYHAVVFGGGTKVEIK
>
EIVLTQSPATLSLSPGERATITCRASQYVGSYLSWYQQKPGQAPRLLIYDASNRASGIPA
RFSGSGSGTDFTLTISSLEPEDFAVYYCQVWDSDHAVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWGSNHASLFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWGSTARVAFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWNSTPPVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWSSSPPVIFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWSSSPPVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWSSNHAVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWGSNPPVAFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWDSTPPVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWDSNPPVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQGYSSNDGVIFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWGSNHSVVFGGGTKVEIK
>
EIVLTQSPATLSLSPGERATITCRASQYVGSYLNWYQQKPGQAPRLLIYDAANRATGIPA
RFSGSGSGTDFTLTISSLEPEDFAVYYCQVWDSNHSVVFGGGTKVEIK
>
EIVLTQSPATLSLSPGERATITCRASQYVGSYLNWYQQKPGQAPRLLIYDASNRAAGIPA
RFSGSGSGTDFTLTISSLEPEDFAVYYCQVWDSSPPVVFGGGTKVEIK
>
EIVLTQSPATLSLSPGERATITCRASQYVGSYLNWYQQKPGQAPRLLIYDASNRATGIPA
RFSGSGSGTDFTLTISSLEPEDFAVYYCQVWDSSSAVVFGGGTKVEIK
>
EIVLTQSPATLSLSPGERATITCRASQYVGSYLNWYQQKPGQAPRLLIYDASNRATGIPA
RFSGSGSGTDLTLTISSLEPEDFAVYYCQVWDSSPPVVFGGGTKVEIK
LIST OF VH-CDR1 SEQUENCES
>
KASGYTFSSYGMYWVR
>
KASGYTFSSYSIYWVR
>
KASGYTFSRQGFTWVR
>
KASGYTFSSYSFSWVR
>
KASGYTFSRYGINWVR
>
KASGYTFSRYGISWVR
>
KASGYTFSSYTNYWVR
>
KASGYTFSRYSYYWVR
>
KASGYTFSSYANTWVR
>
KASGYTFSSYGYYWVR
>
KASGYTFSRYAINWVR
>
KASGYTFSRYGIYWVR
>
KASGYTFSRYGYYWVR
>
KASGYTFSRYAYNWVR
>
KASGYTFSRYGFSWVR
>
KASGYTFSRYAFNWVR
>
KASGYTFSSYGYYWVR
>
KASGYTFSRYTITWVR
>
KASGYTFSRYSFIWVR
>
KASGYTFSRYTFYWVR
>
KASGQTFSSYGFNWVR
>
KASGYTFSRYANYWVR
>
KASGYTFSRYSNSWVR
>
KASGYTFSSYSITWVR
>
KASGYTFSSYTYTWVR
>
KASGYTFSSYSYYWVR
>
KASGYTFSSYGYSWVR
>
KASGYTFSRYTFSWVR
>
KASGYTFSSYGFSWVR
>
KASGYTFSSYGQSWVR
>
KASGYTFSSYAISWVR
>
KASGYTFSSYTYNWVR
>
KASGYTFSSYTISWVR
>
KASGYTFSRYTFNWVR
>
KGQRLPPRYGYYWVR
>
KASGYTYSRYTFSWVR
>
KASDYTFSSYGNNWVR
>
KASGYTFSSYGISWVR
>
KASGYTFSSYTINWVR
>
KASGFTFSAYGMYWVR
>
KASGFTFSDYGMYWVR
>
KASGFTFSSYQMSWVR
>
KASGFTFSYYYMYWVR
>
KASGFTFTAYGMYWVR
>
KASGFTFTSYQMSWVR
>
KASGFTFTSYWMSWVR
>
KASGLTFTDYGMYWVR
>
KASGYTFSYYGMYWVR
>
KASGYTFTDYGMYWVR
>
KASGYTFTNYGMNWVR
>
KASGYTFTSYGMYWVR
>
KASGYTFTSYWMSWVR
>
QASGYTFSSYGMYWVR
>
EASGFTFSSYGMYWVR
>
EASGFSFSNYGMYWVR
>
AASGYSFSAYYIHWVR
>
KASGFSFSSYGIYWVR
>
AASGYSFSTFGIYWVR
>
EASGFTFSDYGIHWVR
>
AASGYTFSDFGIYWVR
>
QASGYSFTTYGMYWVR
>
AASGFTFTNYGMYWVR
>
KASGYTFSYFGIYWVR
>
TASGFTFSNYGMYWVR
>
TASGYSFTAFGMYWVR
>
KASGFTFSSYGMYWVR
>
KASGYSFSAYGMYWVR
>
EASGYTFTSFQMHWVR
>
AASGYTFSSFSMYWVR
>
QASGYTFTNYGMYWVR
>
QASGYTFTAFGMYWVR
>
KASGFSFTSYGMYWVR
>
QASGFTFSTYGIYWVR
>
KASGFSFSSYGMYWVR
>
EASGYSFSNFGMYWVR
>
TASDFSFSTFSMYWVR
>
AASGYSFTSFGIYWVR
>
QASGFSFSDYYMSWVR
>
AASGYSFSDYGIYWVR
>
KASGYSFSTYYMYWVR
>
EASGYTFSAFQIYWVR
>
QASGFSFSNFYMYWVR
>
KASGFTFSAFGIYWVR
>
KASGYTFSTYGMYWVR
>
AASGYSFSTYGIYWVR
>
TASGFSFSAYGMYWVR
>
AASGFSFSNYYMYWVR
>
EASGYTFTSYGMYWR
>
KASGYTFSDFAMYWVR
>
KASGYSFTNFGMYWVR
>
AASGYSFSYYGMYWVR
>
EASGYSFSAFGIYWVR
>
KTSGYTFSAFQIYWVR
>
KASGYTFSSYGMYWMR
LIST OF VH-CDR2 SEQUENCES
>
WIGWIDPGSGGTKYNEKFKGKAT
>
WIGWIDPGNGGTRYNQKFQSKAT
>
WIGWIDPGNGGTRYNEKFKGKAT
>
WIGYIDPGSGGTKYNQKFQGKAT
>
WIGRIDPGNGGTRYNEKFKGKAT
>
QIGWIDPGSGGTRYNQKFKSKAT
>
WIGYIDPGSGGTRYNQKFQGKAT
>
WIGYIDPGSGGTKYNQKFQSKAT
>
WIGWIDPGNGGTSYNQKFKSKAT
>
WIGWIDPGSGGTRYNQKFKGKAT
>
WIGWIDPGSGGTRYNEKFKGQAT
>
WIGYIDPGSGGTRYNEKFKGKAT
>
WIGYIDPGNGGTSYNEKFKGKAT
>
WIGRIDPGSGGTRYNQQFQGKAT
>
WIGYIDPGSGGTRYNEKFQGKAT
>
WIGWIDPGSGGTRYNEKFQSKAT
>
WIGYIDPGSGGTKYNEKFQGKAT
>
WIGYIDPGSGGTKYNQKFKGKAT
>
WIGYIDPGSGGTRYNQKFKGKAT
>
WIGWIDPGSGGTRYNQKFKSKAT
>
WIGRIDPGSGGTSYNEKFQSKAT
>
WIGRIDPGNGGTRYNQKFQSKAT
>
WIGYIDPGSGGTKYNEKFKGKAT
>
WIGWIDPGSGGTRYNEKFKSKAT
>
WIGWIDPGNGGTRYNQQFKGKAT
>
WIGWIDPGSGGTRYNEKFEGKAT
>
WIGRIDPGSGGTRYNQKFQGKAT
>
WIGRIDPGSGGTKYNEKFKGKAT
>
WIGYIDPGNGGTRYNEQFKGKAT
>
WIGWIDPGNGGTRYNEQFQGKAT
>
WIGRIDPGSGGTRYNEKFQGKAT
>
WIGRIDPGSGGTRYNEKFQSKAT
>
WIGYIDPGNGGTNYNQKFQSKAT
>
WIGYIDPGNGGTRYNQQFQGKAT
>
WIGWIDPGSGGTRYNQKFQSKAT
>
WIGYIDPGNGGTRYNEKFQSKAT
>
WIGYIDPGSGGTRYNQKFKSKAT
>
WIGRIDPGNGGTRYNEKFQGKAT
>
WIGWIDPGNGGTKYNQKFQSKAT
>
WIGWIDPGSGGTRYNEKFQGKAT
>
WIGRIDPSNGGTKYNQKFKGKAT
>
WIGRINPNSGGTKYNEKFKGKAT
>
WIGRIDPGSGGTKYNQKFKGKAT
>
WVGRISPSGGSTYYADSVKGRFT
>
WIGRINPGSGGTKYDEKFKGRAT
>
WIGWINPDSGSTKYAEKFKGRAT
>
WIGRIDPSSGGTKYNQKFKGKAT
>
WVGRISPGGGTTYYADSVKGRFT
>
WIGRINPGSGGTNYDEKFKGRAT
>
WIGRINPKQRWFIKYNQKFKGKVT
>
WIGLDRPRQRWHQLNEKFQGRAT
>
SIGRIDPGSGGTKYNEKFQGRAT
>
WIGLDRPKQRVGTKYNQKFQGRVT
>
WIGLDRPRQRWPKYNEKFKGRAT
>
WIGLDRPKQRWHQYNEKFQGRAT
>
WIGSDRPRQRVAPKYNQKFKGRAT
>
WIGRIDPSSGGTKYNQKFQGRVT
>
WIGLDRPKQRGTKYNQKFQGRAT
>
WIGSDRPRQRWIQVQRKFKGRAT
>
WIGRIDPNSGGTKYNEKFQGRAT
>
WIGRINPSSGGTNYNEQFQGRAT
>
WIGSDRPRQRWHQVQPKVQGRAT
>
WIGRIDPNSGGTKQNEKFQGRAT
>
SIGRIDPGSGGTKYNQKFQGRAT
>
WIGLDRPRQRWHQVNEKFQGRAT
>
WIGRIDPGNGGTKYNEKFKGKVT
>
WIGRIDPGSGGTKYNEKFQGKVT
>
WIGQDRPKYGGTKYNEKFKGRVT
>
WIGRINPSNGGTKYNEKFKGRAT
>
WIGSDRPRQRWGTKYNEKFQGRVT
>
WIGWINPSNGGTKYNEKFKGRAT
>
WIGSDQPRQGGTKYNEKFKGRAT
>
WIGSDRPRQGGTKYNEKFKDRVT
>
WIGSDRPKQRGTKYNEKFKGKVT
>
SIGRINPGSGGTKYNEKFQGRAT
>
WIGSDRPKQRWHQVNEKFQGKVT
>
WIGRIDPDSGGTKYNEKFQGRAT
>
WIGRIDPGNGGTKYNEKFKGKAT
>
WIGRIDPGSGGTKYNEKFQGRAT
>
WIGRINPDNGGTKYNEKFQGRVT
>
WIGRDQPRQGGTNYNEKFQGRAT
>
WIGRINPNNGGTKYNEKFQGKAT
>
WIGSDRPRQGGTKYNEKFKGRVT
>
WIGRIDPGSGGTKYNEKFQGRVT
>
WIGSDRPKQRWAPKYNEKFKGRAT
>
WIGSDQPRQRWHQVQPKVQGRVT
>
WIGRNPGNGGTKQNEKFKGRAT
>
WIGRINPSSGGTKYNEKFKGRAT
>
WIGRINPNSGGTKYNEKFKGRVT
>
WIGRIDPGSGGTKYNQKFKGRTT
>
WIGWIDPGSGGTKYQDKFKGKAT
>
WIGWIDPGSGGTKYQEKFKGKVT
>
WIGWIDPGSGGTKYQEKFKGKAT
>
WIGWIDPGSGGTNYQEKFKGKAT
>
WIGWIDPGSGGTNYQQKFKGKAT
>
WIGWIDPGSGGTKYADKFKGKAT
>
WIGWIDPGSGGTKYEEKFKGKAT
>
WIGWIDPGSGGTKYNDKFKGKAT
>
WIGWIDPGSGGTKYYEKFKGKAT
>
WIGWIDPGSGGTKYEDKFEGKAT
>
WIGWIDPGSGGTNYTQKFKGKAT
>
WIGWIDPGSGGTKYSHKFKGKAT
LIST OF VH-CDR3 SEQUENCES
>
CARERYGYYFDYWGQ
>
CARARVGYSLDYWGQ
> CARYRVGYSLDYWGQ
>
CARQRVGYSLDYWGQ
>
CARANDGYSFDYWGQ
>
CARARVGYSFDYWGQ
>
CARSRVGYSFDYWGQ
>
CARYRSGYSLDYWGQ
>
CARERVGYSLDYWGQ
>
CARHRVGYSLDFWGQ
>SEQ 381
CARYHYGYSFDYWGQ
>
CARYHDGYSFDYWGQ
>
CARERFAYYLDYWGQ
>
CARSRDGYYFDYWGQ
>
CARARDGYSFDYWGQ
>
CARERAGYYLDYWGQ
>
CARHRVGYYFDYWGQ
>
CARANVGYSFDYWGQ
> CARARVG?SFDYWG
>
CARSHVGYYFDYWGQ
> CARHRVGYSLDYWGQ
>
CARSRVGYSLDYWGQ
>
CARYRDGYSFDYWGQ
>
CARDRVGYSLDYWGQ
>
CARQRVGYNLDYWGQ
>
CARQRVGYSLDYWGE
>
CARSRDGYSLDYWGQ
>
CARYRSGYYLDYWGQ
>
CARYRVGYSFDYWGQ
>
CARSRDGYSFDYWGQ
>
CARARDGYSLDYWGQ
>
CARHRVGYSFDYWGQ
>
CARADVGYSFDYWQ
>
CARGFVYYSFDYWGQ
>
CARGFIYYSFDYWGQ
>
CARGFLYYSFDYWGQ
>
CARGRIYYSFDYWGQ
>
CARGRFYYSFDYWGQ
>
CARGRLYYSLDYWGQ
>
CARGFIYYDFDYWGQ
>
CARGKIYYSFDYWGQ
>
CARGYLYYSFDYWGQ
CARGFIYYSLDYWGQ
>
CARGNLYYSLDYWGQ
>
CSRGKVGYSLDYWGQ
>
CARGSIGYNLDYWGQ
>
CARGRDGYSLDYWGQ
>
CAREKYGYYFDYWGQ
>
CARYKSGWYFDYWGQ
>
CARYRSGYYFDYWGQ
>
CARYKSGYYFDYWGQ
>
CAREKSGYYFDYWGQ
>
CARERSGYYFDYWGQ
LIST OF VK_CDR1
>
ITCRASQYVGTYLNWYQ
>
ITCRASQYVGSYLNWYQ
>
ITCRASQYIGSYLNWYQ
>
ITCRASQAISNYLTWYQ
>
ITCRASQYVGSYLTWYQ
>
ITCRASQDVSNYLNWYQ
>
ITCQASQYVGSYLSWYQ
>
ITCRASQYVGSYLSWYQ
LIST OF VK_CDR2
>
LIYDASNRNGIP
>
LIYAASSLQSGVP
>
LIYDAANRATGIP
>
LIYDASNRAAGIP
>
LIYDASNRASGIP
LIST OF VK_CDR3
>
YYCQVWDSSPPVAFGG
>
YYCQVWDSSPPVVFGG
>
YYCQVWDSSPPLVFGG
>
YYCQVWDSSPPLAFGG
>
YYCQAWDSSPPVVFGG
>
YYCQVWDSSPPVMFGG
> YYCQGWDSSPTFGG
>
YYCQVWDNSPPVVFGG
>
YYCQAWDSSPPVTFGD
>
YYCQQSGSYLTFGG
>
YYCQVWDSYHAVVFGG
>SEQ 448
YYCQVWGSYHAVMFGG
>
YYCQVWGSYHSVMFGG
>
YYCQVWDTDHSVVFGG
>
YYCQVWDTDHAVAFGG
>
YYCQVWDSSHSVIFGG
>
YYCQVWDSYPPVVFGG
>
YYCQVWDSDHAVVFGG
>
YYCQVWGSNHASLFGG
>
YYCQVWGSTARVAFGG
>
YYCQVWNSTPPVVFGG
>
YYCQVWSSSPPVTFGG
>SEQ
YYCQVWSSSPPVVFGG
>
YYCQVWSSNHAVVFGG
>
YYCQVWGSNPPVAFGG
>
YYCQVWDSTPPVVFGG
>
YYCQVWDSNPPVVFGG
>
YYCQGYSSNDGVIFGG
>
YYCQVWGSNHSVVFGG
>
YYCQVWDSNHSVVFGG
>
YYCQVWDSSSAVVFGG

What is claimed:

1. An isolated antibody or antigen binding fragment thereof, comprising at least one CDR and one of a variant junctional region (vJR) flanking the N (JR_{N}) and/or C (JR_{C}) terminal of said CDR, wherein each of JRN and JRC region comprises from 1-3 amino acids, and wherein at least one of said vJR comprises
a) at least one framework residue position in one of said JRN and JRC region comprises a variant amino acid that is selected from a group of up to of from 1-6 different amino acids; and,
b) said CDR comprises at least one variant amino acid residue that comprises any of the naturally occurring amino acids.

2. The antibody of claim 1, further comprising at least one additional CDR, wherein said CDR comprises a JRN and JRC region.

3. An isolated nucleic acid molecule encoding an immunoglobulin chain of the antibody or fragment of claim 1.

4. An isolated vector that comprises the nucleic acid of claim 3.

5. The vector according to claim 4 that is a replicable expression vector.

6. A host cell comprising the vector according to claim 4.

7. A plurality of antibody variable domains, wherein each of the antibody variable domain comprises comprising at least one CDR and one of a variant junctional region (vJR) flanking the N (JR_{N}) and/or C (JR_{C}) terminal of said CDR, wherein each of JR_{N} and JR_{C} region comprises from 1-3 amino acids, and wherein at least one of said vJR comprises
a) at least one framework residue position in one of said JRN and JRC region comprises a variant amino acid that is selected from a group of up to of from 1-6 different amino acids; and,
b) said at least one CDR comprises at least one variant amino acid residue that comprises any of the naturally occurring amino acids.

8. A method of generating a composition comprising a plurality of variant antibody molecules or antigen binding fragments thereof, wherein the method comprises generating a plurality of polypeptides comprising:
(i) CDRL1 comprising a first consensus hypervariable sequence or variant thereof comprising substitution at one or more positions compared to a corresponding consensus hypervariable sequence;
(ii) CDRL2 comprising a second consensus hypervariable sequence or variant thereof comprising substitution at one or more positions compared to a corresponding consensus hypervariable sequence;
(iii) CDRL3 comprising a third consensus hypervariable sequence or variant thereof; and,
(iv) a variant framework sequence designated JRNCDR1JRC wherein said variant framework sequence comprises substituting at one or more amino acid residues in at least one of said JR_{N} or JR_{C} relative to a corresponding consensus.

9. A method of selecting for an antigen binding variable domain that binds to a target antigen from a library of antibody variable domains comprising:
(a) contacting the plurality of antibody variable domains of claim 7 with a target antigen;
(b) separating one or more polypeptides that specifically bind to the target antigen from polypeptides that do not specifically bind to the target antigen, recovering the one or more polypeptides that specifically bind to the target antigen, and incubating the one or more polypeptides that specifically bind to the target antigen in a series of solutions comprising decreasing amounts of the target antigen in a concentration from about 0.1 nM to about 1000 nM; and,
(c) selecting the one or more polypeptides that specifically bind to the target antigen and that can bind to the lowest concentration of the target antigen or that have an affinity of about 0.1 nM to about 200 nM.

10. A method of selecting for a variant antibody that binds to a target antigen from a library of antibodies; comprising:
(a) isolating one or more antibodies that specifically bind to the target antigen by contacting a library comprising a plurality of antibody variable domains of claim 7 with an immobilized target antigen under conditions suitable for binding;
(b) separating the one or more antibodies that specifically bind to the target antigen from antibodies that do not specifically bind to the target antigen, and recovering the one or more variant antibodies that specifically bind to the target antigen to obtain a subpopulation enriched for the one or more antibodies that specifically bind to the target antigen; and,
(c) optionally, repeating steps (a)-(b) at least twice, each repetition using the subpopulation enriched for the one or more variant antibodies that specifically bind to the target antigen obtained from the previous round of selection.

11. The method of claim 10, further comprising: (d) incubating the subpopulation with a concentration of labeled target antigen in the range of about 0.1 nM to about 1000 nM to form a mixture, under conditions suitable for binding;
(e) contacting the mixture with an immobilized agent that binds to the label on the target antigen;
(f) detecting the one or more polypeptides that specifically bind to the labeled target antigen, and recovering the one or more variant antibodies that specifically bind to the labeled target antigen from the labeled target antigen; and,
(g) optionally, repeating steps (d) to (f) at least twice, each repetition using the subpopulation enriched for the one or more variant antibodies that specifically bind to the labeled target antigen obtained from the previous round of selection, and using a lower concentration of labeled target antigen than the previous round of selection.

12. The method of claim 11, further comprising adding an excess of unlabeled target antigen to the mixture and incubating the mixture for a period of time sufficient to recover one or more variant antibody that specifically bind to the target antigen with low affinity.

13. A method of isolating one or more variant antibodies that specifically bind to a target antigen with high affinity, comprising:
(a) contacting a library comprising a plurality of antibodies of claim 7 with a target antigen at a concentration of at least about 0.1 nM to about 1000 nM to isolate one or more variant antibodies that specifically bind to the target antigen;
(b) recovering the one or more variant antibodies that specifically bind to the target antigen from the target antigen to obtain a subpopulation enriched for the one or more polypeptides that specifically bind to the target antigen; and,
(c) optionally repeating steps (a) and (b) at least twice, each repetition using the subpopulation obtained from the previous round of selection and using a decreased concentration of target antigen from that used in the previous round to isolate one or more variant antibodies that bind specifically to the target antigen at the lowest concentration of target antigen.
